SEQUENCE LISTING (115 sequences)

SEQ ID NO: 1 - DNA, 42 bp, Artificial Sequence
  Chemically Synethesized
aggccagagcattcgaacgtgatagatgtgtataagagacag

SEQ ID NO: 2 - DNA, 42 bp, Artificial Sequence
  Chemically Synethesized
aggccagagcattcgaaacatcgagatgtgtataagagacag

SEQ ID NO: 3 - DNA, 42 bp, Artificial Sequence
  Chemically Synethesized
aggccagagcattcgatgcctaaagatgtgtataagagacag

SEQ ID NO: 4 - DNA, 42 bp, Artificial Sequence
  Chemically Synethesized
aggccagagcattcgagtggtcaagatgtgtataagagacag

SEQ ID NO: 5 - DNA, 42 bp, Artificial Sequence
  Chemically Synethesized
aggccagagcattcgaccactgtagatgtgtataagagacag

SEQ ID NO: 6 - DNA, 42 bp, Artificial Sequence
  Chemically Synethesized
aggccagagcattcgacattggcagatgtgtataagagacag

SEQ ID NO: 7 - DNA, 42 bp, Artificial Sequence
  Chemically Synethesized
aggccagagcattcgcagatctgagatgtgtataagagacag

SEQ ID NO: 8 - DNA, 42 bp, Artificial Sequence
  Chemically Synethesized
aggccagagcattcgcatcaagtagatgtgtataagagacag

SEQ ID NO: 9 - DNA, 42 bp, Artificial Sequence
  Chemically Synethesized
aggccagagcattcgcgctgatcagatgtgtataagagacag

SEQ ID NO: 10 - DNA, 42 bp, Artificial Sequence
  Chemically Synethesized
aggccagagcattcgacaagctaagatgtgtataagagacag

SEQ ID NO: 11 - DNA, 42 bp, Artificial Sequence
  Chemically Synethesized
aggccagagcattcgctgtagccagatgtgtataagagacag

SEQ ID NO: 12 - DNA, 42 bp, Artificial Sequence
  Chemically Synethesized
aggccagagcattcgagtacaagagatgtgtataagagacag

SEQ ID NO: 13 - DNA, 42 bp, Artificial Sequence
  Chemically Synethesized
aggccagagcattcgaacaaccaagatgtgtataagagacag

SEQ ID NO: 14 - DNA, 42 bp, Artificial Sequence
  Chemically Synethesized
aggccagagcattcgaaccgagaagatgtgtataagagacag

SEQ ID NO: 15 - DNA, 42 bp, Artificial Sequence
  Chemically Synethesized
aggccagagcattcgaacgcttaagatgtgtataagagacag

SEQ ID NO: 16 - DNA, 42 bp, Artificial Sequence
  Chemically Synethesized
aggccagagcattcgaagacggaagatgtgtataagagacag

SEQ ID NO: 17 - DNA, 42 bp, Artificial Sequence
  Chemically Synethesized
aggccagagcattcgaaggtacaagatgtgtataagagacag

SEQ ID NO: 18 - DNA, 42 bp, Artificial Sequence
  Chemically Synethesized
aggccagagcattcgacacagaaagatgtgtataagagacag

SEQ ID NO: 19 - DNA, 42 bp, Artificial Sequence
  Chemically Synethesized
aggccagagcattcgacagcagaagatgtgtataagagacag

SEQ ID NO: 20 - DNA, 42 bp, Artificial Sequence
  Chemically Synethesized
aggccagagcattcgacctccaaagatgtgtataagagacag

SEQ ID NO: 21 - DNA, 42 bp, Artificial Sequence
  Chemically Synethesized
aggccagagcattcgacgctcgaagatgtgtataagagacag

SEQ ID NO: 22 - DNA, 42 bp, Artificial Sequence
  Chemically Synethesized
aggccagagcattcgacgtatcaagatgtgtataagagacag

SEQ ID NO: 23 - DNA, 42 bp, Artificial Sequence
  Chemically Synethesized
aggccagagcattcgactatgcaagatgtgtataagagacag

SEQ ID NO: 24 - DNA, 42 bp, Artificial Sequence
  Chemically Synethesized
aggccagagcattcgagagtcaaagatgtgtataagagacag

SEQ ID NO: 25 - DNA, 42 bp, Artificial Sequence
  Chemically Synethesized
aggccagagcattcgagatcgcaagatgtgtataagagacag

SEQ ID NO: 26 - DNA, 42 bp, Artificial Sequence
  Chemically Synethesized
aggccagagcattcgagcaggaaagatgtgtataagagacag

SEQ ID NO: 27 - DNA, 42 bp, Artificial Sequence
  Chemically Synethesized
aggccagagcattcgagtcactaagatgtgtataagagacag

SEQ ID NO: 28 - DNA, 42 bp, Artificial Sequence
  Chemically Synethesized
aggccagagcattcgatcctgtaagatgtgtataagagacag

SEQ ID NO: 29 - DNA, 42 bp, Artificial Sequence
  Chemically Synethesized
aggccagagcattcgattgaggaagatgtgtataagagacag

SEQ ID NO: 30 - DNA, 42 bp, Artificial Sequence
  Chemically Synethesized
aggccagagcattcgcaaccacaagatgtgtataagagacag

SEQ ID NO: 31 - DNA, 42 bp, Artificial Sequence
  Chemically Synethesized
aggccagagcattcggactagtaagatgtgtataagagacag

SEQ ID NO: 32 - DNA, 42 bp, Artificial Sequence
  Chemically Synethesized
aggccagagcattcgcaatggaaagatgtgtataagagacag

SEQ ID NO: 33 - DNA, 42 bp, Artificial Sequence
  Chemically Synethesized
aggccagagcattcgcacttcgaagatgtgtataagagacag

SEQ ID NO: 34 - DNA, 42 bp, Artificial Sequence
  Chemically Synethesized
aggccagagcattcgcagcgttaagatgtgtataagagacag

SEQ ID NO: 35 - DNA, 42 bp, Artificial Sequence
  Chemically Synethesized
aggccagagcattcgcataccaaagatgtgtataagagacag

SEQ ID NO: 36 - DNA, 42 bp, Artificial Sequence
  Chemically Synethesized
aggccagagcattcgccagttcaagatgtgtataagagacag

SEQ ID NO: 37 - DNA, 42 bp, Artificial Sequence
  Chemically Synethesized
aggccagagcattcgccgaagtaagatgtgtataagagacag

SEQ ID NO: 38 - DNA, 42 bp, Artificial Sequence
  Chemically Synethesized
aggccagagcattcgccgtgagaagatgtgtataagagacag

SEQ ID NO: 39 - DNA, 42 bp, Artificial Sequence
  Chemically Synethesized
aggccagagcattcgcctcctgaagatgtgtataagagacag

SEQ ID NO: 40 - DNA, 42 bp, Artificial Sequence
  Chemically Synethesized
aggccagagcattcgcgaacttaagatgtgtataagagacag

SEQ ID NO: 41 - DNA, 42 bp, Artificial Sequence
  Chemically Synethesized
aggccagagcattcgcgactggaagatgtgtataagagacag

SEQ ID NO: 42 - DNA, 42 bp, Artificial Sequence
  Chemically Synethesized
aggccagagcattcgcgcatacaagatgtgtataagagacag

SEQ ID NO: 43 - DNA, 42 bp, Artificial Sequence
  Chemically Synethesized
aggccagagcattcgctcaatgaagatgtgtataagagacag

SEQ ID NO: 44 - DNA, 42 bp, Artificial Sequence
  Chemically Synethesized
aggccagagcattcgctgagccaagatgtgtataagagacag

SEQ ID NO: 45 - DNA, 42 bp, Artificial Sequence
  Chemically Synethesized
aggccagagcattcgctggcataagatgtgtataagagacag

SEQ ID NO: 46 - DNA, 42 bp, Artificial Sequence
  Chemically Synethesized
aggccagagcattcggaatctgaagatgtgtataagagacag

SEQ ID NO: 47 - DNA, 42 bp, Artificial Sequence
  Chemically Synethesized
aggccagagcattcgcaagactaagatgtgtataagagacag

SEQ ID NO: 48 - DNA, 42 bp, Artificial Sequence
  Chemically Synethesized
aggccagagcattcggagctgaaagatgtgtataagagacag

SEQ ID NO: 49 - DNA, 42 bp, Artificial Sequence
  Chemically Synethesized
aggccagagcattcggatagacaagatgtgtataagagacag

SEQ ID NO: 50 - DNA, 42 bp, Artificial Sequence
  Chemically Synethesized
aggccagagcattcggccacataagatgtgtataagagacag

SEQ ID NO: 51 - DNA, 60 bp, Artificial Sequence
  Chemically Synethesized
gcctgtccgcggaagcagtggtatcaacgcagagtacaacgtgatatccacgtgcttgag

SEQ ID NO: 52 - DNA, 60 bp, Artificial Sequence
  Chemically Synethesized
gcctgtccgcggaagcagtggtatcaacgcagagtacaaacatcgatccacgtgcttgag

SEQ ID NO: 53 - DNA, 60 bp, Artificial Sequence
  Chemically Synethesized
gcctgtccgcggaagcagtggtatcaacgcagagtacatgcctaaatccacgtgcttgag

SEQ ID NO: 54 - DNA, 60 bp, Artificial Sequence
  Chemically Synethesized
gcctgtccgcggaagcagtggtatcaacgcagagtacagtggtcaatccacgtgcttgag

SEQ ID NO: 55 - DNA, 60 bp, Artificial Sequence
  Chemically Synethesized
gcctgtccgcggaagcagtggtatcaacgcagagtacaccactgtatccacgtgcttgag

SEQ ID NO: 56 - DNA, 60 bp, Artificial Sequence
  Chemically Synethesized
gcctgtccgcggaagcagtggtatcaacgcagagtacacattggcatccacgtgcttgag

SEQ ID NO: 57 - DNA, 60 bp, Artificial Sequence
  Chemically Synethesized
gcctgtccgcggaagcagtggtatcaacgcagagtaccagatctgatccacgtgcttgag

SEQ ID NO: 58 - DNA, 60 bp, Artificial Sequence
  Chemically Synethesized
gcctgtccgcggaagcagtggtatcaacgcagagtaccatcaagtatccacgtgcttgag

SEQ ID NO: 59 - DNA, 60 bp, Artificial Sequence
  Chemically Synethesized
gcctgtccgcggaagcagtggtatcaacgcagagtaccgctgatcatccacgtgcttgag

SEQ ID NO: 60 - DNA, 60 bp, Artificial Sequence
  Chemically Synethesized
gcctgtccgcggaagcagtggtatcaacgcagagtacacaagctaatccacgtgcttgag

SEQ ID NO: 61 - DNA, 60 bp, Artificial Sequence
  Chemically Synethesized
gcctgtccgcggaagcagtggtatcaacgcagagtacctgtagccatccacgtgcttgag

SEQ ID NO: 62 - DNA, 60 bp, Artificial Sequence
  Chemically Synethesized
gcctgtccgcggaagcagtggtatcaacgcagagtacagtacaagatccacgtgcttgag

SEQ ID NO: 63 - DNA, 60 bp, Artificial Sequence
  Chemically Synethesized
gcctgtccgcggaagcagtggtatcaacgcagagtacaacaaccaatccacgtgcttgag

SEQ ID NO: 64 - DNA, 60 bp, Artificial Sequence
  Chemically Synethesized
gcctgtccgcggaagcagtggtatcaacgcagagtacaaccgagaatccacgtgcttgag

SEQ ID NO: 65 - DNA, 60 bp, Artificial Sequence
  Chemically Synethesized
gcctgtccgcggaagcagtggtatcaacgcagagtacaacgcttaatccacgtgcttgag

SEQ ID NO: 66 - DNA, 60 bp, Artificial Sequence
  Chemically Synethesized
gcctgtccgcggaagcagtggtatcaacgcagagtacaagacggaatccacgtgcttgag

SEQ ID NO: 67 - DNA, 60 bp, Artificial Sequence
  Chemically Synethesized
gcctgtccgcggaagcagtggtatcaacgcagagtacaaggtacaatccacgtgcttgag

SEQ ID NO: 68 - DNA, 60 bp, Artificial Sequence
  Chemically Synethesized
gcctgtccgcggaagcagtggtatcaacgcagagtacacacagaaatccacgtgcttgag

SEQ ID NO: 69 - DNA, 60 bp, Artificial Sequence
  Chemically Synethesized
gcctgtccgcggaagcagtggtatcaacgcagagtacacagcagaatccacgtgcttgag

SEQ ID NO: 70 - DNA, 60 bp, Artificial Sequence
  Chemically Synethesized
gcctgtccgcggaagcagtggtatcaacgcagagtacacctccaaatccacgtgcttgag

SEQ ID NO: 71 - DNA, 60 bp, Artificial Sequence
  Chemically Synethesized
gcctgtccgcggaagcagtggtatcaacgcagagtacacgctcgaatccacgtgcttgag

SEQ ID NO: 72 - DNA, 60 bp, Artificial Sequence
  Chemically Synethesized
gcctgtccgcggaagcagtggtatcaacgcagagtacacgtatcaatccacgtgcttgag

SEQ ID NO: 73 - DNA, 60 bp, Artificial Sequence
  Chemically Synethesized
gcctgtccgcggaagcagtggtatcaacgcagagtacactatgcaatccacgtgcttgag

SEQ ID NO: 74 - DNA, 60 bp, Artificial Sequence
  Chemically Synethesized
gcctgtccgcggaagcagtggtatcaacgcagagtacagagtcaaatccacgtgcttgag

SEQ ID NO: 75 - DNA, 60 bp, Artificial Sequence
  Chemically Synethesized
gcctgtccgcggaagcagtggtatcaacgcagagtacagatcgcaatccacgtgcttgag

SEQ ID NO: 76 - DNA, 60 bp, Artificial Sequence
  Chemically Synethesized
gcctgtccgcggaagcagtggtatcaacgcagagtacagcaggaaatccacgtgcttgag

SEQ ID NO: 77 - DNA, 60 bp, Artificial Sequence
  Chemically Synethesized
gcctgtccgcggaagcagtggtatcaacgcagagtacagtcactaatccacgtgcttgag

SEQ ID NO: 78 - DNA, 60 bp, Artificial Sequence
  Chemically Synethesized
gcctgtccgcggaagcagtggtatcaacgcagagtacatcctgtaatccacgtgcttgag

SEQ ID NO: 79 - DNA, 60 bp, Artificial Sequence
  Chemically Synethesized
gcctgtccgcggaagcagtggtatcaacgcagagtacattgaggaatccacgtgcttgag

SEQ ID NO: 80 - DNA, 60 bp, Artificial Sequence
  Chemically Synethesized
gcctgtccgcggaagcagtggtatcaacgcagagtaccaaccacaatccacgtgcttgag

SEQ ID NO: 81 - DNA, 60 bp, Artificial Sequence
  Chemically Synethesized
gcctgtccgcggaagcagtggtatcaacgcagagtacgactagtaatccacgtgcttgag

SEQ ID NO: 82 - DNA, 60 bp, Artificial Sequence
  Chemically Synethesized
gcctgtccgcggaagcagtggtatcaacgcagagtaccaatggaaatccacgtgcttgag

SEQ ID NO: 83 - DNA, 60 bp, Artificial Sequence
  Chemically Synethesized
gcctgtccgcggaagcagtggtatcaacgcagagtaccacttcgaatccacgtgcttgag

SEQ ID NO: 84 - DNA, 60 bp, Artificial Sequence
  Chemically Synethesized
gcctgtccgcggaagcagtggtatcaacgcagagtaccagcgttaatccacgtgcttgag

SEQ ID NO: 85 - DNA, 60 bp, Artificial Sequence
  Chemically Synethesized
gcctgtccgcggaagcagtggtatcaacgcagagtaccataccaaatccacgtgcttgag

SEQ ID NO: 86 - DNA, 60 bp, Artificial Sequence
  Chemically Synethesized
gcctgtccgcggaagcagtggtatcaacgcagagtacccagttcaatccacgtgcttgag

SEQ ID NO: 87 - DNA, 60 bp, Artificial Sequence
  Chemically Synethesized
gcctgtccgcggaagcagtggtatcaacgcagagtacccgaagtaatccacgtgcttgag

SEQ ID NO: 88 - DNA, 60 bp, Artificial Sequence
  Chemically Synethesized
gcctgtccgcggaagcagtggtatcaacgcagagtacccgtgagaatccacgtgcttgag

SEQ ID NO: 89 - DNA, 60 bp, Artificial Sequence
  Chemically Synethesized
gcctgtccgcggaagcagtggtatcaacgcagagtaccctcctgaatccacgtgcttgag

SEQ ID NO: 90 - DNA, 60 bp, Artificial Sequence
  Chemically Synethesized
gcctgtccgcggaagcagtggtatcaacgcagagtaccgaacttaatccacgtgcttgag

SEQ ID NO: 91 - DNA, 60 bp, Artificial Sequence
  Chemically Synethesized
gcctgtccgcggaagcagtggtatcaacgcagagtaccgactggaatccacgtgcttgag

SEQ ID NO: 92 - DNA, 60 bp, Artificial Sequence
  Chemically Synethesized
gcctgtccgcggaagcagtggtatcaacgcagagtaccgcatacaatccacgtgcttgag

SEQ ID NO: 93 - DNA, 60 bp, Artificial Sequence
  Chemically Synethesized
gcctgtccgcggaagcagtggtatcaacgcagagtacctcaatgaatccacgtgcttgag

SEQ ID NO: 94 - DNA, 60 bp, Artificial Sequence
  Chemically Synethesized
gcctgtccgcggaagcagtggtatcaacgcagagtacctgagccaatccacgtgcttgag

SEQ ID NO: 95 - DNA, 60 bp, Artificial Sequence
  Chemically Synethesized
gcctgtccgcggaagcagtggtatcaacgcagagtacctggcataatccacgtgcttgag

SEQ ID NO: 96 - DNA, 60 bp, Artificial Sequence
  Chemically Synethesized
gcctgtccgcggaagcagtggtatcaacgcagagtacgaatctgaatccacgtgcttgag

SEQ ID NO: 97 - DNA, 60 bp, Artificial Sequence
  Chemically Synethesized
gcctgtccgcggaagcagtggtatcaacgcagagtaccaagactaatccacgtgcttgag

SEQ ID NO: 98 - DNA, 60 bp, Artificial Sequence
  Chemically Synethesized
gcctgtccgcggaagcagtggtatcaacgcagagtacgagctgaaatccacgtgcttgag

SEQ ID NO: 99 - DNA, 60 bp, Artificial Sequence
  Chemically Synethesized
gcctgtccgcggaagcagtggtatcaacgcagagtacgatagacaatccacgtgcttgag

SEQ ID NO: 100 - DNA, 60 bp, Artificial Sequence
  Chemically Synethesized
gcctgtccgcggaagcagtggtatcaacgcagagtacgccacataatccacgtgcttgag

SEQ ID NO: 101 - DNA, 19 bp, Artificial Sequence
  Chemically Synethesized
ctgtctcttatacacatct

SEQ ID NO: 102 - DNA, 34 bp, Artificial Sequence
  Chemically Synethesized
gtctcgtgggctcggagatgtgtataagagacag

SEQ ID NO: 103 - DNA, 34 bp, Artificial Sequence
  Chemically Synethesized
agtcgtacgccgatgctgtctcttatacacatct

SEQ ID NO: 104 - DNA, 30 bp, Artificial Sequence
  Chemically Synethesized
cgaatgctctggcctctcaagcacgtggat

SEQ ID NO: 105 - DNA, 66 bp, Artificial Sequence
  Chemically Synethesized
aatgatacggcgaccaccgagatctacacgcctgtccgcggaagcagtggtatcaacgcagagtac

SEQ ID NO: 106 - DNA, 54 bp, Artificial Sequence
  Chemically Synethesized
caagcagaagacggcatacgagattcgccttagtctcgtgggctcggagatgtg

SEQ ID NO: 107 - DNA, 54 bp, Artificial Sequence
  Chemically Synethesized
caagcagaagacggcatacgagatctagtacggtctcgtgggctcggagatgtg

SEQ ID NO: 108 - DNA, 54 bp, Artificial Sequence
  Chemically Synethesized
caagcagaagacggcatacgagatttctgcctgtctcgtgggctcggagatgtg

SEQ ID NO: 109 - DNA, 54 bp, Artificial Sequence
  Chemically Synethesized
caagcagaagacggcatacgagatgctcaggagtctcgtgggctcggagatgtg

SEQ ID NO: 110 - DNA, 54 bp, Artificial Sequence
  Chemically Synethesized
caagcagaagacggcatacgagataggagtccgtctcgtgggctcggagatgtg

SEQ ID NO: 111 - DNA, 54 bp, Artificial Sequence
  Chemically Synethesized
caagcagaagacggcatacgagatcatgcctagtctcgtgggctcggagatgtg

SEQ ID NO: 112 - DNA, 54 bp, Artificial Sequence
  Chemically Synethesized
caagcagaagacggcatacgagatgtagagaggtctcgtgggctcggagatgtg

SEQ ID NO: 113 - DNA, 54 bp, Artificial Sequence
  Chemically Synethesized
caagcagaagacggcatacgagatcctctctggtctcgtgggctcggagatgtg

SEQ ID NO: 114 - DNA, 37 bp, Artificial Sequence
  Chemically Synethesized
gcctgtccgcggaagcagtggtatcaacgcagagtac

SEQ ID NO: 115 - DNA, 34 bp, Artificial Sequence
  Chemically Synethesized
catcggcgtacgactagatgtgtataagagacag

(19) United States
(12) Patent Application Publication
(10) Pub. No.: US 2024/0279712 A1
(43) Pub. Date: Aug. 22, 2024

(54) HIGH-SPATIAL-RESOLUTION EPIGENOMIC PROFILING

(71) Applicant: YALE UNIVERSITY, New Haven, CT (US)

(72) Inventors: Rong Fan, Cheshire, CT (US); Yanxiang Deng, New Haven, CT (US)

(73) Assignee: YALE UNIVERSITY, New Haven, CT (US)

(21) Appl. No.: 18/270,684

(22) PCT Filed: Dec. 30, 2021
(86) PCT No.: PCT/US2021/065669

§ 371 Date: Jun. 30, 2023

Related U.S. Application Data
(60) Provisional application No. 63/132,659, filed on Dec. 31, 2020.

(51) Int. Cl. C12Q 1/6806; B01L 3/00; G06T 7/00

(52) U.S. Cl. CPC C12Q 1/6806; B01L 3/502707; G06T 7/0012; B01L 2300/0883; G06T 2207/10056

(57) ABSTRACT

Provided herein are compositions and methods for producing a high resolution spatial epigenomic map of a biological sample.

### CROSS REFERENCE TO RELATED APPLICATIONS

This application claims priority to U.S. Provisional Patent Application, No. 63/132,659, filed Dec. 31, 2020 which is hereby incorporated by reference herein in its entirety.

### BACKGROUND OF THE INVENTION

It has been widely recognized that epigenetic mechanisms are critical in normal development and disease development. It is essential to analyze all relevant epigenetic alterations in the original tissue samples and ideally with spatial location information as well because it is the difference of epigenetic program differentially activated in different cells within a tissue that gives rise to diverse cell types and the organization into functional tissues or organs. In addition, such analysis should be done at the whole genome scale in an unbiased manner in order to gain a complete picture of epigenetic states in each cell in the tissue and to discover new mechanisms which cannot be explored with targeted detection of epigenetic sites. However, such analysis is not possible with any existing technologies. The state-of-art epigenomic profiling is still largely based on bulk tissue samples or the sample containing tens of thousands of cells. A single-cell assay for transposase-accessible chromatin using sequencing (scATAC-seq) profiling method has been developed but it has rather limited coverage or total # of reads per cell. None of the current methods are able to provide spatial information.

There is thus a need in the art for systems and methods for spatial epigenomic profiling. The present invention addresses this unmet need in the art.

### SUMMARY OF THE INVENTION

In one embodiment, the invention relates to a method, comprising:
(a) delivering to a region of interest in a tissue sample mounted on a substrate a transposase and a linker adaptor sequence;
(b) delivering to the region of interest a first set of barcoded polynucleotides, wherein the barcoded polynucleotides comprise a first region for ligation to the linker adaptor sequence, a second unique region for spatial barcoding and a third linker region for ligation to a region of the second barcode or a universal ligation linker, wherein the first set of barcoded polynucleotides is delivered through a first microfluidic device clamped to the region of interest;
(c) delivering to the region of interest ligation reagents to join the ligation adaptor to the barcoded polynucleotides of the first set;
(d) delivering to the region of interest a second set of barcoded polynucleotides, wherein the barcoded polynucleotides comprise a first region for ligation to the linker region of the first barcode or a universal ligation linker, a second unique region for spatial barcoding and a third ligation region comprising a sequence for recognition by a primer for DNA amplification, wherein the second set of barcoded polynucleotides is delivered through a second microfluidic device clamped to the region of interest, wherein the second microfluidic device is oriented on the region of interest perpendicular to the direction of the microchannels of the first microfluidic device;
(e) delivering to the region of interest ligation reagents to join barcoded polynucleotides of the first set to barcoded polynucleotides of the second set;
(f) imaging the region of interest to produce a sample image;
(g) delivering to the region of interest lysis buffer or denaturation reagents to produce a lysed or denatured tissue sample; and
(h) extracting the DNA from the lysed or denatured tissue sample.

In one embodiment, the method further comprises a step of permeabilizing the tissue sample prior to delivering the transposase and linker adaptor sequence.

In one embodiment, step (a) comprises delivering to the region of interest in a tissue sample mounted on a substrate (i) a primary antibody specific for binding to an epigenomic marker of interest (ii) a secondary antibody and (iii) a transposase and a linker adaptor sequence.

In one embodiment, the primary antibody is selected from whole antibodies, Fab antibody fragments, F(ab′)₂ antibody fragments, monospecific Fab₂ fragments, bispecific Fab fragments, trispecific Fab₃ fragments, single chain variable fragments (scFvs), bispecific diabodies, trispecific diabodies, scFv-Fc molecules, nanobodies, and minibodies.

In one embodiment, the epigenomic marker is H2AK5ac, H2AK9ac, H2BK120ac, H2BK12ac, H2BK15ac, H2BK20ac, H2BK5ac, H2Bub, H3, H3ac, H3K14ac, H3K18ac, H3K23ac, H3K23me2, H3K27me1, H3K27me2, H3K36ac, H3K36me1, H3K36me2, H3K4ac, H3K56ac, H3K79me1, H3K79me3, H3K9acS10ph, H3K9me2, H3S10ph, H3T11ph, H4, H4ac, H4K12ac, H4K16ac, H4K5ac, H4K8ac, H4K91ac, H3F3A, H3K27me3, H3K36me3, H3K4me1, H3K79me2, H3K9me1, H3K9me2, H3K9me3, H4K20me1, H2AFZ, H3K27ac, H3K4me2, H3K4me3, or H3K9ac.

In one embodiment, the method further comprises delivering to the biological sample a ligation linker sequence, wherein the ligation linker is
a) a nucleic acid molecule comprising a sequence complementary to the ligation linker sequence of the ligation adaptor associated with the transposon and a sequence complementary to the ligation linker sequence of the barcoded polynucleotides of the first set; or
b) a nucleic acid molecule comprising a sequence complementary to the ligation linker sequence of the barcoded polynucleotides of the first set and a sequence complementary to the ligation linker sequence of the barcoded polynucleotides of the second set.

In one embodiment, the method further comprises step (i) sequencing the DNA to produce DNA reads. In one embodiment, the method further comprises constructing a spatial map of the tissue section by matching the spatially addressable barcoded conjugates to corresponding sequencing reads. In one embodiment, the method further comprises identifying the anatomical location of the nucleic acids by correlating the spatial map to the sample image.

In one embodiment, the tissue section mounted on a slide is produced by sectioning a formalin fixed paraffin embedded (FFPE) tissue, optionally into a 5-10 μm section and mounting the tissue section onto a substrate, optionally a poly-L-lysine-coated slide;

applying to the tissue section a wash solution, optionally a xylene solution, to deparaffinize the tissue section;
applying to the tissue section a rehydration solution to rehydrate the tissue section;
applying to the tissue section an enzymatic solution to permeabilize the tissue section; and
applying formalin to the tissue section to post-fix the tissue section.

In one embodiment, the first and/or second microfluidic device is fabricated from polydimethylsiloxane (PDMS).

In one embodiment, the first and/or second microfluidic device comprises 10 to 1000 microchannels.

In one embodiment, the first and/or second microfluidic device comprises serpentine microchannels.

In one embodiment, the method further comprises delivering to the region of interest a third set of barcoded polynucleotides, wherein the third set of barcoded polynucleotides is delivered to specific zones, such that each zone distinguishes a specific region of overlap of the first and second barcode sequences; wherein the third set of barcoded polynucleotides are delivered directly to the tissue section, optionally through a set of holes in a device clamped to the substrate, wherein each hole is positioned directly above a zone of overlap of the first and second barcode sequences.

In one embodiment, the first set of barcoded polynucleotides is delivered through the first microfluidic device using a negative pressure system and/or the second set of barcoded polynucleotides is delivered through the second microfluidic device using a negative pressure system.

In one embodiment, the lysis buffer or denaturation reagents are delivered directly to the tissue section, optionally through a hole in a device clamped to the substrate, wherein the hole is positioned directly above the region of interest.

In one embodiment, the first and/or second set of barcoded polynucleotides comprises at least 10 barcoded polynucleotides.

In one embodiment, the imaging is with an optical or fluorescence microscope.

In one embodiment, the substrate is selected from the group consisting of a glass slide and a plastic slide.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts DBiT-seq for spatially resolved transcriptome and protein mapping. (FIG. 1A) Schematic workflow. (FIG. 1B) Microfluidic device used to barcode 50×50 tissue pixels (10 μm). (FIG. 1C) Compare # of genes and UMIs detected by DBiT-seq and other technologies. (FIG. 1D) Spatial mapping of the eye field development in an E10 mouse embryo. (FIG. 1E) Spatial expression of select genes reveals the optic vesicle and a single-layer of melanocytes in retinal pigmented epithelium (RPE). (FIG. 1F) Computational analysis of combined scRNA-seq and DBiT-seq data reveals that DBiT-seq tissue pixels (10 μm) are dominated by single-cell transcriptomes. (FIG. 1G) Spatial expression patterns of different gene clusters further identified different tissue types.

FIG. 2 depicts a schematic diagram of hsrATAC-seq: high-spatial-resolution assay of chromatin accessibility by sequencing using DBiT for spatial mapping of epigenetic states in tissues.

FIG. 3A and FIG. 3B depict exemplary diagrams of other spatial epigenomics profiling technologies. Schematic to show the modification of Tn5 chemistry, for example, for spatial CHIP-seq (FIG. 3A) or spatial methylome-seq (FIG. 3B).

FIG. 4 depicts artificial human embryos generated in a microfluidic system. This system can be readily adopted to generate these samples for spatial epigenomics mapping of embryonic development.

FIG. 5A through FIG. 5E depict the design of hsrChST-seq. (FIG. 5A) Schematic of the Cut&Tag protocol to be performed directly on a tissue slide. (FIG. 5B) Primary antibody detects specific histone modification and the Tn5 transposon complex can be covalently conjugated to this antibody or through binding to a secondary antibody. (FIG. 5C) Design of the linker sequence incorporated in the Tn5 transposome complex. (FIG. 5D) Workflow to spatially deliver barcodes A and B to the tissue pixels (tixels) and the chemistry workflow to amplify and sequence the sample. (FIG. 5E) Two microfluidic chips used for cross flow barcoding of A and B to create a 2D lattice of address codes directly in the tissue section.

FIG. 6A through FIG. 6C depicts an ultra-large mappable area hsrChSTseq via tissue zone barcoding. (FIG. 6A) Current device capable of mapping one sample per slide. (FIG. 6B) Proposed microfluidic device design to achieve high sample throughput. (FIG. 6C) Large microwell array for barcoding 24 tissue sections per run. (FIG. 6C) Design of a “macro”fluidic chip for cross-flow barcoding of >100 samples per run.

FIG. 7A and FIG. 7B depict schematic depiction of MSD bone marrow spatial mapping. Low grade MDS with mixed lineage dysplasia (MLD) (FIG. 7A) and high-grade MDS with excess blasts (EB2) (FIG. 7B) histologic sections with overlay of DBITseq grid with 10 μm pixels. Enlarged examples depict expected relation of sequencing pixels to cellular architecture with erythroblastic island in (FIG. 7A) and blasts in relation to fat cell and a dysplastic megakaryocyte (FIG. 7B).

FIG. 8 depicts experimental results for hsrCUT&Tag on E11 mouse embryo using a microfluidic device with 50 channels, each 50 μm wide. The left panel demonstrates a size distribution of fragments resulting from exposure to Tn5 transposase, following spatial barcoding and crosslink reversal. The periodic nature of fragment sizes at around 10 base pairs is related to the double helix structure of DNA executing one turn every 10 base pairs. The longer-scale structure is consistent with the structure imposed on genomic DNA by histone structure. The right panel demonstrates the distribution of the average number of unique fragments recovered in each tixel. The median number of fragments of between 1,000-10,000 compares favorably with other methods including scCUT&Tag. The PCR duplication rate of 10.2% indicates that deeper sequencing could produce a higher number of fragments per cell. The structure in the distribution could be related to the existence of multiple cell types within one tixel. The fragment distribution excludes the infrastructure and therefore only shows length of the genomic DNA fragments.

FIG. 9 depicts experimental results demonstrating unsupervised clustering of tixels driven by variation in downregulatory histone modification.

FIG. 10 depicts heat maps of the Chromatin Silencing Score (CSS). The score is calculated by mapping the minimum (0) and maximum histone modification count and mapping it onto the range (−2,2). Each row represents an unsupervised cluster (see FIG. 9). The ordering of the rows attempts to ensure diagonality. The left panel shows the downregulated marker genes (log fold change of <−1; few K27me3) representative of each cluster. A brighter coloring indicates more numerous histone modifications at the H3K27me3 site. Each pixel color indicates the number of H3K27me3 modifications found on that gene in this cluster. The right panel shows the upregulated marker genes (log fold change of >1; many K27me3).

FIG. 11 depicts a spatial map for cluster 1, including the marker gene Foxa2. In the right panel, the x-axis shows genome coordinate, and the y-axis shows the relative number of K27me3 sites between clusters and gene coordinates. The blue cluster at lower left of the middle panel displays few K27me3 sites, and therefore expression of Foxa2 should be up-regulated in those cells.

FIG. 12 depicts an investigation of biological function of marker genes for cluster 1 using Gene Ontology analysis. GeneRatio measures overlap between marker genes in the cluster and genes characteristic of that ontology in the reference database. Size (count) indicates the number of marker genes found in that ontology. And color measures the statistical significance of the overlap. Since clustering is driven by marker genes (identified by K27me3 frequency), highly statistically significant (p-value<0.001) overlap with biological pathways confirms that clusters identified by epigenetic markers correspond to biological functional groups.

FIG. 13 depicts a spatial map for cluster 2, including the marker gene Gata4.

FIG. 14 depicts an investigation of biological function of marker genes for cluster 2 using Gene Ontology analysis.

FIG. 15 depicts a spatial map for cluster 3, including the marker gene Pou3f3.

FIG. 16 depicts an investigation of biological function of marker genes for cluster 3 using Gene Ontology analysis.

FIG. 17 depicts a spatial map for cluster 4, including the marker gene Syt3.

FIG. 18 depicts an investigation of biological function of marker genes for cluster 4 using Gene Ontology analysis.

FIG. 19 depicts a spatial map for cluster 5, including the marker gene Otx2.

FIG. 20 depicts an investigation of biological function of marker genes for cluster 5 using Gene Ontology analysis.

FIG. 21 depicts a spatial map for cluster 6, including the marker gene Nr2e1.

FIG. 22 depicts an investigation of biological function of marker genes for cluster 6 using Gene Ontology analysis.

FIG. 23 depicts a spatial map for cluster 7, including the marker gene Ccdc106.

FIG. 24 depicts a spatial map for cluster 8, including the marker gene Hoxa9.

FIG. 25 depicts a diagram demonstrating the distribution of Hox gene expression in adult humans and mouse embryos.

FIG. 26 depicts a spatial map for cluster 9, including the marker gene Six1.

FIG. 27 depicts an investigation of biological function of marker genes for cluster 9 using Gene Ontology analysis.

FIG. 28 depicts a spatial map for cluster 10, including the marker gene Spats21.

FIG. 29 depicts a spatial map for cluster 11, including the marker gene Hoxc4.

FIG. 30 depicts an investigation of biological function of marker genes for cluster 11 using Gene Ontology analysis.

FIG. 31 depicts a spatial map for cluster 12, including the marker gene Tbx2.

FIG. 32 depicts an investigation of biological function of marker genes for cluster 12 using Gene Ontology analysis.

FIG. 33 depicts experimental results for hsrCUT&Tag on E11 mouse embryo using a microfluidic device with 50 channels, each 50 μm wide. TSS=transcription start site. The left panel depicts a similar structure in size distribution of fragments, corresponding to 10-bp and larger spectral features. The right panel depicts a density scatter plot. X-axis is the log base 10 of the number of unique fragments. The Y-axis shows the transcription starting site enrichment on a linear scale, as measured by the number of H3K4me2 binding sites. A high TSS indicates the protocol has correctly identified a site accessible for transcription. Color corresponds to the density of dots in a neighborhood around each dot.

FIG. 34 depicts unsupervised clustering of tixels driven by variation in upregulatory histone modification. The clusters are not as well differentiated as in the H3K27me3 downregulatory case.

FIG. 35 depicts an analysis of gene activity based on the identification of H3K4me2 binding sites for cluster 1, including Hoxc4.

FIG. 36 depicts an investigation of biological function of marker genes for cluster 1 using Gene Ontology analysis.

FIG. 37 depicts an analysis of gene activity based on the identification of H3K4me2 binding sites for cluster 2, including Vmn1r45.

FIG. 38 depicts an investigation of biological function of marker genes for cluster 2 using Gene Ontology analysis.

FIG. 39 depicts an analysis of gene activity based on the identification of H3K4me2 binding sites for cluster 3, including Tmem119.

FIG. 40 depicts an investigation of biological function of marker genes for cluster 3 using Gene Ontology analysis.

FIG. 41 depicts an analysis of gene activity based on the identification of H3K4me2 binding sites for cluster 4, including Ttyh1.

FIG. 42 depicts an investigation of biological function of marker genes for cluster 4 using Gene Ontology analysis.

FIG. 43 depicts an analysis of gene activity based on the identification of H3K4me2 binding sites for cluster 5, including Basp1.

FIG. 44 depicts an investigation of biological function of marker genes for cluster 5 using Gene Ontology analysis.

FIG. 45 depicts an analysis of the harCUT&Tag data for H3K4me2 and integration with scRNAseq data (MOCA). Assuming that K4me2 is associated with high gene transcription, one should be able to match up-regulated genes with highly differentially expressed genes in the transcriptome. Each dot in this UMAP corresponds with a dot in the previous UMAP, but the clusters are labeled according to the MOCA data rather than the K4me2 data.

FIG. 46 depicts heat maps of cell types from chosen clusters of the preceding UMAP plot.

FIG. 47 depicts an analysis of hsrCUT&Tag data for H3K4me2. When the K4me2 antibody binds to a gene site, it can be determined whether that site is a promoter or enhancer. Therefore, after sequencing, the P & E frequency can be compared. (E comes in multiple flavors, E1, E2, . . . ). x-axis shows gene coordinate. Y axis shows number of K4me2 binding sites. Left and right show two different locations in the genome. Left: correlation between different peaks. Peaks corresponding to non-coding areas are likely places where K4me2 bound to an enhancer or promoter. Right: Relation between peak and gene. Given a peak in K4me2 binding, find the corresponding gene start site.

FIG. 48 depicts an analysis of motif enrichment for cluster 4, containing the Ttyh1 gene.

FIG. 49 depicts diagrams of current chromatin accessibility assays.

FIG. 50 depicts a schematic of stochastic barcoding enabled massively parallel single-cell ATAC-seq. This method does not provide spatial information.

FIG. 51 depicts a diagram of an approach for high resolution and deterministic spatial ATAC-seq.

FIG. 52 depicts step 1 of the hsrATAC-seq experimental workflow using chemistry version 1: Anneal Tn5 sequences with 1st Barcode and UMI and Tn5 binding site 19-bp Mosaic End (ME) bottom strand to assemble Tn5 transposome.

FIG. 53 depicts step 2 of the hsrATAC-seq experimental workflow using chemistry version 1: Flow the 1st direction and perform tagmentation using barcoded Tn5 transposome. There are 3 different products after this step.

FIG. 54 depicts steps 3-5 of the hsrATAC-seq experimental workflow using chemistry version 1. Step 3: Wash and flow the 2nd direction and perform ligation using 2nd barcodes. Step 4: Cell lysis and library amplification. Step 5: Final library structure.

FIG. 55 depicts the sequencing results including fragment size distribution and barcode recovery.

FIG. 56 depicts step 1 of the hsrATAC-seq experimental workflow using chemistry version 2: Anneal Tn5 sequences with 1st linker and Tn5 binding site 19-bp Mosaic End (ME) bottom strand to assemble Tn5 transposome.

FIG. 57 depicts step 2 of the hsrATAC-seq experimental workflow using chemistry version 2: Flow the 1st direction and perform tagmentation using Tn5 transposome.

FIG. 58 depicts steps 3-6 of the hsrATAC-seq experimental workflow using chemistry version 2. Step 3: Wash and perform ligation using 1st barcodes (BC1_1-BC1_50). Step 4: Wash and perform ligation using 2nd barcodes (BC2_1-BC2_50). Step 5: Cell lysis and library amplification. Step 6: Final library structure.

FIG. 59 depicts the sequencing results including fragment size distribution and barcode recovery and TSS enrichment.

FIG. 60 depicts the unique fragments sequences using the hsrATAC-seq experimental workflow using chemistry version 2.

FIG. 61 depicts the UMAP clusters using the hsrATAC-seq experimental workflow using chemistry version 2.

FIG. 62 depicts the gene activity maps using the hsrATAC-seq experimental workflow using chemistry version 2.

FIG. 63 depicts the sequencing results including fragment size distribution and barcode recovery and TSS enrichment from the hsrATAC-seq experimental workflow using chemistry version 2.1 which includes step 0: Permeabilization for 15 minutes; and includes the use of 2× the Tn5 enzyme in step 1.

FIG. 64 depicts the unique fragments sequences using the hsrATAC-seq experimental workflow using chemistry version 2.1.

FIG. 65 depicts the UMAP clusters using the hsrATAC-seq experimental workflow using chemistry version 2.1.

FIG. 66 depicts the gene activity maps using the hsrATAC-seq experimental workflow using chemistry version 2.1.

FIG. 67 depicts the sequencing results including fragment size distribution and TSS enrichment from the hsrATAC-seq experimental workflow performed on NIH-3T3 cells on a glass slide using chemistry version 2.1

FIG. 68 depicts an exemplary signal track for hsrATAC-seq on E11 mouse embryo using 4× (first row) and 2× (second row) Tn5 enzyme. The last row is from hsrATAC-seq on NIH-3T3 cells on glass slide using 1× Tn5 enzyme.

FIG. 69 depicts the sequencing results including fragment size distribution and TSS enrichment from the hsrATAC-seq experimental workflow performed on Mouse brain region 8 using chemistry version 2.2, with a tagmentation time of 30 minutes.

FIG. 70 depicts the sequencing results including fragment size distribution and TSS enrichment from the hsrATAC-seq experimental workflow performed on ME11 cells using chemistry version 2.2, with a tagmentation time of 30 minutes.

FIG. 71A through FIG. 71 Spatial-CUT&Tag: design and validation.

FIG. 71A depicts a schematic workflow. Primary antibody binding, secondary antibody binding, and pA-Tn5 transposition were performed sequentially in tissue sections. Afterwards, two sets of DNA barcodes (A1-A50, B1-B50) were ligated in-situ. After imaging the tissue sample, DNA fragments were released by reversing cross-linking. Library was constructed during polymerase chain reaction (PCR) and then sequenced by next generation sequencing (NGS). FIG. 71B depicts a comparison of number of unique fragments for different histone marks and different microfluidic channel width between the spatial method in this work and other non-spatial chromatin profiling methods. FIG. 71C depicts a comparison of fraction of reads in peaks (FRiP) for different histone marks and different microfluidic channel width between the spatial method in this work and other non-spatial chromatin profiling methods. FIG. 71D depicts a comparison of fraction of mitochondrial reads for different histone marks and different microfluidic channel width between the spatial method in this work and other non-spatial chromatin profiling methods. FIG. 71E depicts an H&E image from an adjacent tissue section of E11 mouse embryo and a region of interest for spatial epigenome mapping with 50 μm pixel size. FIG. 71F depicts the unsupervised clustering analysis and spatial distribution of each cluster for different histone modifications (50 μm pixel size). FIG. 71G depicts the UMAP embedding of unsupervised clustering analysis for each histone modification (50 μm pixel size). Cluster identities and coloring of clusters are consistent with (FIG. 71F). FIG. 71H depicts a LSI projection of ENCODE bulk ChIP-seq data from diverse cell types of the E11.5 mouse embryo dataset onto the spatial-CUT&Tag embedding.

FIG. 72 depicts the chemistry workflow of spatial-CUT&Tag. A tissue section on a standard aminated glass slide was lightly fixed with formaldehyde. Afterwards, primary antibody that binds to the target histone modifications or chromatin-interacting proteins was added, followed by a secondary antibody binding to enhance the tethering of pA-Tn5 transposome. pA-Tn5 transposome was then activated by adding Mg++ and incubating the sample at 37° C. Then, the adapters containing ligation linker 1 were inserted to the cleaved genomic DNA at antibody recognition sites. Afterwards, a set of DNA barcode A solutions were introduced by microchannel-guided flow delivery to perform in situ ligation reaction for appending a distinct spatial barcode Ai (i=1-50) and ligation linker 2. Then, a second set of barcodes Bj (j=1-50) were introduced using another set of microfluidic channels perpendicularly to those in the first flow barcoding step, which were subsequently ligated at the intersections, resulting in a mosaic of tissue pixels, each containing a distinct combination of barcodes Ai and Bj (i=1-50, j=1-50). After DNA fragments were collected by reversing cross-linking, the library construction was completed during PCR amplification.

FIG. 73A and FIG. 73B depicts data demonstrating the size distribution of DNA fragments. FIG. 73A depicts the bioanalyzer data of DNA fragments. FIG. 73B depicts the distribution of fragment lengths.

FIG. 74A and FIG. 74B depicts data demonstrating the evaluation of the extent of tagmentation by free Tn5. FIG. 74A depicts the signal enrichment for different methods around spatial-CUT&Tag H3K27me3 peaks from E11 mouse embryo with 50 μm pixel size. Peaks called from spatial-CUT&Tag were divided into two parts: peaks overlapping with ChIP-seq peaks and peaks not overlapping with ChIP-seq peaks. FIG. 74B depicts a quantitative analysis to determine the extent of tagmentation by free Tn5. The results showed that around 11.5% of peaks that did not overlap with ChIP-seq peaks were overlapped with ATAC-seq peaks, which may correspond of Tn5 insertion events unrelated to the antibody used.

FIG. 75A through FIG. 75I depict data demonstrating the reproducibility of spatial-CUT&Tag. FIG. 75A through FIG. 75C depict the correlation of fragments between replicates per histone mark. Replicate 2 of the H3K27me3 is from bulk experiment. FIG. 75D through FIG. 75F depict MAstyle plot (x-axis; average number of reads; y-axis fold change between replicates) for assessing the replicability. Replicate 2 of the H3K27me3 is from bulk experiment. FIG. 75G depicts an unsupervised clustering analysis and spatial distribution of each cluster for H3K4me3 modifications from two different spatial-CUT&Tag experiments. FIG. 75H depicts an unsupervised clustering analysis and spatial distribution of each cluster for H3K27ac modifications from two different spatial-CUT&Tag experiments. FIG. 75I depicts a Venn diagram showing the overlap of peaks from two different spatial-CUT&Tag experiments.

FIG. 76A and FIG. 76B depicts data demonstrating the benchmarking of peaks called with spatial-CUT&Tag data. FIG. 76A depicts a Venn diagram showing the overlap of peaks called from spatial-CUT&Tag and ENCODE bulk ChIP-seq. FIG. 76B depicts a metagene heatmaps of ChIP-seq and spatial-CUT&Tag signal from liver delineation around peaks that were called from the bulk ChIP-seq dataset.

FIG. 77A and FIG. 77B depicts data demonstrating the unique fragment counts in spatial epigenome mapping of E11 mouse embryos (50 μm pixel size). FIG. 77A depicts the spatial heatmaps showing spatial distribution of unique fragment count per pixel analyzed for three different histone marks (H3K27me3, H3K4me3, and H3K27ac). FIG. 77B depicts UMAP embedding of unsupervised clustering analysis for each histone modification shaded with the number of unique fragments per pixel.

FIG. 78A through FIG. 78I depict data demonstrating the Spatial epigenome mapping and integrative analysis of E11 mouse embryos. FIG. 78A depicts genome browser tracks (left) and spatial mapping (right) of gene silencing by H3K27me3 modification for selected marker genes in different clusters. FIG. 78B depicts genome browser tracks (left) and spatial mapping (right) of gene activity by H3K4me3 modification for selected marker genes in different clusters. FIG. 78C depicts predicted enhancers of Ascl1 (chr10: 87,463,659-87,513,660; mm10) (left) and Kcnq3 (chr15:66,231,223-66,331,224; mm10) (right) from H3K27ac profiling. Cluster of each track corresponds to FIG. 71F. Enhancers validated by in vivo reporter assays are shown between main panels. FIG. 78D and FIG. 78F depict the integration of scRNA-seq from E11.5 mouse embryos (Cao et al., 2019, Nature, 566:496-502) and spatial-CUT&Tag data. Unsupervised clustering of the combined data was colored by different cell types. FIG. 78E and FIG. 78G depict spatial mapping of selected cell types identified by label transferring from scRNA-seq to spatial-CUT&Tag. FIG. 78H depicts a list of all identified cell types in scRNA-seq. FIG. 78I depicts refined clustering of radial glial enabled identification of sub-populations. Scale 15 bar, 1 mm.

FIG. 79A and FIG. 79B depicts data demonstrating the spatial profiling of H3K27me3 modification of E11 mouse embryos (50 μm pixel size). FIG. 79A depicts spatial mapping of gene silencing by H3K27me3 modification for selected marker genes in different clusters (see FIG. 71F). FIG. 79B depicts a GO enrichment analysis of differentially silenced genes in selected clusters (C1 and C8).

FIG. 80A and FIG. 80B depicts data demonstrating the spatial profiling of H3K4me3 modification in E11 mouse embryos with 50 μm pixel size. FIG. 80A depicts spatial mapping of gene activity by H3K4me3 modification for selected marker genes in different clusters (see FIG. 71F). FIG. 80B depicts a GO enrichment analysis of differentially activated genes in selected clusters (C2, C3, and C6).

FIG. 81A and FIG. 81B depicts data demonstrating the spatial profiling of H3K27ac modification in E11 mouse embryos with 50 μm pixel size. FIG. 81A depicts spatial mapping of gene activity by H3K27ac modification for selected marker genes in different clusters (see FIG. 71F). FIG. 81B depicts a GO enrichment analysis of differentially activated genes in selected clusters (C1, C2, and C4).

FIG. 82A and FIG. 82B depicts data demonstrating the motif enrichment of H3K4me3 modification in E11 mouse embryos. FIG. 82A depicts motif enrichment analysis on marker peaks identified in selected clusters (C2—liver, C4 spinal cord). FIG. 82B depicts spatial mapping of transcription factor (TF) motif scores and logo representation of the motif retrieved from the CIS-BP database (Stahl et al., 2016, Science, 353:78-82).

FIG. 83A and FIG. 83B depicts data demonstrating the motif enrichment of H3K27ac modification in E11 mouse embryos. FIG. 83A depicts the motif enrichment analysis on marker peaks identified in selected clusters (C4 for liver, C2 for spinal cord). FIG. 83B depicts the spatial mapping of TF motif scores and logo representation of the motif retrieved from the CIS-BP database (Stahl et al., 2016, Science, 353:78-82).

FIG. 84A through FIG. 84D depict data demonstrating the integrative analysis of scRNA-seq, DBiT-seq and spatial-CUT&Tag. FIG. 84A depicts the spatial mapping of selected cell types identified by label transferring from scRNA-seq to spatial-CUT&Tag (H3K4me3, 50 μm). FIG. 84B depicts the refined clustering of chondrocytes & osteoblasts enabled identification of sub-populations, and genes related to developing teeth (e.g. Barx1) had higher expression in subcluster 2. FIG. 84C depicts the integration of DBiT-seq from E11 mouse brain (Bartosovic et al., 2021, Nature Biotechnology) and spatial-CUT&Tag data (H3K4me3, 50 μm). Cluster identities are consistent with FIG. 71F. FIG. 84D depicts integration of DBiT-seq from E11 mouse brain (Bartosovic et al., 2021, Nature Biotechnology) and spatial-CUT&Tag data (H3K27ac, 50 μm). Cluster identities are consistent with FIG. 71F. Scale bar, 1 mm.

FIG. 85A through FIG. 85D depict data demonstrating the pseudotemporal spatial trajectories in the developing brain. FIG. 85A depicts an H&E image from an adjacent tissue section. FIG. 85B depicts a pseudotemporal reconstruction from the developmental process from radial glia, postmitotic premature neurons, to excitatory neurons plotted in space. FIG. 85C depicts dynamics for selected gene activity based on H3K4me3 along the pseudo-time shown in (FIG. 85B). FIG. 85D depicts a pseudo-time heatmap of gene score changes from radial glia, postmitotic premature neurons, to excitatory neurons. Scale bar, 1 mm.

FIG. 86A through FIG. 86I depict data demonstrating the Spatial epigenome mapping of an immunofluorescence-stained mouse olfactory bulb tissue section at cellular level. FIG. 86A depicts an H&E image of mouse olfactory bulb from an adjacent tissue section and a region of interest for spatial epigenome mapping. FIG. 86B depicts a fluorescent image of nuclear staining with DAPI in a region of interest performed on the same tissue section used for spatial epigenome mapping. FIG. 86C depicts an unsupervised clustering analysis and spatial distribution of each cluster of mouse olfactory bulb by H3K27me3 modification (20 μm pixel size). FIG. 86D depicts spatial mapping (left) of gene silencing by H3K27me3 modification for selected marker genes. In situ hybridization (right) and expression images (middle) of corresponding genes are from the Allen Institute database. FIG. 86E depicts fluorescent images of selected pixels containing single nuclei (DAPI). FIG. 86F depicts a heatmap of chromatin silencing score of selected pixels. FIG. 86G depicts a comparison of number of unique fragments in pixels with non-single nucleus (>1 nucleus) and single nucleus. FIG. 86H depicts a UMAP of unsupervised clustering analysis of selected pixels containing single nuclei. FIG. 86I depicts a UMAP colored by chromatin silencing score for selected genes.

FIG. 87A through FIG. 87K depict data demonstrating the spatial epigenome mapping of E11 mouse embryos with 20 μm pixel size. FIG. 87A depicts an H&E image of an E11 mouse embryo from an adjacent tissue section and a region of interest for spatial epigenome mapping. FIG. 87B depicts an unsupervised clustering analysis and spatial distribution of each cluster of E11 mouse embryo per histone mark. FIG. 87C depicts a UMAP embedding of unsupervised clustering analysis for each histone modification. Cluster identities and coloring of clusters are consistent with (FIG. 87B). FIG. 87D depicts an LSI projection of ENCODE bulk ChIP-seq data from different organs of the E11.5 mouse embryo dataset into the spatial-CUT&Tag embedding. FIG. 87E depicts genome browser tracks (left) and spatial mapping (right) of gene silencing by H3K27me3 modification for selected marker genes in different clusters of the E11 mouse embryo data. FIG. 87F depicts co-embedding of spatial-CUT&Tag data of active histone modifications (H3K4me3 and H3K27ac) in a UMAP space (left) and spatial distribution of each cluster for different histone modifications (right). FIG. 87G and FIG. 87I depict integration of scRNA-seq from E11.5 mouse embryos 19 and spatial-CUT&Tag data. Unsupervised clustering of the combined data was colored by different cell types. FIG. 87H and FIG. 87J depict spatial mapping of selected cell types identified by label transferring from scRNA-seq to spatial-CUT&Tag. FIG. 87K depicts a list of all identified cell types in scRNA-seq. Scale bar, 500 μm.

FIG. 88A and FIG. 88B depicts data demonstrating the spatial profiling of H3K27me3 modification in E11 mouse embryos with 20 μm pixel size. FIG. 88A depicts spatial mapping of gene silencing by H3K27me3 modification for selected marker genes in different clusters. FIG. 88B depicts a GO enrichment analysis of differentially silenced genes in selected clusters (C1, C2, and C4).

FIG. 89A through FIG. 89J depict data demonstrating the spatial epigenome mapping and integrative analysis of P21 mouse brain at cellular level. FIG. 89A depicts a mouse brain tissue section imaged prior to performing spatial-CUT&Tag. The region of interest for spatial epigenome mapping is indicated with a dashed box. FIG. 89B and FIG. 89C depict unsupervised clustering analysis and spatial distribution of each cluster of mouse brain per histone mark (20 μm pixel size). FIG. 89D depicts a spatial mapping of gene activity by H3K4me3 modification for selected marker genes in different clusters. FIG. 89E depicts a spatial mapping of gene silencing by H3K27me3 modification for selected marker genes in different clusters. FIG. 89F depicts a refined clustering process enabled identification of sub-populations in neurons with distinct spatial distributions and marker genes. FIG. 89G depicts integration of scCUT&Tag from mouse brains (Bartosovic et al., 2021, Nature Biotechnology) and spatial-CUT&Tag. FIG. 89H depicts the integration of scRNA-seq from mouse brains (Zeisel et al., 2018, Cell, 174:999-1014.e1022) and spatial-CUT&Tag data. FIG. 89I depicts a list of all identified cell types in scRNA-seq. FIG. 89J depicts spatial mapping of selected cell types identified by label transferring from scRNA-seq to spatial-CUT&Tag. MGL1: Microglia, nonactivated; MSN2: D2 medium spiny neurons, striatum; MOL1: Mature oligodendrocytes; ACTE2: Telencephalon astrocytes, protoplasmic; TEGLU3: Excitatory neurons, cerebral cortex; TEGLU8: Excitatory neurons, cerebral cortex. Scale bar, 500 μm.

FIG. 90A through FIG. 90N depict data demonstrating the spatial profiling of H3K4me3 modification in mouse brain with 20 μm pixel size. FIGS. 90A, C, E, G, I, K, and M depict the spatial mapping of gene activity by H3K4me3 modification for selected marker genes in different clusters. FIGS. 90B, D, F, H, J, L, and N depict gene expression of selected marker genes in different clusters is shown along the cell-type taxonomy. Each row represents one marker gene, and columns represent cell-type taxonomy. Data from the mouse CNS single-cell transcriptomics atlas (Burgess et al., 2019, Nat Rev Genet, 20:317), and from mousebrain.org.

FIG. 91A through FIG. 91J depict data demonstrating the spatial profiling of H3K27me3 modification in mouse brain with 20 μm pixel size. FIGS. 91A, C, E, G, and I depict spatial mapping of gene silencing by H3K27me3 modification for selected marker genes in different clusters. FIGS. 91B, D, F, H, and J depict gene expression of selected marker genes in different clusters is shown along the cell-type taxonomy. Each row represents one marker gene, and columns represent cell-type taxonomy. Data from the mouse CNS single-cell transcriptomics atlas (Burgess et al., 2019, Nat Rev Genet, 20:317), and from mousebrain.org.

FIG. 92 depicts deconvolution of potential H3K4me3/H3K27me3 bivalency in mouse brain. Metagene heatmaps of H3K4me3 (left) and H3K27me3 (right) occupancy at the loci of H3K4me3 peaks called from each cell type. Each x axis shows 10 kb on either side of the marker peaks. Heatmaps show signal across marker regions.

FIG. 93 depicts the chemistry workflow of high-spatial-resolution multi-omics profiling. A tissue section on a standard aminated glass slide was lightly fixed with formaldehyde. Afterwards, a cocktail of antibody-DNA tags (ADTs) were first added to the tissue surface to capture target membrane proteins. After permeabilization, primary antibody binds to the target histone modifications or chromatin-interacting proteins, ADTs for intracellular proteins and ADTs for metabolites were added, followed by a secondary antibody binding for enhancing tethering of pA-Tn5 transposome. pA-Tn5 transposome and Tn5 proteins linked to methylation sensitive restriction enzyme were then activated by adding Mg++ and incubating at 37° C., and adapters containing ligation linker 1 was inserted at antibody bound sites. Then, RT mix combined with ligation linker 1 was added for mRNA capturing, reverse transcription and template switch. Afterwards, a set of DNA barcode A solutions were introduced to perform in situ ligation reaction for appending a distinct spatial barcode Ai (i=1-50) and ligation linker 2. A second set of barcodes Bj (j=1-50) were then introduced perpendicularly to those in the first flow barcoding, which were ligated at the intersections, resulting in a mosaic of tissue pixels, each containing a distinct combination of barcodes Ai and Bj (i=1-50, j=1-50). After DNA fragments and cDNA were collected by reversing cross-linking, PCR amplification and library construction were performed.

FIG. 94a through FIG. 94i depict the spatial-ATAC-seq: design, workflow, and data quality. FIG. 94a depicts the schematic workflow. Tn5 transposition was performed in tissue sections, followed by in-situ ligation of two sets of DNA barcodes (A1-A50, B1-B50). FIG. 94b depicts validation of in-situ transposition and ligation using fluorescent DNA probes. Tn5 transposition was performed in 3T3 cells on a glass slide stained by DAPI (blue). Afterwards, FITC-labeled barcode A is ligated to the adapters on the transposase accessible genomic DNA. Scale bar, 50 μm. FIG. 94c depicts aggregate spatial chromatin accessibility profiles recapitulated published profiles of ATAC-seq in the liver of E13 mouse embryo. FIG. 94d depicts a comparison of number of unique fragments for different protocols and microfluidic channel width between the spatial method in this work and 10× scATAC-seq. FIG. 94e depicts a comparison of fraction of TSS fragments for different protocols and microfluidic channel width between the spatial method in this work and 10× scATAC-seq. FIG. 94f depicts a comparison of fraction of mitochondrial fragments for different protocols and microfluidic channel width between the spatial method in this work and 10× scATAC-seq. FIG. 94g depicts a comparison of insert size distribution of ATAC-seq fragments for different protocols and microfluidic channel width between the spatial method in this work and 10× scATAC-seq. FIG. 94h depicts a comparison of enrichment of ATAC-seq reads around TSSs for different protocols and microfluidic channel width between the spatial method in this work and 10× scATAC-seq. Coloring is consistent with (FIG. 94g) FIG. 94h depicts a scatterplot showing the TSS enrichment score vs unique nuclear fragments per cell for human tonsil.

FIG. 95 depicts a diagram of the chemistry workflow of spatial-ATAC-seq. A tissue section on a standard aminated glass slide was lightly fixed with formaldehyde. Then, Tn5 transposition was performed at 37° C., and the adapters containing ligation linker I were inserted to the cleaved genomic DNA at transposase accessible sites. Afterwards, a set of DNA barcode A solutions were introduced by microchannel-guided flow delivery to perform in situ ligation reaction for appending a distinct spatial barcode Ai (i=1-50) and ligation linker 2. Then, a second set of barcodes Bj (j=1-50) were introduced using another set of microfluidic channels perpendicularly to those in the first flow barcoding step, which were subsequently ligated at the intersections, resulting in a mosaic of tissue pixels, each containing a distinct combination of barcodes Ai and Bj (i=1-50, j=1-50). After DNA fragments were collected by reversing cross-linking, the library construction was completed during PCR amplification.

FIG. 96a and FIG. 96b depict the quality control metrics for spatial ATAC-seq datasets. FIG. 96a depicts a scatterplot showing the TSS enrichment score vs unique nuclear fragments per cell for different protocols and microfluidic channel width. FIG. 96b depicts reproducibility between biological replicates on E13 mouse embryo. Pearson correlation coefficient r=0.95.

FIG. 97a through FIG. 97i depict the spatial chromatin accessibility mapping of E13 mouse embryo. FIG. 97a depicts an unbiased clustering analysis, performed based on chromatin accessibility of all tissue pixels (50 μm pixel size). Overlay of clusters with the tissue image reveals that the spatial chromatin accessibility clusters precisely match the anatomic regions. FIG. 97b depicts UMAP embedding of unsupervised clustering analysis for chromatin accessibility. Cluster identities and coloring of clusters are consistent with (FIG. 97a). FIG. 97c depicts spatial mapping of gene scores for selected marker genes in different clusters and the chromatin accessibility at select genes are highly tissue specific. FIG. 97d depicts the integration of scRNA-seq from E13.5 mouse embryos (Cao et al., 2019, Nature, 566:496-502) and spatial ATAC-seq data. Unsupervised clustering of the combined data was colored by different cell types. FIG. 97e depicts an anatomic annotation of major tissue regions based on the H&E image. FIG. 97f depicts spatial mapping of selected cell types identified by label transferring from scRNA-seq to spatial ATAC-seq data. FIG. 97g depicts pseudotemporal reconstruction from the developmental process from radial glia, postmitotic premature neurons, to excitatory neurons plotted in space. FIG. 97h depicts dynamics for selected gene score along the pseudo-time shown in (FIG. 97g). FIG. 97h depicts a pseudo-time heatmap of TF motifs changes from radial glia, postmitotic premature neurons, to excitatory neurons.

FIG. 98a through FIG. 98g depict a further analysis of spatial chromatin accessibility mapping of E13 mouse embryo, validation with ENCODE, and sub-clustering in liver. FIG. 98a depicts an H&E image from an adjacent tissue section and a region of interest for spatial chromatin accessibility mapping (50 μm pixel size). FIG. 98b depicts an unsupervised clustering analysis and spatial distribution of each cluster. FIG. 98c depicts a UMAP embedding of unsupervised clustering analysis for spatial ATAC-seq. Cluster identities and coloring of clusters are consistent with (FIG. 98b). FIG. 98d depicts an LSI projection of ENCODE bulk ATAC-seq data from diverse cell types of the E13.5 mouse embryo dataset onto the spatial ATAC-seq embedding. FIG. 98e and FIG. 98f depict genome browser tracks (FIG. 98e) and spatial mapping (FIG. 98f) of gene scores for selected marker genes in different clusters. FIG. 98g depicts a refined clustering of fetal liver in E13 mouse embryo enabled identification of sub-populations, and some genes related to hematopoiesis (e.g. Hbb-y, Slc-4a1, Sptb) had higher expression lever in the subcluster 1.

FIG. 99a through FIG. 99j depict spatial mapping of gene scores in E13 mouse embryo and comparison with ISH reference data. FIGS. 88a, c, e, g, and i depict spatial mapping of the gene score for selected genes in E13 mouse embryo. FIG. 99b, d, f, h, and j depict in situ hybridization of selected genes at E13.5 mouse embryo from Allen Developing Mouse Brain Atlas.

FIG. 100 depicts a GO enrichment analysis of spatial ATAC-seq data for E13 mouse embryo. GO enrichment analysis of differentially activated genes in selected clusters (C1, C5 and C6).

FIG. 101a and FIG. 101b depict the gene score along the anterior-posterior axis of the spine. FIG. 101a depicts the spine region of E13 mouse embryo profiled by spatial ATAC-seq. FIG. 101b depicts the selected genes found to form expression gradients along the anterior-posterior axis.

FIG. 102a through FIG. 102c depict the motif enrichment analysis of the E13 mouse embryo data. FIG. 102a depicts a heatmap of spatial ATAC-seq marker peaks across all clusters identified with bias-matched differential testing. FIG. 102b depicts a heatmap of motif hypergeometric enrichment-adjusted P values within the marker peaks of each cluster. FIG. 102c depicts the spatial mapping of selected TF motif deviation scores.

FIG. 103a through FIG. 103d depict an integrative analysis of spatial ATAC-seq and scRNA-seq for E13 mouse embryo and sub-clustering of excitatory neurons. FIG. 103a depicts the spatial mapping of selected cell types identified by label transferring from scRNA-seq to spatial ATAC-seq. FIG. 103b through FIG. 103d depict a refined clustering process enabled identification of sub-populations in excitatory neurons with distinct spatial distributions (FIG. 103b) and marker genes (FIG. 103c, d).

FIG. 104a through FIG. 104p depict the spatial chromatin accessibility mapping of E11 mouse embryo and spatiotemporal analysis. FIG. 104a depicts an unsupervised clustering analysis and spatial distribution of each cluster. Overlay with the tissue image reveals that the spatial chromatin accessibility clusters precisely match the anatomic regions FIG. 104b depicts UMAP embedding of unsupervised clustering analysis for chromatin accessibility. Cluster identities and coloring of clusters are consistent with (FIG. 104a). FIG. 104c depicts spatial mapping of gene scores for selected marker genes in different clusters and the chromatin accessibility at select genes are highly tissue specific. FIG. 104d depicts the integration of scRNA-seq from E11.5 mouse embryos (Cao et al., 2019, Nature, 566:496-502) and spatial ATAC-seq data. Unsupervised clustering of the combined data was colored by different cell types. FIG. 104e depicts an anatomic annotation of major tissue regions based on the H&E image. FIG. 104f depicts spatial mapping of selected cell types identified by label transferring from scRNA-seq to spatial ATAC-seq data. FIG. 104g depicts a pseudotemporal reconstruction from the developmental process from radial glia to excitatory neurons plotted in space. FIG. 104h depicts spatial mapping of gene scores for Notch 1. FIG. 104i depicts dynamics for selected gene score along the pseudo-time shown in (FIG. 104g). FIG. 104j depicts a pseudo-time heatmap of TF motifs changes from radial glia to excitatory neurons. FIG. 104k depicts a pseudo-time heatmap of TF motifs changes in the fetal liver from E11 to E13 mouse embryo. FIG. 104l depicts a differential peak analysis of fetal liver in E13 mouse embryo compared to E11 mouse embryo. FIG. 104m depicts a ranking of enriched motifs in the peaks that are more accessible in the fetal liver of E13 mouse embryo compared to E11 mouse embryo. FIG. 104n depicts a pseudo-time heatmap of TF motifs changes in the excitatory neurons from E11 to E13 mouse embryo. FIG. 104o depicts a differential peak analysis of excitatory neurons in E13 mouse embryo compared to E11 mouse embryo. FIG. 104p depicts a ranking of enriched motifs in the peaks that are more accessible in the excitatory neurons of E13 mouse embryo compared to E11 mouse embryo.

FIG. 105a through FIG. 105f depict a further analysis of spatial chromatin accessibility mapping of E11 mouse embryo and validation with the ENCODE reference data. FIG. 105a depicts an H&E image from an adjacent tissue section and a region of interest for spatial chromatin accessibility mapping (50 μm pixel size). FIG. 105b depicts an unsupervised clustering analysis and spatial distribution of each cluster. FIG. 105c depicts a UMAP embedding of unsupervised clustering analysis for spatial ATAC-seq. Cluster identities and coloring of clusters are consistent with (FIG. 105b). FIG. 105d depicts an LSI projection of ENCODE bulk ATAC-seq data from diverse cell types of the E11.5 mouse embryo dataset onto the spatial ATAC-seq embedding. FIG. 105e, and f depict genome browser tracks (FIG. 105e) and spatial mapping (FIG. 105f) of gene scores for selected marker genes in different clusters.

FIG. 106 depicts a GO enrichment analysis of spatial ATAC-seq data for E11 mouse embryo. GO enrichment analysis of differentially activated genes in selected clusters (C1, C3 and C4).

FIG. 107a through FIG. 107b depict a motif enrichment analysis in E11 mouse embryo. FIG. 107a depicts a heatmap of spatial ATAC-seq marker peaks across all clusters identified with bias-matched differential testing. FIG. 107b depicts the spatial mapping of selected TF motif deviation scores.

FIG. 108 depicts an integrative analysis of spatial ATAC-seq and scRNA-seq for E11 mouse embryo and spatial map visualization of select cell types. Spatial mapping of selected cell types identified by label transferring from scRNA-seq to spatial ATAC-seq. FIG. 109a through FIG. 109i depict the spatial chromatin accessibility mapping of human tonsil with 20 μm pixel size. FIG. 109a depicts an H&E image of a human tonsil from an adjacent tissue section and a region of interest for spatial chromatin accessibility mapping. FIG. 109b depicts an unsupervised clustering analysis and spatial distribution of each cluster. FIG. 109c depicts an anatomic annotation of major tonsillar regions. FIG. 109d depicts spatial mapping of gene scores for selected genes. FIG. 109e depicts the integration of scRNA-seq data (King et al., 2021, bioRxiv, 2021.2003.2016.435578) and spatial ATAC-seq data. Unsupervised clustering of the combined data was colored by different cell types. FIG. 109f depicts a spatial mapping of selected cell types identified by label transferring from scRNA-seq to spatial ATAC-seq data. Scale bar, 500 μm. FIG. 109g depicts a pseudotemporal reconstruction from the developmental process from Naïve B cells to GC B cells plotted in space. FIG. 109h depicts the dynamics for selected gene score along the pseudo-time shown in (FIG. 109g). FIG. 109h depicts a pseudo-time heatmap of TF motifs changes from Naïve B cells to GC B cells.

FIG. 110a and FIG. 110b depict the single-cell mapping of immune cell subsets in human tonsil. FIG. 110a depicts a UMAP of tonsillar immune scRNA-seq reference data (King et al., 2021, bioRxiv, 2021.2003.2016.435578). FIG. 110b depicts a heatmap comparing key marker gene expression across selected immune cell types.

FIG. 111 depicts a spatial chromatin accessibility gene score map in comparison with protein expression in human tonsil. The immunohistochemistry reference data were obtained from the Human Protein Atlas (Uhlén et al., 2015, Science, 347:1260419).

FIG. 112 depicts a motif enrichment analysis of spatial ATAC-seq data for human tonsil. Spatial mapping of motif deviation scores for KLF family transcription factors.

FIG. 113 depicts spatial chromatin accessibility mapping of human tonsil with 20 μm pixel size and visualization of specific marker genes. Spatial mapping of gene scores for selected genes.

### DETAILED DESCRIPTION

The present invention relates generally to systems and methods for spatially resolved epigenomic profiling at single-cell level directly in the original tissue specimen. The presently described systems and methods represents a major leap in the field of epigenomics and potentially a ground-breaking technology to enable a new field of biomedical research with far-reaching impact in developmental biology, cancer research, immunology, cardiovascular disease study, histopathology, and therapeutic discovery.

The present disclosure provides a fundamentally new technology for spatial epigenomics—high resolution and deterministic spatial ATAC-seq (hsrATAC-seq). A microfluidic chip with parallel channels (e.g., 20 or 50 μm in width) is placed directly against a tissue sample on a slide, and in some embodiments clamped to a region of interest using a particular clamping force. Then, in certain embodiments, a fusion protein of hyperactive Tn5 transposase and protein A assembled with a DNA oligo sequence that serves as a ligation linker is added. Activation of the transposase initiates tagmentation, in which the transposase cuts the DNA molecule on either side of the epigenomic marker, and anneals the DNA ligation linker sequence to the cut DNA. Following tagmentation, a first set of unique DNA barcodes (A₁-Aᵢ, wherein ᵢ is an integer between 1 and 1001) are flowed across the channels of the microfluidic chip in a first direction (A), and ligating the first barcode set to the ligation linker, followed by washing, removing the chip, applying a second microfluidic chip, wherein the second microfluidic chip is placed such that the flow direction is perpendicular to the flow direction of the first chip (A) and flowing a second set of unique DNA barcodes (B₁-Bⱼ, wherein ⱼ is an integer between 1 and 1001) are flowed across the channels of the microfluidic chip in a second direction (B) which is perpendicular to the first direction (A), and ligating the second set of barcodes to the first barcode set. Then, the tissue is lysed and spatially barcoded DNA molecules are retrieved, pooled, and amplified by PCR, to prepare a library for NGS sequencing.

In some embodiments, the transposase is linked to a methylation sensitive restriction enzyme. In some embodiments, a primary antibody specific to an epigenomic marker is added prior to addition of a secondary antibody and to the addition of with a transposase and linker sequence. In such embodiments, the methods of the invention can restrict the cleavage and tagementation to specific regions of interest including regions having specific epigenomic markers, allowing for the generation of spatial epigenomic maps.

The data provided herein has demonstrated high-spatial-resolution mapping of the transcriptome and epigenomic markers in mouse embryos. It faithfully detected areas of increased and decreased chromatin silencing or gene activation through detecting areas of increased or decreased histone methylation. The spatial epigenomic map further identifies differential patterns of gene expression during embryonic development. hsrATAC-seq does not require any DNA spot microarray or decoded DNA-barcoded bead array but only a set of reagents. It works for an existing fixed tissue slide, not requiring newly prepared tissue sections that are necessary for other methods (Rodriques et al., 2019, Science, 363:1463-1467; Stahl et al., 2016, Science, 353:78-82). It is highly versatile allowing for the combining of different reagents for multiple omics measurements directly on the tissue slide. Thus, hsrATAC-seq is potentially a platform technology that can be readily adopted by researchers from a wide range of biological and biomedical research fields.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

In some embodiments, the invention provides new methods for high-spatial-resolution, unbiased, epigenomic mapping in intact tissues, which does not require sophisticated imaging but can instead capitalize on the power of high-throughput Next Generation Sequencing (NGS). The present invention relates to compositions and methods for performing hsrATAC-seq.

In one embodiment, the method comprises the steps of: placing a first microfluidic chip with parallel channels (e.g., 20 or 50 μm in width) directly against tissue sample slide to be analyzed, contacting the sample with a transposase assembled with a DNA oligo sequence that serves as a ligation linker, flowing a first set of unique DNA barcodes (A₁-Aᵢ, wherein ᵢ is an integer between 1 and 1001) across the channels of the microfluidic chip in a first direction (A), ligating the first barcode set to the ligation linker, washing, removing the first microfluidic chip, applying a second microfluidic chip, wherein the second microfluidic chip is placed such that the flow direction is perpendicular to the flow direction of the first chip (A), flowing a second set of unique DNA barcodes (B₁-Bⱼ, wherein ⱼ is an integer between 1 and 1001) across the channels of the microfluidic chip in a second direction (B) which is perpendicular to the first direction (A), and ligating the second set of barcodes to the first barcode set. In some embodiments, the method further comprises lysing the cells, retrieving the spatially barcoded DNA molecules and preparing a NGS sequencing library from the spatially barcoded DNA molecules. In one embodiment, the method further includes a step of permeabilization prior to contacting the sample with the primary antibody. For example, in one embodiment, the sample is permeabilized with NP40-Digitonin buffer prior to contacting the sample with the transposase. In one embodiment, the transposase is a fusion protein of hyperactive Tn5 transposase and protein A.

In one embodiment, the method comprises the steps of: placing a first microfluidic chip with parallel channels (e.g., 20 or 50 μm in width) directly against tissue sample slide to be analyzed, contacting the sample with one or more antibodies specific for an epigenomic marker, contacting the sample with a secondary antibody and a transposase assembled with a DNA oligo sequence that serves as a ligation linker, flowing a first set of unique DNA barcodes (A₁-Aᵢ, wherein ᵢ is an integer between 1 and 1001) across the channels of the microfluidic chip in a first direction (A), ligating the first barcode set to the ligation linker, washing, removing the first microfluidic chip, applying a second microfluidic chip, wherein the second microfluidic chip is placed such that the flow direction is perpendicular to the flow direction of the first chip (A), flowing a second set of unique DNA barcodes (B₁-Bⱼ, wherein ⱼ is an integer between 1 and 1001) across the channels of the microfluidic chip in a second direction (B) which is perpendicular to the first direction (A), and ligating the second set of barcodes to the first barcode set. In some embodiments, the method further comprises lysing the cells, retrieving the spatially barcoded DNA molecules and preparing a NGS sequencing library from the spatially barcoded DNA molecules. In one embodiment, the method further includes a step of permeabilization prior to contacting the sample with the primary antibody. For example, in one embodiment, the sample is permeabilized with NP40-Digitonin buffer prior to contacting the sample with the primary antibody. In one embodiment, the transposase is a fusion protein of hyperactive Tn5 transposase and protein A.

In one embodiment, the method of the invention incorporates a DNA ligation adaptor or DNA barcode sequence, or a combination thereof, onto a nucleic acid molecule comprising an epigenomic mark of interest using a “cut and tag” method or “tagmentation.” As used herein, the term “tagmentation” refers to the modification of DNA by a transposome complex comprising transposase enzyme complexed with adaptors comprising transposon end sequence. Tagmentation results in the simultaneous fragmentation of the target DNA molecule comprising the epigenomic mark of interest and ligation of the adaptors to the 5′ ends of both strands of duplex fragments. Following a purification step to remove the transposase enzyme, additional sequences (e.g., barcodes) can be added to the ends of the adapted fragments, for example by PCR, ligation, or any other suitable methodology known to those of skill in the art.

The method of the invention can use any transposase that can accept a transposase end sequence and fragment a target nucleic acid, attaching a transferred end, but not a non-transferred end. A “transposome” is comprised of at least a transposase enzyme and a transposase recognition site. In some such systems, termed “transposomes”, the transposase can form a functional complex with a transposon recognition site that is capable of catalyzing a transposition reaction. The transposase or integrase may bind to the transposase recognition site and insert the transposase recognition site into a target nucleic acid in a process sometimes termed “tagmentation”. In some such insertion events, one strand of the transposase recognition site may be transferred into the target nucleic acid.

Some embodiments can include the use of a hyperactive Tn5 transposase and a Tn5-type transposase recognition site (Goryshin and Reznikoff, J. Biol. Chem., 273:7367 (1998)), or MuA transposase and a Mu transposase recognition site comprising R1 and R2 end sequences (Mizuuchi, K., Cell, 35: 785, 1983; Savilahti, H, et al., EMBO J., 14: 4893, 1995). An exemplary transposase recognition site that forms a complex with a hyperactive Tn5 transposase (e.g., EZ-Tn5™ Transposase, Epicentre Biotechnologies, Madison, Wis.).

More examples of transposition systems that can be used with certain embodiments provided herein include Staphylococcus aureus Tn552 (Colegio et al., J. Bacteriol., 183: 2384-8, 2001; Kirby C et al., Mol. Microbiol., 43: 173-86, 2002), Ty1 (Devine & Boeke, Nucleic Acids Res., 22: 3765-72, 1994 and International Publication WO 95/23875), Transposon Tn7 (Craig, NL, Science. 271: 1512, 1996; Craig, N L, Review in: Curr Top Microbiol Immunol., 204:27-48, 1996), Tn/O and IS10 (Kleckner N, et al., Curr Top Microbiol Immunol., 204:49-82, 1996), Mariner transposase (Lampe D J, et al., EMBO J., 15: 5470-9, 1996), Tc1 (Plasterk R H, Curr. Topics Microbiol. Immunol., 204: 125-43, 1996), P Element (Gloor, G B, Methods Mol. Biol., 260: 97-114, 2004), Tn3 (Ichikawa & Ohtsubo, J Biol. Chem. 265:18829-32, 1990), bacterial insertion sequences (Ohtsubo & Sekine, Curr. Top. Microbiol. Immunol. 204: 1-26, 1996), retroviruses (Brown, et al., Proc Natl Acad Sci USA, 86:2525-9, 1989), and retrotransposon of yeast (Boeke & Corces, Annu Rev Microbiol. 43:403-34, 1989). More examples include ISS, Tn10, Tn903, IS911, and engineered versions of transposase family enzymes (Zhang et al., 2009, PLOS Genet. 5:e1000689. Epub 2009 Oct. 16; Wilson C. et al (2007) J. Microbiol. Methods 71:332-5).

In one embodiment, the transposase is hyperactive Tn5 transposase tethered to protein A.

In one embodiment, the transposase is linked to a methylation sensitive restriction enzyme. Methylation sensitive restriction enzymes (MSREs) include, but are not limited to, Aat II, Acc II, Aor13H I, Aor51H I, BspT104 I, BssH II, Cfr10 I, Cla I, Cpo I, Eco52 I, Hae II, Hap II, Hha I, Mlu I, Nae I, Not I, Nru I, Nsb I, PmaC I, Psp1406 I, Pvu I, Sac II, Sal I, Sma I, and SnaB I.

In one embodiment, the tagmentation reaction is allowed to proceed for at least 10 minutes, at least 15 minutes, at least 20 minutes, at least 25 minutes, at least 30 minutes or for more than 30 minutes prior to flowing the first barcode set through the fluidic microchip.

In one embodiment, the concentration of transposome used for the tagementation reaction is between 1 μl and 20 μl. For example, in one embodiment, an 8 μl Tn5 transposome is assembled comprising 2 μl DNA oligo, 4 μl EZ-Tn5 Transposase (1 U/μl), and 2 μl glycerol). Before the tagmentation reaction, the Tn5 transposome is mixed with Tagment DNA buffer, 1×PBS, 10% Tween-20, 1% Digitonin to a total of 200 μl. In one embodiment, tagmentation is performed using a reaction time of at least 15, at least 20, at least 25, at least 30 or more than 30 minutes. In one embodiment, tagmentation is performed using 8 μl Tn5 transposome with a reaction time of 30 minutes.

### Epigenomic Marker Identification

In some embodiments, the methods of the invention include barcoding a nucleic acid molecule containing an epigenomic marker of interest in a biological sample. In some embodiments, the method includes the use of a primary antibody specific for binding to the epigenomic marker of interest. Non-limiting examples of antibodies include whole antibodies, Fab antibody fragments, F(ab′)₂ antibody fragments, monospecific Fab₂ fragments, bispecific Fab₂ fragments, trispecific Fab₃ fragments, single chain variable fragments (scFvs), bispecific diabodies, trispecific diabodies, scFv-Fc molecules, nanobodies, and minibodies.

In one embodiment, the primary antibody for use in the methods of the invention is specific for an epigenomic marker. Exemplary epigenomic markers that can be identified using the method of the invention include, but are not limited to, H2AK5ac, H2AK9ac, H2BK120ac, H2BK12ac, H2BK15ac, H2BK20ac, H2BK5ac, H2Bub, H3, H3ac, H3K14ac, H3K18ac, H3K23ac, H3K23me2, H3K27me1, H3K27me2, H3K36ac, H3K36me1, H3K36me2, H3K4ac, H3K56ac, H3K79me1, H3K79me3, H3K9acS10ph, H3K9me2, H3S10ph, H3T11ph, H4, H4ac, H4K12ac, H4K16ac, H4K5ac, H4K8ac, H4K91ac, H3F3A, H3K27me3, H3K36me3, H3K4me1, H3K79me2, H3K9me1, H3K9me2, H3K9me3, H4K20me1, H2AFZ, H3K27ac, H3K4me2, H3K4me3, and H3K9ac. Exemplary primary antibodies specific for epigenomic markers include, but are not limited to: (accession numbers from encodeproject.org) ENCAB841KJH, ENCAB000AOZ, ENCAB000APA, ENCAB000AOY, ENCAB000ARP, ENCAB000AQJ, ENCAB000ASI, ENCAB000AOS, ENCAB000AOR, ENCAB000APJ, ENCAB000API, ENCAB000ARU, ENCAB050QKP, ENCAB000AQK, ENCAB000AOT, ENCAB928LTI, ENCAB788ZME, ENCAB928HBB, ENCAB417DUO, ENCAB000AHF, ENCAB296TBH, ENCAB000APH, ENCAB000APG, ENCAB000ARW, ENCAB188IXL, ENCAB039IRN, ENCAB000AOK, ENCAB000AOL, ENCAB960XYH, ENCAB000ARX, ENCAB000ARY, ENCAB000ASZ, ENCAB602YNP, ENCAB205THQ, ENCAB375PDS, ENCAB931TIC, ENCAB961FBP, ENCAB750SJL, ENCAB453MST, ENCAB592AAE, ENCAB638MGM, ENCAB382YEO, ENCAB127FOW, ENCAB790SCK, ENCAB000ASH, ENCAB000ASJ, ENCAB121PMJ, ENCAB470FGK, ENCAB056ZFO, ENCAB000AOM, ENCAB000AOO, ENCAB000AON, ENCAB231VKB, ENCAB458UGW, ENCAB502YEA, ENCAB000ANJ, ENCAB829JCF, ENCAB002YEX, ENCAB093UKQ, ENCAB376DXS, ENCAB783AQT, ENCAB062SHF, ENCAB172ZWF, ENCAB638TXJ, ENCAB113TJV, ENCAB630GBO, ENCAB000AQQ, ENCAB529WLG, ENCAB150MLG, ENCAB255ALZ, ENCAB862RIQ, ENCAB327ADQ, ENCAB000AQT, ENCAB413BOQ, ENCAB498DNV, ENCAB093TAW, ENCAB151HMS, ENCAB000ARR, ENCAB000ARQ, ENCAB846BDR, ENCAB864KQT, ENCAB647DFQ, ENCAB000ART, ENCAB000ARS, ENCAB000APB, ENCAB494QXU, ENCAB723WFC, ENCAB984FPK, ENCAB738OTL, ENCAB844TLA, ENCAB771AMN, ENCAB643NJW, ENCAB219DGO, ENCAB155VEG, ENCAB036YAO, ENCAB268VLH, ENCAB009VWX, ENCAB000AQY, ENCAB266AZH, ENCAB000AUP, ENCAB000AQZ, ENCAB000ANB, ENCAB000ATC, ENCAB000ASA, ENCAB694MYM, ENCAB000AUT, ENCAB900FRR, ENCAB000ASD, ENCAB000ASC, ENCAB000ASB, ENCAB000AXZ, ENCAB000AXS, ENCAB323UEU, ENCAB000ADT, ENCAB169CDD, ENCAB782COR, ENCAB000ATF, ENCAB000ANC, ENCAB000ARI, ENCAB000ARJ, ENCAB000BLC, ENCAB000BLA, ENCAB000BLB, ENCAB910BYC, ENCAB773ECH, ENCAB570ZTO, ENCAB261ELA, ENCAB661HUV, ENCAB405MHV, ENCAB582RBY, ENCAB000ARD, ENCAB000AQW, ENCAB211WTE, ENCAB861ENQ, ENCAB000ADV, ENCAB360BDG, ENCAB523NUQ, ENCAB000AQB, ENCAB000BKT, ENCAB000APZ, ENCAB000AQC, ENCAB000AQD, ENCAB000ASN, ENCAB000ADU, ENCAB000AQE, ENCAB000ATB, ENCAB000AUW, ENCAB000AQF, ENCAB000AND, ENCAB000AQG, ENCAB000ARH, ENCAB000BKX, ENCAB000BSH, ENCAB543RHW, ENCAB027VOE, ENCAB539BDB, ENCAB969VGQ, ENCAB256MFX, ENCAB093ZAC, ENCAB663IEY, ENCAB650MWL, ENCAB472HKJ, ENCAB000ADW, ENCAB249ROX, ENCAB644AJI, ENCAB491AYZ, ENCAB000ARZ, ENCAB000APR, ENCAB000APS, ENCAB000ADX, ENCAB000ATH, ENCAB000AYB, ENCAB378MIH, ENCAB845ARK, ENCAB000AQU, ENCAB208AUK, ENCAB000ANE, ENCAB000ARE, ENCAB000APP, ENCAB000APO, ENCAB775EVT, ENCAB483QLF, ENCAB913CFY, ENCAB627HBE, ENCAB001LDA, ENCAB000AOQ, ENCAB000ANI, ENCAB000ANH, ENCAB000AQP, ENCAB004CMB, ENCAB352FQM, ENCAB180QII, ENCAB000APT, ENCAB000ANP, ENCAB681ELK, ENCAB449CFZ, ENCAB778TBN, ENCAB172IHG, ENCAB929ZIJ, ENCAB027OJQ, ENCAB769IVA, ENCAB164QXS, ENCAB890YOB, ENCAB691OYV, ENCAB499JWV, ENCAB292IFT, ENCAB130GEM, ENCAB369JSU, ENCAB003LHL, ENCAB000ANQ, ENCAB679IZV, ENCAB048FFK, ENCAB000AUR, ENCAB000APW, ENCAB000APV, ENCAB000APY, ENCAB000APU, ENCAB000AXW, ENCAB000APX, ENCAB000ANX, ENCAB000ANY, ENCAB000ATI, ENCAB000AQS, ENCAB000ARG, ENCAB000ARF, ENCAB972UJU, ENCAB027NDF, ENCAB343QJE, ENCAB000ANZ, ENCAB000AUS, ENCAB000AQV, ENCAB629MIV, ENCAB000AQI, ENCAB000BKS, ENCAB000ASY, ENCAB000AOU, ENCAB000BSK, ENCAB721ICQ, ENCAB343GLF, ENCAB749NPH, ENCAB943WPC, ENCAB661VDQ, ENCAB101KHB, ENCAB974EBC, ENCAB372RPK, ENCAB502OHI, ENCAB557LLB, ENCAB088TFM, ENCAB037IXK, ENCAB003HJF, ENCAB793BZS, ENCAB228OWC, ENCAB000ADS, ENCAB654QHT, ENCAB000AQM, ENCAB137OAB, ENCAB000AQN, ENCAB000APD, ENCAB000APF, ENCAB000APE, ENCAB000APC, ENCAB000ANA, ENCAB000BKR, ENCAB000BSI, ENCAB749UMK, ENCAB638ANC, ENCAB813FEB, ENCAB492DPX, ENCAB346FTT, ENCAB420YAH, ENCAB716RFU, ENCAB382AVR, ENCAB367DWC, ENCAB413RSR, ENCAB000AOP, ENCAB000ADY, ENCAB000ASO, ENCAB000AUX, ENCAB000ANF, ENCAB000BSJ, ENCAB725RFE, ENCAB610CEF, ENCAB008SYM, ENCAB170RJO, ENCAB582RSV, ENCAB385IEP, ENCAB081ENJ, ENCAB902NZL, ENCAB848NER, ENCAB682XRE, ENCAB388GOH, ENCAB884CKI, ENCAB000ARL, ENCAB008TOZ, ENCAB513PLB, ENCAB000ARB, ENCAB000ARO, ENCAB000ARC, ENCAB000ARA, ENCAB000ARK, ENCAB000ASG, ENCAB000AUU, ENCAB000ARM, ENCAB000ARN, ENCAB140BWE, ENCAB000ANU, ENCAB000AQR, ENCAB000AAA, ENCAB000ANL, ENCAB000APM, ENCAB000APL, ENCAB000ANG, ENCAB000ATA, ENCAB000AUV, ENCAB000APN, ENCAB000ANV, ENCAB000BKU, ENCAB000BKY, ENCAB000BLG, ENCAB000BLD, ENCAB000BLJ, ENCAB000BLH, ENCAB000BLE, ENCAB000BLI, ENCAB000BLF, ENCAB874PYE, ENCAB237XGS, ENCAB261POO, ENCAB576XIU, ENCAB851GAY, ENCAB000AOX, ENCAB000ANM, ENCAB000ANK, ENCAB000ANN, ENCAB000ANO, and ENCAB000ARV.

### Barcoded Polynucleotides

In some embodiments, the methods relate to contacting a sample with at least one set of barcoded polynucleotides. In some embodiments, the methods relate to contacting a sample with at least two sets of barcoded polynucleotides. In some embodiments, the number of unique barcoded polynucleotides in a set corresponds to the number of channels on a microfluidic chip. Therefore, in various embodiments, a set of barcoded polynucleotides comprises 5 to 1000 unique barcode sequences.

Non-limiting examples of barcoded polynucleotides (e.g., barcoded DNA) of the present disclosure a provided in Example 7. In some embodiments, barcoded polynucleotides (e.g., of a first set of barcoded polynucleotides) include two ligation linker sequences, and a spatial barcode sequence, wherein the spatial barcode sequence is flanked on either side by a ligation linker sequence. In some embodiments, barcoded polynucleotides (e.g., of a second set of barcoded polynucleotides) include a ligation linker sequence, a spatial barcode sequence, and a sequence complementary to a PCR primer.

In one exemplary embodiment, for use with a microfluidic chip comprising 50 microchannels, a set of barcoded polynucleotides comprises 50 barcoded polynucleotides. Exemplary sets of 50 barcoded polynucleotides comprise set “A” barcodes of Example 7, comprising SEQ ID NO: 1-SEQ ID NO:50. In one exemplary embodiment, for use with a microfluidic chip comprising 50 microchannels, a second set of barcoded polynucleotides comprises set “B” barcodes of Example 7, comprising SEQ ID NO:51-SEQ ID NO:100.

A ligation linker sequence is any sequence complementary to a sequence of a ligation adaptor sequence or universal ligation linker, as provided herein. The length of a ligation linker sequence may vary. For example, a ligation linker sequence may have a length of 5 to 50 nucleotides (e.g., 5 to 40, 5 to 30, 5 to 20, 5 to 10, 10 to 50, 10 to 40, 10 to 30, or 10 to 20 nucleotides). In some embodiments, a ligation linker sequence may have a length of 5, 10, 15, 20, 25, 30, 35, 40, 45, or 50 nucleotides. Longer ligation linker sequences are contemplated herein. In some embodiments, a ligation linker sequence of a barcoded polynucleotide of one set (e.g., a first set) differ (e.g., have a different composition of nucleotides and/or a different length) from a ligation linker sequence of a barcoded polynucleotide of another set (e.g., a second set).

A barcode sequence is a unique sequence that can be used to distinguish a barcoded polynucleotide in a biological sample from other barcoded polynucleotides in the same biological sample. A spatial barcode sequence is a barcode sequence that is associated with a particular location in a biological sample (e.g., a tissue section mounted on a slide). The concept of “barcodes” and appending barcodes to nucleic acids and other proteinaceous and non-proteinaceous materials is known to one of ordinary skill in the art (see, e.g., Liszczak G et al. Angew Chem Int Ed Engl. 2019 Mar. 22; 58(13):4144-4162). Thus, it should be understood that the term “unique” is with respect to the molecules of a single biological sample and means “only one” of a particular molecule or subset of molecules of the sample. Thus, a “pixel” (also referred to as a “patch) comprising a unique spatially addressable barcoded conjugate (or a unique subset of spatially addressable barcoded conjugates) is the only pixel in the sample that includes that particular unique barcoded polynucleotide (or unique subset of barcoded polynucleotides), such that the pixel (and any molecule(s) within the pixel) can be identified based on that unique barcoded conjugate (or a unique subset of barcoded conjugates).

For example, in some embodiments, the polynucleotides of subset A1 (of Barcode A) are coded with a specific barcode sequence, while the polynucleotides of subsets A2, A3, A4, etc. are each coded with a different barcode sequence, each barcode specific to the subset. Likewise, the polynucleotides of subset B1 (of Barcode B) are coded with a specific barcode sequence, while the polynucleotides of subsets B2, B3, B4, etc. are each coded with a different barcode sequence, each barcode specific to the subset. Thus, each overlapping patch, which includes a unique combination of Barcode A subsets and Barcode B subsets, contains a unique composite barcode (Barcode A+Barcode B). For example, an overlapping pixel (patch) containing A1+B1 barcodes is uniquely coded relative to its neighboring overlapping patches, which contain A2+B1 barcodes, A1+B2 barcodes, A2+B2 barcodes, etc.

The length of a spatial barcode sequence may vary. For example, a spatial barcode sequence may have a length of 5 to 50 nucleotides (e.g., 5 to 40, 5 to 30, 5 to 20, 5 to 10, 10 to 50, 10 to 40, 10 to 30, or 10 to 20 nucleotides). In some embodiments, a spatial barcode sequence may have a length of 5, 10, 15, 20, 25, 30, 35, 40, 45, or 50 nucleotides. Longer spatial barcode sequences are contemplated herein.

Exemplary barcode sequences that can be added to a nucleic acid molecule according to the method of the invention include, but are not limited to, a nucleic molecule comprising a nucleotide sequence of SEQ ID NO: 1-100. In one embodiment, the method includes adding a first “A” barcode sequence and a second “B” barcode sequence. In one embodiment, the “A” barcode sequence comprises a nucleotide sequence of SEQ ID NO: 1-50, and the “B” barcode sequence comprises a nucleotide sequence of SEQ ID NO: 51-100.

In one embodiment, the method of the invention further comprises contacting the sample with one or more additional barcode sequence (e.g., a “zone” barcode sequence to distinguish specific regions or “zones” of a larger surface.) Therefore, in various embodiments, the methods include sequential ligation of at least one, two, three, four, five, or more than five unique barcode sequences to a target nucleic acid molecule. In one embodiment, each barcoded polynucleotide set comprises at least 10 barcoded polynucleotides.

### Universal Ligation Linkers

Also provided herein are universal ligation linkers, which may be a polynucleotide, for example, that includes (i) a first nucleotide sequence that is complementary to and/or binds to the linker sequence of the barcoded polynucleotides of a first set of barcoded polynucleotides, and (ii) a second nucleotide sequence that is complementary to and/or binds to the linker sequence of the barcoded polynucleotides of a second set of barcoded polynucleotides. The purpose of the universal ligation linkers is to serve as a bridge to join barcoded polynucleotides from two different sets (e.g., the first set comprising two ligation linker sequences flanking a spatial barcode sequence, and the second set comprising a ligation linker sequence, a spatial barcode sequence, and a sequence complementary to a PCR primer). The length of a universal ligation linker may vary. For example, a universal ligation linker may have a length of 10 to 100 nucleotides (e.g., 10 to 90, 10 to 80, 10 to 70, 10 to 60, 10 to 50, 10 to 40, 10 to 30, 10 to 20, 20 to 100, 20 to 90, 20 to 80, 20 to 70, 20 to 60, 20 to 50, 20 to 40, or 20 to 30 nucleotides). In some embodiments, a universal ligation linker may have a length of 10, 15, 20, 25, 30, 35, 40, 45, 50, 55, 60, 65, 70, 75, 80, 85, 90, 95, or 100 nucleotides. Longer universal ligation linkers are contemplated herein.

The universal ligation linkers are typically added to a biological sample following the delivery of aset of barcoded polynucleotides, although, in some embodiments, universal ligation linkers are annealed to the barcoded polynucleotides prior to delivery.

In some embodiments, the ligation adapter or universal ligation linker added to the 5′ and/or 3′ end of a nucleic acid during the method of the invention includes, but are not limited to, a nucleic molecule comprising a nucleotide sequence of SEQ ID NO: 103 or SEQ ID NO:104, or a fragment thereof. In some embodiments, the ligation adapter or universal ligation linker added to the 5′ and/or 3′ end of a nucleic acid during the method of the invention includes, but are not limited to, a nucleic molecule for hybridization to a nucleotide sequence of SEQ ID NO: 103 or SEQ ID NO: 104, or a fragment thereof.

### Methods

In some embodiments, the methods comprise delivering to a biological tissue a first set of barcoded polynucleotides. A first set may include any number of barcoded polynucleotides. In some embodiments, a first set include 5 to 1000 barcoded polynucleotides. For example, a first set may comprise 5 to 900, 5 to 800, 5 to 700, 5 to 600, 5 to 500, 5 to 400, 5 to 300, 5 to 200, 5 100, 10 to 1000, 10 to 900, 10 to 800, 10 to 700, 10 to 600, 10 to 500, 10 to 400, 10 to 300, 10 to 200, 20 to 1000, 20 to 900, 20 to 800, 20 to 700, 20 to 600, 20 to 500, 20 to 400, 20 to 300, 20 to 200, 50 to 1000, 50 to 900, 50 to 800, 50 to 700, 50 to 600, 50 to 500, 50 to 400, 50 to 300, or 50 to 200 barcoded polynucleotides. More than 1000 barcoded polynucleotides in a first set are contemplated herein.

In some embodiments, the method further includes a step of permeabilization prior to delivering the first set of barcoded polynucleotides, for example, through the first microfluidic device. Thus, in some embodiments, the methods comprise delivering to a biological tissue permeabilization reagents (e.g., detergents such as Triton-X 100 or Tween-20). In some embodiments, the methods comprise delivering to a biological tissue a first set of barcoded polynucleotides, and then delivering to the biological tissue permeabilization reagents.

In some embodiments, the methods comprise delivering to the biological sample a second set of barcoded polynucleotides. A second set may include any number of barcoded polynucleotides. In some embodiments, a second set include 5 to 1000 barcoded polynucleotides. For example, a first set may comprise 5 to 900, 5 to 800, 5 to 700, 5 to 600, 5 to 500, 5 to 400, 5 to 300, 5 to 200, 5 100, 10 to 1000, 10 to 900, 10 to 800, 10 to 700, 10 to 600, 10 to 500, 10 to 400, 10 to 300, 10 to 200, 20 to 1000, 20 to 900, 20 to 800, 20 to 700, 20 to 600, 20 to 500, 20 to 400, 20 to 300, 20 to 200, 50 to 1000, 50 to 900, 50 to 800, 50 to 700, 50 to 600, 50 to 500, 50 to 400, 50 to 300, or 50 to 200 barcoded polynucleotides. More than 1000 barcoded polynucleotides in a second set are contemplated herein.

In some embodiments, the methods comprise joining barcoded polynucleotides of the first set to barcoded polynucleotides of the second set. In some embodiments, the methods comprise exposing the biological sample to a ligation reaction, thereby producing a two-dimensional array of spatially addressable barcoded conjugates bound to molecules of interest, wherein the spatially addressable barcoded conjugates comprises a unique combination of barcoded polynucleotides from the first set and the second set.

In some embodiments, the methods comprise imaging the biological sample to produce a sample image. An optical microscope or a fluorescence microscope, for example, may be used to image the sample.

### Sequencing

In some embodiments, the methods include a sequencing step. For example, next generation sequencing (NGS) methods (or other sequencing methods) may be used to sequence the nucleic acid molecules recovered following cell lysis. In some embodiments, the methods comprise preparing an NGS library in vitro. Thus, in some embodiments, the methods comprise sequencing the library of barcoded nucleic acid molecules to produce sequencing reads. Other sequencing methods are known, and an example protocol is provided herein.

In some embodiments, the methods comprise constructing a spatial epigenomic map of the biological sample by matching the spatially addressable barcoded conjugates to corresponding sequencing reads. In some embodiments, the methods comprise identifying the location of the molecules of interest by correlating the spatial epigenomic map to the sample image.

### Multi-Omic Methods

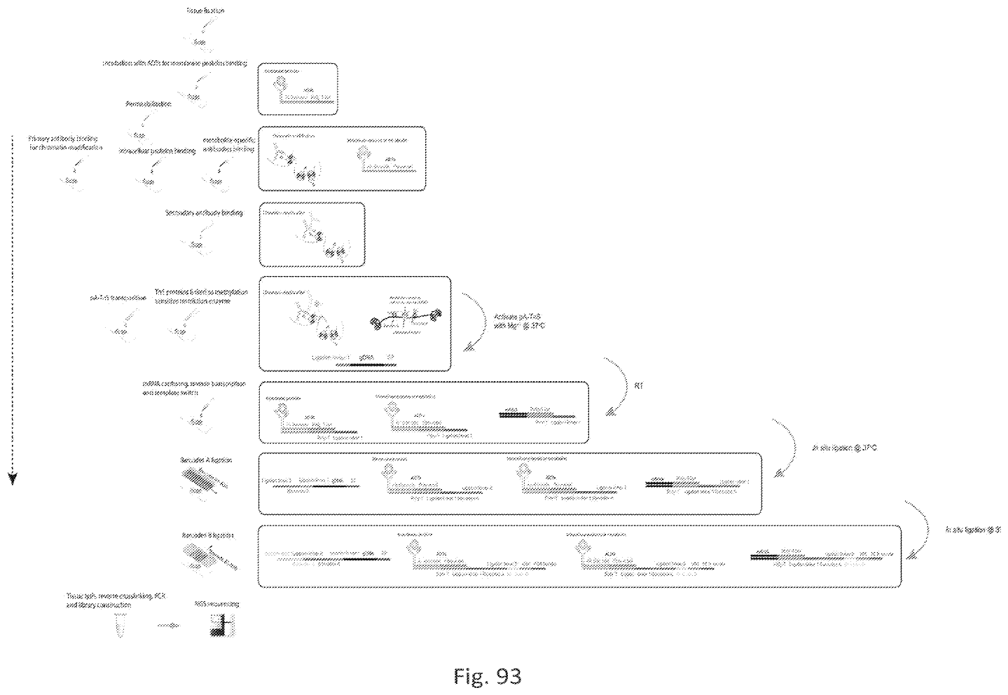

In some embodiments, the spatial epigenomic mapping combined with one or more additional spatial-omic mapping method, including, but not limited to spatial protein or spatial RNA analysis. Exemplary additional spatial-omics methods that can be incorporated with the methods of the invention include, but are not limited to, those described in U.S. patent application Ser. No. 17/036,401 and in Liu et al, 2020, Cell, 183(6): 1665-1681 each of which is incorporated by reference herein in its entirety. FIG. 93 provides a detailed experimental workflow for the combination of spatial epigenomic mapping combined with spatial protein or RNA analysis.

### HSR Microfluidic-Based Systems

To achieve high spatial resolution in a biological context, a detector (e.g., microfluidic device) should profile single cells and resolve spatial features small enough to meaningfully image patterns in the spatial arrangement of single cells and groups of cells. An exemplary high spatial resolution microfluidic based system that can be utilized for the methods of the invention is described in detail in U.S. patent application Ser. No. 17/036,401 and in Liu et al, 2020, Cell, 183(6): 1665-1681 each of which is incorporated by reference herein in its entirety.

Single-Cell Resolution. A detector can profile single cells if the detectors' pixels are of approximately equal or smaller size than the cells. Given mammalian cell sizes that range from approximately 5-20 microns (μm) in length, this entails utilizing a detector with pixels of approximately the same length. Although cell sizes vary within samples, and some cells may be larger and some smaller than detector pixels with a constant size, the inventors have found that by combining optical imaging with digital spatial reconstruction they can select those pixels that circumscribe a single cell in order to achieve true single-cell resolution, even if only for subset of a reconstructed image.

Imaging Multicellular Motifs. In addition to profiling individual cells, it is also useful to consider the ability of an imaging detector to resolve spatial features as being determined by the center-center distance between imaging pixels. This perspective becomes more relevant when examining structures or motifs comprising groups of cells rather than individual cells, such as developing organoids in mouse embryos, as shown in the Examples provided herein.

The standard criterion used in data processing in both the time and spatial domains is the Nyquist Criterion, which dictates that given a center-center distance of a certain number of microns, a detector can faithfully reproduce imaged spatial features only down to approximately twice that center-center distance. Given mammalian cell sizes that range from approximately 5-20 μm and that typically neighbor each other face-to-face, features of cell neighborhoods should vary over distances equal to one or more cell lengths. Thus, to resolve these features, a the HSR detector provided herein, in some embodiments, includes pixels with center-center distance between pixels of not more than several cell lengths, e.g., 10-50 μm.

Imaging systems with pixel sizes and center-center distances much larger than these values cannot profile single cells or resolve features characteristic of cells or multicellular features and therefore do not display HSR. For example, a detector with pixels with size of 1 millimeter would probe distance scales of size 1-2 mm or larger and would not resolve single cells or multicellular features. As the present disclosure described elsewhere herein, pixels much smaller than this range (e.g., less than one micron) result in unsuitable detectors because their mappable area becomes extremely small and logistical tasks (including reagent loading and delivery) become impractical to carry out. The inventors have found that there is a critical range for high-throughput HSR detection with channel width and pitch (near the region of interest) between approximately 2.5-50 μm, for example.

### Microfluidic Devices

Microfluidic devices (e.g., chips) may be used, in some embodiments, to deliver barcoded polynucleotides to a biological sample in a spatially defined manner. A system based on crossed microfluidic channels, such as those described here, have several key parameters that largely determine the spatial resolution and mappable area of the device. These include (1) the number of microfluidic channels (η/eta); (2) the microchannel width (ω/omega), measured in microns, i.e., the width of the open space in each microfluidic channel (tissue beneath these open spaces is imaged); and (3) microchannel pitch (Δ/delta), measured in microns, i.e., the width of the closed space between the end of one channel and the start of another channel (tissue beneath these closed spaces is not imaged).

In some embodiments, the microfluidic devices provided herein include multiple microchannels characterized by a certain width, depth, and pitch. In some embodiments, the microfluidic devices of the invention achieve high spatial resolution at the single-cell level.

In one embodiment, the system of the invention comprises two microfluidic devices. For example, in one embodiment, a first device flows reagents left to right and is drawn as a series of rows, and a second device flows reagents from top to bottom and is drawn as a series of columns. The pixels of the detector comprise the overlap areas between the two sets of shapes, and as can be seen in the drawing such a geometry endows the squares with edge length ω microns. As an illustrative example, assume a detection scheme that utilizes microfluidic devices with η=50, ω=10 microns, and Δ=10 microns. In some embodiments, the detector will feature pixels that are squares with edge length 10 microns, and the distance between squares in the horizontal and vertical directions is equal to 20 microns. This means it can profile single cells that are approximately 10 microns or larger and resolve spatial features (e.g., characteristics of cell neighborhoods) that are 40 microns or larger. In some embodiments, such microfluidic-based detectors will display certain performance characteristics determined by the design and the design parameters, including, but not limited to, the ability to profile individual cells; a minimum length scale of spatial feature reproduction; and the size of the mappable area.

Number of microchannels. In some embodiments, a first set of barcoded polynucleotides is delivered through a first microfluidic chip that comprises parallel microchannels positioned on a surface of the biological sample. In some embodiments, a first microfluidic chip comprises at least 5, at least 10, at least 20, at least 30, at least 40, or at least 50 parallel microchannels. In some embodiments, a first microfluidic chip comprises 5, 10, 20, 30, 40, or 50 parallel microchannels. In some embodiments, a first microfluidic chip comprises 5-1000 parallel microchannels (e.g., 5-10, 5-25, 5-50, 5-75, 10-25, 10-50, 10-75, 10-1000, 25-500, 25-200, 25-100, 50-200, or 50-100 parallel microchannels). In some embodiments, a second set of barcoded polynucleotides is delivered through a second microfluidic chip that comprises parallel microchannels that are positioned on the biological sample perpendicular to the direction of the microchannels of the first microfluidic chip. In some embodiments, a second microfluidic chip comprises at least 5, at least 10, at least 20, at least 30, at least 40, or at least 50 parallel microchannels. In some embodiments, a second microfluidic chip comprises 5-1000 parallel microchannels (e.g., 5-10, 5-25, 5-50, 5-75, 10-25, 10-50, 10-75, 10-1000, 25-500, 25-200, 25-100, 50-200, or 50-100 parallel microchannels).

Microchannel width. In some embodiments, a microchannel has a width of at least 5 μm (e.g., at least 5 μm, at least 10 μm, at least 15 μm, at least 20 μm, at least 25 μm, at least 30 μm, at least 35 μm, at least 40 μm, or at least 50 μm). In some embodiments, a microchannel has a width of 10 μm, 15 μm, 20 μm, 25 μm, 30 μm, 35 μm, 40 μm, 50 μm or more than 50 μm. In some embodiments, a microchannel has a width of 5 μm to 1000 μm (e.g., 10-500 μm, 10-100 μm, 20-200 μm, 20-100 μm).

In some embodiments, the microchannels have variable width. Variable channel width eases fluid flow through the microfluidic channels. For example, in one embodiment, a 50 μm device features 100 μm channels which shrink to 50 μm only near the region of interest. As another example, a 20 μm device's channels shrink to 100, 50, and then 20 μm near the region of interest. As yet another example, a 10 μm device's channels range from 100, 50, 25, and then 10 μm near the region of interest.

In some embodiments, a microchannel has a width of 20 μm to 1000 μm near the inlet and outlet ports and a width of 5 μm to 100 μm near the region of interest. For example, a microchannel may have a width of 100 μm near the inlet and outlet ports and width of 50 μm near the region of interest. As another example, a microchannel may have a width of 100 μm near the inlet and outlet ports and width of 20 μm near the region of interest. In some embodiments, a microchannel has a width of 50, 60, 70, 80, 90, 100, 110, 120, 130, 130, 140, or 150 μm near the inlet and outlet ports. In some embodiments, a microchannel has a width of 10, 20, 30, 40, or 50 μm near the region of interest.

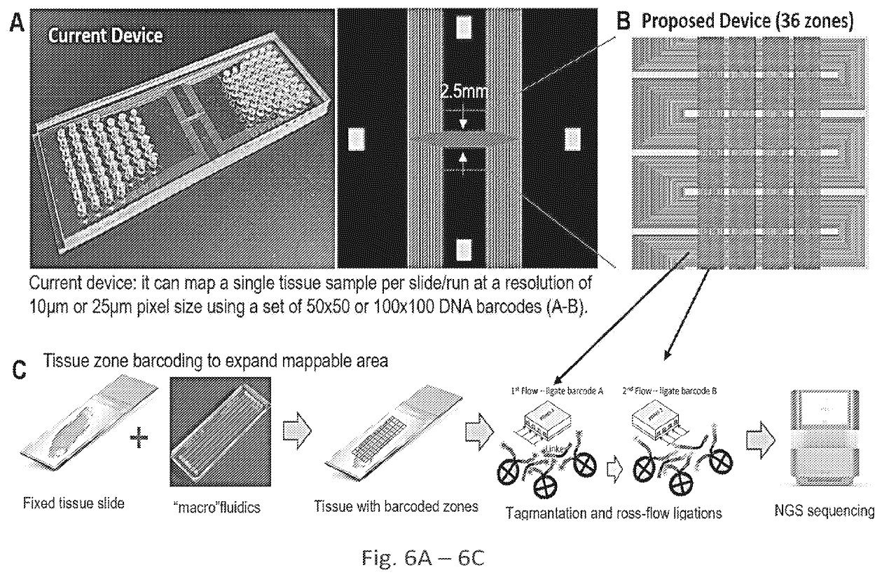

In some embodiments, the microchannels are serpentine, allowing for the fluid to flow back and forth across a sample in a pattern (see e.g., FIG. 6B). Use of serpentine microchannels can be used to apply a specific barcode sequence in a repeated pattern across a sample. In some embodiments a serpentine microfluidic device is combined with a non-serpentine microfluidic device which flows a second set of barcodes in a straight pattern and a third method of applying barcodes to specific non-overlapping zones, such that each tixel comprises a unique set of barcodes.

Microchannel height. In one embodiment, the microchannel height is approximately equal (e.g., within 10%) to the microchannel width. In some embodiments, a microchannel has a height of at least 10 μm (e.g., at least 15 μm, at least 20 μm, at least 25 μm, at least 30 μm, at least 35 μm, at least 40 μm, or at least 50 μm). In some embodiments, a microchannel has a height of 10 μm, 15 μm, 20 μm, 25 μm, 30 μm, 35 μm, 40 μm, or 50 μm). In some embodiments, a microchannel has a height of 10 μm to 150 μm (e.g., 10-125 μm, 10-100 μm, 25-150 μm, 25-125 μm, 25-100 μm, 50-150 μm, 50-125 μm, or 50-100 μm). These heights have been tested and shown to be sufficient to provide clearance above dust or tissue blockages, for example, and low enough to provide good sufficient rigidity and to prevent deformation of the channel during clamping and flow.

In some embodiments, a microchannel has a width of 10 μm and a height of 12-15 μm. In other embodiments, a microchannel has a width of 25 μm and a height of 17-22 μm. In yet other embodiments, a microchannel has a width of 50 μm and a height of 20-100 μm.

Microchannel pitch. The pitch is the distance between microchannels of a microfluidic device (e.g., chip). In some embodiments, the pitch of a microfluidic device is at least 10 μm (e.g., at least 15 μm, at least 20 μm, at least 25 μm, at least 30 μm, at least 35 μm, at least 40 μm, or at least 50 μm). In some embodiments, the pitch of a microfluidic device is at 10 μm, 15 μm, 20 μm, 25 μm, 30 μm, 35 μm, 40 μm, or 50 μm. In some embodiments, the pitch of a microfluidic device is at 10 μm to 150 μm (e.g., 10-125 μm, 10-100 μm, 25-150 μm, 25-125 μm, 25-100 μm, 50-150 μm, 50-125 μm, or 50-100 μm).

### Negative Pressure Systems

Many microfluidics platforms utilize positive pressure via syringe pumps, peristaltic pumps, and other types of positive pressure pumps whereby fluid is pumped from a reservoir into the device. Generally, a connection is made to interface the reservoir/pump assembly with the microfluidic device; often this takes the form of tubes terminating in pins that plug into inlet ports on the device. However, this type of system requires laborious and time-consuming fine-tuning of the assembly process associated with several drawbacks. For example, if the pins are inserted insufficiently deep into the inlet wells or the pin diameter is too small relative to the ports, then upon activation of the pumps, fluid pressure will eject the tube from the port. As another example, if the pins are inserted excessively deep into the wells, then upon activation of the pumps, fluid pressure will separate the microfluidic device from the glass substrate, resulting in leakage. While epoxying pins into ports and/or bonding the microfluidic device to the substrate via plasma bonding or thermal bonding might address the foregoing drawbacks, these strategies make it difficult to disassemble the system in a non-destructive way, resulting in component loss and are impractical when the substrate contains sensitive material, such as a tissue section, and/or antibodies.

The methods and devices provided herein, by contrast, overcome the drawbacks associated with existing microfluidic platforms by using, in some embodiments, a negative pressure system that utilizes a vacuum to pull liquid through the device from the back, rather than positive pressure to push it through the device from the front. This has several advantages, including, for example, (i) reducing the risk of leakage by pulling together the device and substrate and (ii) increasing efficiency and ease of use—the vacuum can be applied to all outlet ports, unlike pins, which must be inserted individually into each inlet port. Using a negative pressure system saves several hours per run of fine-tuning and pin assembly.

Thus, in some embodiments provided herein, the barcoded polynucleotides are delivered to a region of interest through a microfluidic device (e.g., chip) using negative pressure (vacuum). In some embodiments, delivery of a first set of barcoded polynucleotides is delivered through a first microfluidic device using a negative pressure system. In some embodiments, delivery of a second set of barcoded polynucleotides is delivered through a second microfluidic device using a negative pressure system.

### Inlet and Outlet Ports

In some embodiments the microfluidic devices having a common outlet port are vulnerable to backflow of reagents into the region of interest through incorrect microchannels, particularly during device disassembly. Such backflow can result in incorrect addressing of target molecules, resulting in an incorrect reconstruction of a spatial map of target molecules performed in later steps of the methods (e.g., after sequencing). To limit the possibility of reagent backflow, the microfluidic devices provided herein, in some embodiments, include microchannels that each have its own inlet port and outlet port. For example, in one embodiment, a microchannel device comprising 50 microchannels has 50 inlet ports and 50 outlet ports. In one embodiment, a microchannel device comprising 100 microchannels has 100 inlet ports and 100 outlet ports.

### Clamping

During initial experiments used to test the microfluidic devices and methods provided herein, frequent leakage of reagents occurred between channels on the region of interest. Convention clamping mechanisms proved cumbersome and introduced difficulties in addressing inlet and outlet ports. To address the issues identified, a new clamping mechanism was developed, which combines specific clamping parameters including localized clamping and specific clamping forces. A range of clamping forces was investigated—in some instances, the clamping force was insufficient to prevent leaks, and in other cases the clamping force was so great that flow was significantly reduced or even stopped entirely in some or all microchannels. Without being bound by theory, it was though that the was due to the channel cross section being deformed by the clamping force, reducing the cross-sectional area and making the channels more vulnerable to blockages due, for example, either to dust or the tissue occupying the entire microchannel.

Microfluid chips, in some embodiments, are fabricated from polydimethylsiloxane (PDMS). Other substrates may be used.

### Samples

In some embodiments, a sample is a biological sample. Non-limiting examples of biological samples include tissues, cells, and bodily fluids (e.g., blood, urine, saliva, cerebrospinal fluid, and semen). The biological sample may be adult tissue, embryonic tissue, or fetal tissue, for example. In some embodiments, a biological sample is from a human or other animal. For example, a biological sample may be obtained from a murine (e.g., mouse or rat), feline (e.g., cat), canine (e.g., dog), equine (e.g., horse), bovine (e.g., cow), leporine (e.g., rabbit), porcine (e.g., pig), hircine (e.g., goat), ursine (e.g., bear), or piscine (e.g., fish). Other animals are contemplated herein.

In some embodiments, a biological sample is fixed, and thus is referred to as a fixed biological sample. Fixation (e.g., tissue fixation) refers to the process of chemically preserving the natural state of a biological sample, for example, for subsequent histological analysis. Various fixation agents are routinely used, including, for example, formalin (e.g., formalin fixed paraffin embedded (FFPE) tissue), formaldehyde, paraformaldehyde and glutaraldehyde, any of which may be used herein to fix a biological sample. Other fixation reagents (fixatives) are contemplated herein.

In some embodiments, the biological sample is a tissue. In some embodiments, the biological sample is a cell. A biological sample, such as a tissue or a cell, in some embodiments, is sectioned and mounted on a surface, such as a slide. In such embodiments, the sample may be fixed before or after it is sectioned. In some embodiments, the fixation process involves perfusion of the animal from which the sample is collected.

### Kits

Also provided herein are kits for producing a high resolution spatial epigenomic map of a biological sample, for example. In some embodiments, the kits comprise a ligation linker sequence, a first set of barcoded polynucleotides, and a second set of barcoded polynucleotides.

In some embodiments, the kits comprise a (i) a primary antibody that specifically binds to an epigenomic marker of interest, (ii) a secondary antibody and (iii) a protein A tethered transposase. In one embodiment the protein A tethered transposon is preloaded with a ligation adaptor sequence.

In some embodiments, the kits comprise at least one reagent selected from tissue fixation reagents, reverse transcription reagents, ligation reagents, polymerase chain reaction reagents, template switching reagents, and sequencing reagents.

In some embodiments, the kits comprise tissue slides (e.g., glass slides).

In some embodiments, the kits comprise at least one microfluidic chip that comprises parallel or serpentine microchannels.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: A System for High-Throughput Spatial Epigenomic Mapping

Despite the recent breakthroughs in massively parallel single-cell sequencing that have revolutionized biomedical research, it is becoming increasingly recognized that spatial information of single cells in their tissue context is essential for a true mechanistic understanding of novel biology and disease pathogenesis. However, these associations are often missing in single-cell omics data. A new field “spatial transcriptomics” has emerged to address this challenge with all early attempts based on single-molecule fluorescence in situ hybridization(smFISH). This technique evolved rapidly from detecting a handful of genes to near transcriptome-wide measurement via repeated hybridization and imaging cycles. A recent first-in-class demonstration such as Slide-seq (Rodriques et al.) and HDST (Vickovic et al.) used Next Generation Sequencing (NGS) for unbiased reconstruction of high-spatial-resolution (˜10 μm) transcriptome map. However, to investigate the mechanism underlying spatial organization of different cell types and functions in their tissue context, it is necessary is to examine not only gene expression but also epigenetic underpinnings, such as chromatin accessibility and modification, at single-cell resolution. This capability would enable novel causative mapping of the Central Dogma of Molecular Biology from epigenome to transcriptome and proteome in individual cells with broad implications for how tissues organize, a grand challenge in modern biology.

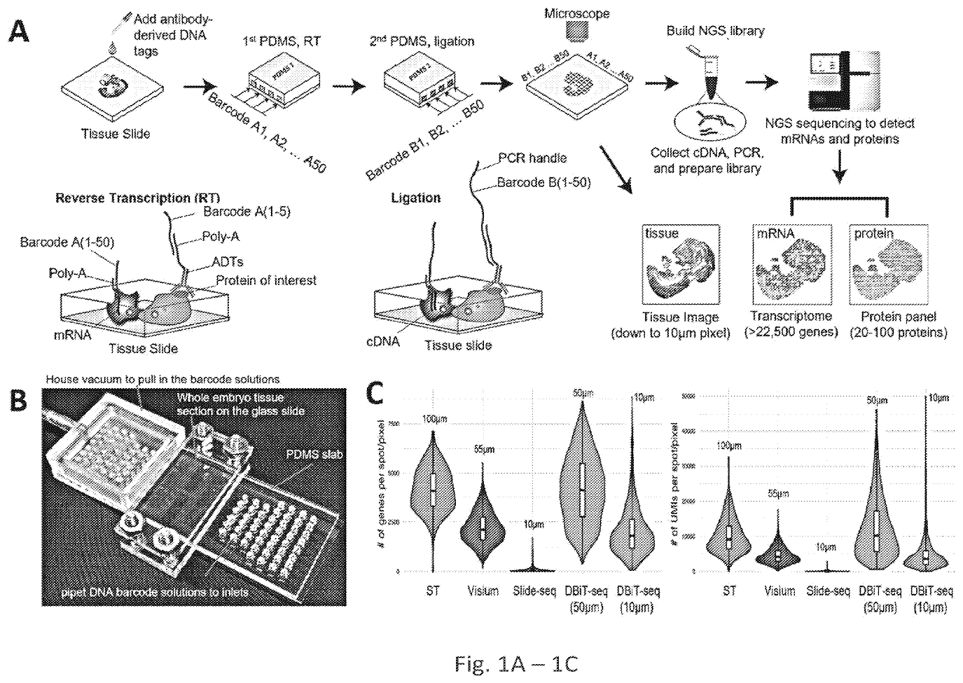

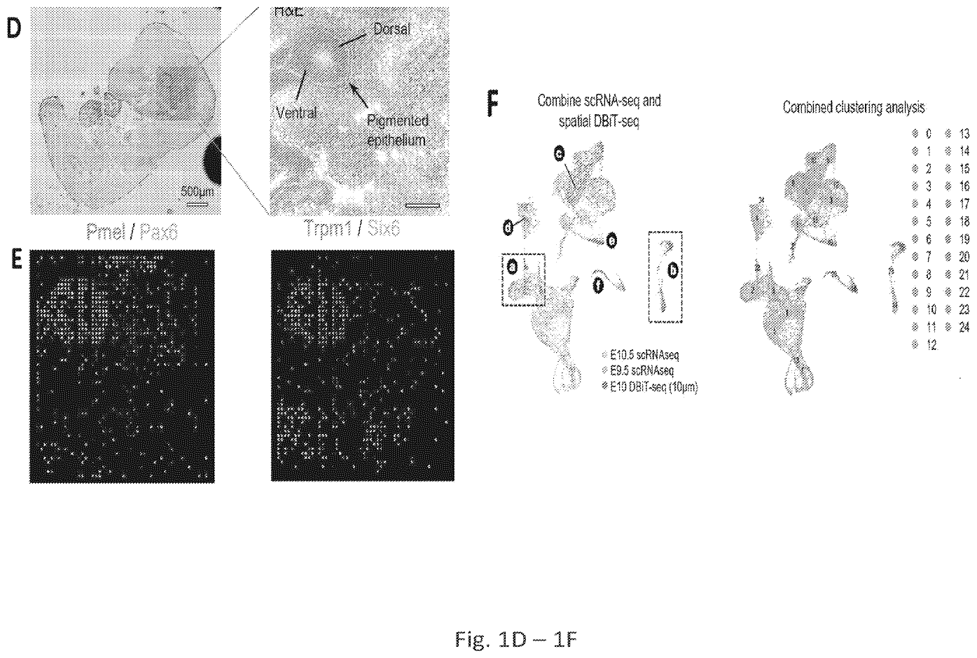

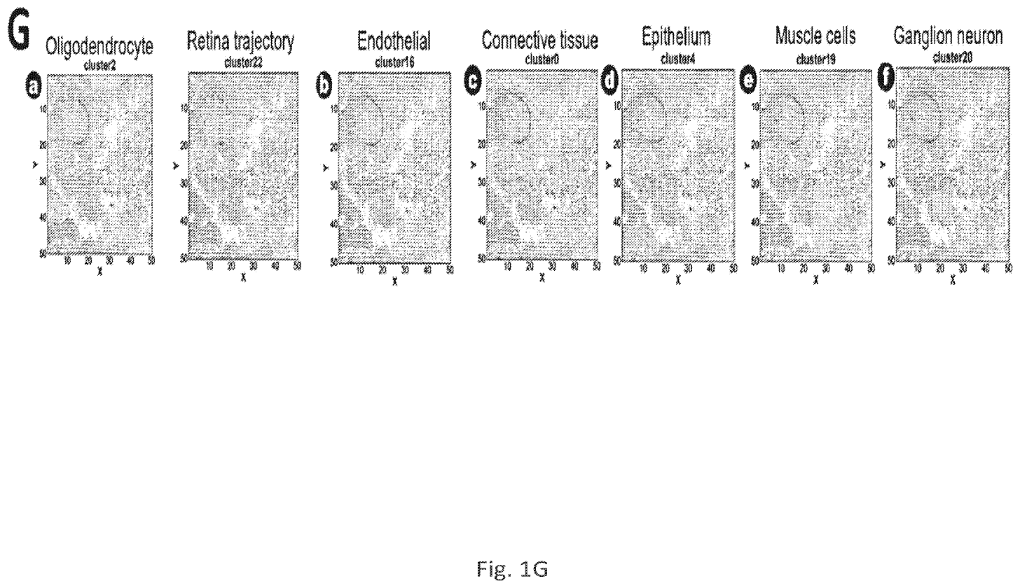

A system for high-throughput spatial epigenomic mapping via major innovations in microfluidics engineering, molecular barcoding, and NextGen sequencing. Recently, microfluidic Deterministic Barcoding in Tissue for spatially resolved sequencing (DBiT-seq) of whole transcriptome and a panel of 22 proteins has been developed at a resolution of ˜10 μm pixel size (FIG. 1) (Liu et al., 2020, Cell, 183:1665-1681). This in-tissue barcoding is unique in that it is highly versatile and enables in situ barcoding of other biomolecular information such as epigenetic states. This in-tissue deterministic barcoding approach was used to develop a novel in situ transposase tagmentation chemistry to realize high-spatial-resolution (˜10 μm) epigenomic and transcriptomic mapping. By imaging the location of individual nuclei in the tissue slide (PFA-fixed or FFPE), the tissue pixels containing single nuclei are unambiguously identified, allowing for single-cell-resolution spatially-resolved epigenomic and transcriptomic mapping using NGS sequencing. To date, NGS-based spatial transcriptomics is still in its infancy. NGS-based epigenomic spatial mapping at single-cell resolution appears to be inaccessible for the near future. However, this technology, if fully realized, could transform multiple biomedical research fields including developmental biology, neuroscience, immunology, oncology, and clinical pathology, thus empowering scientific discovery and translational medicine in human health and disease.

A scheme for deterministic barcoding in tissue for spatially resolved mRNA and protein mapping via a novel microfluidic technique (FIG. 1A), has been developed. Tissue slides are stained with a cocktail of DNA-antibody conjugates similar to single-cell CITE-seq. Subsequently, a polydimethylsiloxane (PDMS) microfluidic chip is placed on the tissue slide using a clamp. A set of barcode oligo solutions are pipetted into the inlets of the chip and pulled in by house vacuum (FIG. 1B). These oligomers contain a poly-T sequence for detecting mRNAs and distinct row barcodes A1 to A50 for spatial identification of co-localized cells. During this first flow barcoding step, reverse transcription is conducted in situ on the tissue to synthesize 1st strand cDNA. Subsequently, the PDMS chip is removed and a second microfluidic chip placed orthogonally in the same position to introduce column barcodes B1 to B50 which are ligated to the first set of barcodes A1 to A50 at the intersections, giving rise to a 2D array of barcoded tissue pixels. Afterwards, barcoded cDNAs are recovered, purified, and PCR amplified to prepare NGS sequencing libraries. Pixels are marked by unique row+column barcodes, resulting in spatial barcodes AiBj (i=1-50, j=1-50) of all pixels and the corresponding transcripts or proteins, that allows to construct a spatial omics map at near single-cell level (10 μm pixel size). DBiT-seq outperforms other emerging spatial RNA-seq techniques, including ST (spot size: 100 μm), 10× Genomics's Visium (55 μm), and Slide-seq (10 μm) (FIG. 1C). Comparable gene counts (>2,000 genes) per spot were shown with Visium, but with much higher spatial resolution (10 μm vs 55 μm). This technique outperformed Slide-seq, which has comparable resolution (10 μm) but suffers low sensitivity (˜150 genes/spot), limiting its capability to profile cell states. DBiT-seq was validated with a PFA-fixed mouse embryo tissue on a region of interest corresponding to the eye field development (FIG. 1D). By assembling the pixels for selected genes like Pme1, Pax6, Trpm1, and Six6, known to be important in embryonic eye development, the spatial expression map was able to differentiate the eye field and, impressively, a single-cell-layer of melanocytes lining the optic vesicle. Computational integration of scRNA-seq and DBiT-seq data allows for accurate cell type identification in spatial pixels and revealed that most spatial tissue pixels (10 μm) are dominated by single-cell transcriptomes (FIG. 1F). Unsupervised clustering analysis revealed 25 cell subsets, each of which can be unambiguously mapped back to spatial expression via integrated computational analysis of single-cell and spatial sequencing data (FIG. 1G). This barcoding approach has demonstrated superiority in spatial transcriptomics. Here, this barcoding strategy is translated to enable fist-of-its-kind spatial epigenomic sequencing, combined with spatial transcriptomic, and is applied to 3D mapping of human lymph nodes in normal physiology and hematologic cancers.

Development of Devices and Chemistry for hsrATAC-Seg at Single-Cell Resolution.

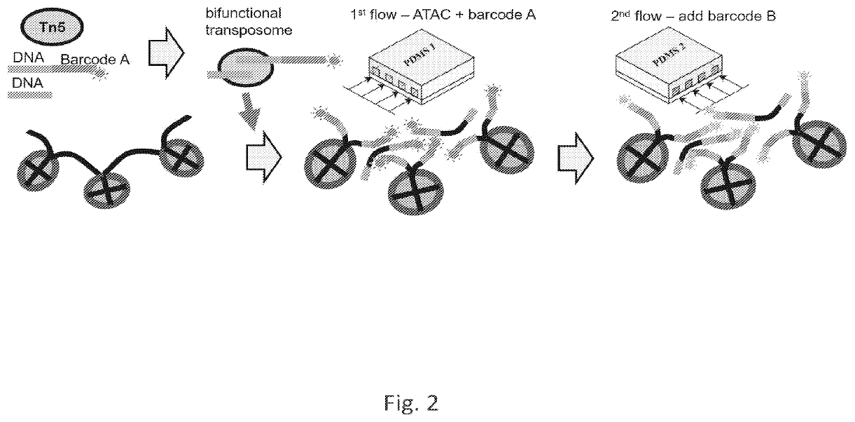

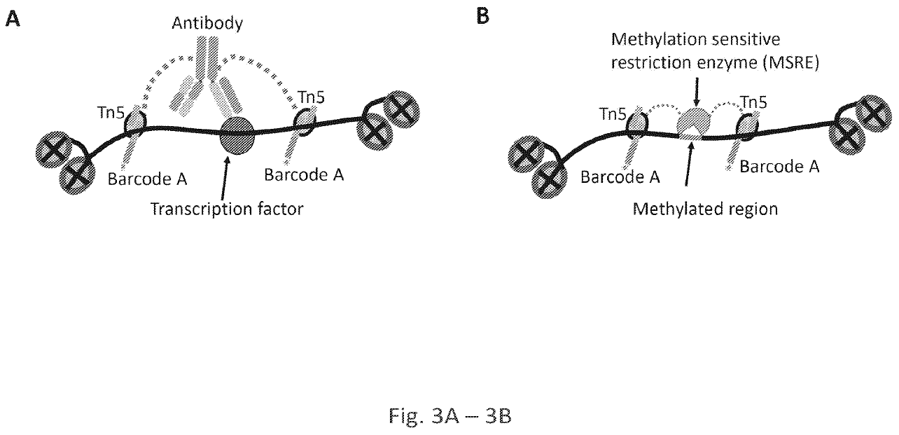

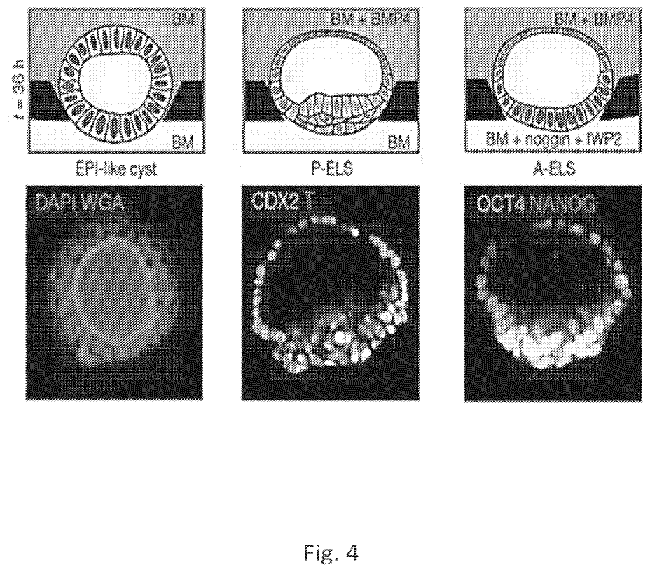

A similar microfluidic cross-flow barcoding device was developed (FIG. 1A) to conduct spatially resolved tissue barcoding with the key question of how to barcode chromatin state or accessibility. Herein a transposome-based DNA tagmentation chemistry as schematically illustrated in FIG. 2 is proposed. Two DNA oligomers were incorporated into the transposase Tn5 with one oligomer linked to DNA barcode sequence Ai(i=1-50). Then, barcode A-incorporated Tn5 enzyme is flowed using a microfluidic channel array chip (channel width: ˜10 μm) to the tissue slide and subsequently barcode B was flowed in the orthogonal direction using a separate microfluidic chip, during which in situ ligation is conducted to yield a 2D array of spatial barcodes AiBj (i=1-100, j=1-100). This barcode is linked to digested genomic DNA strands at the Tn5 transposase cutting sites giving spatial barcodes to exposed DNA strands in fixed tissues. Recently, tissue fixing, cross-linking, and permeabilization was tested by imaging Tn5 cutting sites in situ and a condition was found to permeabilize only nuclear membrane but not mitochondria in tissue specimens. After the entire hsrATAC-seq workflow is performed, the same tissue slide can be used for optical or fluorescence imaging (e.g., DAPI nuclear staining), allowing to precisely correlate nuclear boundaries with the spatial tissue pixels, such that the pixels containing single nuclei can be unambiguously identified. Less than 65% of the pixels (10 μm) in the mouse embryo tissue studied contained single nuclei. Therefore, the data generated in this project is actually a single-cell-resolution spatial ATAC-seq map, with direct supervision of NGS barcodes by true positive single cells as identified via imaging. Recently, the feasibility of the biochemistry workflow was demonstrated, with no spatial barcodes, to perform transposome digestion in tissue and collect recovered DNA fragments for NGS (FIG. 3). Computational analysis revealed that the reads are enriched at the transcription start sites (TSS), which is highly encouraging. A titration experiment was used to optimize Tn5 transposome concentration for optimal cutting efficiency and to adjust the channel with for different tissue types. hsrATAC-seq was validated using a layer of adherent cells (e.g., NIH3T3) cultured on a glass slide and compare hsrATAC-seq to well-validated scATAC-seq databases. hsrATAC-seq was further validated with mouse embryonic tissue sections from E8-E12, for which scATAC-seq data are readily available.

### Spatial Chromatin Modification State Profiling, Spatial CHIP-Seq and Spatial DNA Methylation Sequencing.

The customized Tn5 transposase with DNA-barcode patterning approach served as a basis to develop other spatial epigenetic mapping technologies by modifying the function of Tn5 to recognize different epigenetic features. To map the binding sites of transcription factors (TFs), Tn5 is covalently linked to an antibody against the TF of interest (FIG. 3A) using SANH-SFB coupling reaction. Then, this complex is assembled with barcode A oligomers, deactivated, and flowed through the microfluidic channels to bind TFs in tissue. Afterwards, Tn5 enzymes are reactivated to perform tagmentation to incorporate barcode A at the TF binding region. Finally, barcode B can be added and ligated similar to that in FIG. 2, which gives a full spatial barcode AB to construct the spatial TF binding site map. For spatial methylome sequencing, two Tn5 proteins are linked to a methylation sensitive restriction enzyme (MSRE) (FIG. 3B) and the delivery of this complex binds to DNA methylation sites to enable the profiling of DNA methylation in individual nuclei in tissue. Incorporation of this linked Tn5 technique reconstructs a spatially-resolved single-cell-resolution DNA methylome map. Therefore, this approach enables the spatially resolved mapping of a wide range of epigenetic features at single-cell resolution.

### Mapping Spatial Epigenomic Profiles of Single Cells in Situ in the Tissue Section of Artificial Human Embryo Models.

Embryonic development is a highly dynamic and fast-paced tissue morphogenesis process precisely controlled by epigenetic changes at each stage. Much has been known in mouse embryogenesis via combing the results from different studies over years. However, it remains poorly understood about human embryo development especially in early organogenesis due to ethics regulations and the lack of samples. Recently, artificial embryos derived from human pluripotent stem cells (hPSCs) were reported that recapitulated early embryogenesis using a microfluidic system. In this project, this approach is used to generate artificial human embryos at different time points of early stages (1-4 weeks) and apply the aforementioned high-spatial-resolution epigenomics atlas technologies in conjunction with DBiT-seq that provides matched spatial mRNA & protein data to investigate the spatiotemporal dynamics of human embryonic organogenesis in 3D and at the genome scale. This provides unprecedented insights to improving the understanding of human developmental mechanisms and the relationship between developmental defects, diseases, and potential interventions.

### Example 2: Development and validation of hsrChST-seq

A chemistry workflow has been developed to implement in-tissue barcoding of chromatin using DNA barcode-incorporated Tn5 transposome, which is further tagged to specific antibodies for different histone modifications. It is performed directly on the native tissue sample to yield spatially barcoded tissue pixels followed by NGS to construct a spatial chromatin state map. The technology is validated using mouse embryo tissue samples to compare cell types identified by the hsrChST-seq method vs. those identified by publicly available single-cell sequencing data. It is also validated with cancer cell lines (i.e., GM12878 lymphoblastoid cells) well characterized by the NIH ENCODE consortium.

### Experimental Procedure.

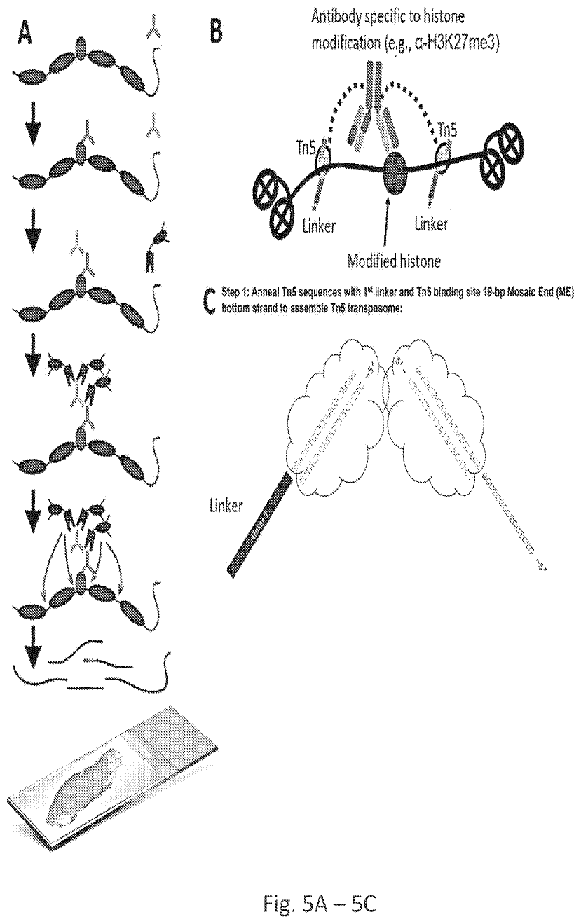

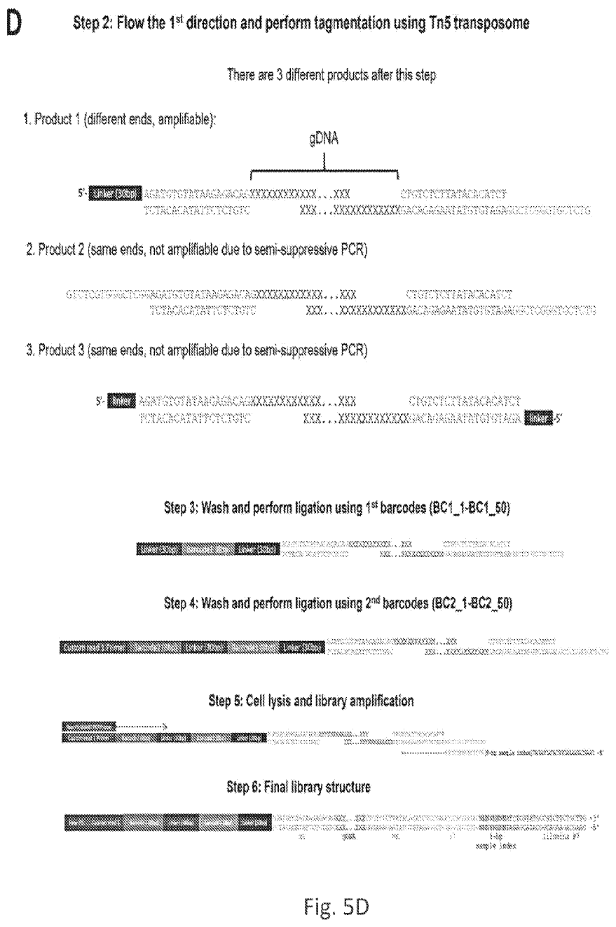

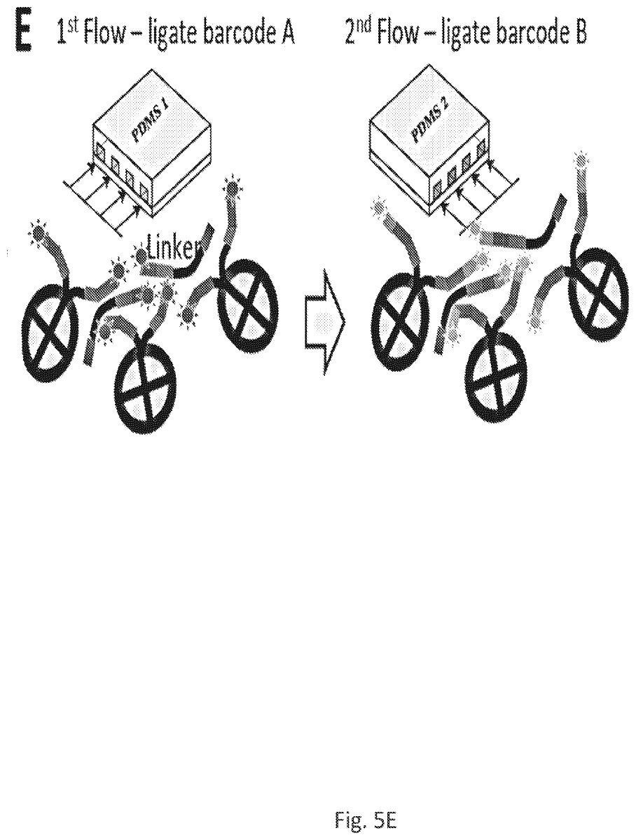

A chromatin cut-and-tag protocol (FIG. 5A) is used to label the tissue in situ with primary antibodies recognizing different histone modifications such as H3K27me3 or H3K4me3. The secondary antibody tethered with transposase Tn5 is assembled with a unique DNA oligo sequence that serves as the ligation linker (FIGS. 5B and 5C). After application of this primary anti-human histone antibody and the modified secondary antibody-Tn5 transposome complex, the whole tissue section is labeled in situ with the ligation linkers at the site of genomic DNA sequence corresponding to the specific histone modifications. The unique in-tissue cross-flow barcoding approach is used to conduct spatially resolved tissue pixels containing spatial DNA barcodes via two flow ligation steps. The first flow incorporates row address DNA barcodes Ai (i=1-50) to the linker sequence through templated ligation. Then, the second flow will introduces a different set of barcodes (the column address DNA barcodes Bi(i=1-50) in the orthogonal direction using a separate microfluidic chip, during which in situ ligation is conducted to yield a 2D array of tissue pixels, each contains a unique spatial barcode AiBj (i=1-100, j=1-100). This barcode sequence is linked to the DNA strands at the Tn5 transposase cutting sites determined by the antibody against a specific chromatin state (e.g. H3K27me3). Finally, the DNA fragments released are sequenced using paired end NGS in Illumina Next-Seq. Read1 corresponds to the spatial address code AiBj and read2 contains the DNA sequence at the site of histone modification of interest. Recently, tissue fixing, cross-linking, and permeabilization by imaging Tn5 cutting sites were tested in situ and a condition to permeabilize only nuclear membrane but not mitochondria in tissue specimens was found. After the entire hsrChST-seq workflow is performed, the same tissue slide can be used for optical or fluorescence imaging (e.g., DAPI nuclear staining), further allowing for precisely correlating nuclear boundaries with the spatial tissue pixels, such that the pixels containing single nuclei can be unambiguously identified. According to a preliminary test, >70% of the pixels (10 μm) in the mouse tissue sample contain single nuclei. Therefore, the data generated is actually a single-cell-resolution spatial chromatin state map. Recently, the Tn5 biochemistry workflow was conducted despite no spatial barcodes applied to the transposome digestion in tissue, and the tagmentated DNA fragments for NGS were collected. Computational analysis revealed that the reads were indeed enriched at the chromatin sites.

### Expansion to Spatial Co-Mapping of Chromatin State and Transcriptome.

In order to link gene expression profile to epigenetic underpinning in individual tissue pixels (tixels) and single cells, the workflows for spatial mRNA mapping is combined with the developed method of hsrChST-seq in a single experiment to realize spatially resolved co-mapping of chromatin epigenome and mRNA transcriptome at the cellular level and in the tissue context. Again, this technology is validated with the tissue samples by comparing cell types identified by single-cell sequencing data from ENCODE.

### Experimental Procedure

The in-tissue barcoding approach is unique in that it does not require prefabricated capture or detection probe array but only use a set of reagents flowed through the microfluidic channels on a tissue slide. Thus, reagents for hsrChST-seq are directly combined with hsrRNA-seq via co-flowing both reagents in the same microfluidic channels to realize spatial epigenome and transcriptome co-sequencing. A method for single-cell level mapping of gene expression in relation to epigenetic states in the tissue context and at the genome scale is thus developed by leveraging the ability to conduct high-resolution optical imaging on the same tissue slide and computational deconvolution of sequencing data. To validate this technology, well characterized E8-E12 mouse embryo tissue sections (PFA and FFPE) are used to perform spatial-omic sequencing and integrate the data with scRNA-seq and scChIP-seq data, both publicly available. This study generates numerous new insights to better understand embryonic development and early organogenesis at an unprecedented level. The customized immuno-tagging and Tn5 transposase with in tissue barcoding serves as a basis for the development of other spatial epigenetic mapping technologies by modifying the function of Tn5 to recognize different epigenetic features. For example, to map the binding sites of transcription factors (TFs), Tn5 is covalently linked to an antibody against the TF of interest using SANH-SFB coupling reaction. Then, this complex is assembled with barcode A oligomers, deactivated, and flowed through the microfluidic channels to bind TFs in tissue. Afterwards, Tn5 enzymes are reactivated to perform tagmentation to incorporate barcode A at the TF binding region. Finally, barcode B is added and ligated similar to that in FIG. 5E, which gives a full spatial barcode AB to construct the spatial transcription factor binding site map in tissue.

Data analyses to connect spatial gene expression to epigenetic modifications are performed with Seurat package (V2.3.0) in R (V3.4.1). It is used to identify differentially expressed genes in single cells. Quality control criteria for clustering analysis include: 1) expression of more than 1,000 genes and fewer than 5,000 genes; 2) low expression of mitochondrial genes (<10% of total counts in a cell). Principal component analysis (PCA) and t-Distributed Stochastic Neighbor Embedding (t-SNE) is used to discover cellular heterogeneity. The Monocle package (V2.6.4) is used to analyze single cell pseudo-time trajectories to examine cell differentiation trajectory and phenotypic transition. For comparison between samples across different developmental stages or different conditions/groups, student t-test is used to assess the correlation. For other comparisons, statistical analysis (one-way ANOVA) with significant difference is assumed for p<0.05. Multivariate linear mixed modeling is also performed with sampling condition adjusted. Akaike information criterion (AIC) and Bayesian information criterion (BIC) are utilized for model selection.

### Large-Area Spatial Mapping of Clinical Tissue Histology Samples.

In order to further increase the mappable area required for clinical histology specimens (centimeter scale) and also increase the sample throughput, reduce the operation time, and the cost per sample, which are all critical to wide-spread adoption of this technology in biomedical and clinical studies, a microfluidic tissue zone barcoding method is developed to significantly increase the mappable area by 10 times to 2 cm×2 cm or to simultaneously analyze ˜96 tissue samples on a tissue microarray slide.

### Experimental Procedures.

In order to further increase throughput and lower the sample preparation time & cost, which is critical to wide-spread adoption in basic and translational research, an approach to barcode “macroscopic” zones of a tissue section is developed, each of which can be analyzed with hsrChST-seq but together cover a much larger area of tissue mapped per experiment. To do so, another ligation step is performed after the cross-flow barcoding of tissue pixels (AiBj, i and j=1-50) such that all “zones” are pooled and sequenced together while still allowing each tissue pixel to be traced back to a specific tissue region. First, the microfluidic device is redesigned (FIG. 6A) to make the channels turn back and forth in a serpentine fashion (FIG. 6B) to cover the whole tissue section (2 cm×2 cm). After initial tissue pixel barcoding (pixels AiBj, I and j=1-50), the tissue “zone” barcode is added. One method to add a “zone” bacode comprised using a large square well array gasket to directly pipet the zone barcode reagents to the tissue region. A second method includes designing the “macro”-fluidic chip to perform cross-flow tissue zone barcoding (i.e., using a different set of DNA barcodes named, i.e., AA1-AA15 and BB1-BB10 to barcode >100 tissue zones, which can increase the mappable area from current technique at 2 mm×2 mm to a 10× larger area of 2 cm×2 cm) (FIG. 6C). Afterwards, chromatin DNA sequences from all 100 zones are be retrieved together for PCR amplification and sequencing to achieve ultra-large-area epigenomic mapping. It can be also applied to tissue microarrays to increase sample throughput and reduce cost. All these are critical for wide-spread adoption of hsrChST-seq in the medical or clinical settings.

Mapping Human Bone Marrow Niches in Patients with Blood Cancers.

Myelodysplastic Syndromes (MDS), is a cancer of the hematopoietic stem cells (HSC) on the rise in recent years, uncurable with chemo or targeted therapy, and may progress to acute myeloid leukemia and eventually death. Deep molecular, epigenomic, and phenotypic atlas mapping of primary MDS sheds light on contextual MDS pathogenesis and the role of the MDS immune microenvironment and help discover novel targeted therapeutics for MDS and potentially other blood cancers.

Validation of patient bone marrow (BM) clot sections for hsrChST-seq and hsrRNA-seq: “Clot sections” that maintain and capture the BM architecture: bone marrow aspiration dislodges BM “particles” devoid of trabecular bone but with preservation of the hematopoiesis/vascular/stromal BM niche are available for research on. Standard tissue histopathology protocols are used to process these samples for the study.

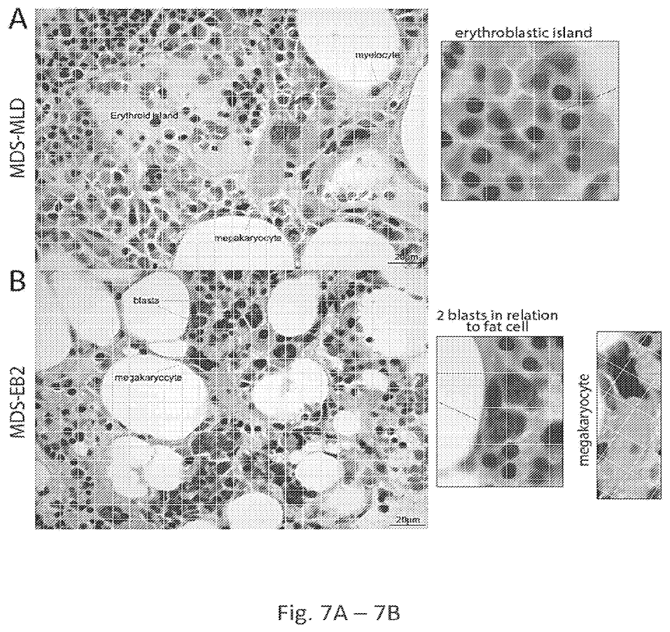

Spatial mapping of MDS BM immune microenvironment. First, to validate preservation of cellular composition and architecture of clot sections, including BM microenvironment cells (stromal cells, endothelial cells, fat cells, T-, B- and NK cells, etc.), normal and MDS corresponding BM biopsy and clot sections with are stained for defining markers of the individual cell populations: CD34 (blasts and endothelial cells), CD3, CD19, CD56 (T-, B, NK), nestin (mesenchymal stromal cells), CXCL12 (CXCL12-abundant reticular (CAR) cells) in conjunction with markers for myeloid subsets, such as CD33 (myeloid progenitors), CD71 (erythroid progenitors), CD68 (macrophages). Next, m hsrChST-seq and hsrRNS-seq are performed on MDS clot sections. A 10 μm grid and 50×50 barcodes are used for hsrChST-seq (FIG. 7). Examples are shown for cell-to-pixel and pixel-to-pixel relationships for low grade (FIG. 7A) and high-grade (FIG. 7B) MDS are shown.

Distinguishing Malignant and Non-Malignant Hematopoietic Stem/Progenitor Cells (HSPCs) and Decoding their Respective Microenvironment.

HsrChST-seq and hsrRNA-seq are performed on MDS and aged/gender-matched control BM. Since genomic DNA sequencing is performed at the sites of chromatin modifications, the same data can be used to differentiate a subset of driving mutations to differentiate malignant (cancerous) vs non-malignant HSPCs. Alternatively, mutation-specific probes are designed to capture recurrent, sample-specific hot-spot mutations to identify mutant versus normal hematopoietic cells and mutant hematopoietic versus non-mutated stromal/microenvironmental cells. Together, this deep molecular (epigenetic and transcriptional), genotypic, phenotypic data of primary MDS at high-spatial resolution will shed light on contextual MDS pathogenesis and the role of the MDS immune-microenvironment, and potentially lead to development of novel targeted therapeutics for MDS and other related blood cancers.

### Example 3: High Resolution Spatial Epigenetics Via Deterministic in Situ Barcoding

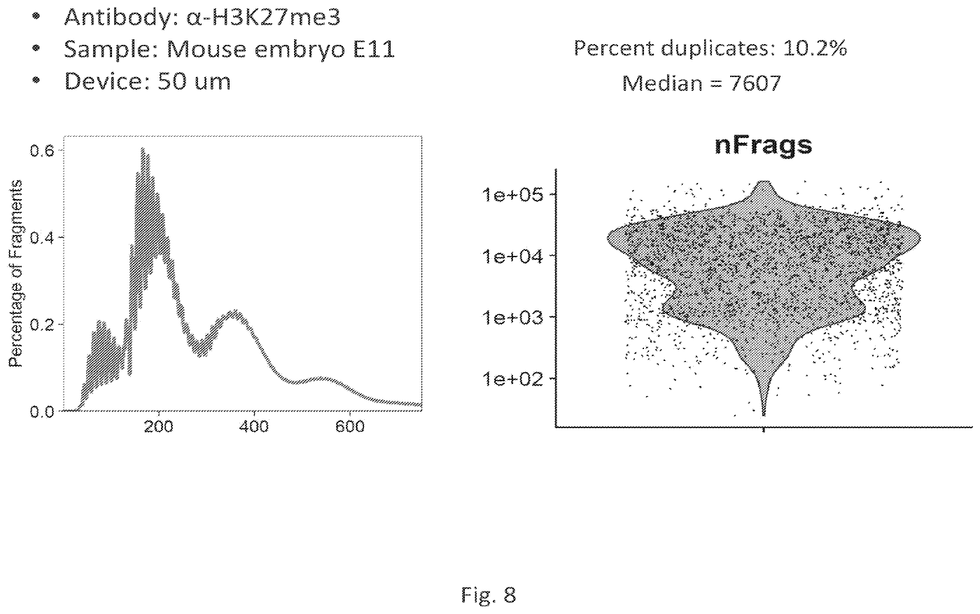

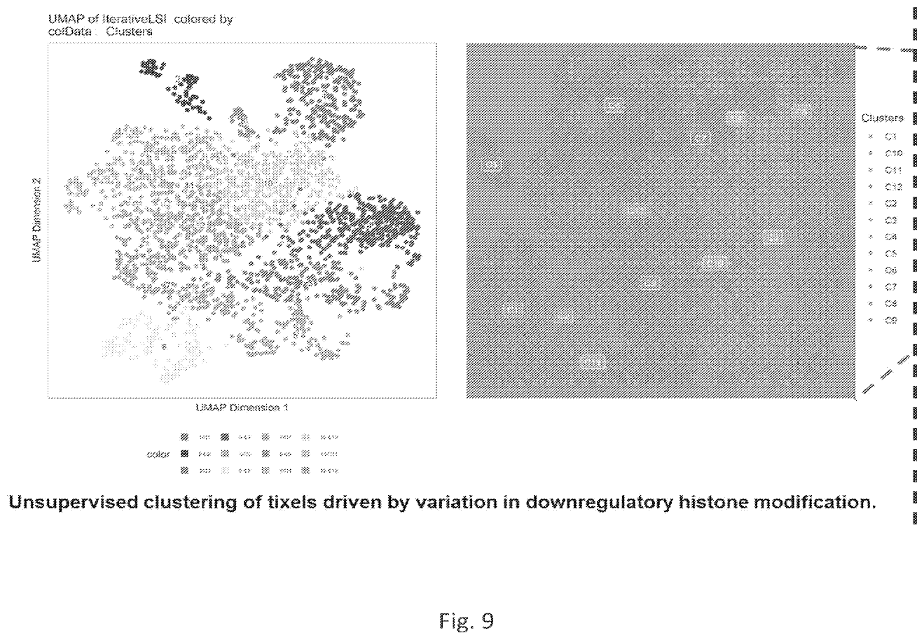

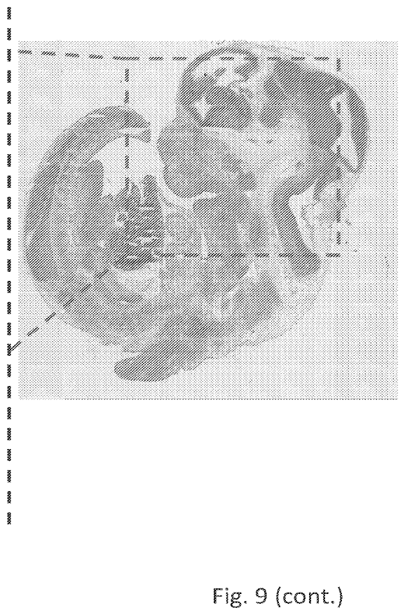

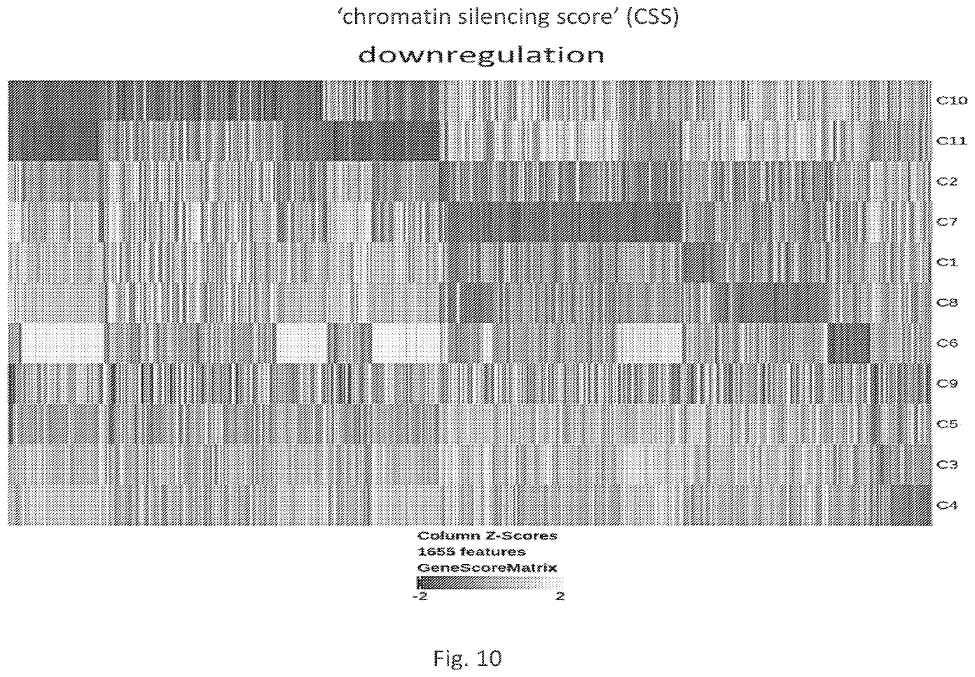

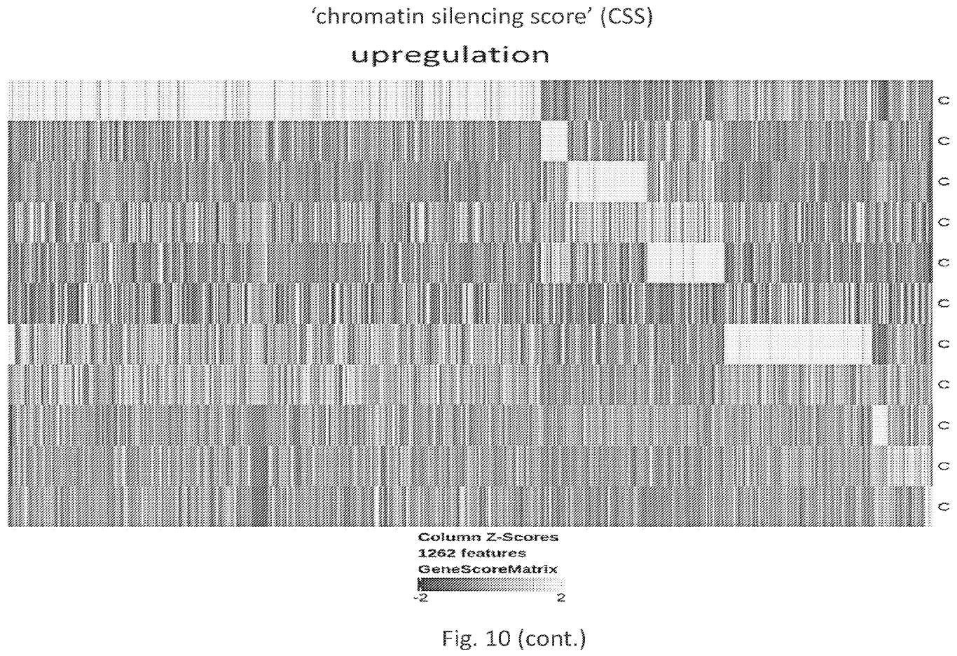

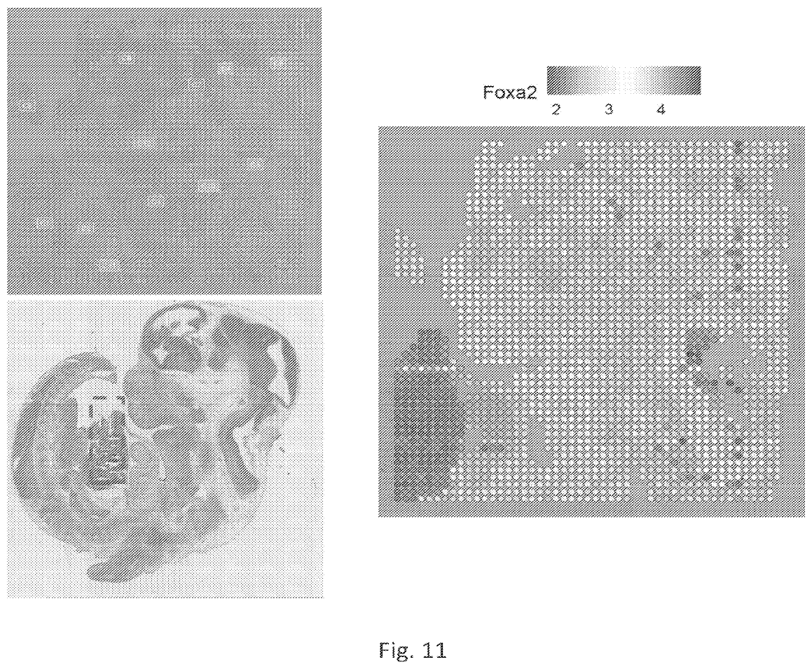

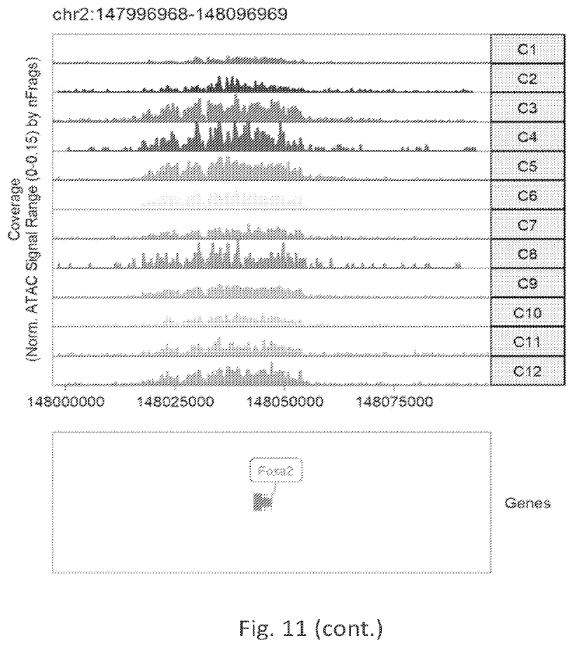

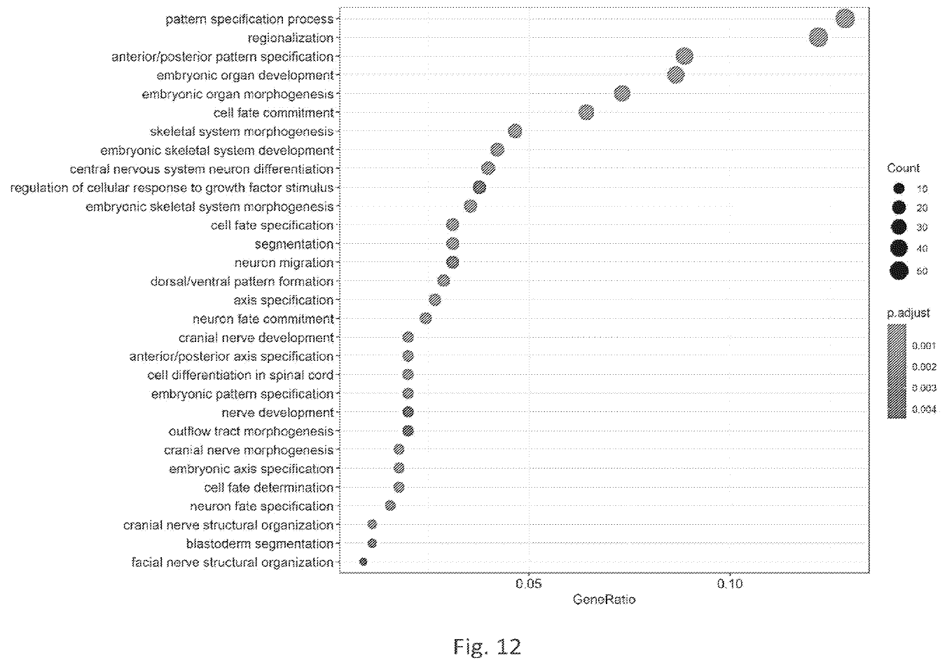

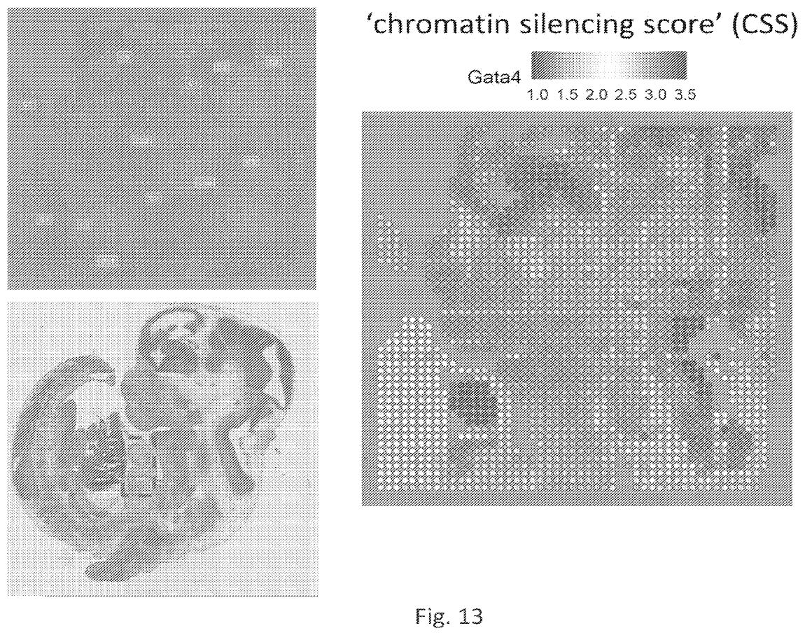

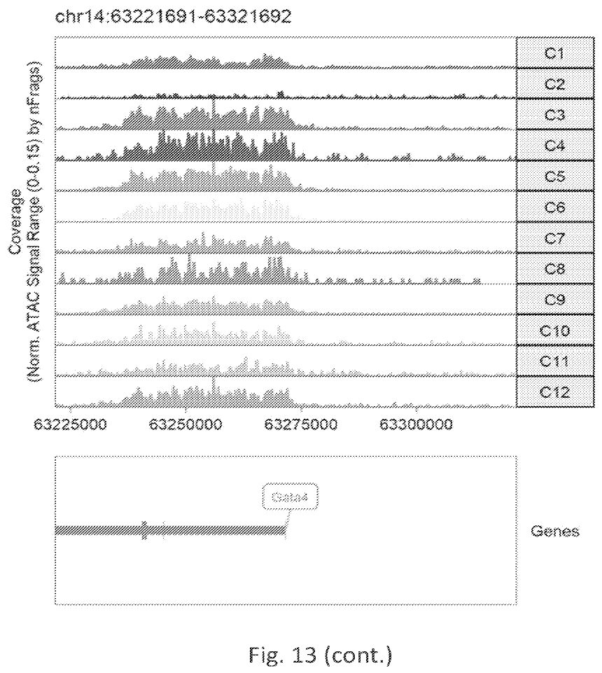

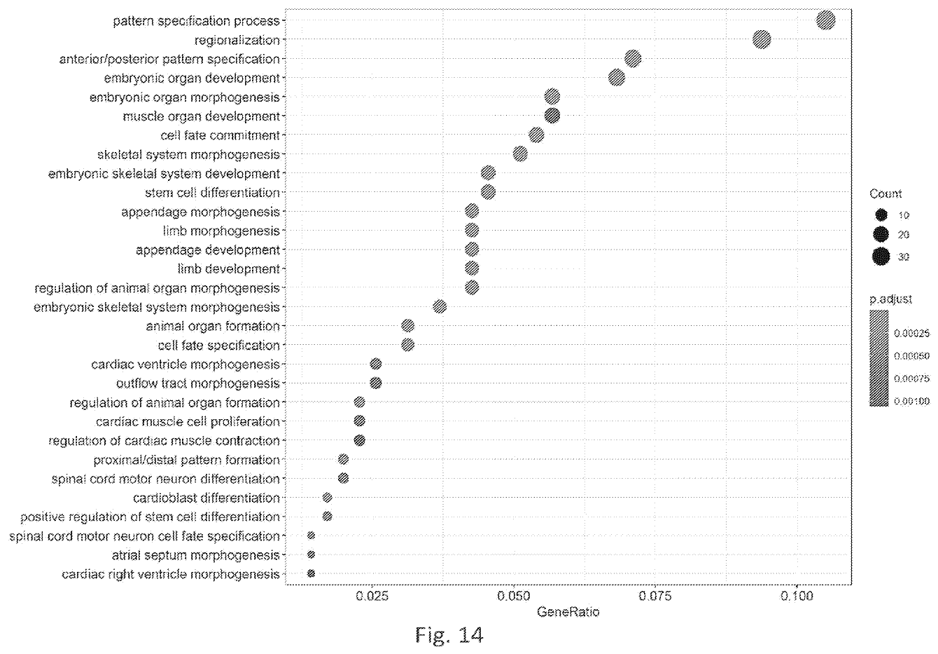

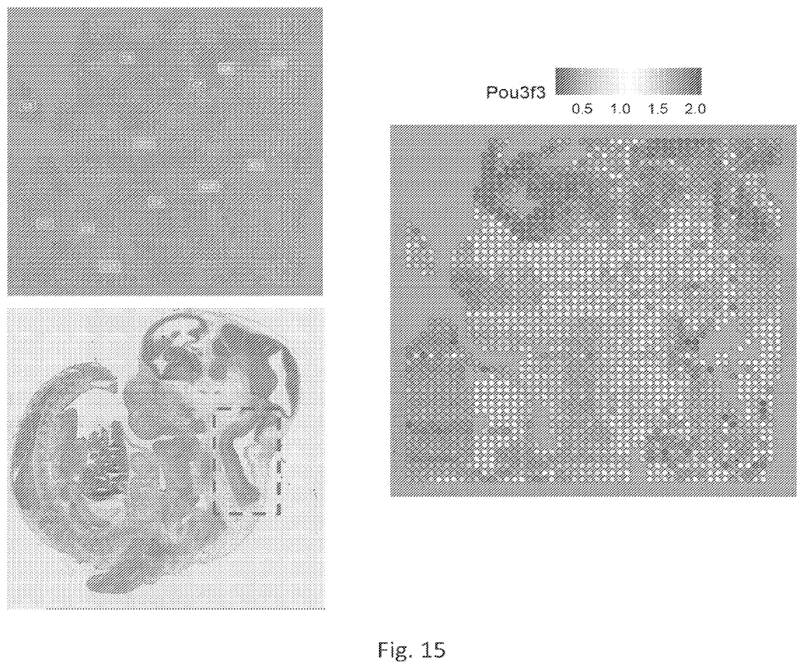

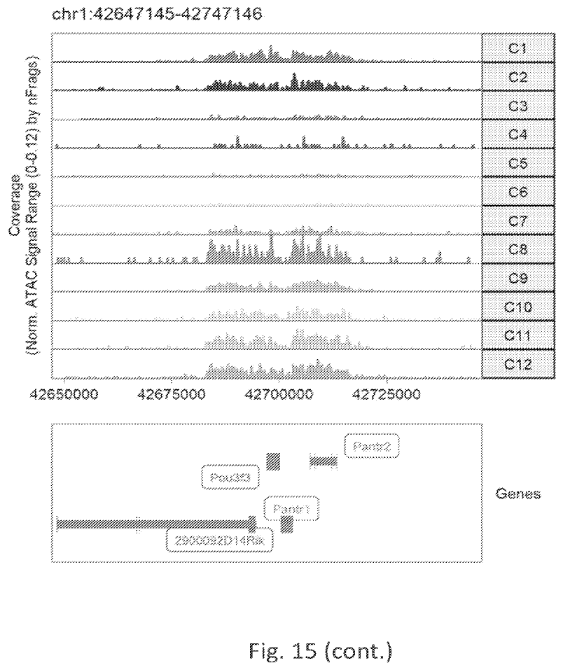

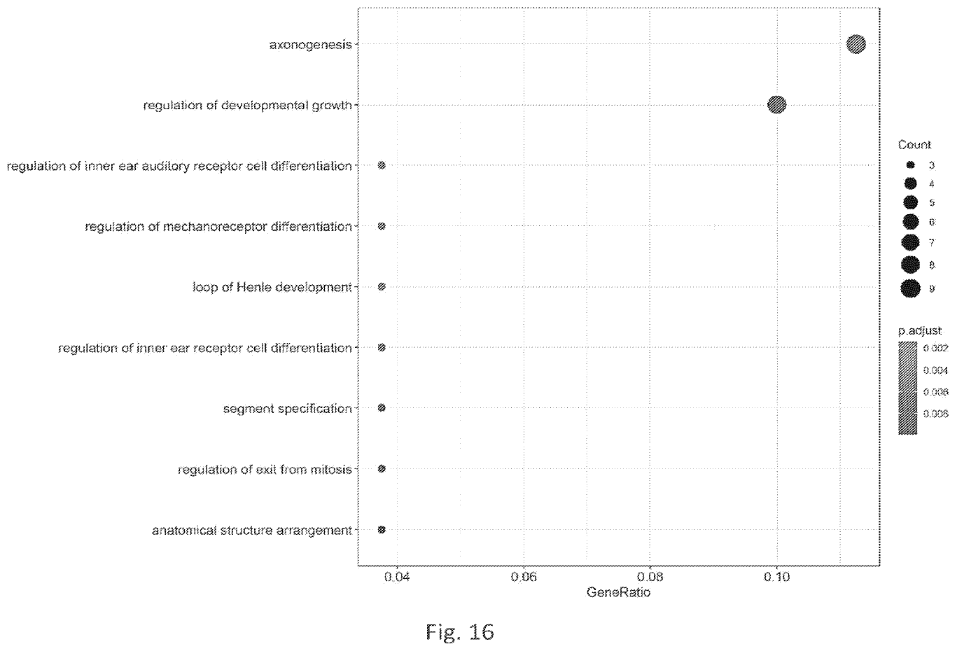

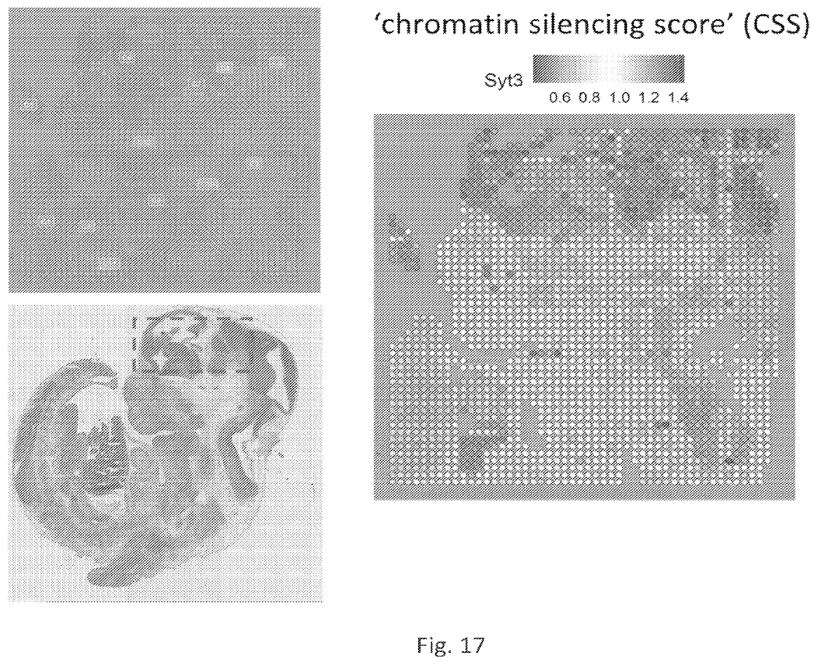

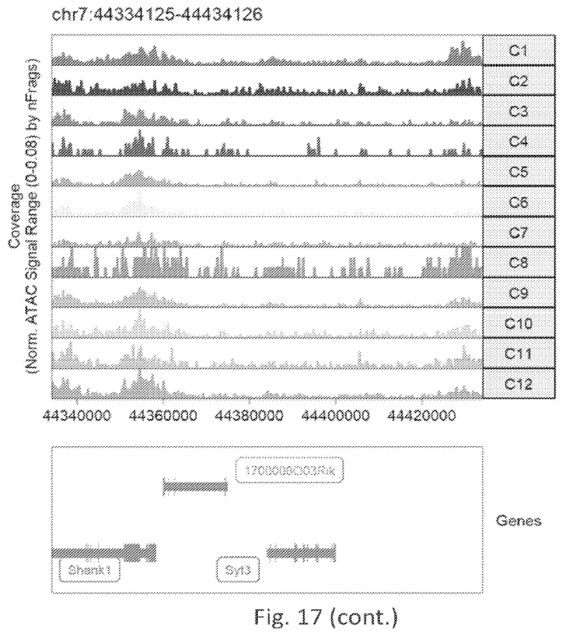

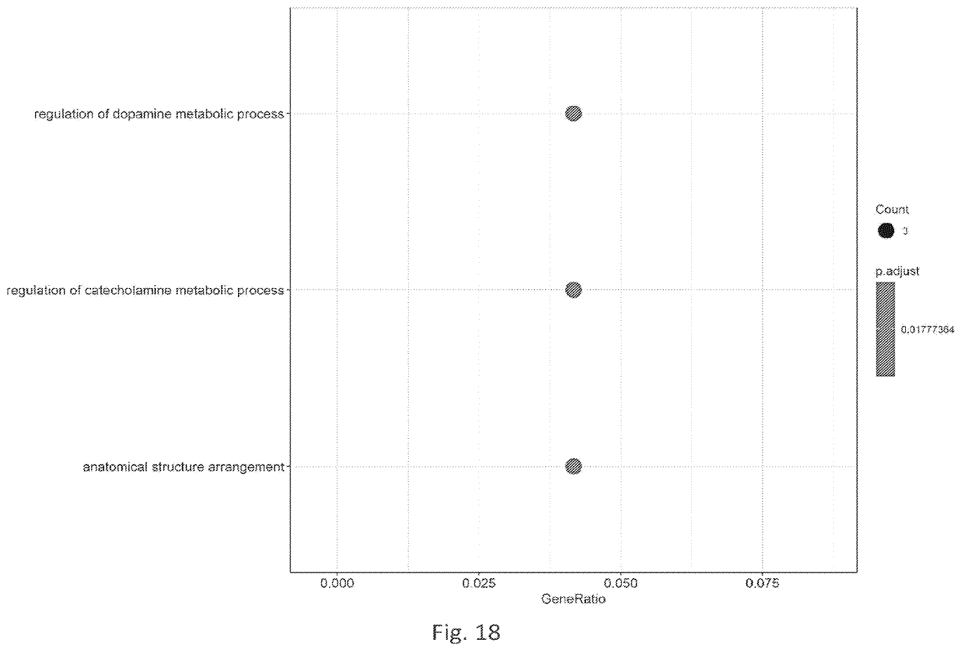

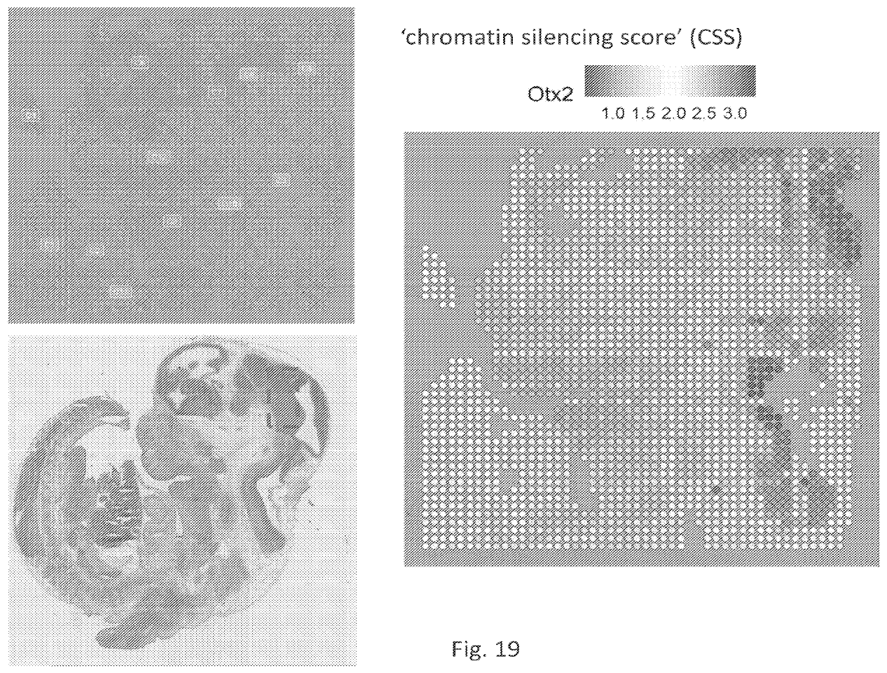

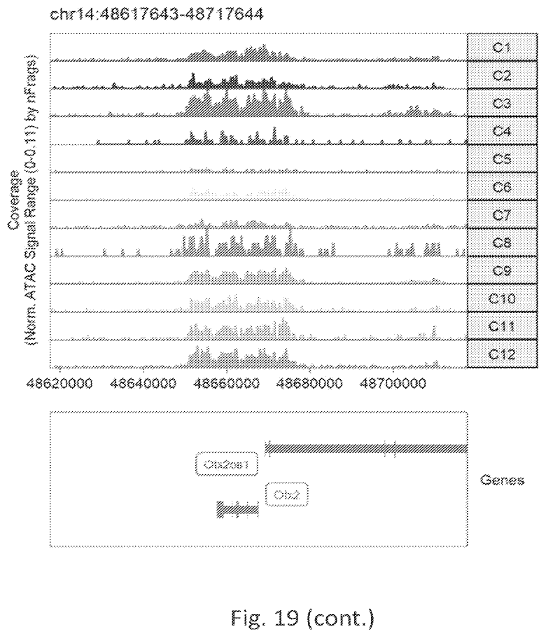

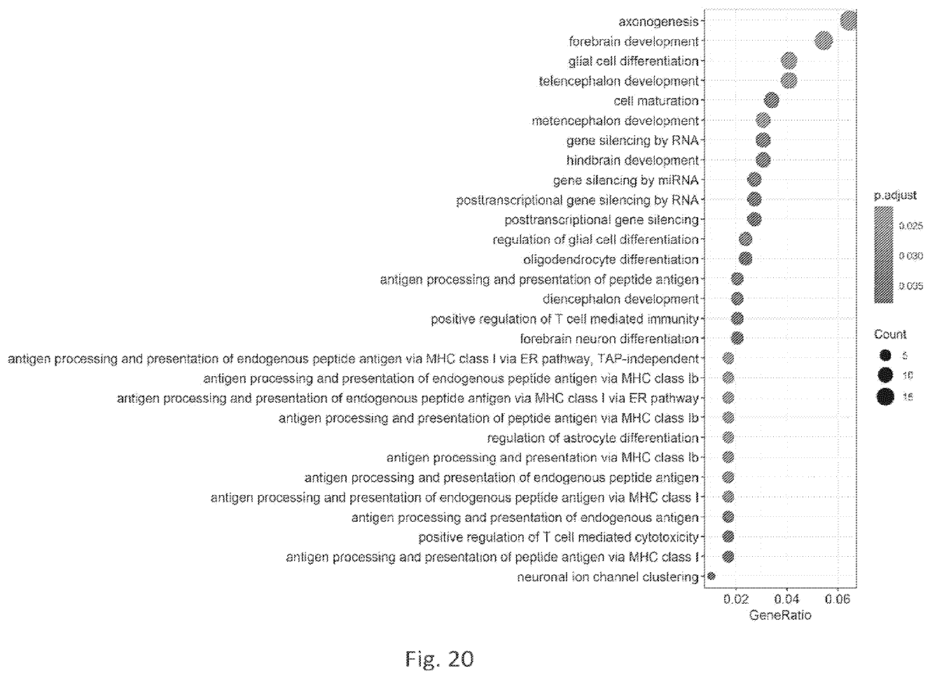

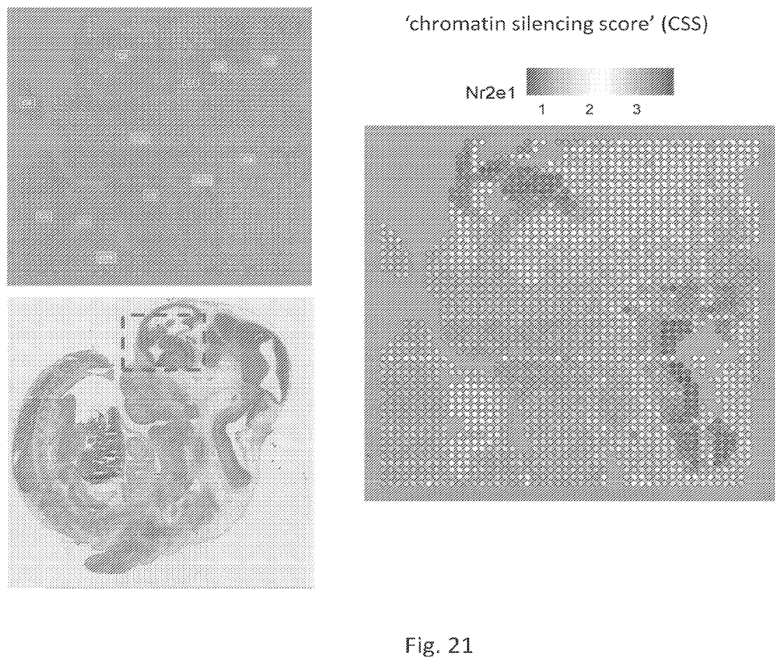

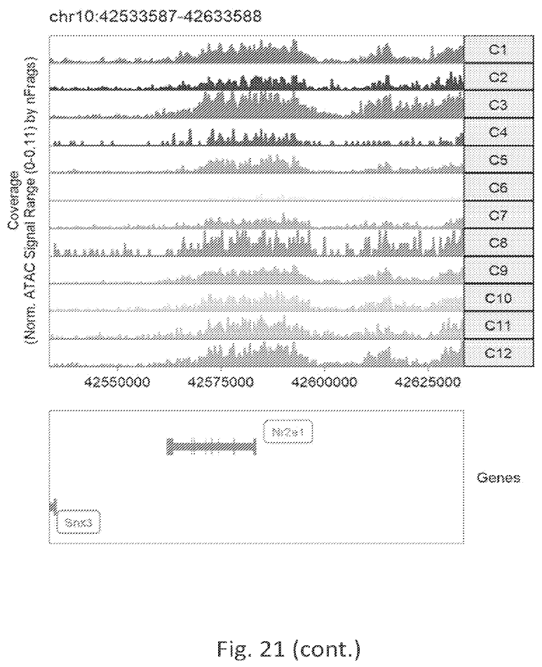

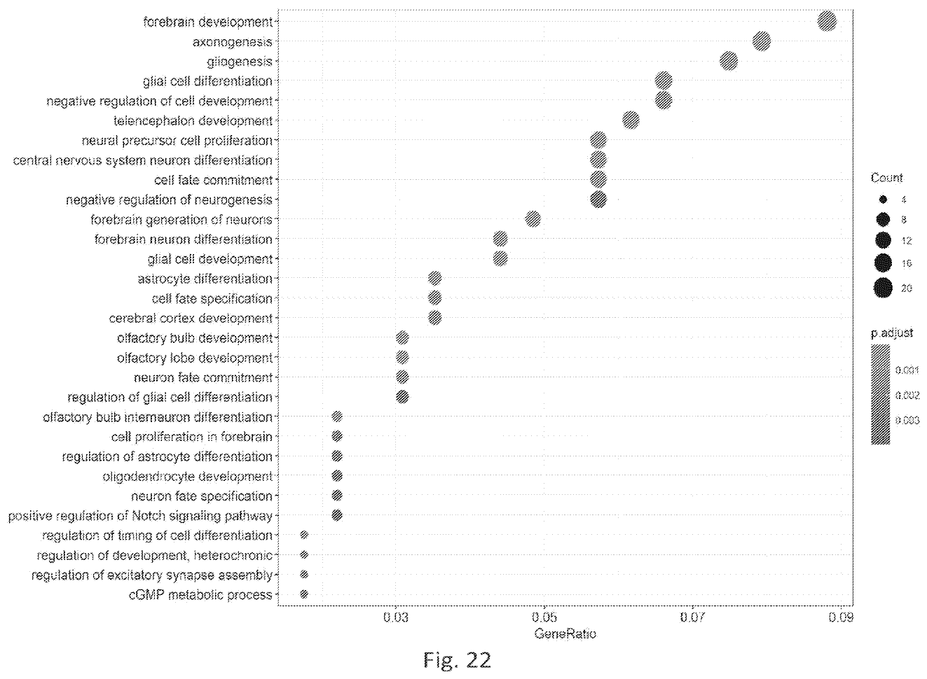

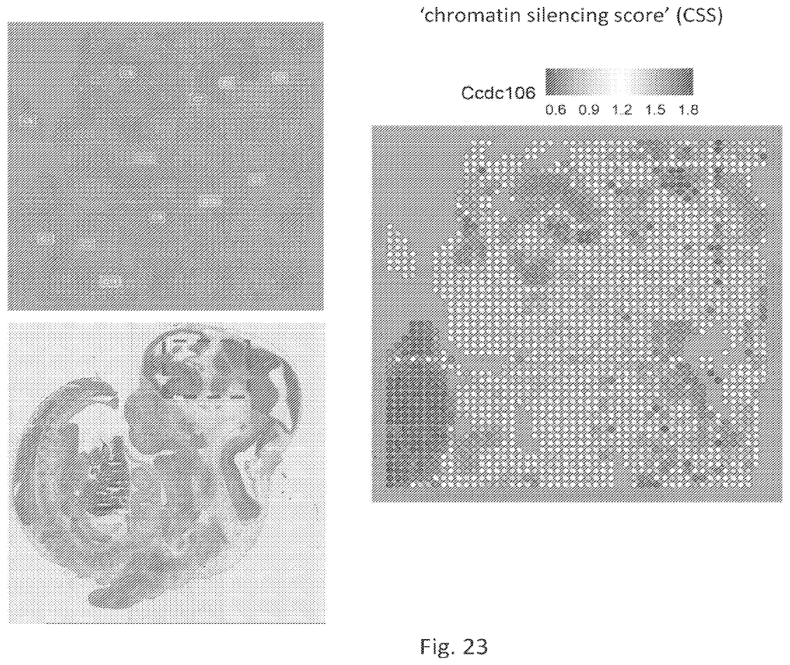

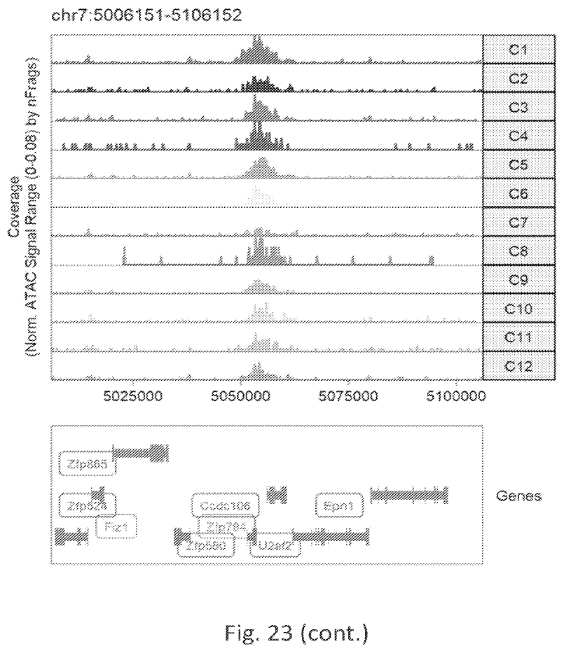

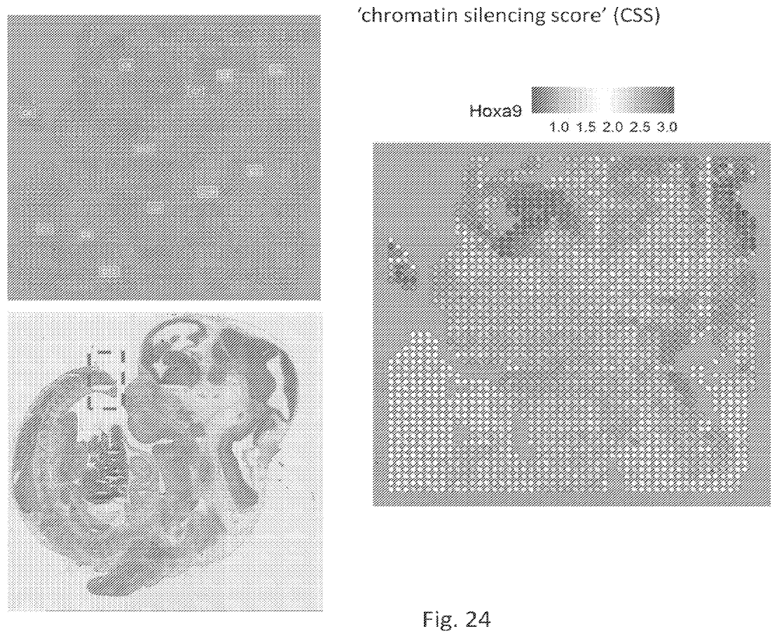

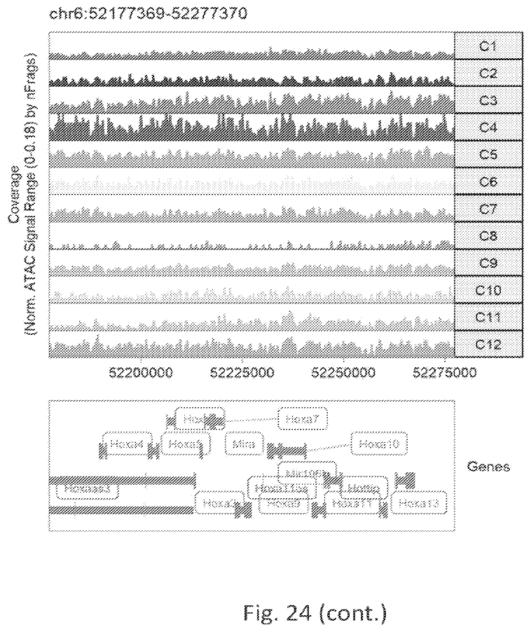

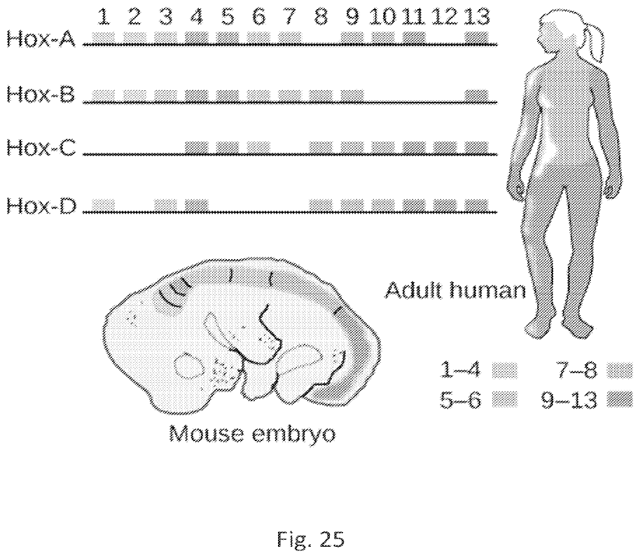

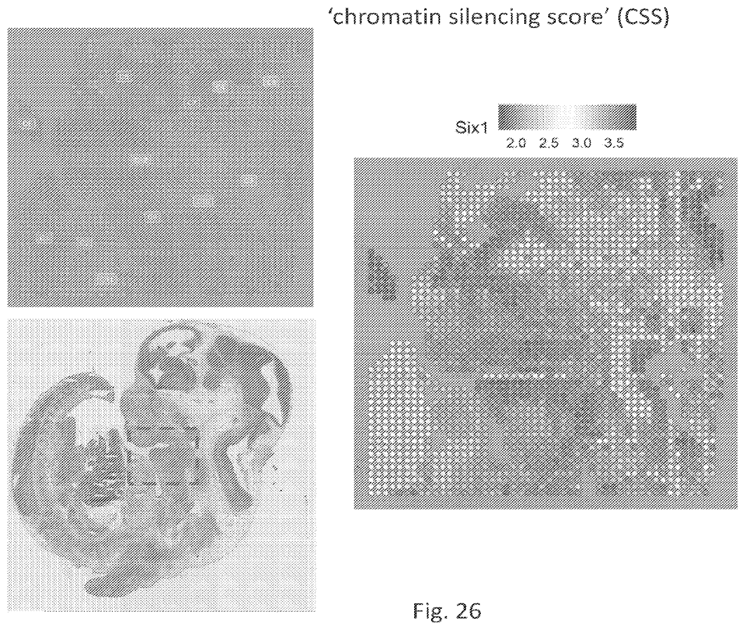

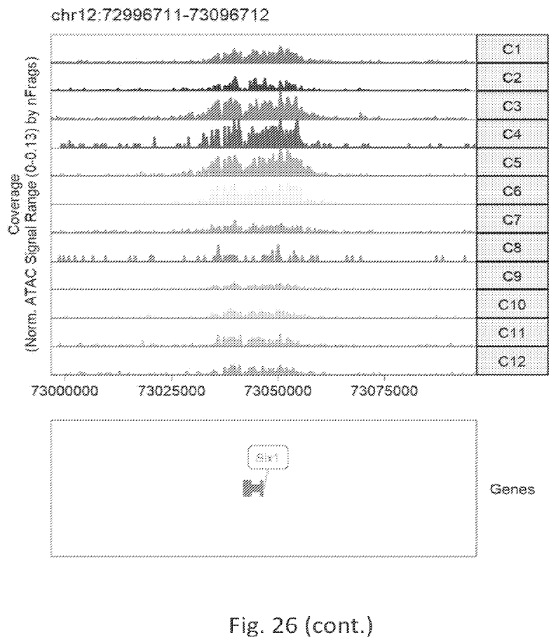

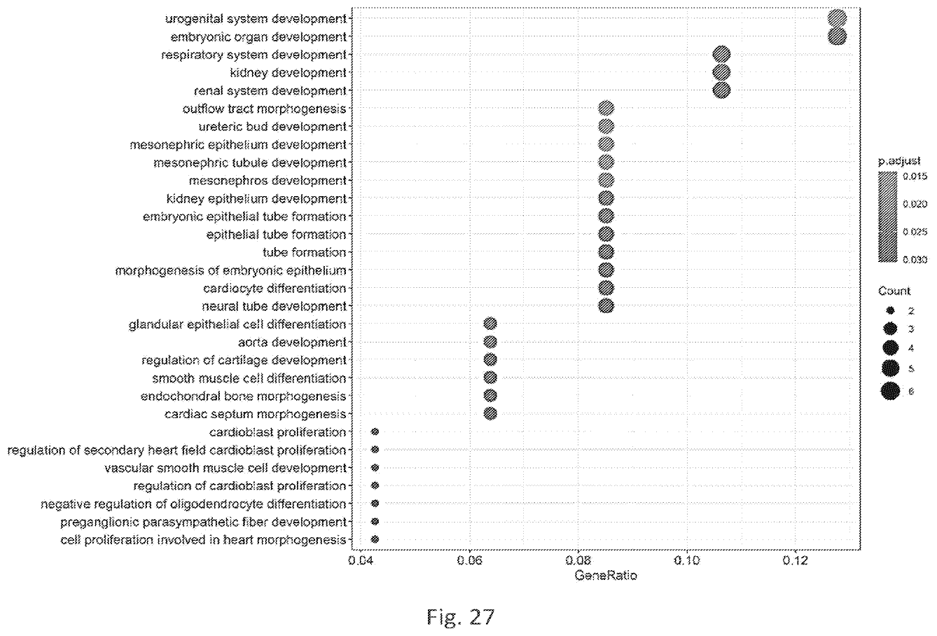

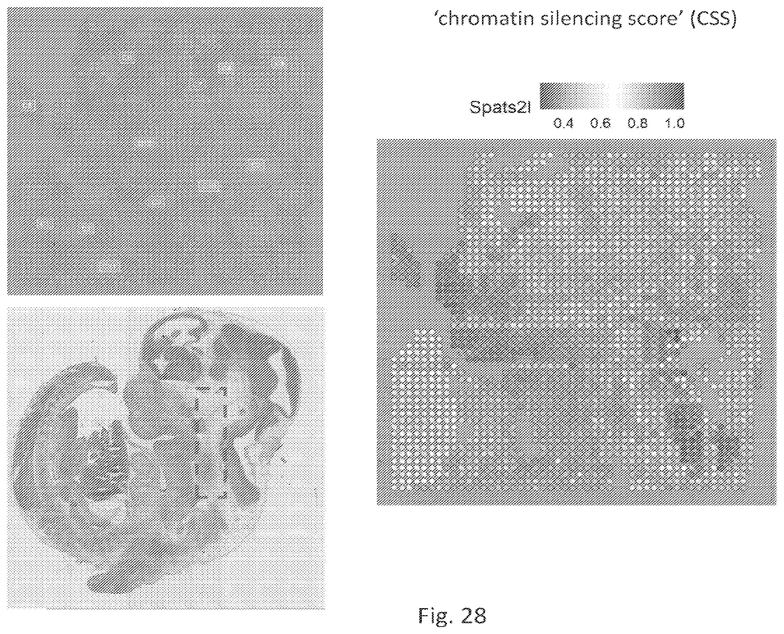

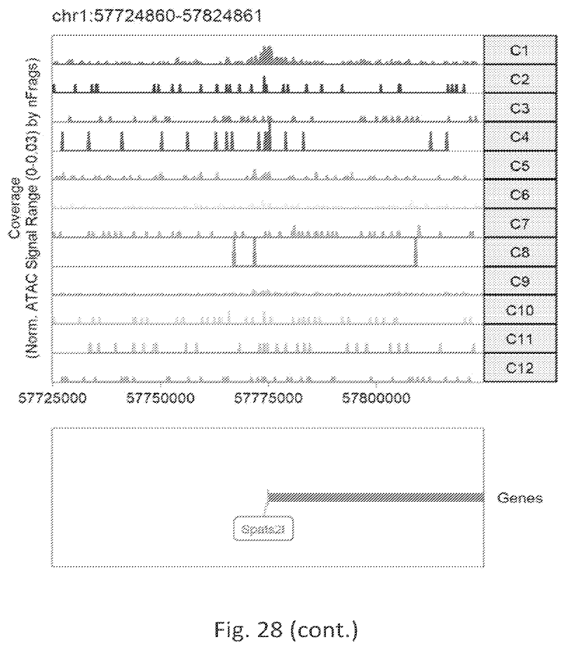

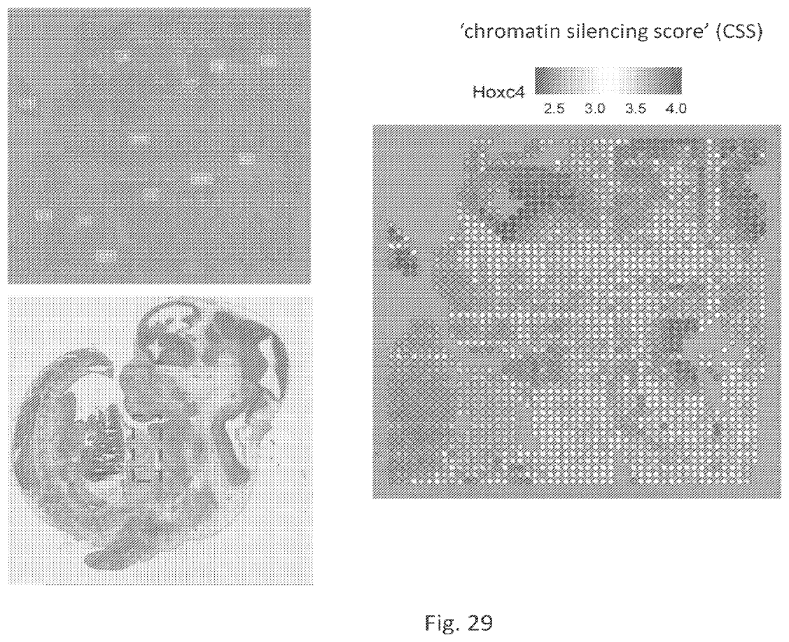

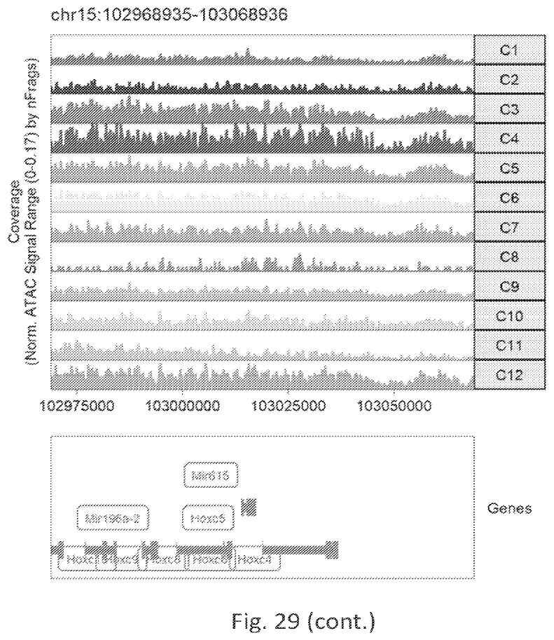

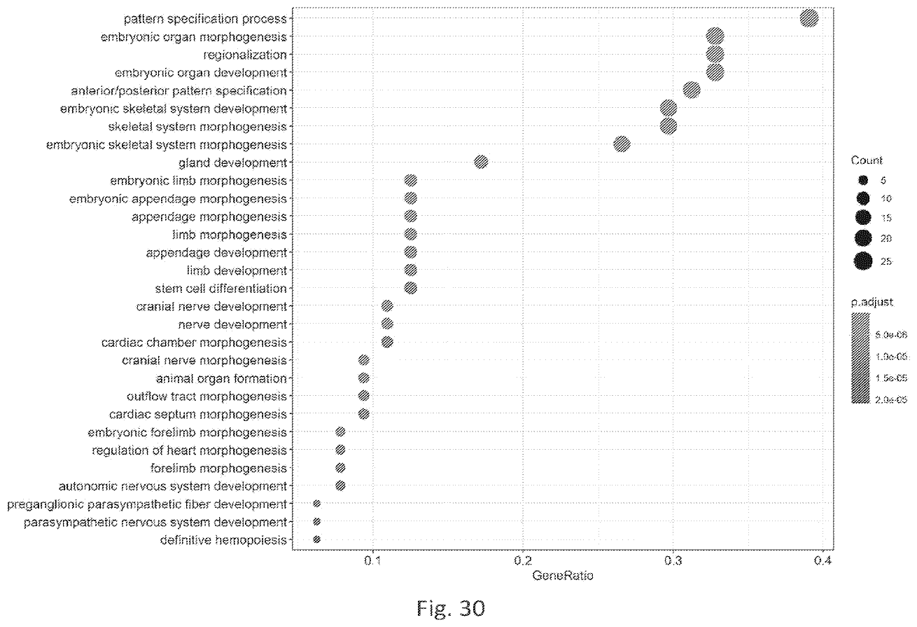

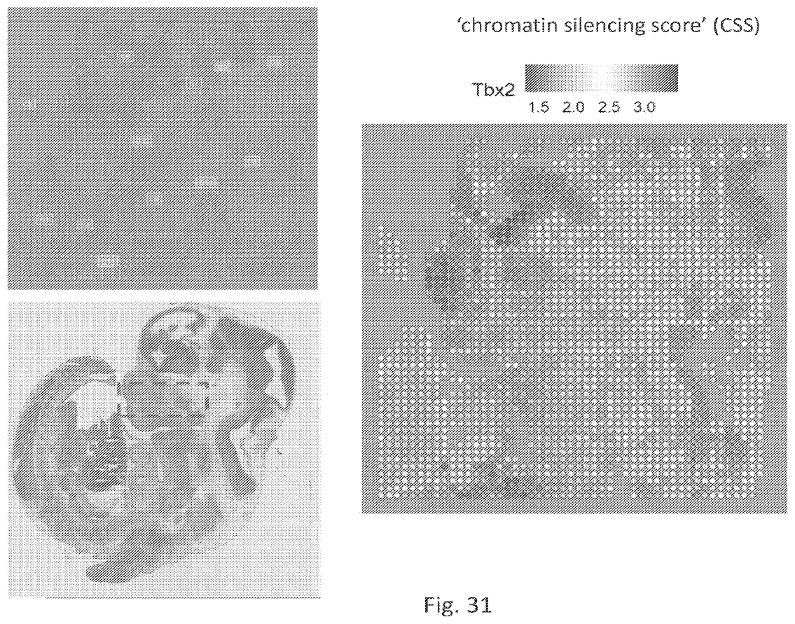

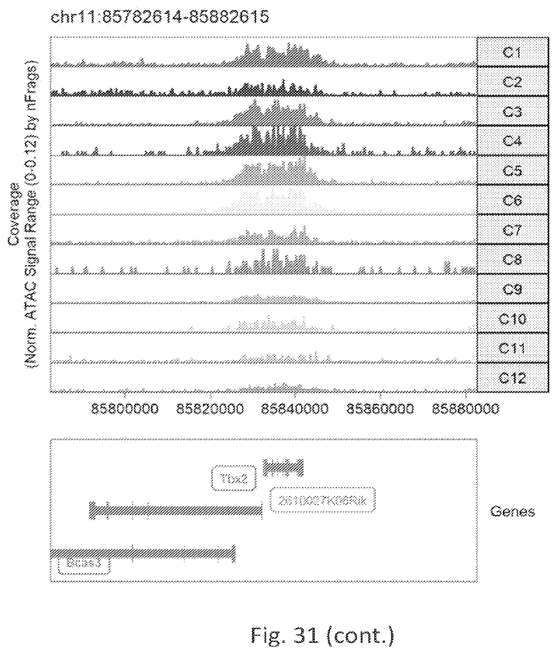

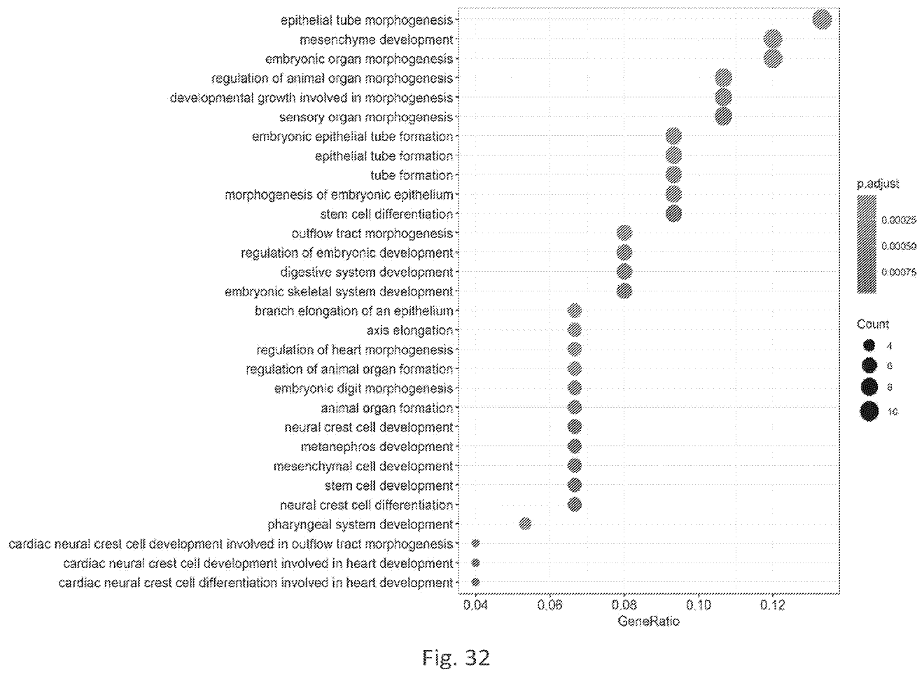

FIGS. 8 through 32 demonstrate the use of the system of the invention to spatially identify the H3K27me3 epigenomic marker, and genes that are activated or silenced due to an increase or decreased level of H3K27me3.

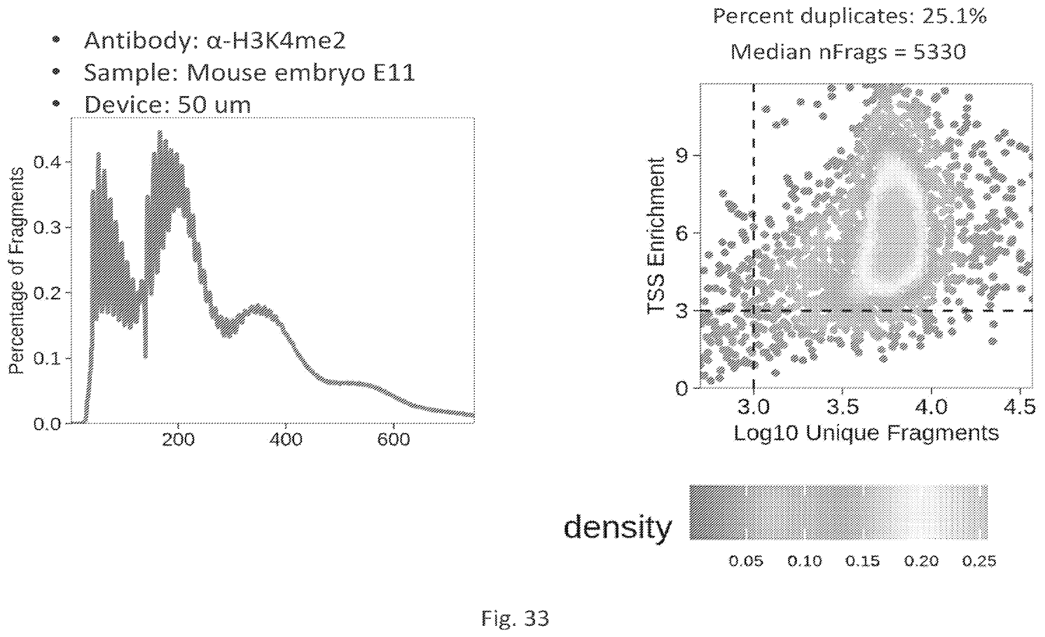

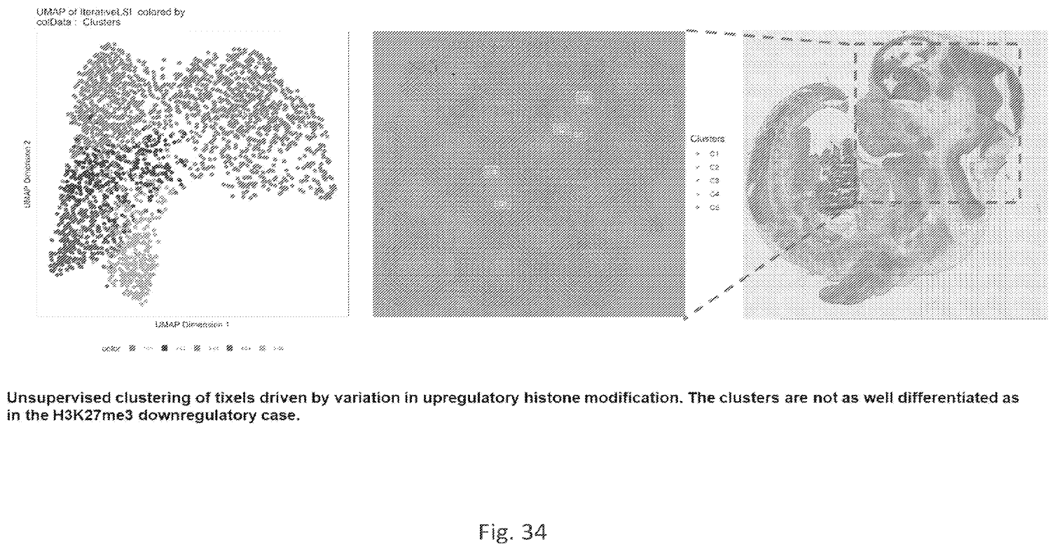

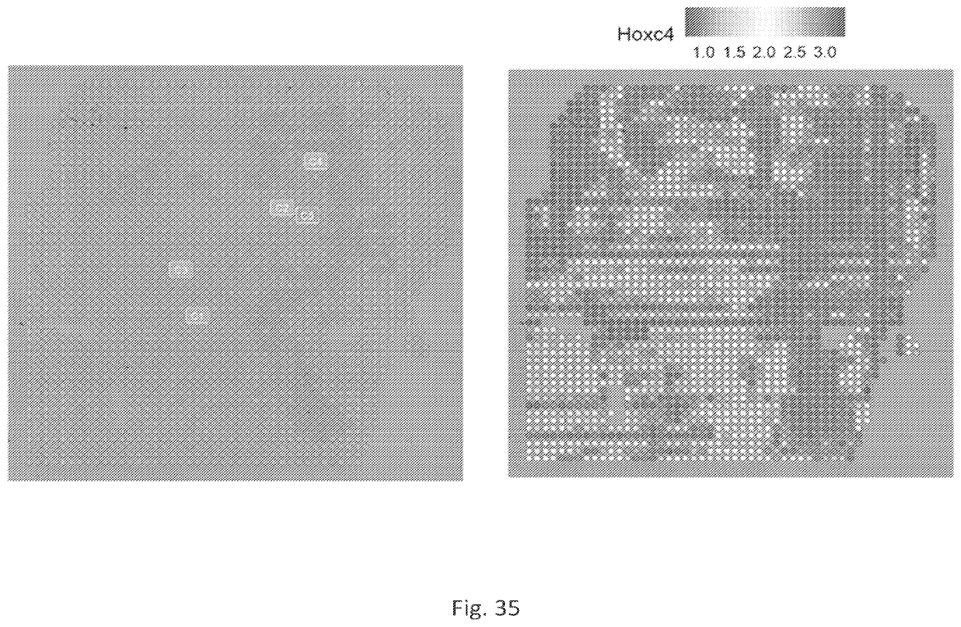

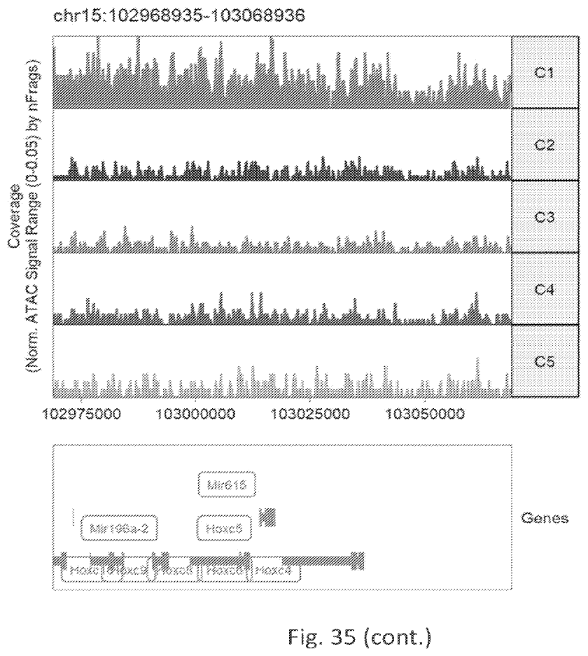

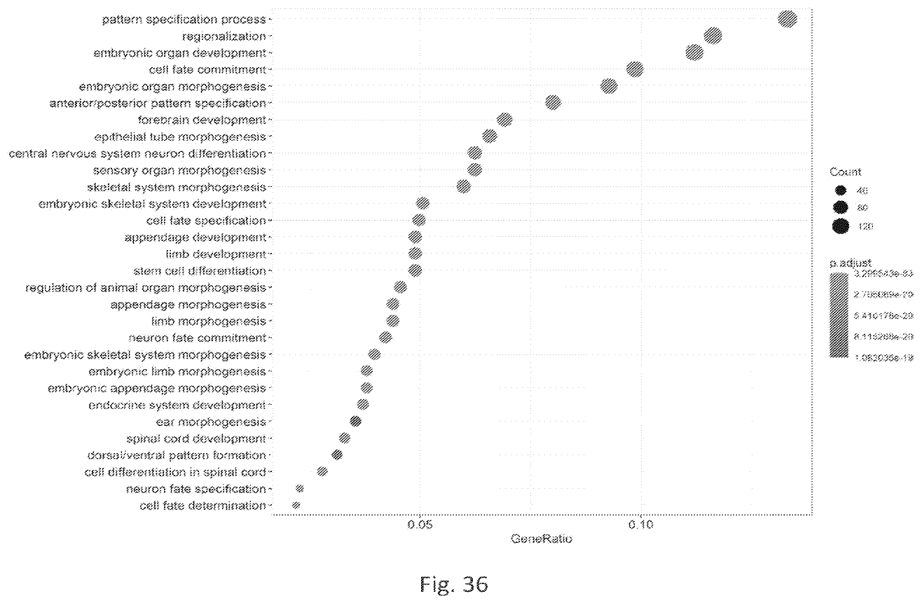

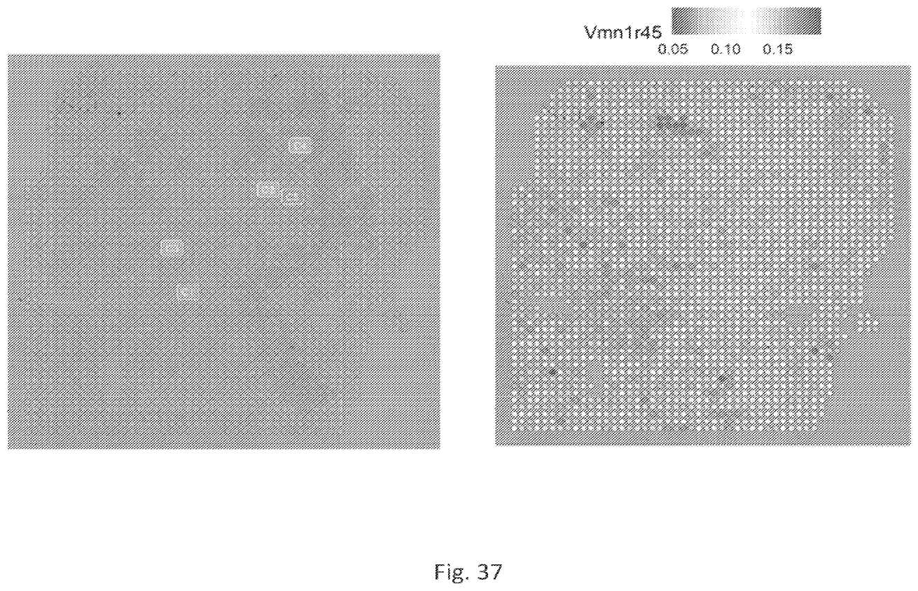

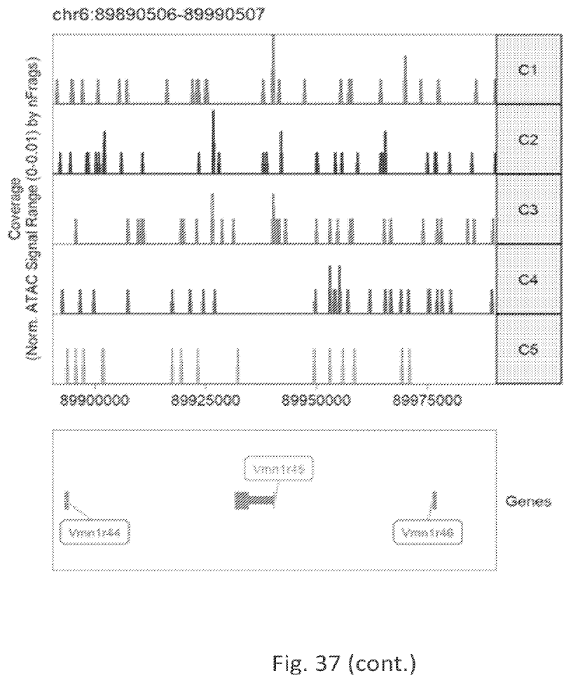

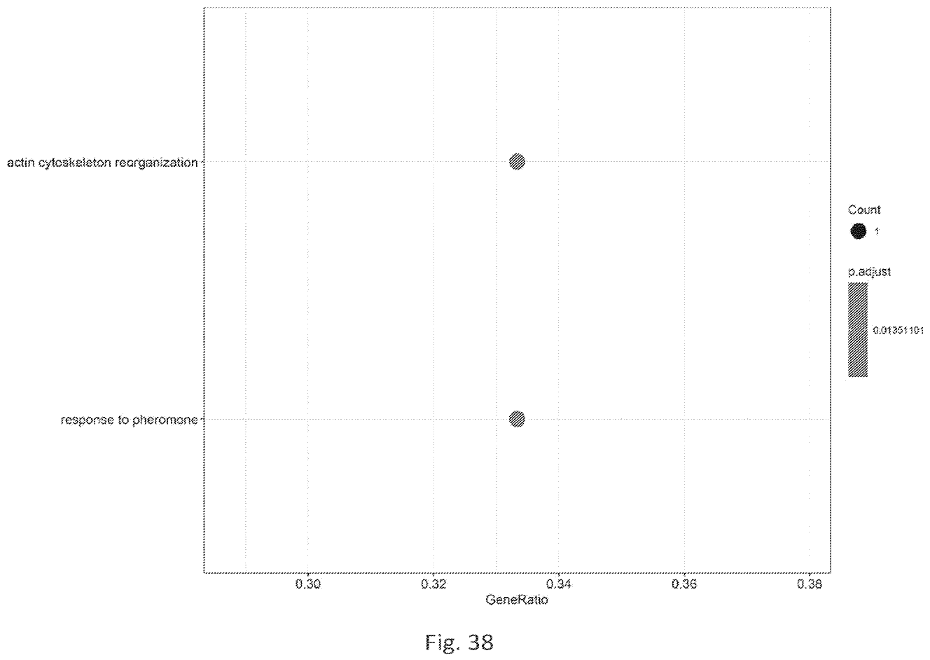

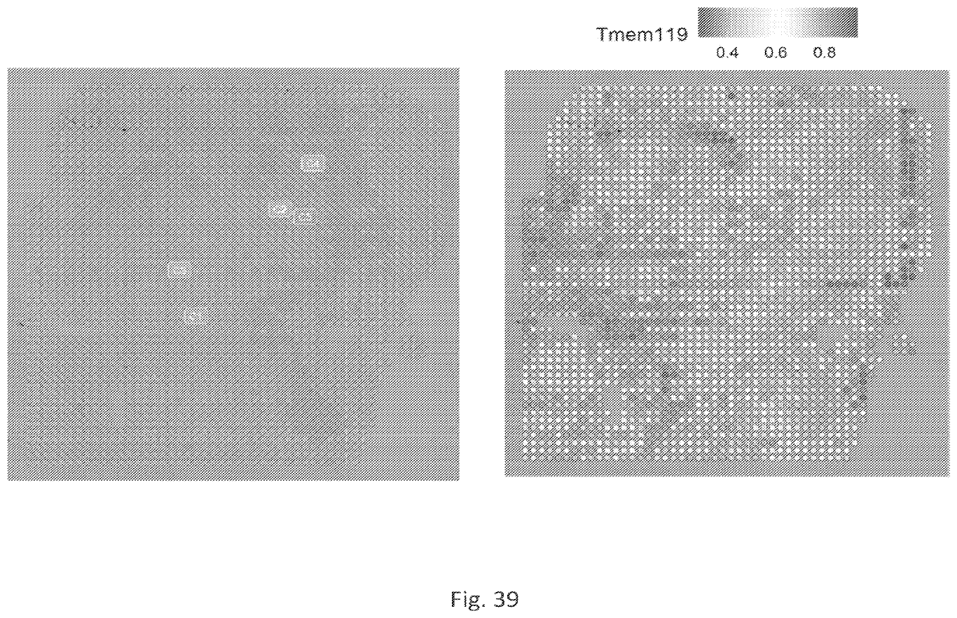

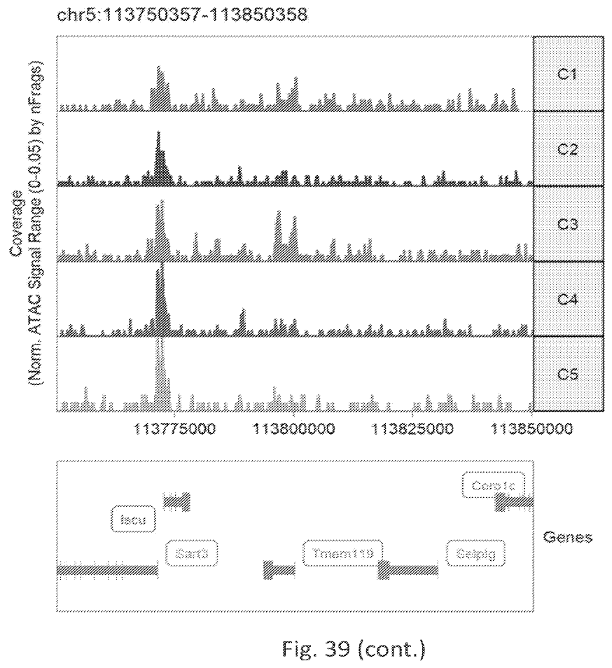

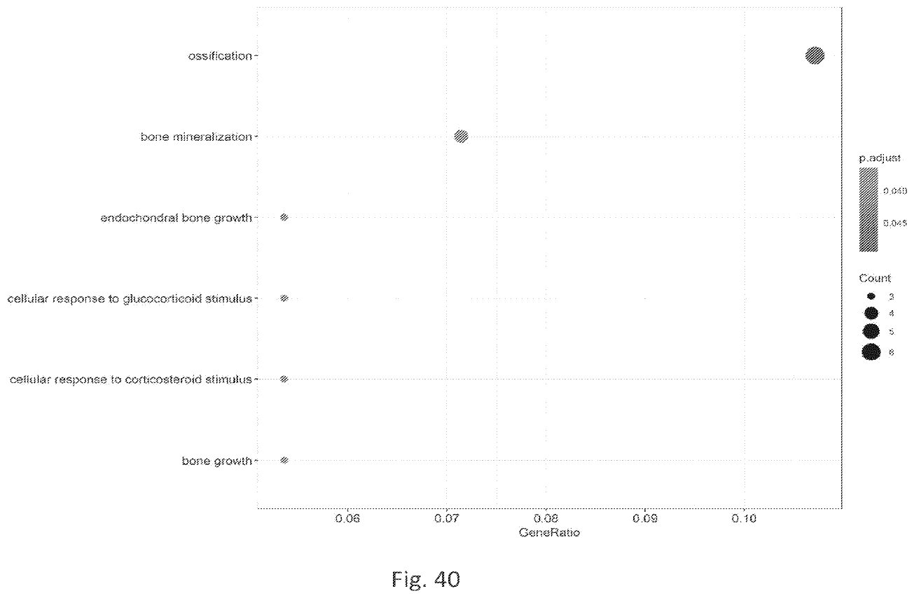

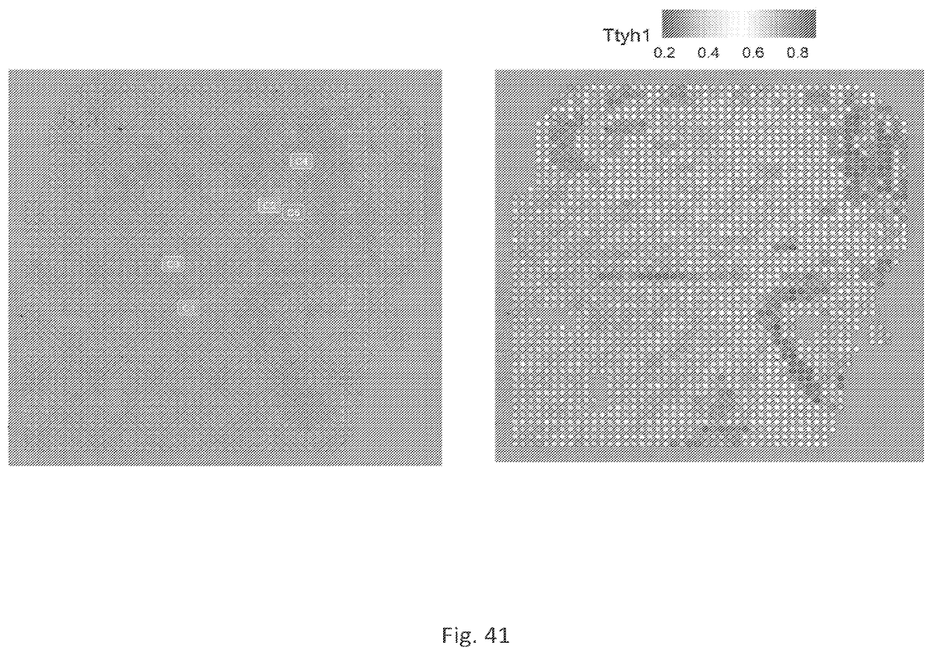

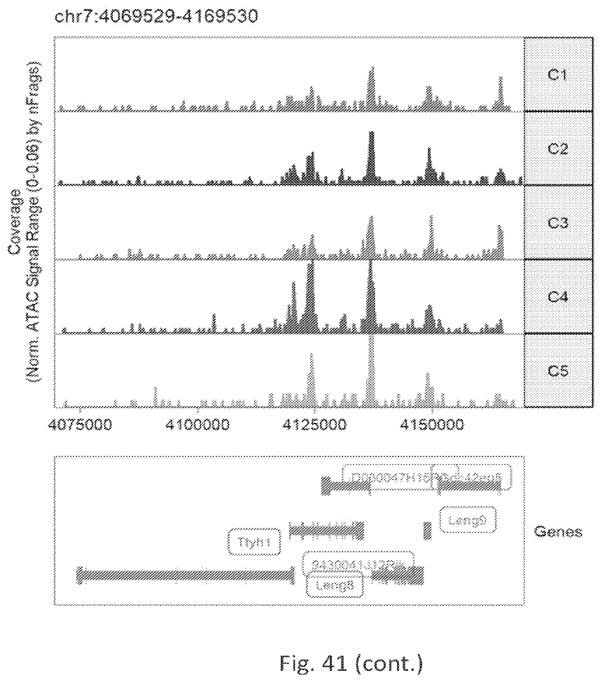

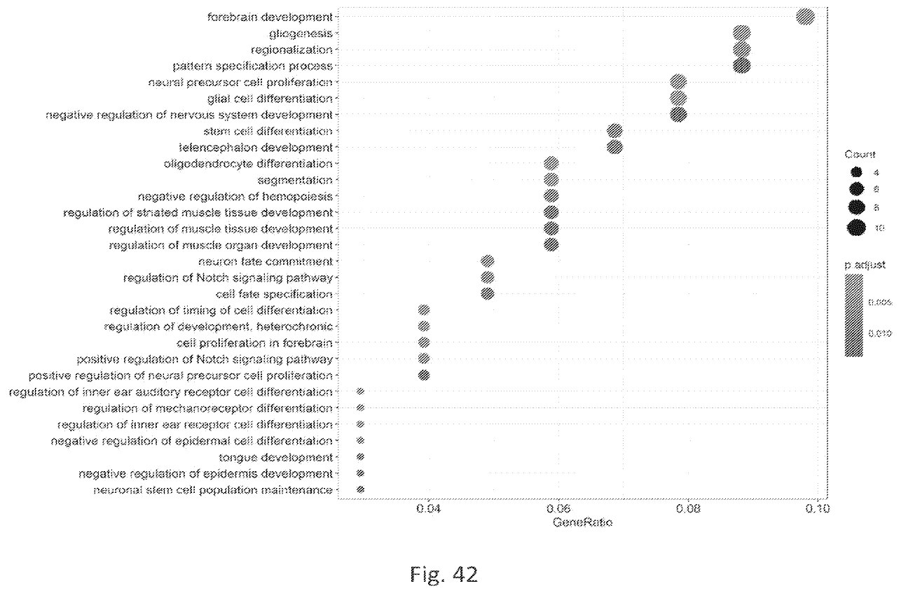

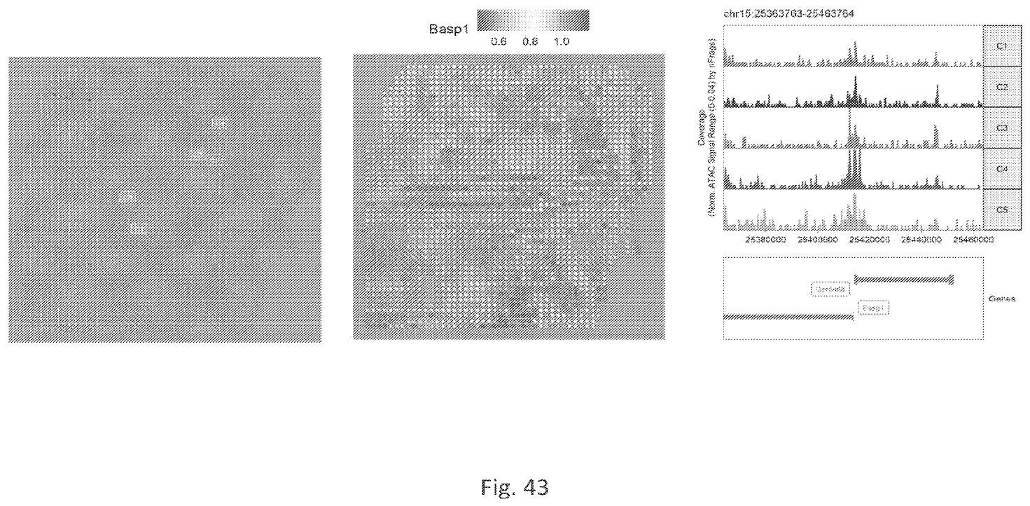

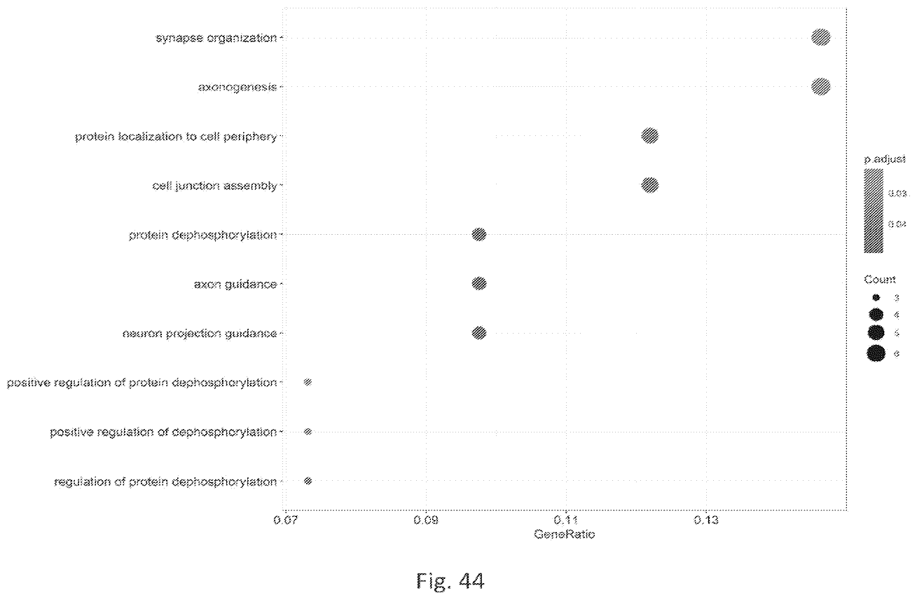

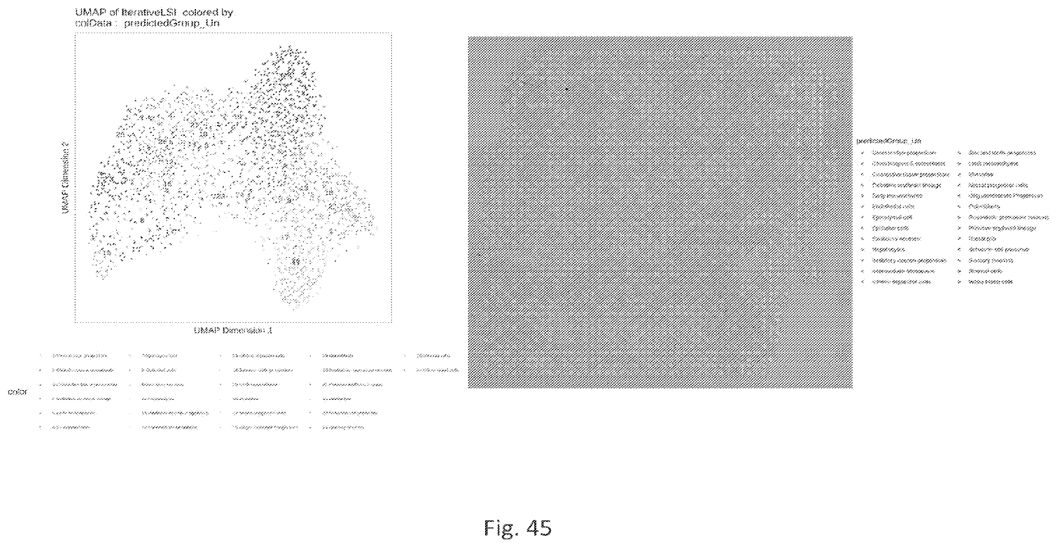

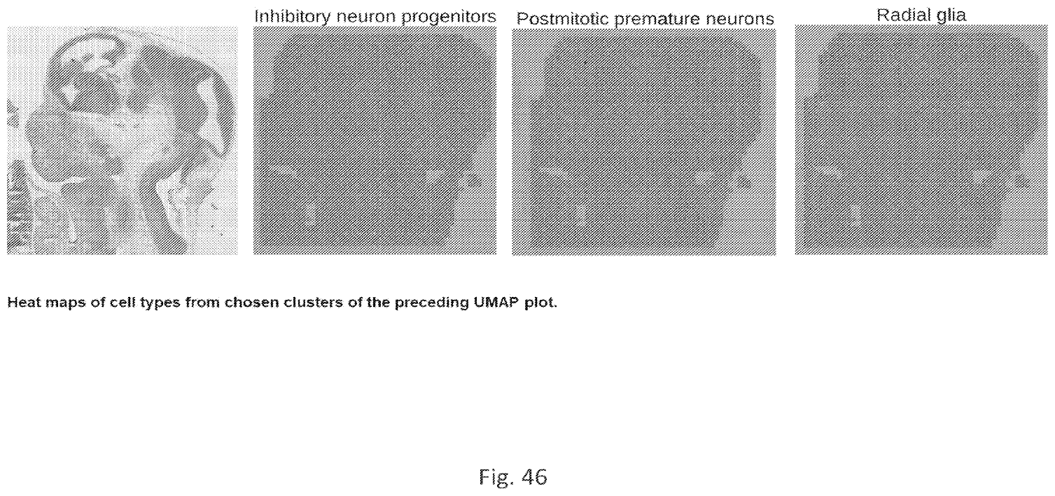

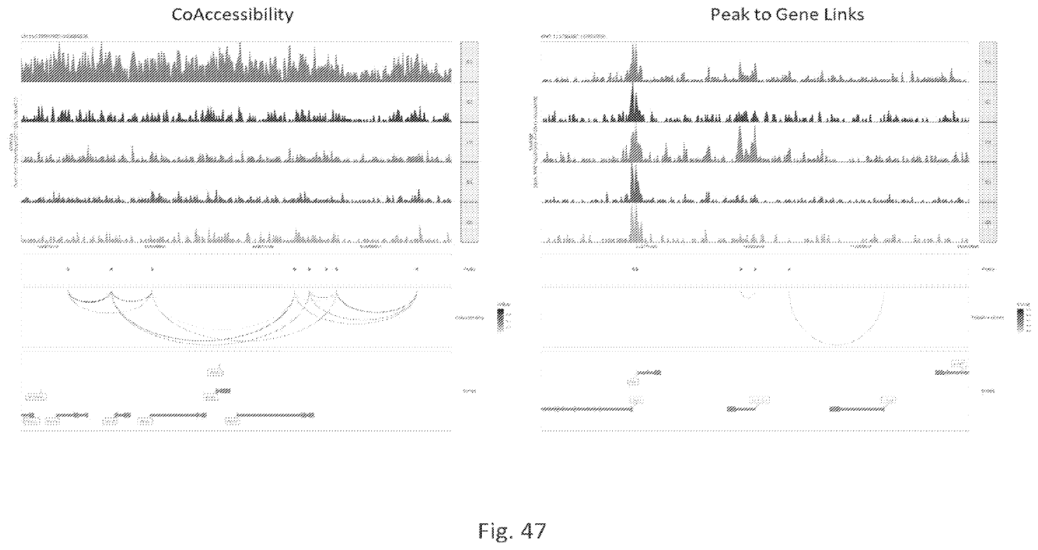

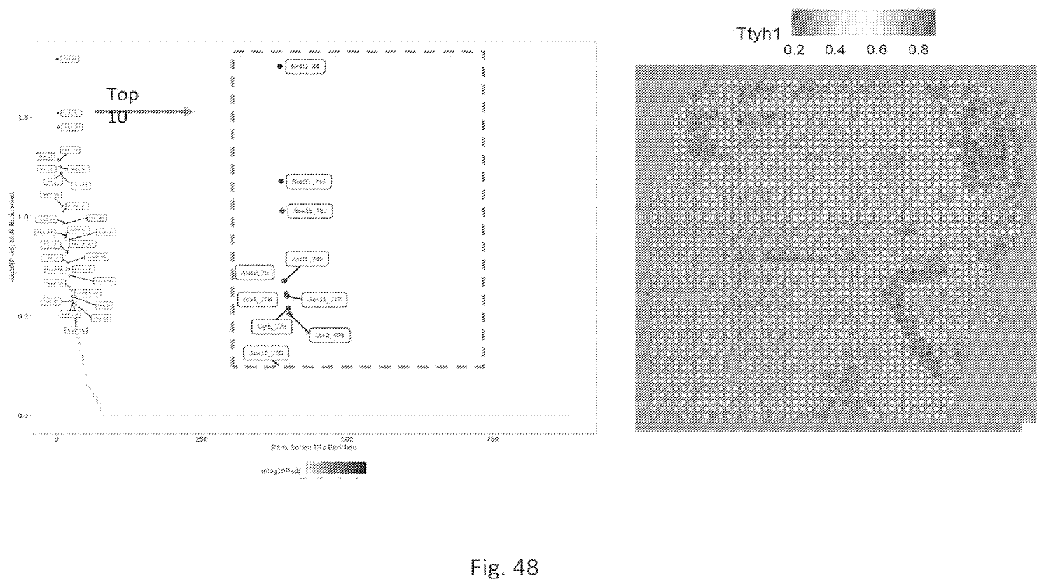

FIGS. 33 through 48 demonstrate the use of the system of the invention to spatially identify the H3K4me2 epigenomic marker, and genes that are activated or silenced due to an increase or decreased level of H3K4me2.

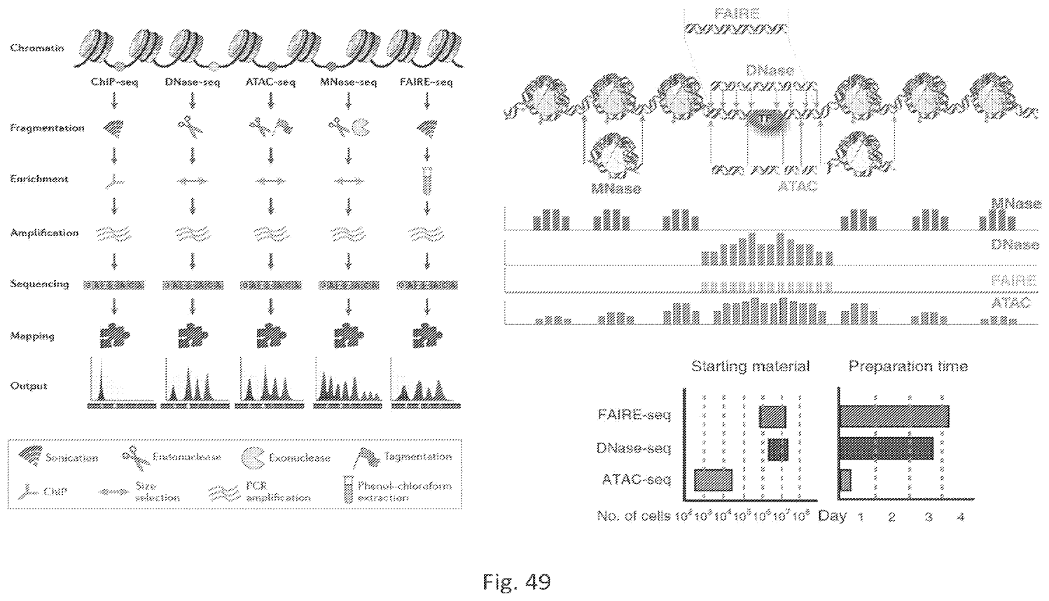

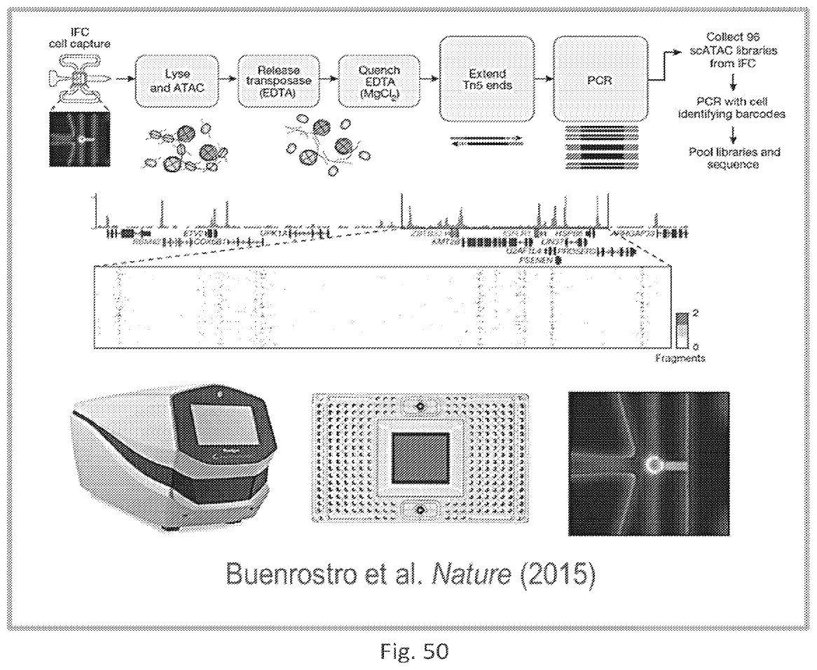

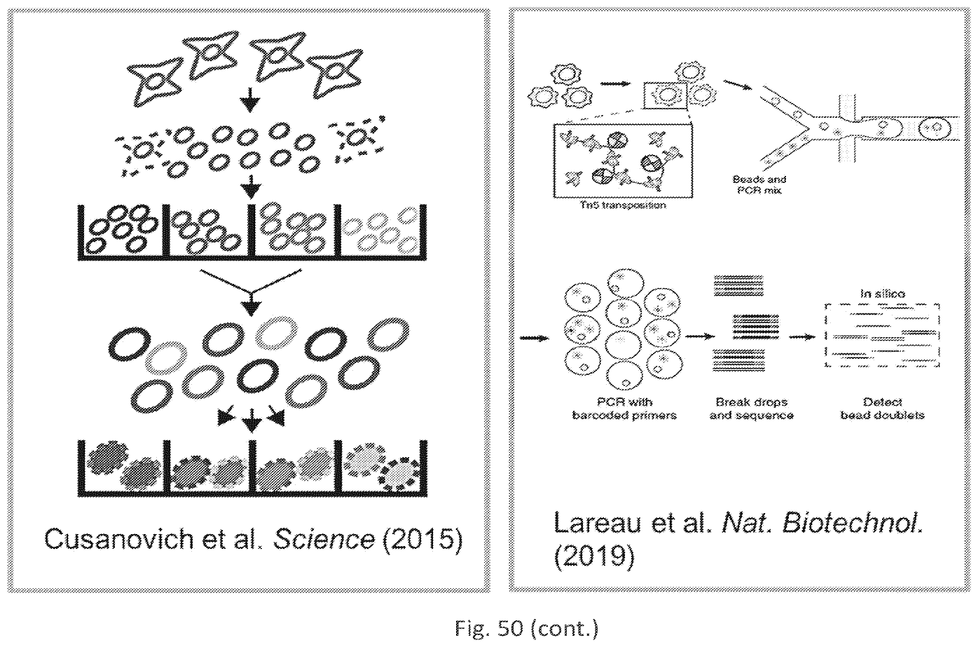

FIGS. 49 and 50 depicts previous methods of determining chromatin accessibility. These methods are not able to provide spatial information.

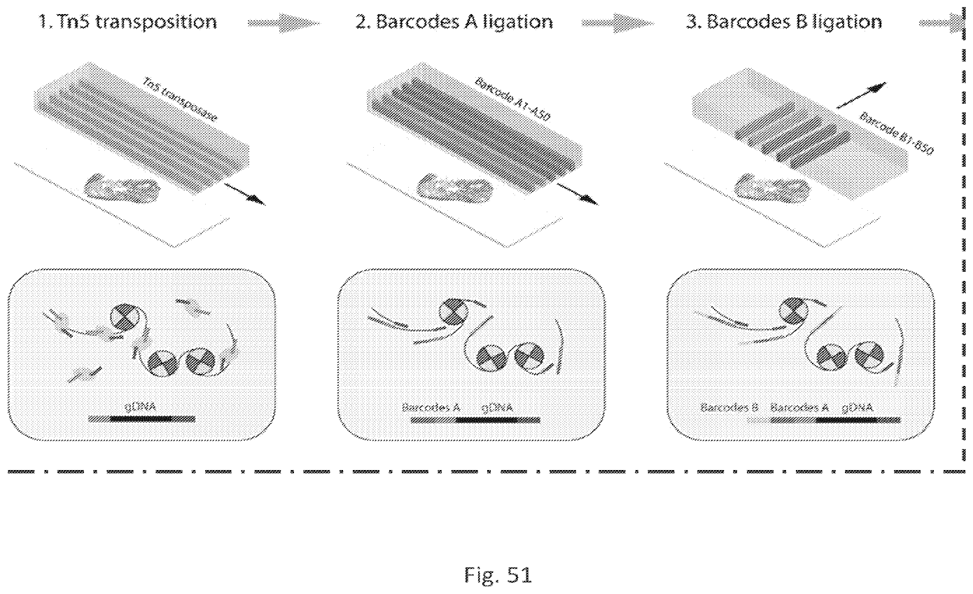

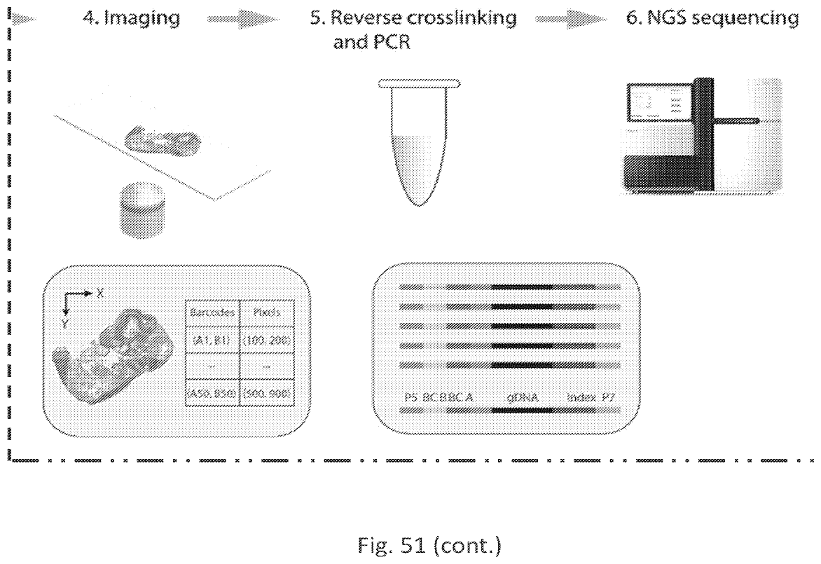

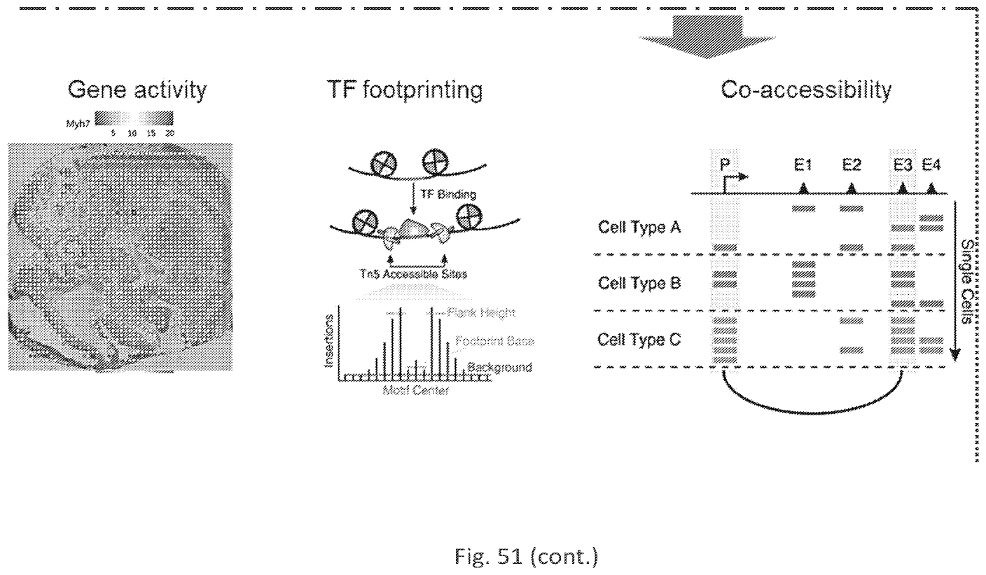

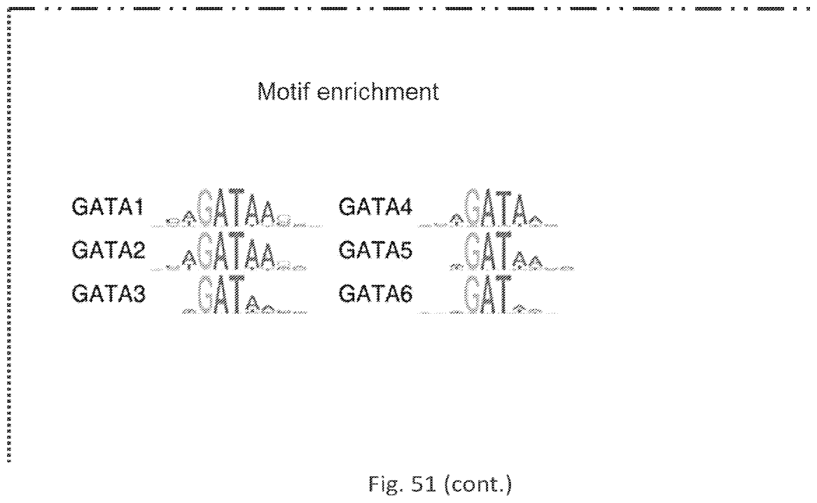

FIG. 51 depicts a schematic diagram of the hsrATAC-seq method.

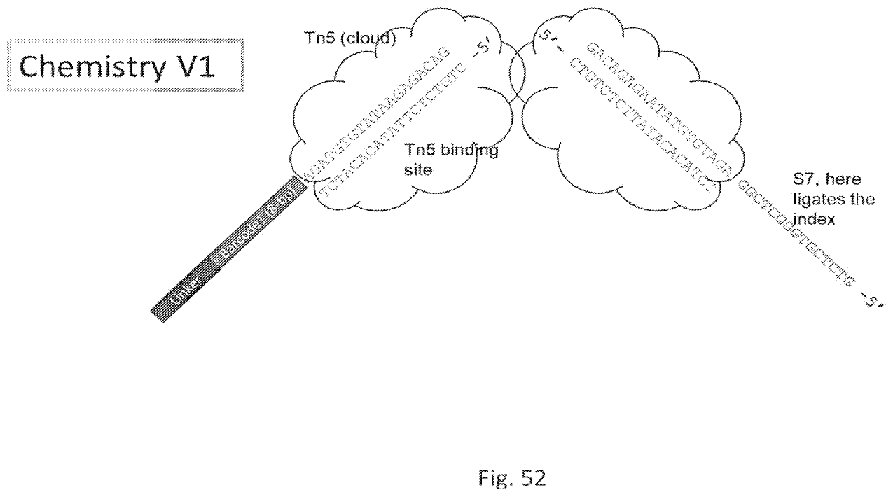

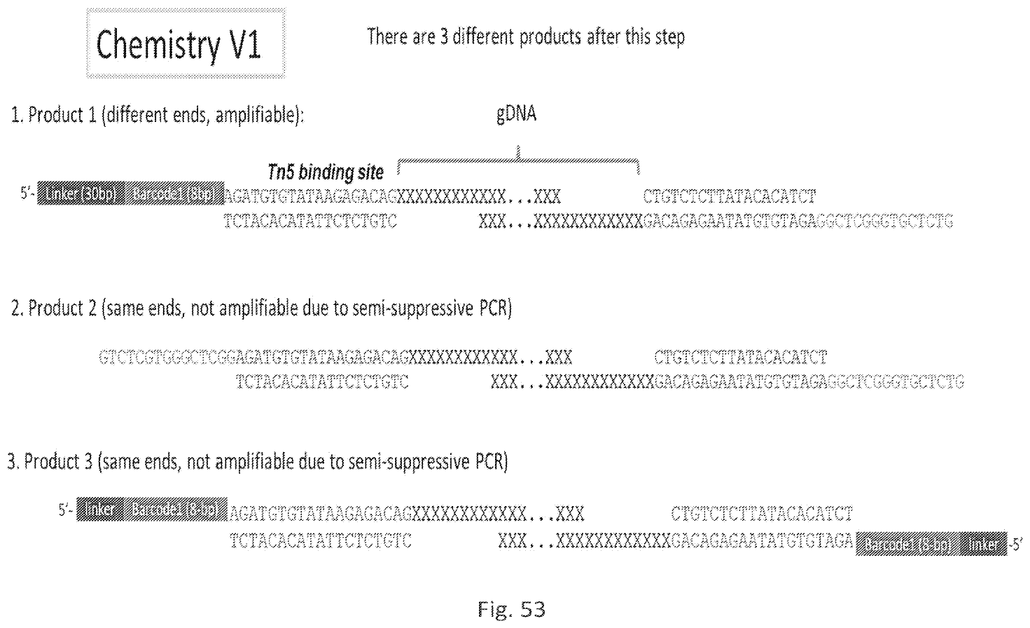

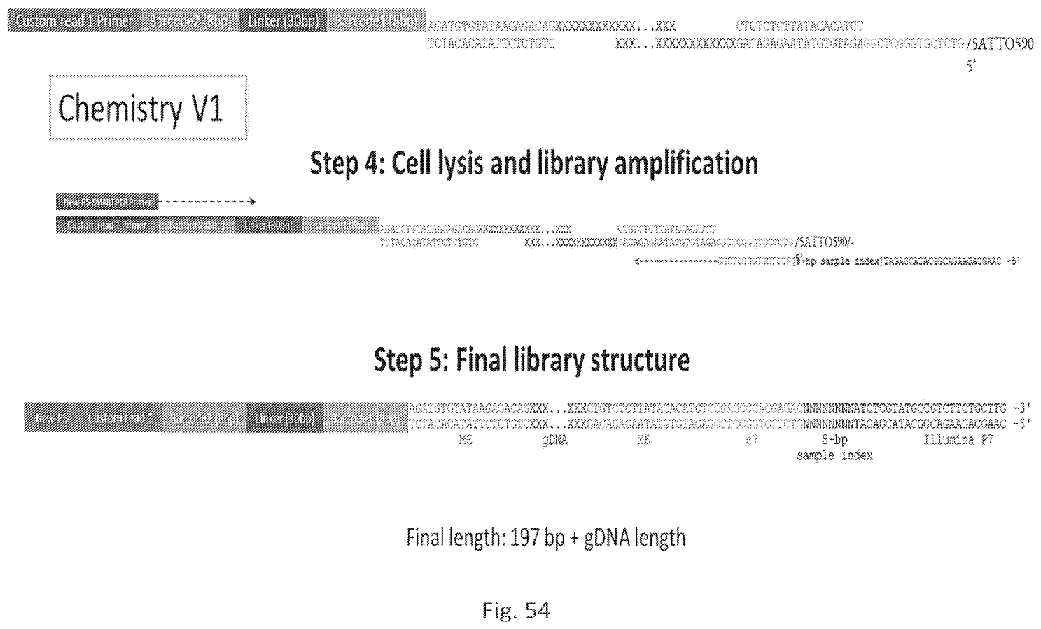

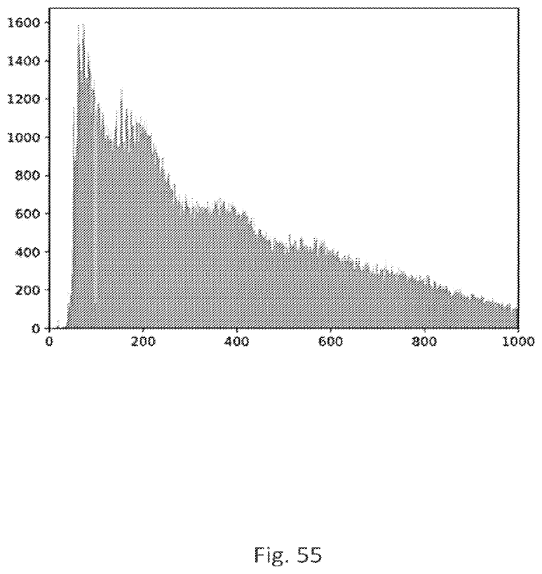

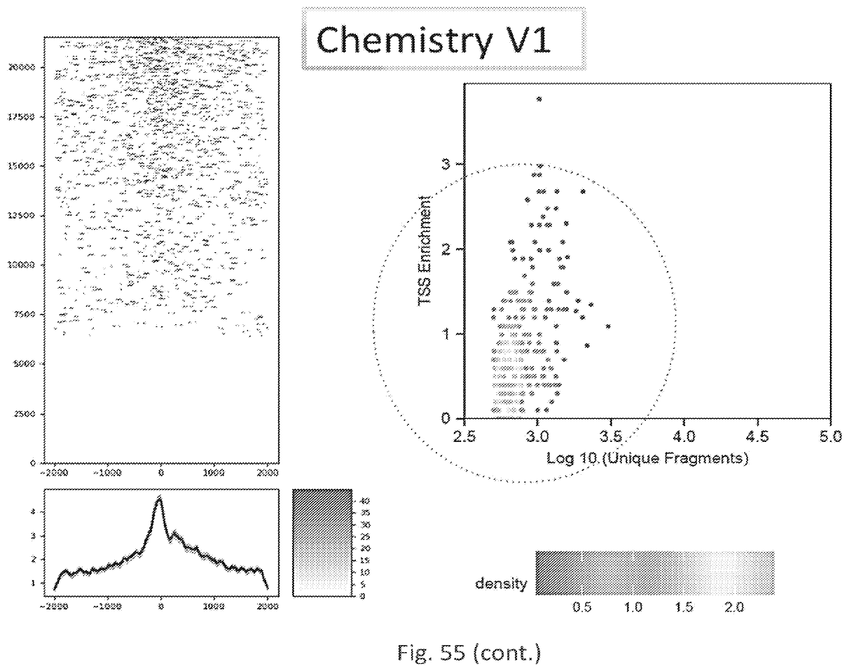

FIGS. 52-55 depict results generated with the first version of the hsrATAC-seq method (hsrATAC-seq v1).

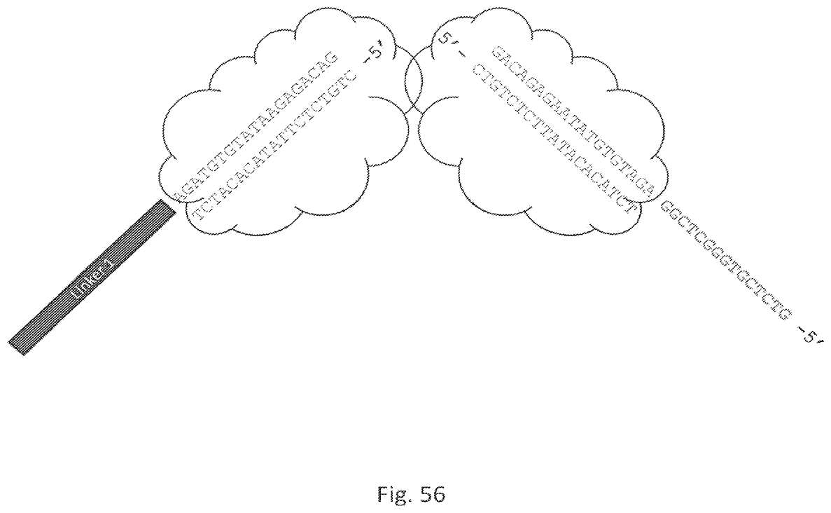

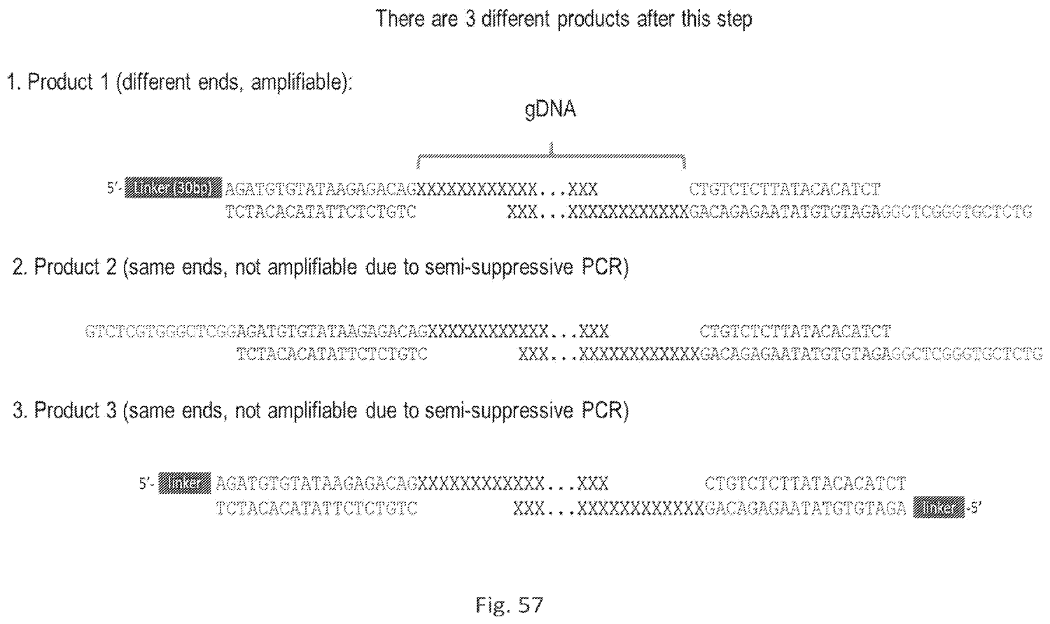

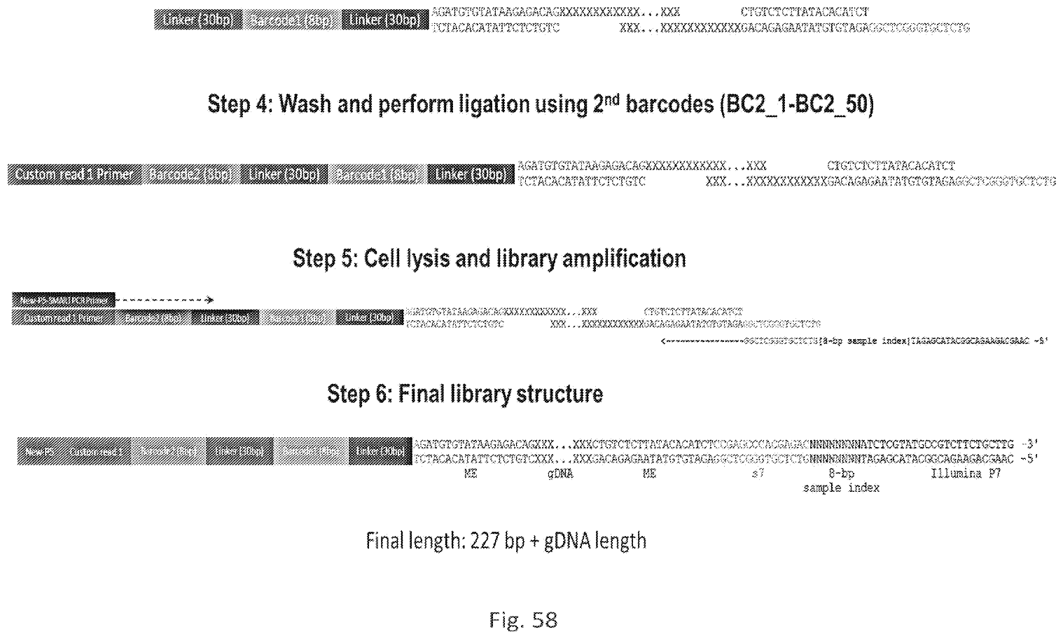

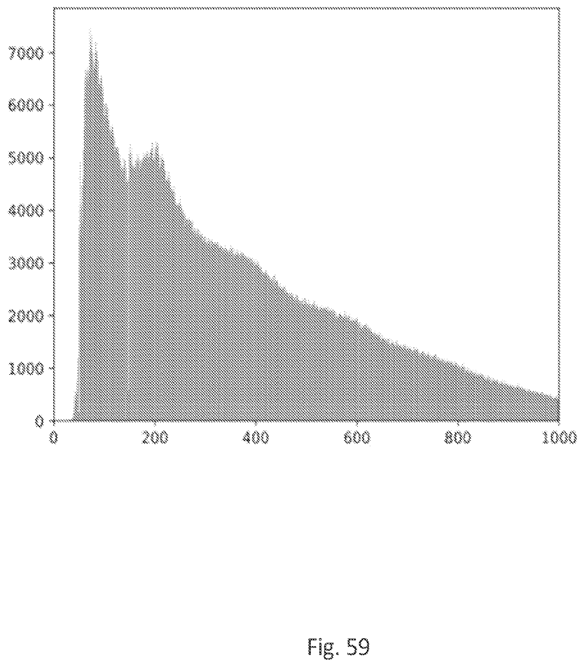

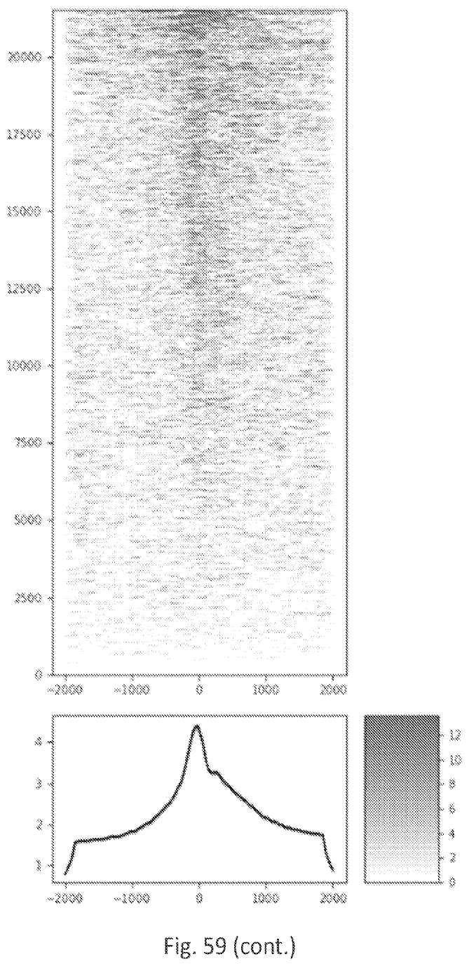

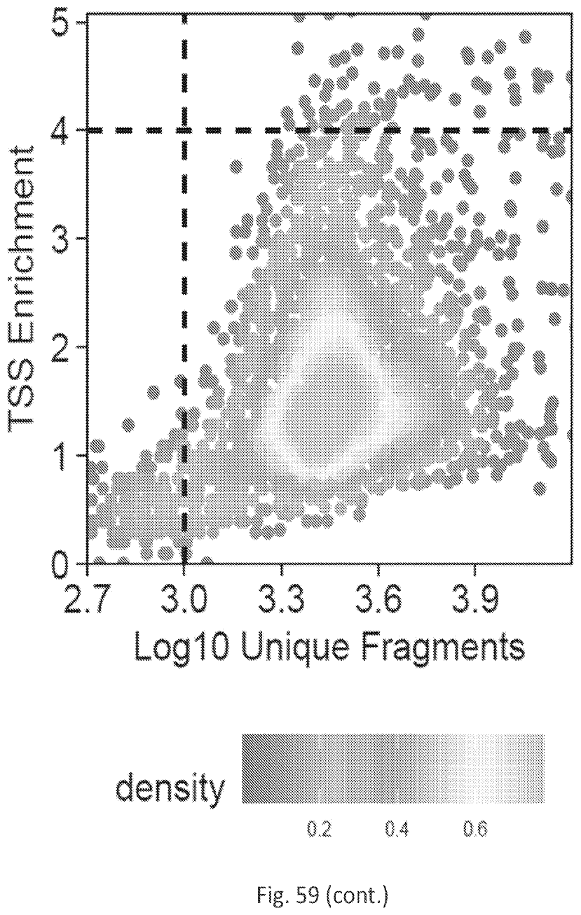

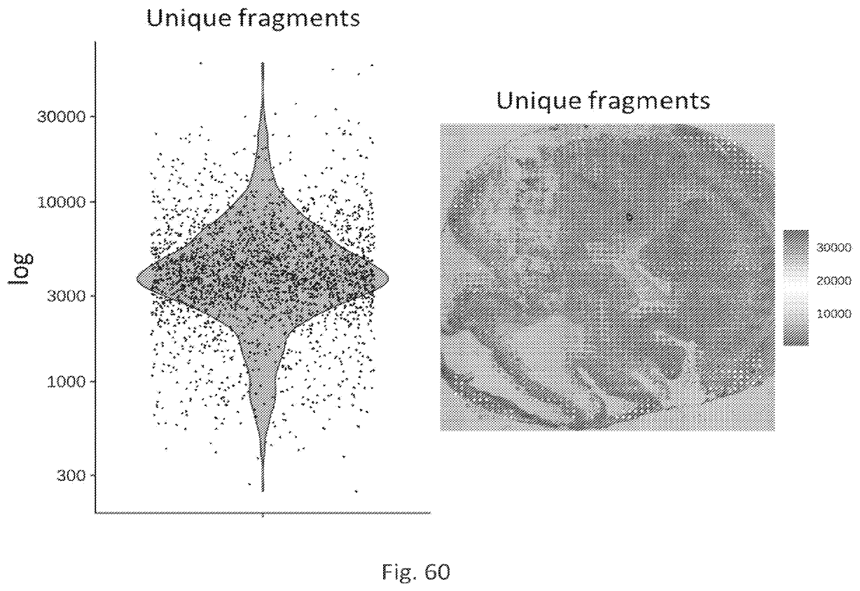

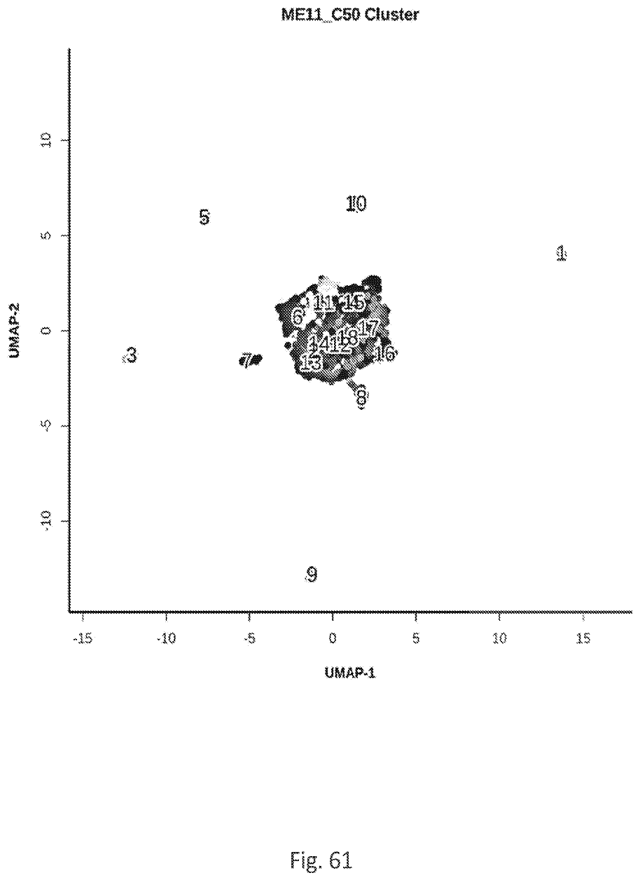

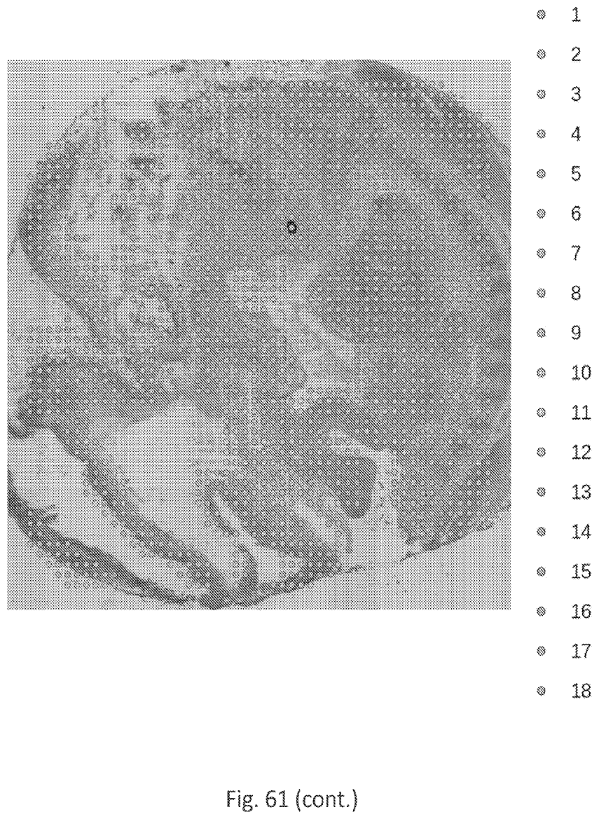

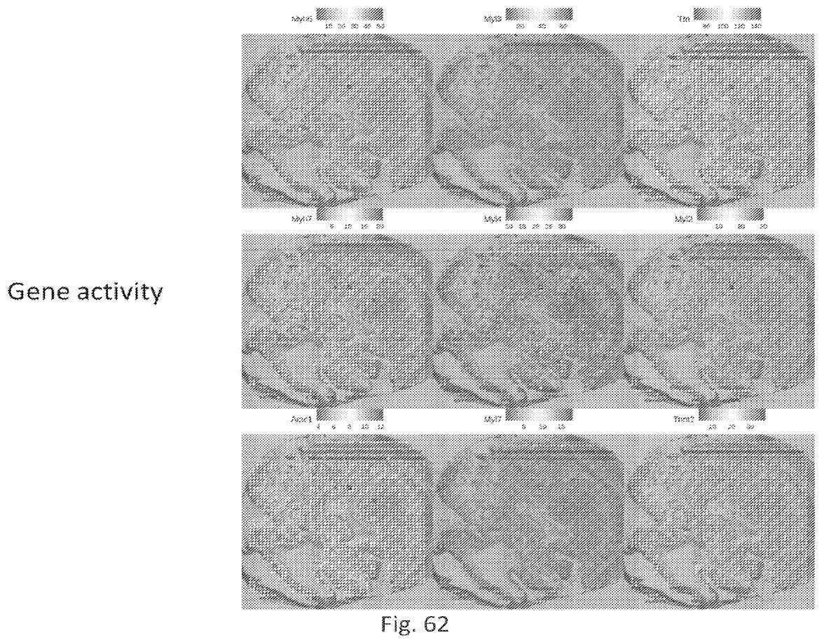

FIG. 56-62 depict results generated with the second version of the hsrATAC-seq method (hsrATAC-seq v2).

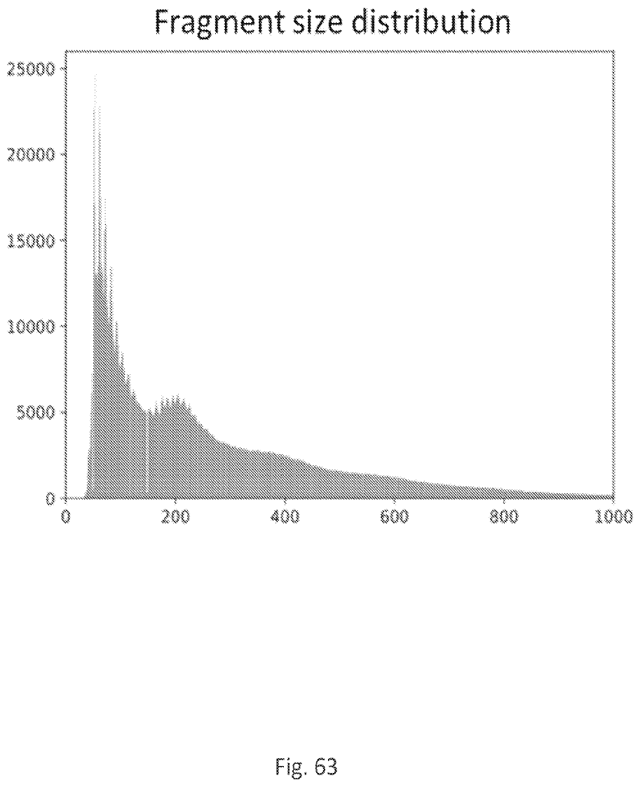

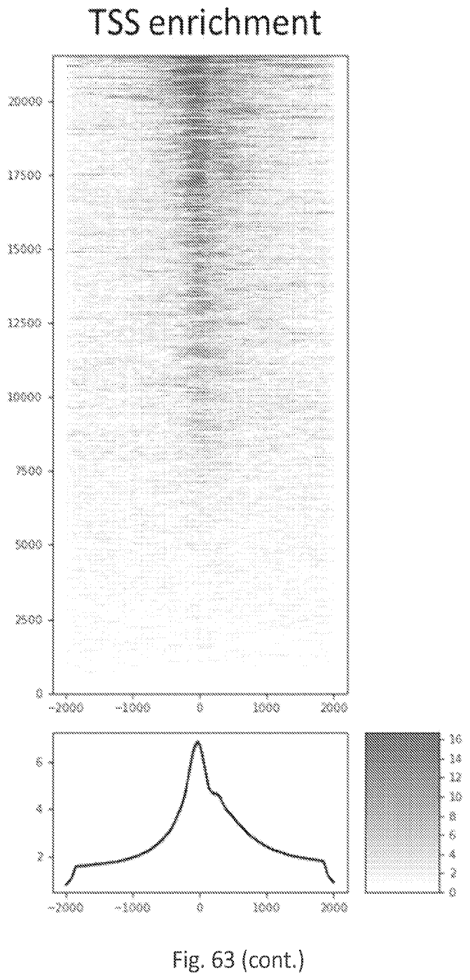

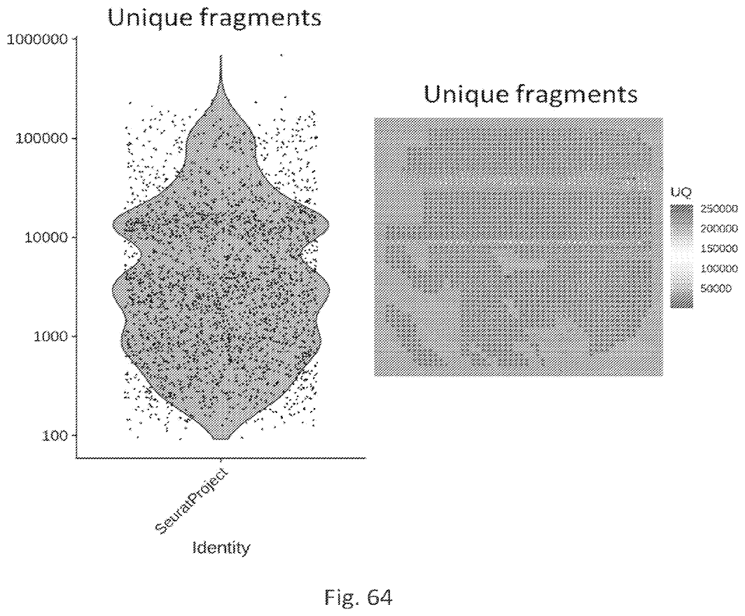

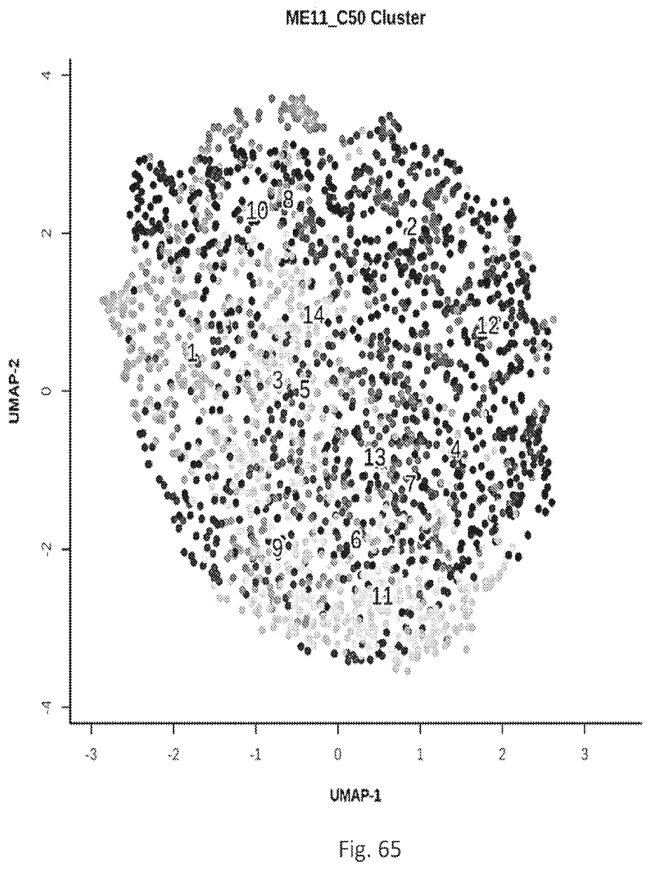

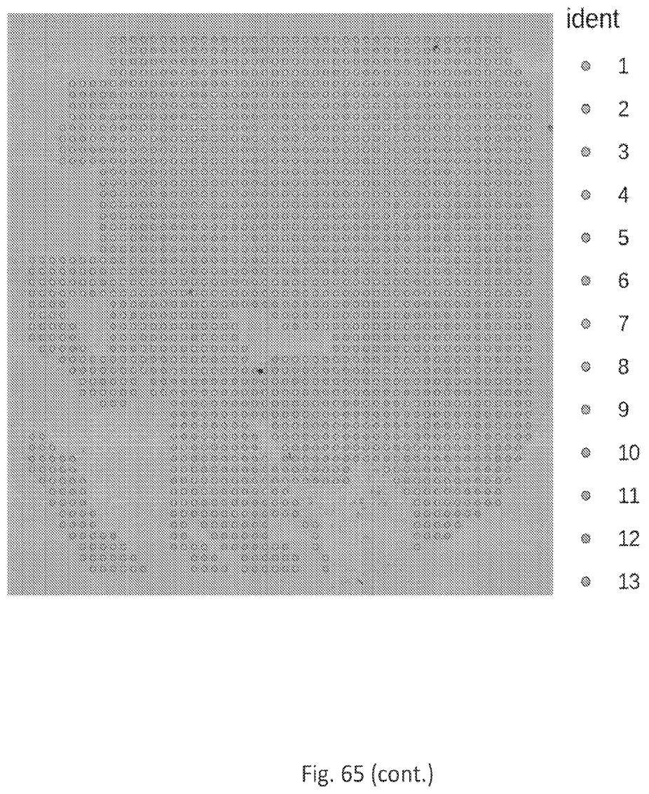

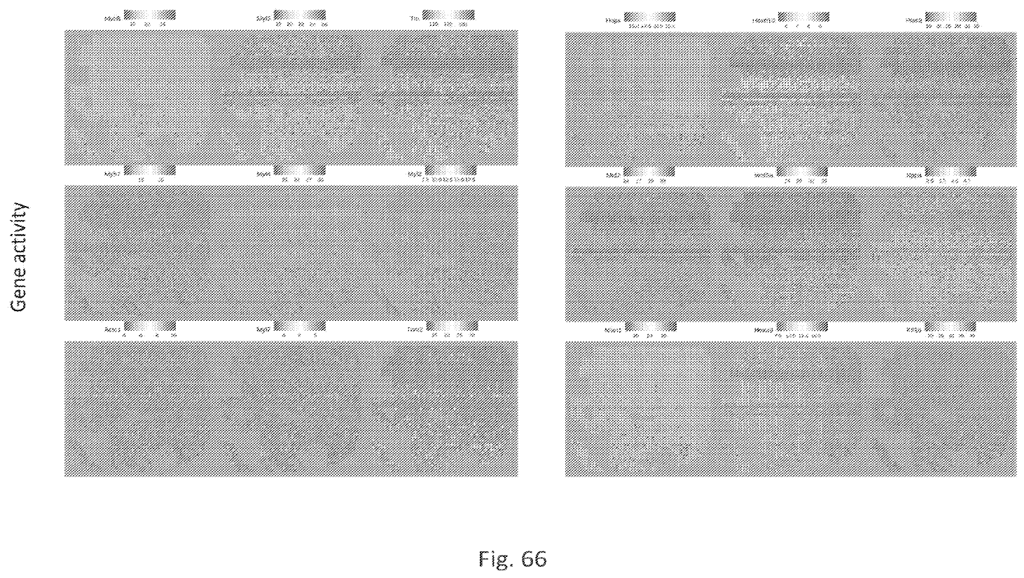

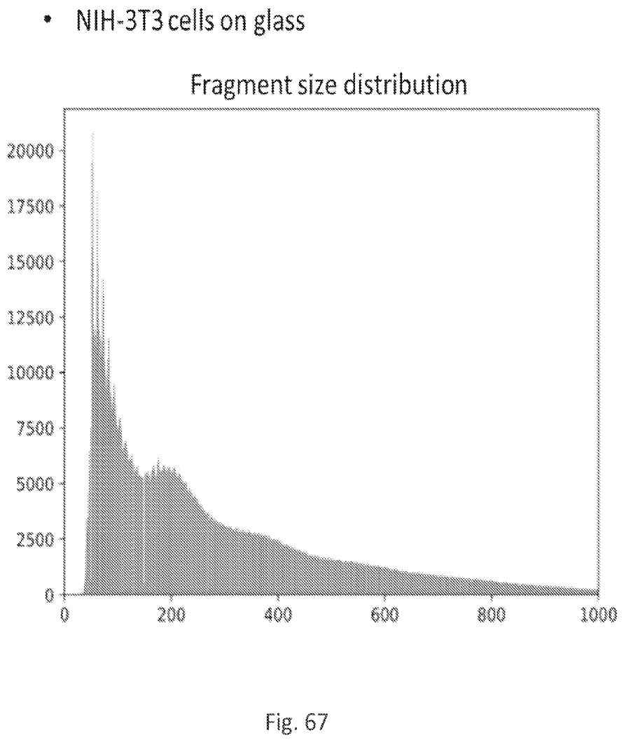

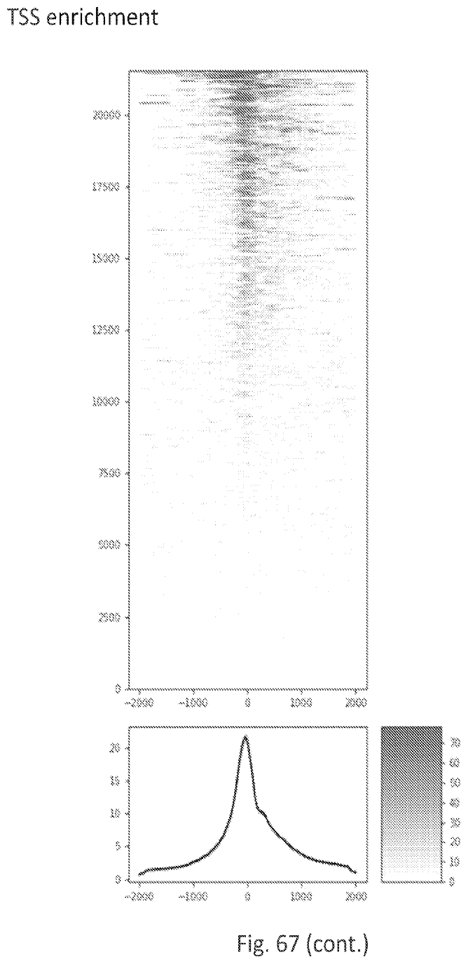

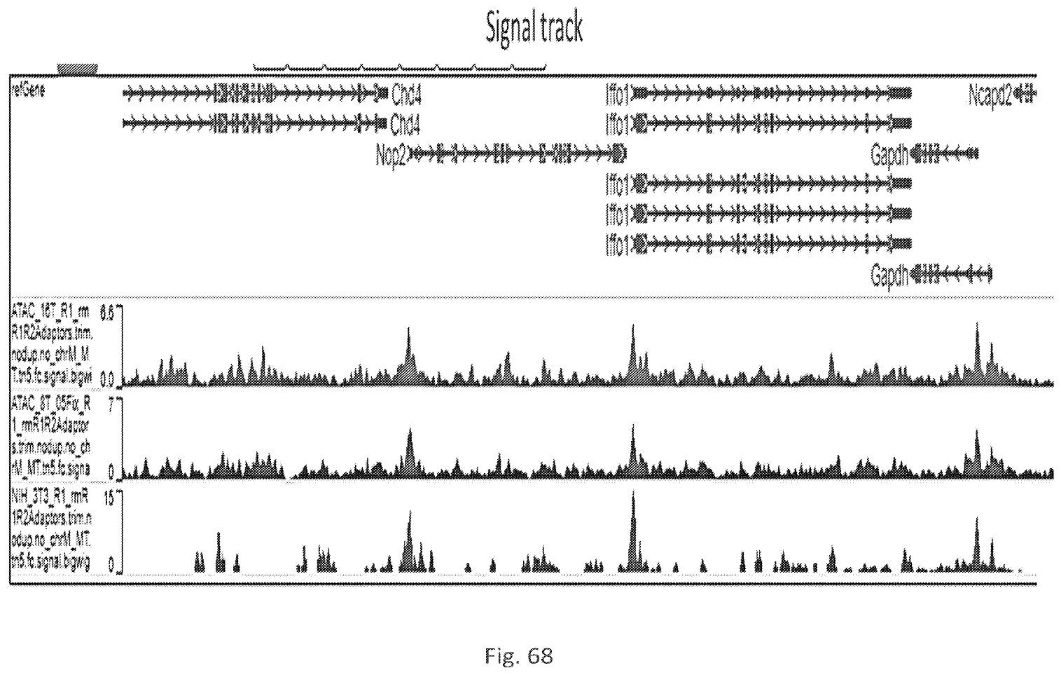

FIG. 63-68 depict results generated with an optimization of the second version of the hsrATAC-seq method (hsrATAC-seq v2.1).

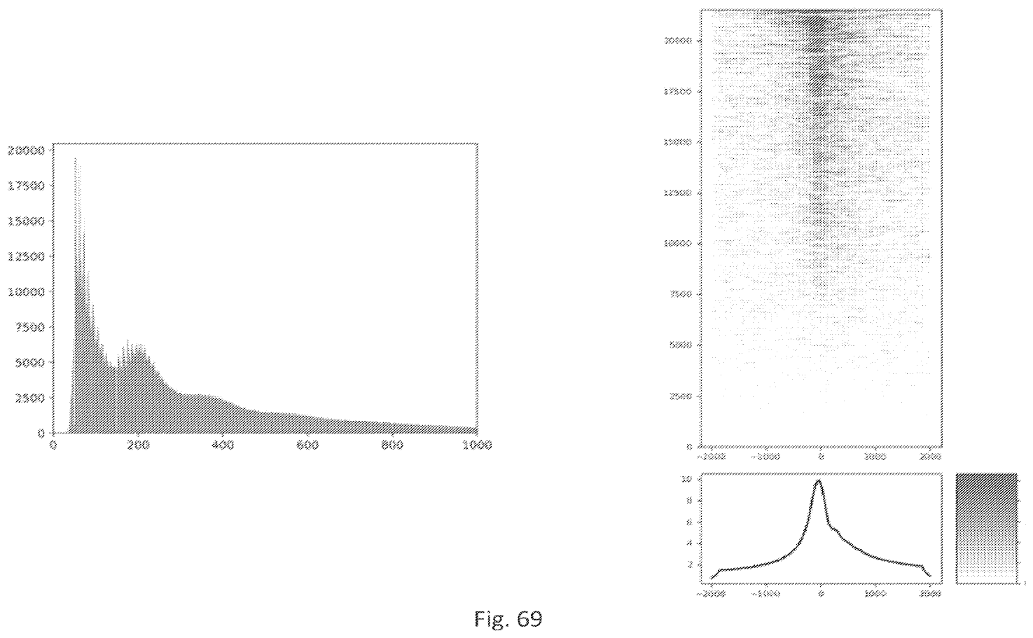

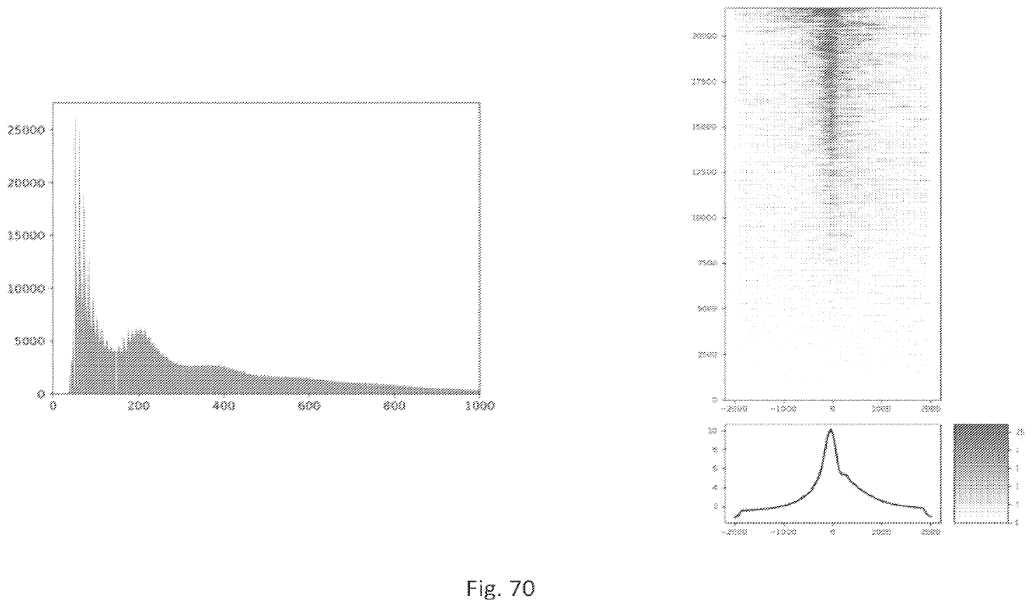

FIG. 69-70 depict results generated with an optimization of the 2.1 version of the hsrATAC-seq method (hsrATAC-seq v2.2).

### Example 4: Spatial-CUT&Tag: Spatially Resolved Chromatin Modification Profiling at Tissue Scale and Cellular Level

The data presented herein describe the profiling of chromatin states in situ in tissue sections with high spatial resolution. Although spatial-CUT&Tag exclusively focused on the tissue mapping of chromatin states, integration with other spatial assays such as transcriptome and proteins is feasible with the microfluidic in tissue barcoding approach by combining reagents for DBiT-seq (Liu et al., 2020, Cell, 183:1665-1681.e1618) and spatial-CUT&Tag in the same microfluidic channels to achieve spatial multi-omics profiling. Moreover, the mapping area of spatial-CUT&Tag could be further increased by increasing the number of barcodes (e.g. 100×100) or using a serpentine microfluidic channel design without the need to increase the number of DNA barcodes. Spatial-CUT&Tag is an NGS-based approach, which is unbiased and genome-wide for mapping biomolecular mechanisms in the tissue context. This capability would enable novel discovery of causative relationships throughout the Central Dogma of molecular biology from epigenome to transcriptome and proteome in individual cells with broad implications in how tissues organize and how diseases develop. The versatility and scalability of this method may accelerate the mapping of chromatin states at large tissue scale and cellular level to significantly enrich cell atlases with spatially resolved epigenomics, adding a new dimension to spatial biology.

The materials and methods are now described

### Animals

The mouse line Sox 10:Cre-RCE:LoxP (EGFP), on a C57BL/6xCD1 mixed genetic background, was used for experiments on P21 mice. It was generated by crossing Sox 10:Cre animals (Kelsey et al., 2017, Science, 358:69-7522) (The Jackson Laboratory mouse stock number #025807) on a C57BL/6j genetic background with RCE:loxP (enhanced green fluorescent protein (EGFP)) animals (Nguyen et al., 2018, Frontiers in Cell and Developmental Biology, 6) (The Jackson Laboratory mouse stock number #32037-JAX) on a C57BL/6xCD1 mixed genetic background. Breedings of females with a hemizygous Cre allele with males lacking the Cre allele (while the reporter allele was kept in hemizygosity or homozygosity in both females and males) resulted in labeling the oligodendrocyte lineage with EGFP. Mice, free of common viral pathogens, ectoparasites, endoparasites and mouse bacterial pathogens, were housed to a maximum number of 5 per cage in individually ventilated cages (IVC sealsafe GM500, Tecniplast). The cages were equipped with hardwood bedding (TAPVEI), nesting material, shredded paper, gnawing sticks and a cardboard box shelter (Scanbur). Mice received regular chew diet and water using a water bottle that was changed weekly. Cages were changed every other week in a laminar air-flow cabinet. General housing parameters such as relative humidity, temperature, and ventilation follow the European convention for the protection of vertebrate animals used for experimental and other scientific purposes treaty ETS 123. The following light/dark cycle was used: dawn 6:00-7:00, daylight 7:00-18:00, dusk 18:00-19:00, night 19:00-6:00.

### Tissue Preparation and Sectioning:

Embryonic tissue samples were purchased commercially. Mouse C57 Embryo Sagittal Frozen Sections (Zyagen, MF-104-11-C57) and Mouse C57 Olfactory bulb Coronal Frozen Sections (Zyagen, MF-201-01-C57) were prepared by Zyagen (San Diego, CA). Embryos were snapped frozen in OCT blocks, sectioned at a thickness of 7-10 μm and mounted at the center of poly-L-lysine coated glass slides (Electron Microscopy Sciences, 63478-AS). The tissues sections used for 50 μm experiments are from the same mouse embryo, and the tissues sections used for 20 μm experiments are from another mouse embryo.

Juvenile (P21) mice were sacrificed by anesthesia with ketamine (120 mg/kg of body weight) and xylazine (14 mg/kg of body weight), and subsequent transcranial perfusion with cold oxygenated artificial cerebrospinal fluid aCSF (87 mM NaCl, 2.5 mM KCl, 1.25 mM NaH2PO4, 26 mM NaHCO₃, 75 mM Sucrose, 20 mM Glucose, 1 mM CaCl2*2H2O and 2 mM MgSO4*7H2O in dH2O). The brains were isolated from the skull, embedded in Tissue-Tek® O.C.T. compound (Sakura) and snap frozen using a mixture of dry ice and ethanol.

The brains were coronally cryosectioned into 10 μm sections (in 1:8 series) and collected on poly-L-lysine coated glass slides (Electron Microscopy Sciences, 63478-AS). The samples were stored at −80° C. until further use.

### Microfluidic Device Fabrication and Assembly

The molds of microfluidic devices were fabricated using photo lithography. SU-8 negative photoresist (Microchem, SU-2010, SU-2025) was spin-coated on a silicon wafer (WaferPro, C04004) following manufacturer's guidelines. The feature height of 50-μm-wide microfluidic channel device was ˜50 μm, and ˜23 μm for 20-μm-wide device. Chrome photomasks (Front Range Photomasks) were used during UV exposure.

Microfluidic devices were then fabricated using soft lithography. Polydimethylsiloxane (PDMS) was prepared by mixing base and curing agent at a 10:1 ratio (Ellsworth Adhesives, 184 SIL ELAST KIT 3.9 KG). PDMS was then added over the SU-8 masters. After degassing in the vacuum for 30 min, the PDMS was cured at 65° C. for 2 hours. The solidified PDMS slab was cut out and the inlet and outlet holes were punched to complete the fabrication.

### DNA Barcodes and Other Key Reagents

DNA oligos used for PCR and preparation of sequencing library were listed in Table 1, DNA barcode sequences were listed in Table 3 (Example 7), and all other key reagents used were listed as Table 2.

### H&E Staining

The slide with frozen tissue section was first kept at room temperature for 10 minutes before a subsequent 10-minute fixation with 4% formaldehyde. Next, 500 μL of isopropanol was added to the tissue and incubated for 1 minute. After the isopropanol was removed, the tissue was left to air dry. Staining with 1 mL of hematoxylin (Sigma) was performed at room temperature for 7 minutes. Afterward, the slide was washed with DI water and incubated in 1 mL of bluing reagent (Sigma, 0.3% acid alcohol) for 2 minutes at room temperature. Finally, after an additional rinse with DI water, the tissue slide was stained with eosin for 2 minutes and rinsed again with DI water. The stained tissue section was imaged using EVOS (Thermo Fisher EVOS fl) at a magnification of 20×.

### Transposome Preparation

Unloaded pA-Tn5 transposase was purchased from Diagenode (C01070002), and the transposome was assemble by following manufacturer's guidelines. The oligonucleotides used during transposome assembly were:

Tn5ME-A:
(SEQ ID NO: 115)
5′-/5Phos/CATCGGCGTACGACTAGATGTGTATAAGAGACAG-3′
Tn5ME-B:
(SEQ ID NO: 102)
5′-GTCTCGTGGGCTCGGAGATGTGTATAAGAGACAG-3′
Tn5MErev:
(SEQ ID NO: 101)
5′-/5Phos/CTGTCTCTTATACACATCT-3′

### Spatial-CUT&Tag

The slide with frozen tissue section was brought to room temperature by 10-minute incubation. Then, the tissue was fixed with 0.2% formaldehyde for 5 minutes and quenched with 1.25 M glycine for 5 min at room temperature. After the fixation, tissue was washed twice with 1 mL Wash Buffer (20 mM HEPES pH 7.5; 150 mM NaCl; 0.5 mM Spermidine; 1 tablet Protease inhibitor cocktail) and rinsed with DI water. The tissue section was then permeabilized for 5 minutes with NP40-Digitonin Wash Buffer (0.01% NP40, 0.01% Digitonin in wash buffer). After removing the NP40-Digitonin Wash Buffer, primary antibody (Table 2) (1:50 dilution in antibody buffer (2 mM EDTA and 0.001% BSA in NP40-Digitonin Wash Buffer) was added followed by incubation overnight at 4° C. The primary antibody was then removed, and secondary antibody (Table 2) (1:50 dilution in NP40-Digitonin Wash Buffer) was added followed by incubation at room temperature for 30 minutes. Unbound antibodies were removed using Wash buffer for 5 minutes. A 1:100 dilution of pA-Tn5 adapter complex in 300-wash buffer was added followed by 1-hour incubation at room temperature. Excess pA-Tn5 protein was removed using 300-wash buffer (20 mM HEPES pH 7.5; 300 mM NaCl; 0.5 mM Spermidine; 1 tablet Protease inhibitor cocktail) for 5 minutes. Next, Tagmentation buffer (10 mM MgCl2 in 300-wash buffer) was added followed by incubation at 37° C. for 1 hour. To stop tagmentation, 40 mM EDTA was added after removing Tagmentation buffer, which was incubated at room temperature for 5 minutes. After removing EDTA, the tissue section was washed with 1× NEBuffer 3.1 for 5 minutes.

To ligate barcodes A in situ, the 1st PDMS device was placed on top of the tissue slide with the region of interest covered, followed by imaging with 10× objective (Thermo Fisher EVOS fl microscope) for alignment in the downstream analysis. Afterwards, the tissue slide and PDMS device were clamped tightly with an acrylic clamp. The ligation mix was prepared in a 1.5 mL tube using 72.4 μL of RNase free water, 27 μL of T4 DNA ligase buffer, 11 μL T4 DNA ligase, and 5.4 μL of 5% Triton X-100.

DNA barcodes A were first annealed with ligation linker 1 by adding 10 μL of each DNA Barcode A (100 μM), 10 μL of ligation linker (100 μM) and 20 μL of 2× annealing buffer (20 mM Tris, pH 7.5-8.0, 100 mM NaCl, 2 mM EDTA). Ligation reaction solution (50 tubes) was prepared by combining 2 μL of ligation mix, 2 μL of 1×NEBuffer 3.1 and 1 μL of each DNA barcode A (A1-A50, 25 μM). The solution was then loaded into each of the 50 channels with vacuum. The chip was kept in a wet box and incubated at 37° C. for 30 minutes. After washing by flowing 1×NEBuffer 3.1 for 5 minutes, the clamp and PDMS were removed. The slide was quickly dipped in water and dried with air.

To ligate barcode B, the 2nd PDMS slab with channels perpendicular to the 1 st PDMS was attached to the dried slide with care. A brightfield image was taken (EVOS at a magnification of 10×) and the clamp was used to press the PDMS against the tissue. Next, 115.8 μL of ligation mix was prepared. DNA barcodes B were first annealed with ligation linker 2 by adding 10 μL of each DNA Barcode B (100 μM), 10 μL of ligation linker (100 μM) and 20 μL of 2× annealing buffer (20 mM Tris, pH 7.5-8.0, 100 mM NaCl, 2 mM EDTA). Ligation reaction solution (50 tubes) was prepared by combining 2 μL of ligation mix, 2 μL of 1×NEBuffer 3.1 and 1 μL of each DNA barcode B (B1-B50, 25 μM). The solution was again loaded into each of the 50 channels with vacuum. The chip was kept in a wet box and incubated at 37° C. for 30 minutes. After washing by flowing 1×DPBS for 5 minutes, the clamp and 2nd PDMS were removed. The tissue section was dipped in water and air dried before taking the final brightfield image (EVOS at a magnification of 10×).

Fluorescent staining of tissue sections with common nucleus staining dyes can be performed before tissue digestion to facilitate the identification of tissue region of interest. Working solution mixture of DAPI were added on top of the tissue and then incubate at room temperature for 20 minutes, followed by washing twice with 1×PBS. Images of the tissue were taken using EVOS microscope with 10× objective and DAPI Light Cube.

Afterwards, the tissue region of interest was covered with a square PDMS well gasket and then washed twice with TAPS wash buffer (10 mM TAPS, 0.2 mM EDTA) before loading of lysis solution (0.1% SDS, 10 mM TAPS). Lysis was performed at 60° C. for 2 hours in a wet box. The tissue lysate was then collected into a 200 μL PCR tube and incubate at 65° C. with rotation for another 1 hour.

To construct the library, lysates were distributed into PCR tubes (5 μL each) before the addition of 15 μL Triton neutralization solution (0.67% Triton-X100), 2 L of 10 μM new P5 PCR primer, 2 μL of 10 μM i7 primers, and 25 μL NEBnext PCR Master Mix into each tube. Then, PCR was performed using the following program: initial incubation at 58° C. for 5 minutes, followed by incubations at 72° C. for 5 min and 98° C. for 30 s, 12 cycles at 98° C. for 10 s, and incubation at 60° C. for 10 s, followed by the final incubation at 72° C. for 1 min. To remove remaining PCR primers, the PCR product was purified by 1.3× Ampure XP beads using the standard protocol and eluted in 10 mM Tris-HCl pH 8. Before sequencing, the size distribution and concentration of the library were quantified by an Agilent Bioanalyzer High Sensitivity Chip. NGS sequencing was then performed using a HiSeq 4000 sequencer with pair-end 150 bp mode with custom read 1 primer.

### Data Preprocessing

Read 1 was first filtered by two constant linker sequences (linker 1 and linker 2). Then filtered sequences were processed to cellranger atac format (10× Genomics), where the new Read 1 was genome sequences and the new Read 2 includes barcodes A and barcodes B. Resulting fastq files were aligned to the mouse genome (mm10), filtered for duplicates and counted using Cell Ranger ATAC v1.2, which generated the BED like fragments file for downstream analysis. The fragments file contains tissue location info (barcode A×barcode B) and fragments info on the genome. To calculate the FRiP in spatial-CUT&Tag data, peaks were called from each sample using MACS2 (Denisenko et al., 2020, Genome Biology, 21:130). A preprocessing pipeline was developed using Snakemake workflow management system, which is shared at github.com/dyxmvp/spatial-CUT&Tag.

### Data Visualization

Microscope images were taken with channels on top for each experiment. By overlaying the channel images with tissue images, the pixel locations were identified. Pixels were first identified on tissue with manual selection from microscope image using Adobe Illustrator (github.com/rongfan8/DBiT-seq), and a custom python script was used to generate metadata files that were compatible with Seurat workflow for spatial datasets.

The fragments file was then read into ArchR as a tile matrix in 5 kb genome binning size, and pixels not on tissue were removed based on the metadata file generated from the previous step. Data normalization and dimensionality reduction was performed using Latent Semantic Indexing (LSI) (iterations=2, resolution=0.2, varFeatures=25000, dimsToUse=1:30, sampleCells=10000, n.start=10), followed by graph clustering and Uniform Manifold Approximation and Projection (UMAP) embeddings (nNeighbors=30, metric=cosine, minDist=0.5) (Han et al., 2017, Nucleic Acids Res, 45).

Chromatin silencing score (CSS) and gene activity score (GAS) were calculated using Gene Score model in ArchR and Gene Score Matrix was generated for downstream analysis. Marker regions/genes for each cluster were identified using the getMarkerFeatures and getMarkers function in ArchR (testMethod=“wilcoxon”, cutOff=“FDR<=0.05”), and gene scores imputation was implemented with addImputeWeights function for data visualization. Peaks were called using the MACS2. Motif enrichment and motif deviations were calculated using peakAnnoEnrichment and addDeviationsMatrix function in ArchR. GO enrichment analysis was implemented using the clusterProfiler package (qvalueCutoff=0.05) (van den Brink et al., 2017, Nature Methods, 14:935-936). To map the data back to the tissue section, results obtained in ArchR were loaded to Seurat V3.2 for spatial data visualization (Lake et al., 2018, Nat Biotechnol, 36:70-80; Larsson et al., 2021, Nat Methods, 18:15-18).

To project bulk ChIP-seq data, raw sequence data aligned to mm10 (BAM files) from ENCODE were downloaded. After reads were counted in 5 kb tiled genomes using getCounts function in chrom VAR (Rodriques et al., 2019, Science, 363:1463-1467), the bulk projection function was then used in ArchR.

Cell type identification and pseudo-scRNA-seq profiles was added through integration with scRNA-seq reference data (Hu et al., 2016, Genome Biol, 17). FindTransferAnchors function (Seurat V3.2 package) was used to align pixels from spatial-CUT&Tag with cells from scRNA-seq by comparing the spatial-CUT&Tag gene score matrix with the scRNA-seq gene expression matrix. GeneIntegrationMatrix function in ArchR was used to add cell identities and pseudo-scRNA-seq profiles.

To compute per-cell motif activity, chrom VAR (Rodriques et al., 2019, Science, 363:1463-1467) was run with addDeviationsMatrix using the cisbp motif set after a background peak set was generated using addBgdPeaks. Pseudotemporal reconstruction was implemented by addTrajectory function in ArchR. The codes outlining how the downstream analysis was performed are available at github.com/dyxmvp/spatial-CUT-Tag.

### Integrative Data Analysis and Cell Type Identification

Cell type identification and pseudo-scRNA-seq profiles was added through integration with and scRNA-seq reference data (Hu et al., 2016, Genome Biol, 17). Pixels from spatial-CUT&Tag were aligned with cells from scRNA-seq by comparing the spatial-CUT&Tag gene score matrix with the scRNA-seq gene expression matrix, which was performed using the FindTransferAnchors function from the Seurat V3.2 package. Afterwards, cell identities and pseudo-scRNA-seq profiles were added using addGeneIntegrationMatrix function in ArchR. Data from 20 μm spatial-CUT&Tag P21 mouse brain were integrated with single-cell CUT&Tag data using CCA implemented in Seurat v3. 5 kb H3K4me3 and H3K27me3 matrices were used for the integration. Related codes were shared at github.com/dyxmvp/spatial-CUT-Tag.

Data Quality Comparison with Other Techniques

To compare with other techniques, published data was downloaded: scCUT&Tag: GSE163532.

ENCODE (bulk): Public bulk ChIP-seq datasets were downloaded from ENCODE (H3K27me3, H3K4me3 and H3K27ac from mouse embryos E11.5).

Mouse organogenesis cell atlas (MOCA): oncoscape.v3.sttrcancer.org/atlas.gs.washington.edu.mouse.rna/downloads

Mouse Brain Atlas: mousebrain.org/DBiT-seq:

GSE137986 (Mouse embryo Brain E11 10 μm resolution).

The experimental results are now described

Chromatin state is of great importance in determining the functional output of the genome and is dynamically regulated in a cell type-specific manner (Schwartzman et al., 2015, Nature Reviews Genetics 16, 716-726; Kelsey et al., 2017, Science, 358:69; Carter et al., 2020, Nature Reviews Genetics; Gorkin et al., 2020, Nature 583, 744-751; Deng et al., 2019, Annual Review of Biomedical Engineering, 21:365-393). Despite the recent breakthroughs in massively parallel single-cell sequencing (Macosko et al., 2015, Cell, 161:1202-1214; Klein et al., 2015, Cell, 161:1187-1201; Cao et al., 2019, Nature, 566:496-502; Gierahn et al., 2017, Nat Methods, 14:395-398; Bose et al., 2015, Genome Biol, 16:120; Dura et al., 2019, Nucleic Acids Res, 47:e16; Fan et al., 2015, Science, 347:1258367) that also enabled the profiling of epigenome in individual cells (Rotem et al., 2015, Nature Biotechnology, 33:1165-1172; Grosselin et al., 2019, Nature Genetics, 51:1060-1066; Bartosovic et al., 2021, Nature Biotechnology; Wu et al., 2021, Nature Biotechnology; Mezger et al., 2018, Nat Commun 9, 3647; Han et al., 2017, Nucleic Acids Res, 45; Hu et al., 2016, Genome Biol, 17; Ma et al., 2020, Cell, 183:1103-1116 e1120; Lake et al., 2018, Nat Biotechnol, 36:70-80; Kelsey et al., 2017, Science 358, 69-75), it is becoming increasingly recognized that spatial information of single cells in the original tissue context is equally essential for the mechanistic understanding of biological processes and disease pathogenesis. However, these associations are missing in current single-cell epigenomics data. Furthermore, tissue dissociation in single-cell technologies may preferentially select certain cell types or perturb cellular states as a result of the dissociation or other environmental stresses (Nguyen, 2018, Frontiers in Cell and Developmental Biology, 6; Denisenko et al., 2020, Genome Biology, 21:130; van den Brink et al., 2017, Nature Methods 14, 935-936).

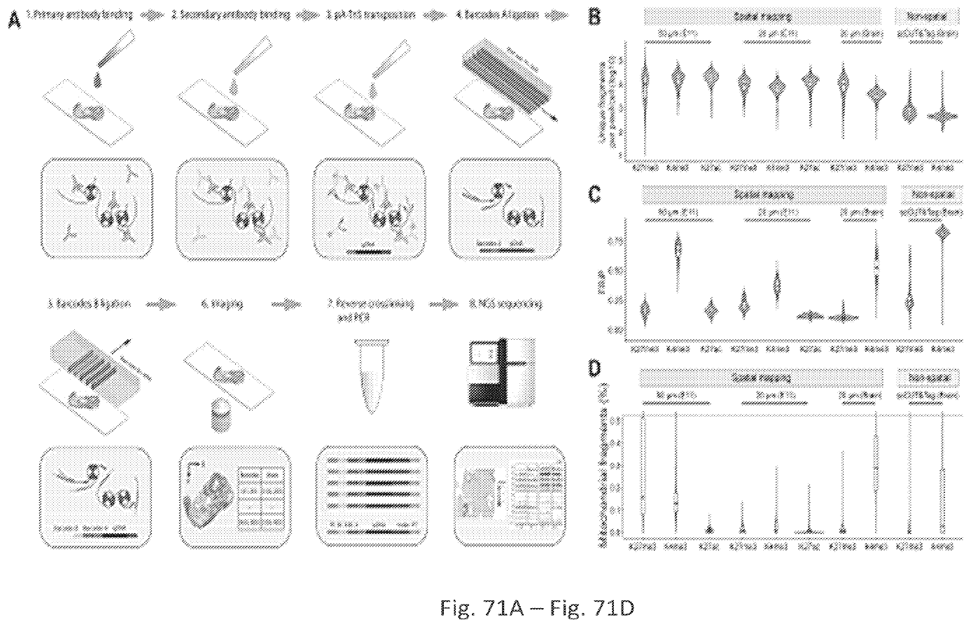

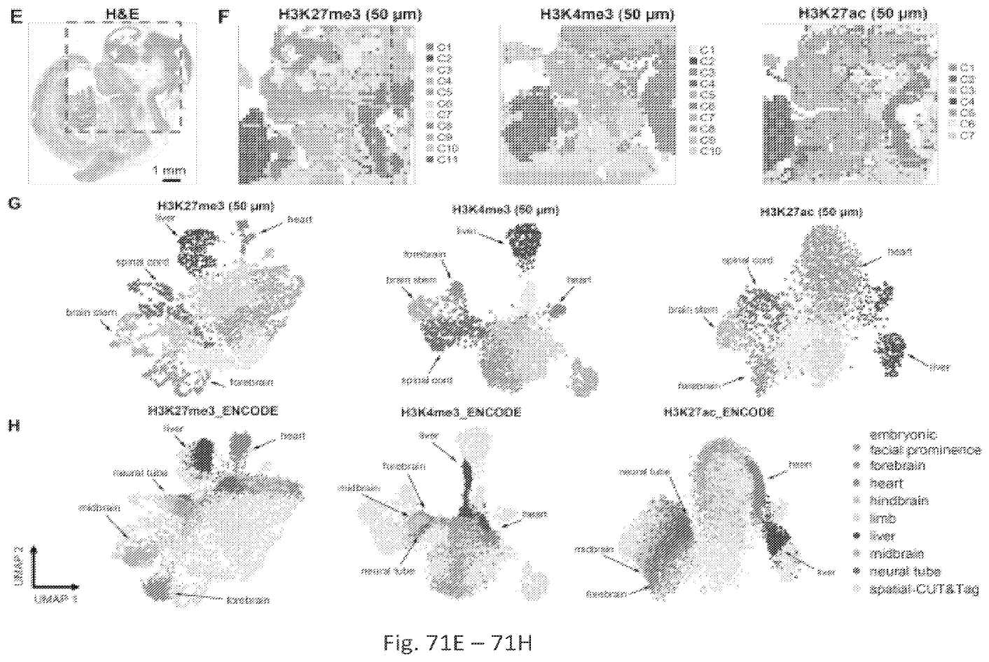

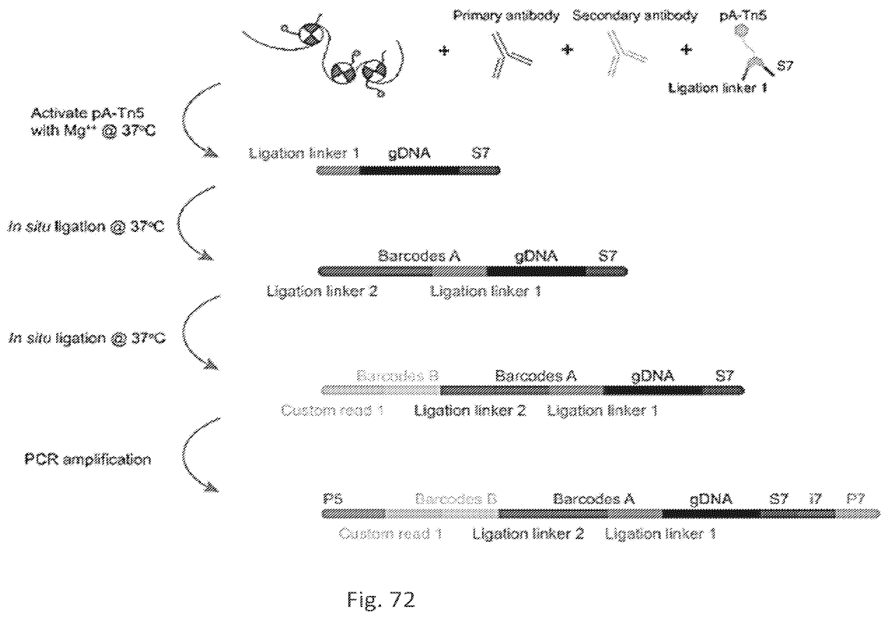

Spatially resolved transcriptomics emerged to address this challenge (Larsson et al., 2021, Nat Methods, 18:15-18; Rodriques et al., 2019, Science, 363:1463-1467; Stahl et al., 2016, Science, 353:78-82; Burgess et al., 2019, Nat Rev Genet, 20:317; Vickovic et al., 2019, Nat Methods). Recently, it was extended to the co-mapping of transcriptome and a panel of proteins via deterministic barcoding in tissue (DBiT-seq) (Liu et al., 2020, bioRxiv, 2020.2010.2013.338475; Liu et al., 2020, Cell 183, 1665-1681.e1618). As of today, it remains unreachable to conduct spatially resolved epigenome sequencing in an intact tissue section. Herein, a first-of-its-kind technology for spatial chromatin modification profiling named spatial-CUT&Tag is reported, which combines the concept of in tissue deterministic barcoding with the Cleavage Under Targets and Tagmentation (CUT&Tag) chemistry (Kaya-Okur et al., 2019, Nature Communications, 10:1930; Henikoff et al., 2020, eLife, 9:e63274) (FIG. 71A and FIG. 72). First, a tissue section on a standard aminated glass slide was lightly fixed with 0.2% formaldehyde. Antibody against the target histone modification was added, followed by a secondary antibody binding to enhance the tethering of pA-Tn5 transposome. By adding Mg⁺⁺ to activate the transposome in tissue, adapters containing a ligation linker were inserted to genomic DNA at the histone mark antibody recognition sites. Then, a set of DNA barcode A solutions were introduced to the tissue section via microchannel-guided delivery (Lu et al., 2015, P Natl Acad Sci USA, 112: E607-E61535) to perform in situ ligation for appending a distinct spatial barcode Ai (i=1-50). Afterwards, a second set of barcodes Bj (j=1-50) were flowed on the tissue surface in microchannels perpendicularly to those in the first flow barcoding step. These barcodes were then ligated at the intersections, resulting in a two-dimensional (2D) mosaic of tissue pixels, each of which contains a distinct combination of barcodes Ai and Bj (i=1-50, j=1-50). The tissue slide being processed could be imaged before, during, or after each flow barcoding step such that the tissue morphology can be correlated with the spatial epigenomics map. After forming a spatially barcoded tissue mosaic, DNA fragments were collected by cross-link reversal and amplified by polymerase chain reaction (PCR) to complete library construction. In order to increase the yield and signal-to-noise ratio from fresh frozen tissue sections, the optimized protocol for spatial-CUT&Tag included (1) bulk transposition followed by sequential DNA barcode ligation rather than using DNA spatial barcode inserted Tn5 transposition (FIG. 71A and FIG. 72) and (2) light fixation (0.2% formaldehyde).

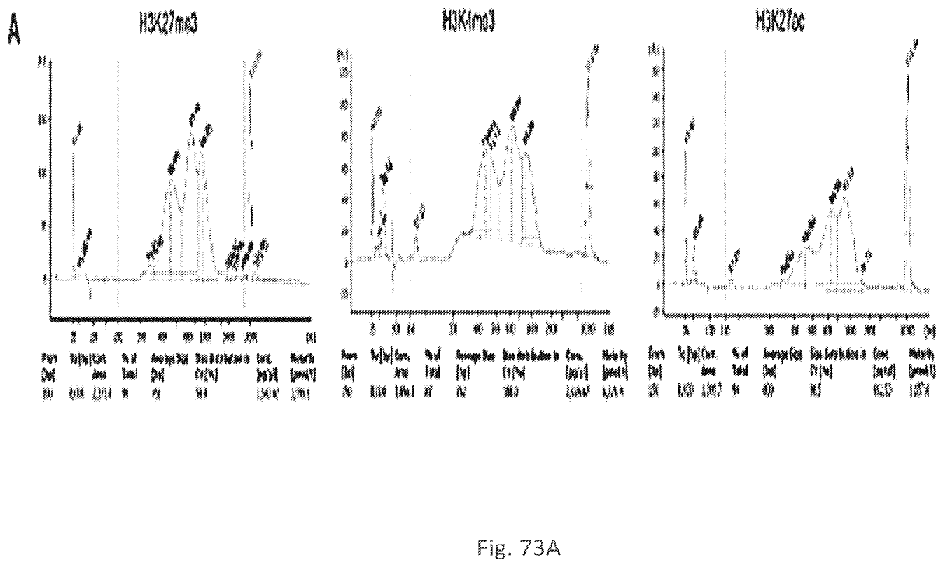

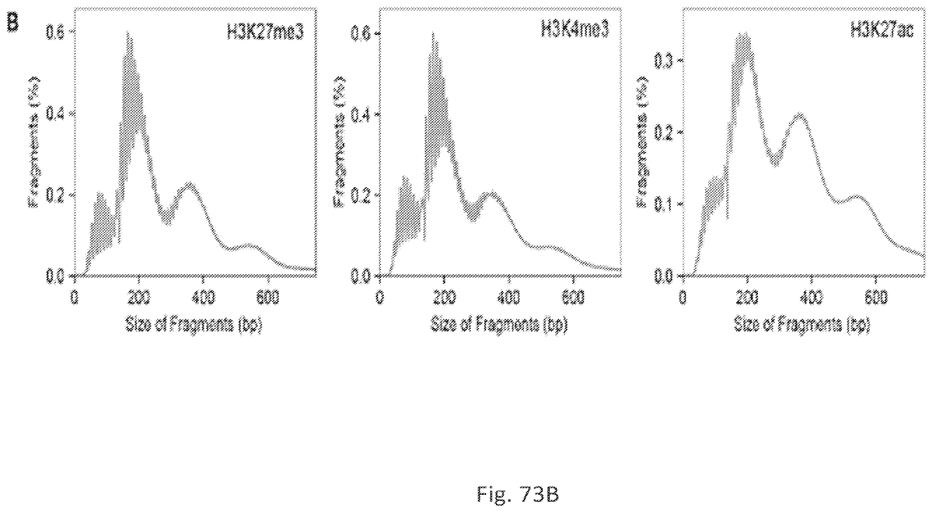

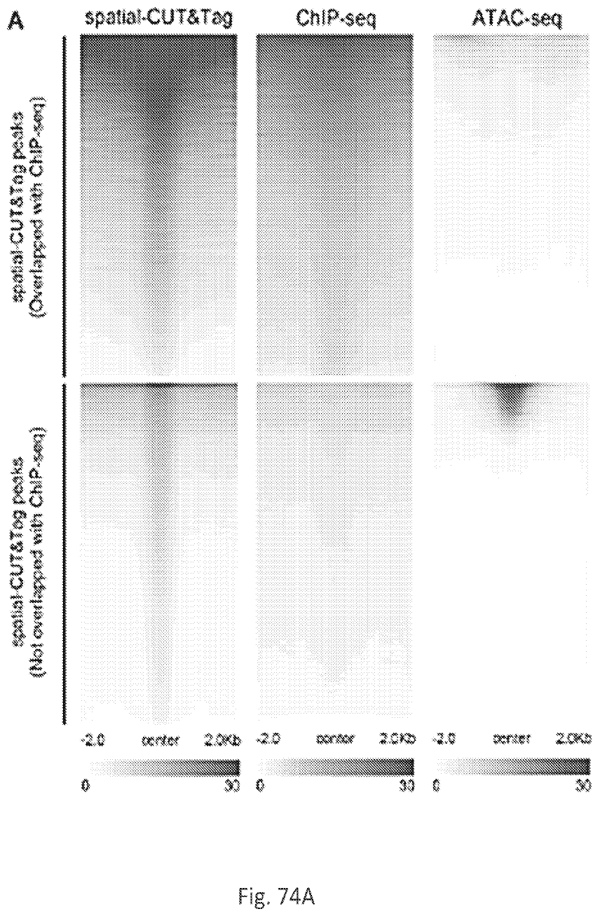

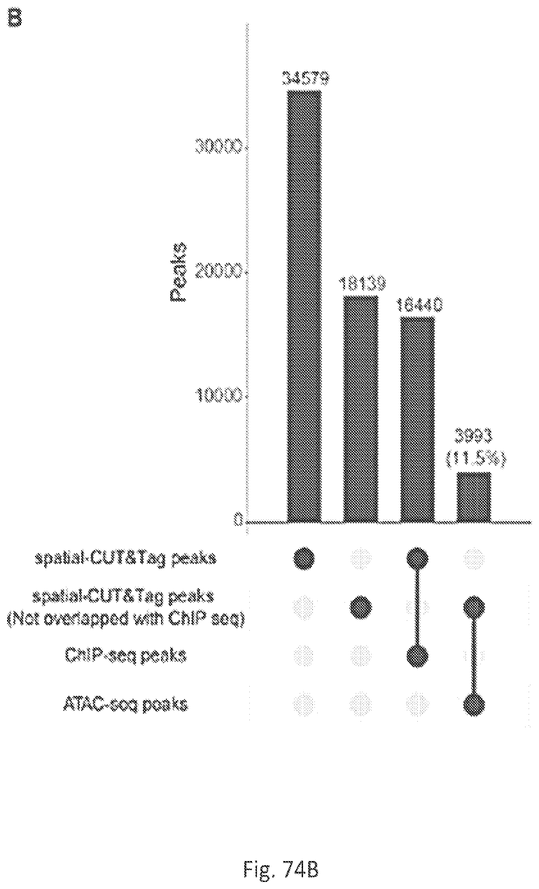

Spatial-CUT&Tag was then performed with antibodies against H3K27me3 (repressing loci), H3K4me3 (activating promoters) and H3K27ac (activating enhancers and/or promoters) in E11 mouse embryos. The quality of spatial epigenome sequencing data was assessed based on the total number of unique fragments, fraction of reads in peaks (FRiP) per pixel, and fraction of mitochondrial reads per pixel (FIG. 71B to D). In spatial-CUT&Tag experiments with 50 μm pixel size, a median of 9,788 (H3K27me3), 16,777 (H3K4me3), or 19,721 (H3K27ac) unique fragments per pixel was obtained of which 16% (H3K27me3), 67% (H3K4me3), or 16% (H3K27ac) of fragments fell within peak regions, indicating high coverage of genomic sequences and a low level of background (as reference, FRiP of bulk CUT&Tag of E11 mouse embryo with H3K27me3 was ˜24%). In addition, proportion of mitochondrial fragments is low in all datasets (a median of 0.16% (H3K27me3), 0.13% (H3K4me3), or 0.01% (H3K27ac) of fragments was from mitochondrial reads). In spatial-CUT&Tag experiments with 20 μm pixel size (cellular level), a median of 10,064 (H3K27me3), 7,310 (H3K4me3), or 13,171 (H3K27ac) unique fragments per pixel was obtained of which 20% (H3K27me3), 37% (H3K4me3), or 12% (H3K27ac) of fragments fell within peak regions. The fractions of read-pairs mapping to mitochondria are 0.01% (H3K27me3), 0.02% (H3K4me3), or 0% (H3K27ac). Additionally, the fragment length distribution was consistent with the capture of nucleosomal and subnucleosomal fragments forall modifications (the subnucleosomal fragments may represent background signal from untethered Tn5) (FIG. 73). To measure the extent of tagmentation by free Tn5, the spatial-CUT&Tag H3K27me3 signals were compared to existing ChIP-seq and ATAC-seq reference datasets (Gorkin et al., 2020, Nature, 583:744-751). The results showed that around 11.5% of peaks that did not overlap with ChIP-seq peaks were observed in ATAC-seq peaks (FIG. 74), which may correspond to the Tn5 insertion events unrelated to the antibody used for a given histone mark (Wang et al., 2021, bioRxiv, 2021.2007.2009.451758).

Spatial-CUT&Tag (20 μm pixel size) was also compared to published scCUT&Tag datasets on the same sample (P21 mouse brain) with same antibodies (H3K4me3 and H3K27me3) at the same sequencing depth (Bartosovic et al., 2021, Nature Biotechnology). The results showed that spatial-CUT&Tag detected more unique fragments (H3K27me3: 9,735, H3K4me3: 3,686) than scCUT&Tag (H3K27me3: 682, H3K4me3: 453) (FIG. 71B). However, the FRIP from spatial-CUT&Tag (H3K27me3: 10%, H3K4me3: 53%) is lower than the scCUT&Tag (H3K27me3: 24%, H3K4me3: 82%) (FIG. 71C). One of the potential reasons is that the spatial-CUT&Tag data were generated not from live cells as in scCUT&Tag, but from fresh frozen samples, which has been reported to affect chromatin structures and generate higher background noise (Milani et al., 2016, Scientific Reports, 6:25474).

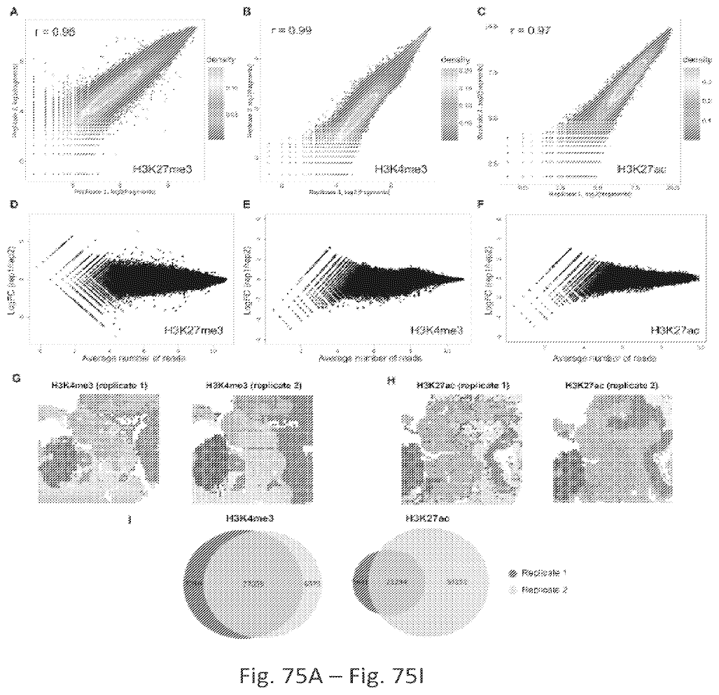

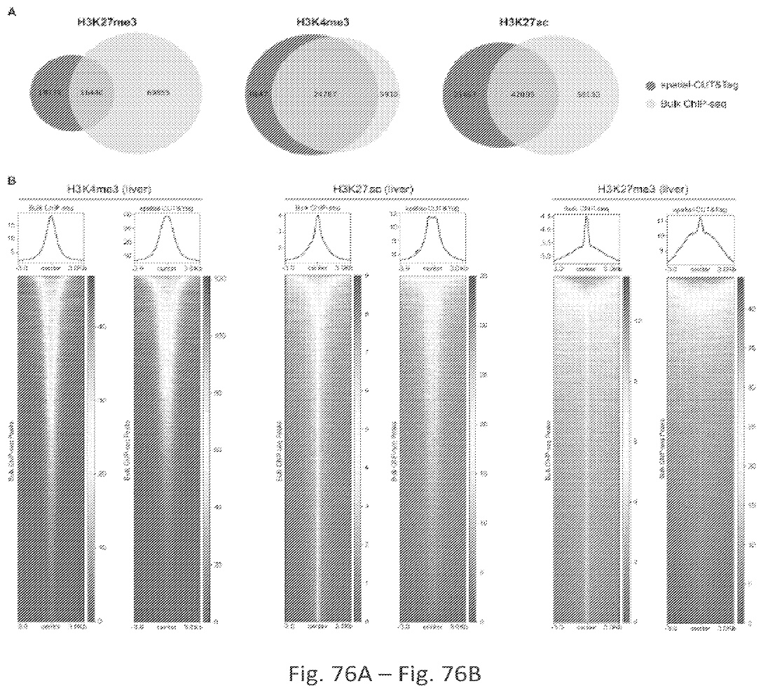

To evaluate the robustness of the method, the reproducibility of replicates from different spatial-CUT&Tag experiments was first validated. The Pearson correlation coefficient was above 0.95 for all experiments (FIG. 75A to F), which demonstrated a consistent performance of spatial-CUT&Tag. In addition, spatial-CUT&Tag was able to reproduce the chromatin state pattern for each histone modification (FIGS. 75G and H), and peaks called from different spatial-CUT&Tag experiments showed strong overlap (FIG. 75I). Furthermore, peaks called from spatial-CUT&Tag aggregate data were compared to the peaks from the ENCODE bulk ChIP-seq data. The results showed a significant overlap between these two datasets (FIG. 76A). The fetal liver region pixels were also extracted from spatial-CUT&Tag and a pseudo-bulk sample was generated, which was compared against bulk fetal liver ENCODE data. The peak-centered heatmap for aggregate spatial-CUT&Tag signal around peaks that were called from the ENCODE bulk datasets was plotted and it was observed that spatial-CUT&Tag yielded high-quality profiles comparable to the reference data (FIG. 76B).

To identify cell types de novo by chromatin states, a cell by tile matrix was generated for the different modifications by aggregating reads in 5 kilobase bins across the genome (Bartosovic et al., 2021, Nature Biotechnology; Wu et al., 2021, Nature Biotechnology) in the E11 mouse embryo spatial-CUT&Tag experiments. Latent sematic indexing (LSI) and uniform manifold approximation and projection (UMAP) were then applied for dimensionality reduction and embedding, followed by Louvain clustering using the ArchR package (Granja et al., 2021, Nature Genetics, 53:403-411). Mapping the clusters back to the spatial location identified spatially distinct patterns that agreed with the tissue histology in a H&E-stained adjacent tissue section (FIG. 71E to G, FIG. 77). Cluster 1 (H3K27me3) and cluster 6 (H3K4me3) represent the heart in the mouse embryo. Cluster 2 (H3K27me3 and H3K4me3) and cluster 4 (H3K27ac) are specific to the liver region. Cluster 8 (H3K27me3), cluster 3 (H3K4me3) and cluster 1 (H3K27ac) are associated with the forebrain, while cluster 9 (H3K27me3), cluster 5 (H3K4me3) and cluster 3 (H3K27ac) with the brainstem, including midbrain. Cluster 11 (H3K27me3), cluster 8 (H3K4me3) and cluster 2 (H3K27ac) are present in more posterior regions of the central nervous system, as the spinal cord. These results have never been observed directly in the tissue section and demonstrated that spatial-CUT&Tag could resolve epigenetically controlled major tissue structures with high spatial resolution.

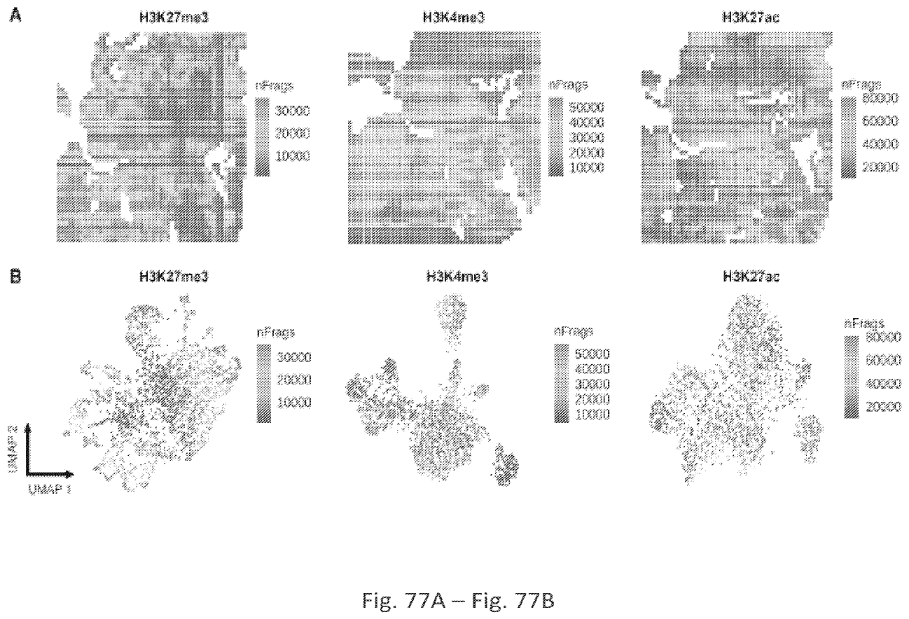

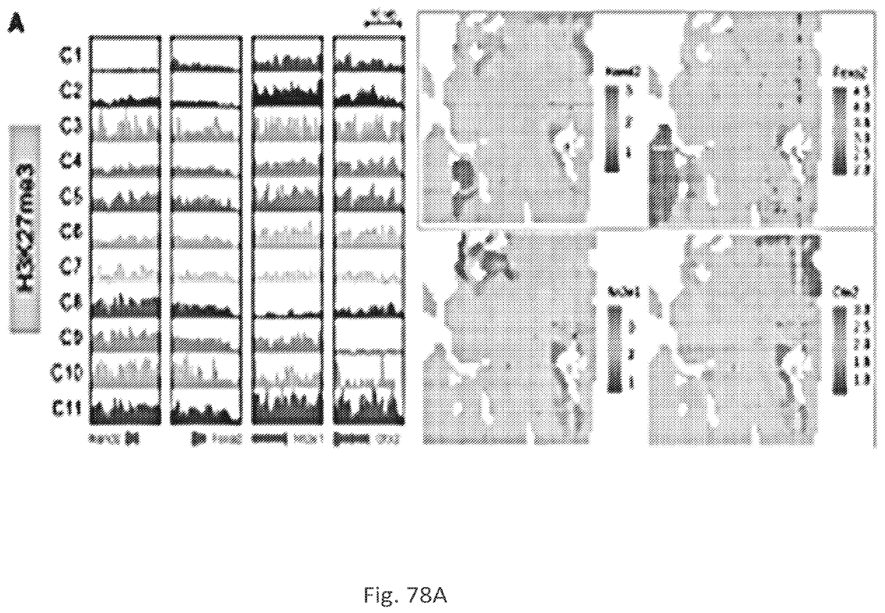

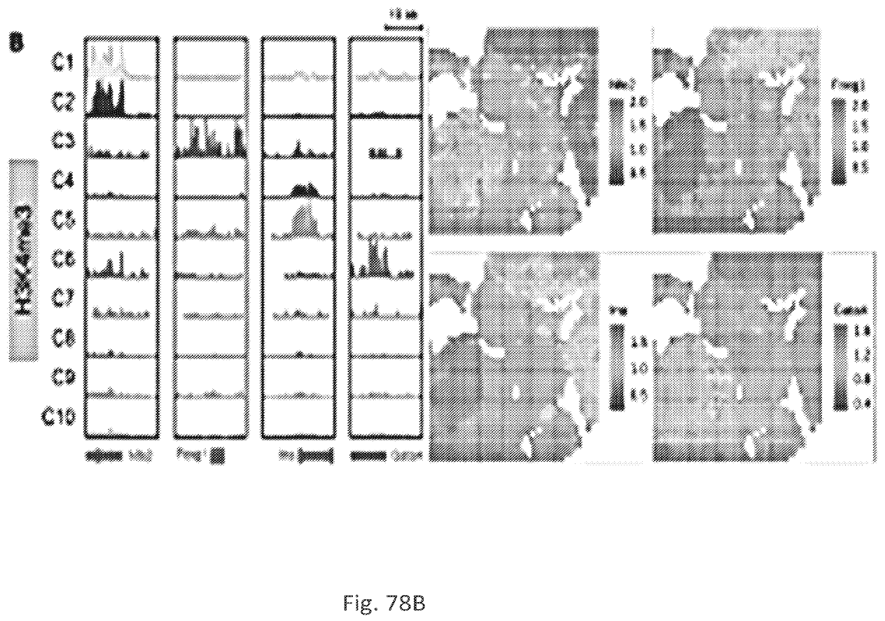

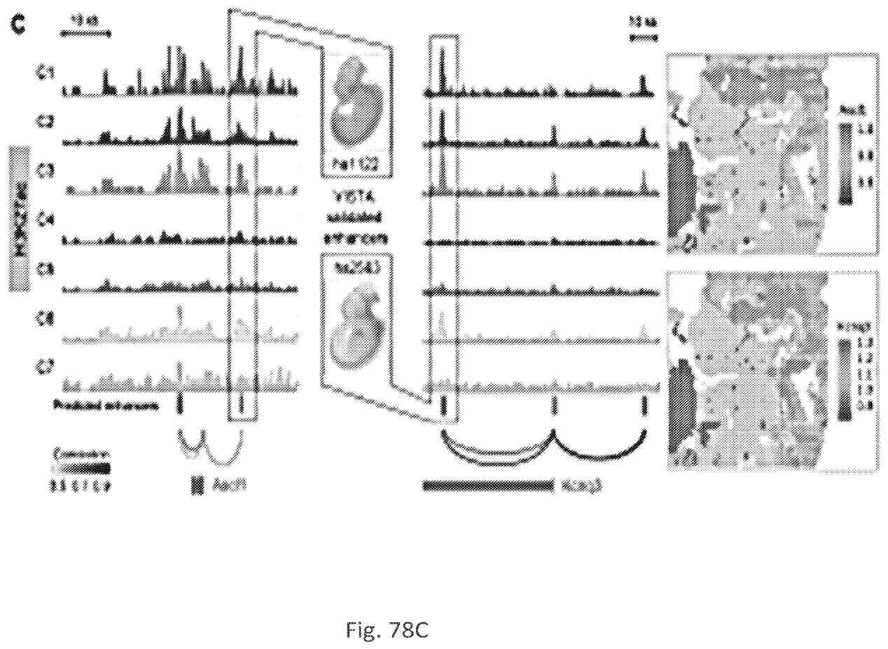

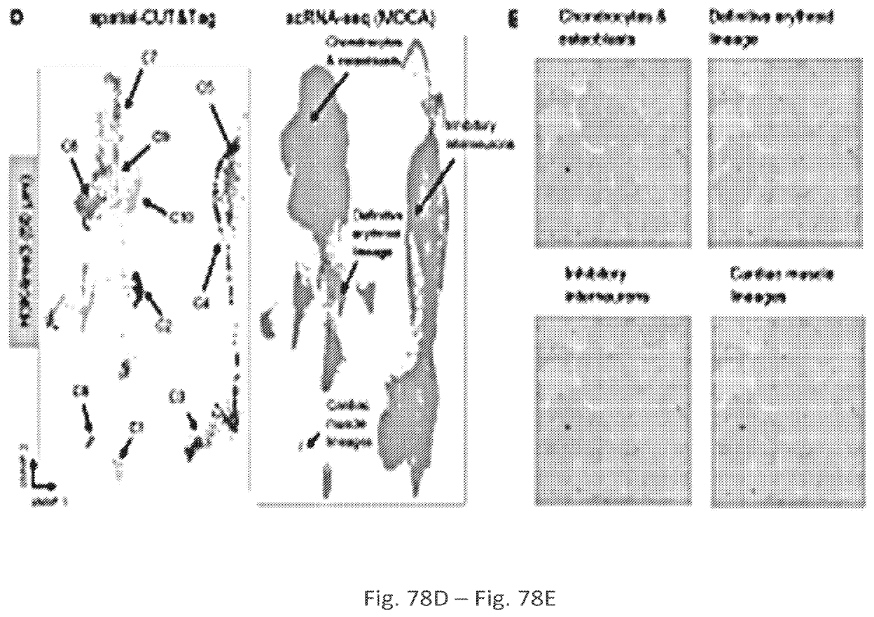

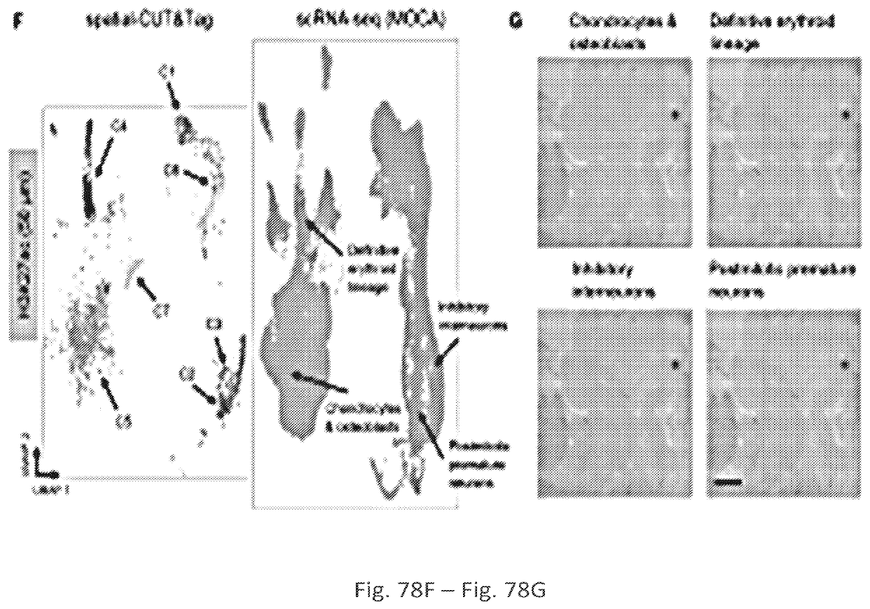

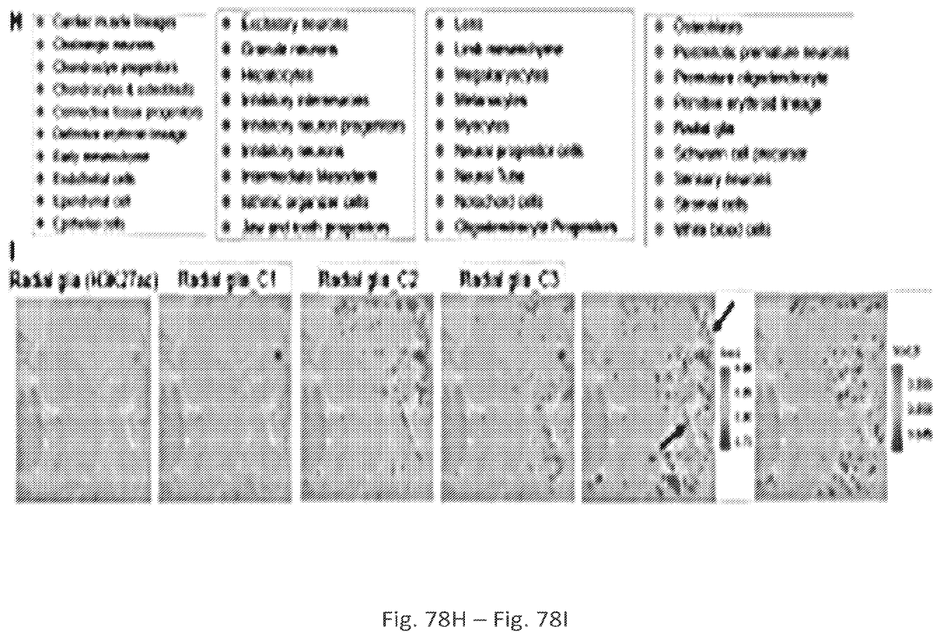

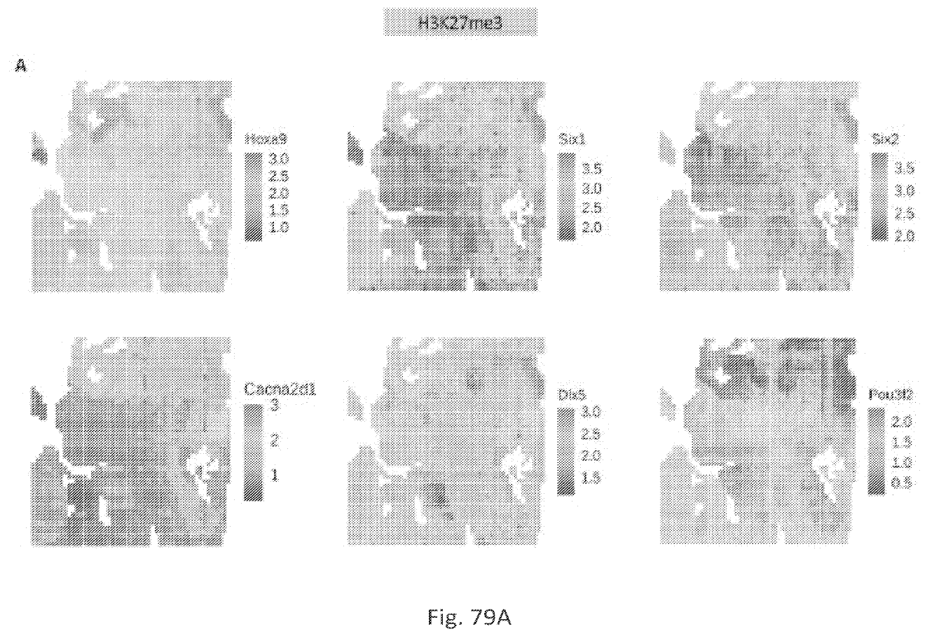

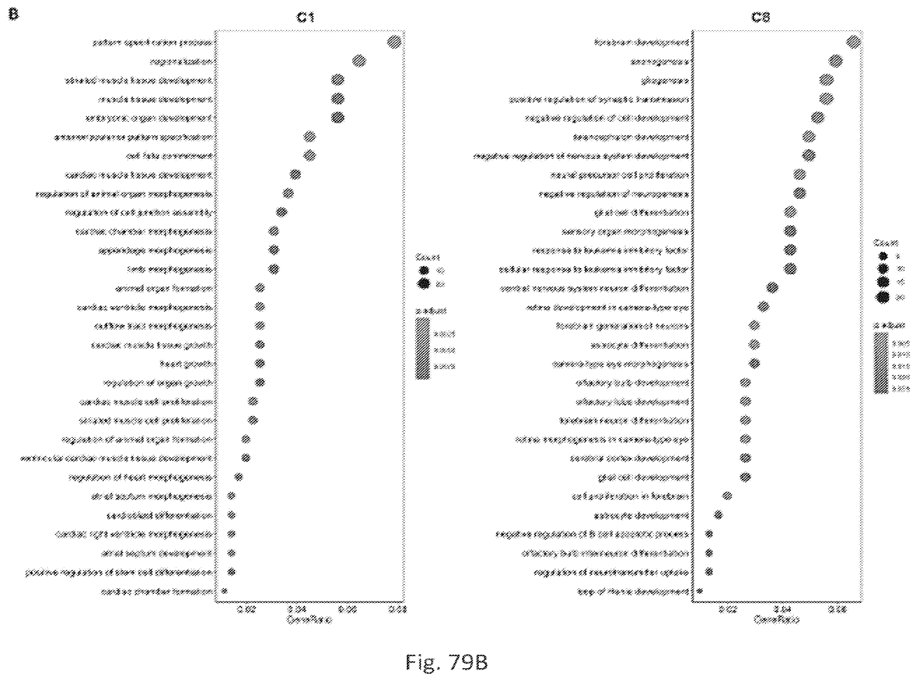

To benchmark spatial-CUT&Tag data, UMAP transform function was used to project the ENCODE organ-specific ChIP-seq data onto the UMAP embedding (Gorkin et al., 2020, Nature, 583:744-751; Granja et al., 2021, Nature Genetics, 53:403-411). Overall, cluster identification matched well with the ChIP-seq projection (FIGS. 71G and H) and distinguished major cell types in E11 mouse embryo. To further compare spatial-CUT&Tag to known spatial patterning during development, cell type-specific marker genes were examined and the expression of these genes was estimated from the chromatin modification data. For H3K27me3, chromatin silencing score (CSS) was calculated to predict the gene expression based on the overall signal associated with a given locus (16). Active genes should have a low CSS due to the lack of H3K27me3 repressive mark in the vicinity of the marker gene regions (FIG. 78A and FIG. 79A). For example, Hand2, which is required for vascular development and plays an essential role in cardiac morphogenesis (Stelzer et al., 2016, Current Protocols in Bioinformatics, 54:1.30.31-31.30.33), showed a lack of H3K27me3 enrichment in the heart (C1 for H3K27me3).

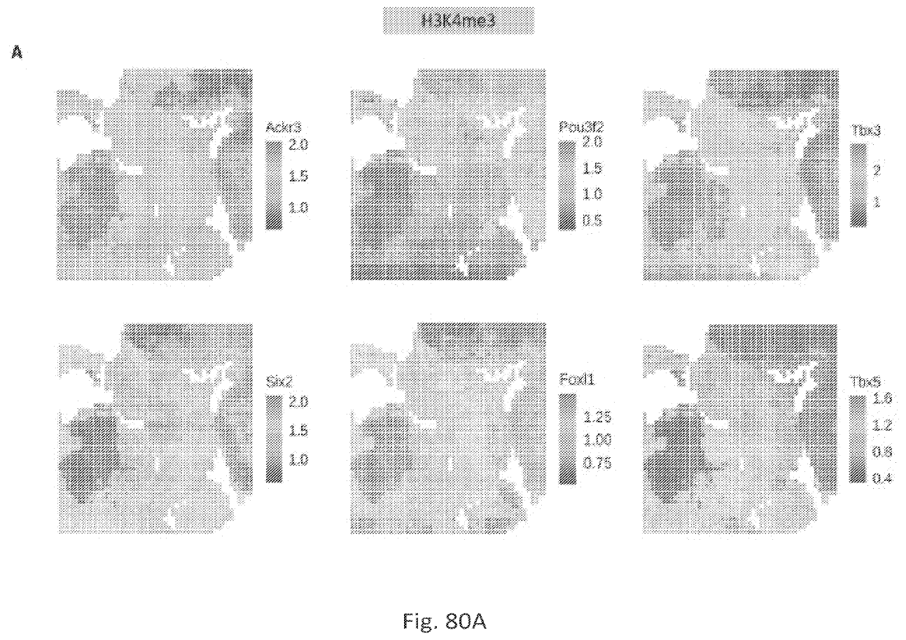

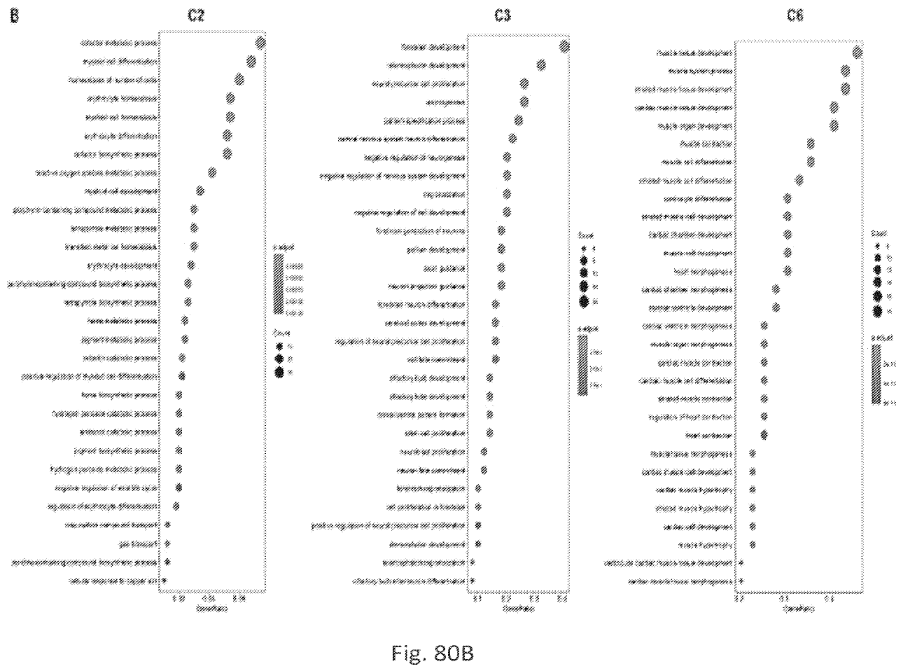

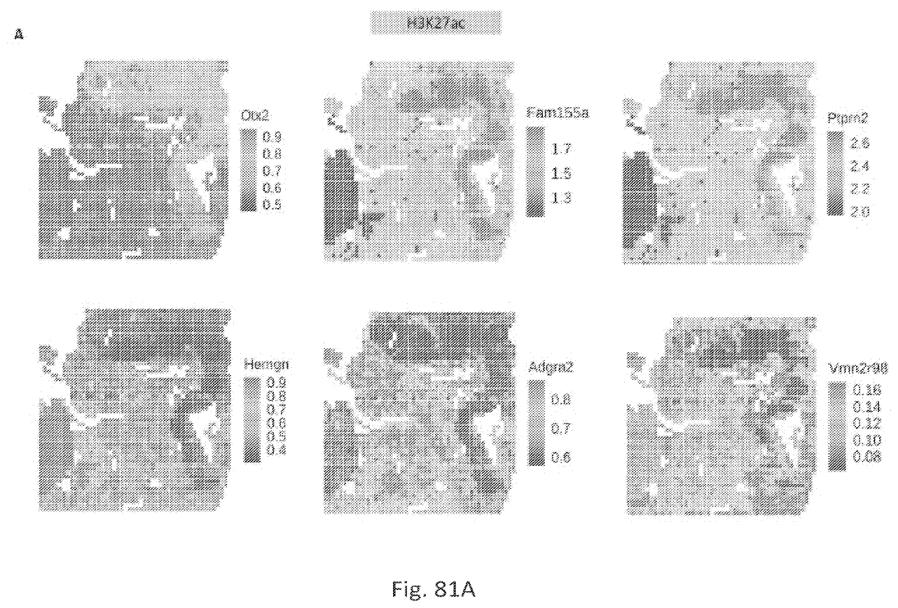

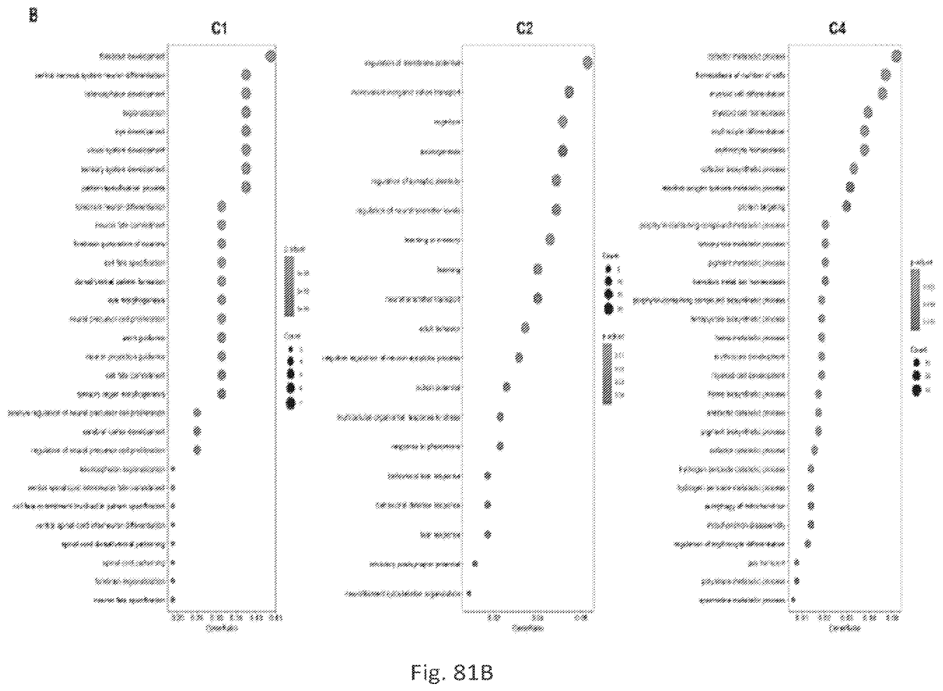

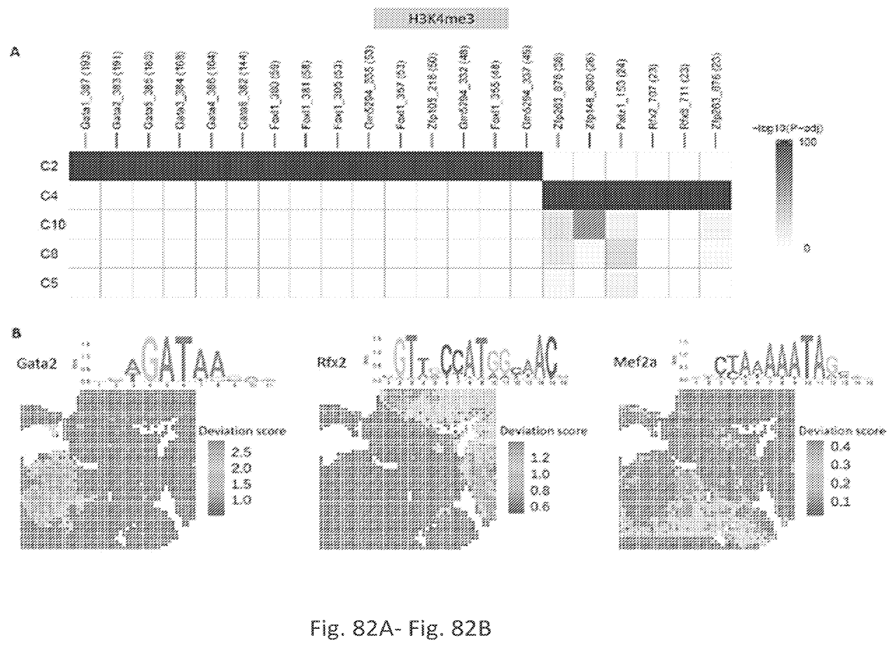

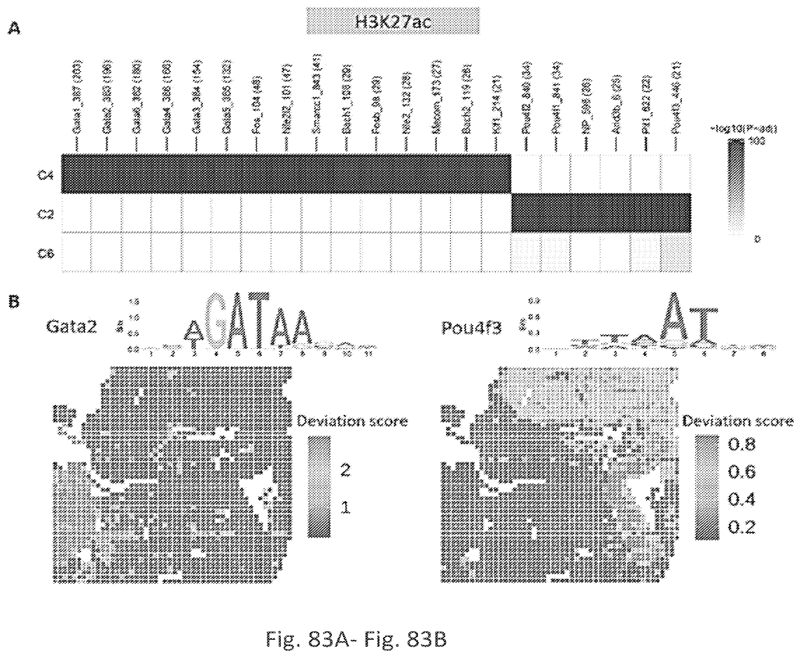

Foxa2, a transcription activator for several liver-specific genes (Stelzer et al., 2016, Current Protocols in Bioinformatics, 54:1.30.31-31.30.33), has low CSS predominately in the liver region (C2). Nr2e1, which correlates with the lack of H3K27me3 modification in the forebrain (C8), is required for anterior brain differentiation and patterning and is also involved in retinal development (Stelzer et al., 2016, Current Protocols in Bioinformatics, 54:1.30.31-31.30.33). Otx2, a transcription factor probably involved in the development of the brain and the sense organs (Stelzer et al., 2016, Current Protocols in Bioinformatics, 54:1.30.31-31.30.33), presents low H3K27me3 at the brainstem cluster (C9). For H3K4me3 and H3K27ac, gene activity score (GAS) was used since they are related to active genes (FIG. 78B, FIG. 80A and FIG. 81A). For example, Nfe 2 and Hemgn, which are essential for regulating erythroid and hematopoietic cell maturation and differentiation (Stelzer et al., 2016, Current Protocols in Bioinformatics, 54:1.30.31-31.30.33), were active in liver and to some extent in the heart (C2 and C6 for H3K4me3). H3K4me3 was highly enriched in the forebrain (C3) at the locus of Foxg1, which plays an important role in the establishment of the regional subdivision of a developing brain and in the development of telencephalon (Stelzer et al., 2016, Current Protocols in Bioinformatics, 54:1.30.31-31.30.33). Ina, which is involved in the morphogenesis of neurons (Stelzer et al., 2016, Current Protocols in Bioinformatics, 54:1.30.31-31.30.33), showed high GAS in brainstem (C5) as well as in the spinal cord (C4). Gata4, which plays a key role in myocardial differentiation and function (Stelzer et al., 2016, Current Protocols in Bioinformatics, 54:1.30.31-31.30.33), was activated extensively in the heart (C6). Gene Ontology (GO) enrichment analysis was conducted for each cluster, and the GO pathways matched well with the anatomical annotation (FIGS. 79B, 80B, and 81B). To understand which regulatory factors are most active across clusters, transcription factor (TF) motif enrichments were calculated in H3K4me3 and H3K27ac modification loci using ArchR (FIGS. 82 and 83). As expected, the most enriched motifs in liver correspond to GATA transcription factors, including the well-studied role of Gata2 in the development and proliferation of hematopoietic cell lineages. Mef2a, which mediates cellular functions in cardiac muscle development, was enriched in the heart region (Stelzer et al., 2016, Current Protocols in Bioinformatics, 54:1.30.31-31.30.33). To predict gene regulatory interactions of enhancers and their target genes across clusters, gene expression measured by scRNA-seq (Cao et al., 2019, Nature, 566:496-502) and H3K27ac modifications were correlated at candidate enhancers using ArchR (FIG. 78C). The correlation-based map predicted experimentally validated enhancer-gene interactions with high spatial resolution. For example, the predicted enhancers of Ascl1 and Kenq3 were enriched in the central nervous system (CNS) (clusters C1,C2, C3 and C6), which are in agreement with the VISTA validated elements (Visel et al., 2007, Nucleic Acids Research, 35:D88-D92).

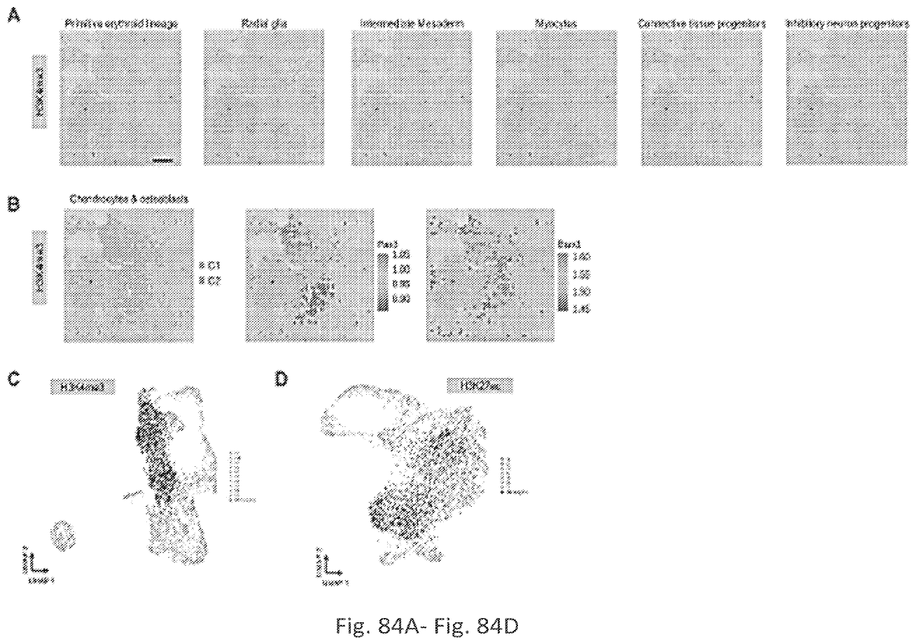

ScRNA-seq data from the Mouse Organogenesis Cell Atlas (Cao et al., 2019, Nature, 566:496-502) was then integrated with spatial-CUT&Tag data (i.e., H3K4me3 and H3K27ac) to identify cell types in spatial epigenome map (FIG. 78D to H, FIG. 84). Spatial tissue pixels were found to conform well into the clusters of single-cell transcriptomes, enabling the transfer of cell type annotations from single-cell transcriptomics data to the spatial pixels in tissue and further to the chromatin modification states.

Several organ-specific cell types were detected (FIGS. 78E and G). For example, the definitive erythroid lineage cells were exclusively enriched in the liver, which is the major hematopoietic organ at this stage of embryonic development (Stelzer et al., 2016, Current Protocols in Bioinformatics, 54:1.30.31-31.30.33). Cardiac muscle cell types were observed only in the heart region in agreement with the anatomical annotation. Chondrocytes & osteoblasts were observed widely in the embryonic facial prominence. Inhibitory interneurons were highly enriched in the brain stem. Postmitotic premature neurons were observed extensively in the spinal cord region. A high-resolution clustering analysis further identified sub-populations of developing neurons with distinct spatial distribution and chromatin state (FIG. 78I, FIG. 84B). For instance, the H3K27ac radial glia could be further subset to three clusters. Genes related to stem cell maintenance in the central nervous system (e.g. Sox1) had higher expression in subcluster 2, which was enriched along the ventricles in the developing brain stem and spinal cord. In contrast, subcluster 3 cells were in the spinal cord parenchyma, while subcluster 1 cells were mainly outside the CNS, and thus might represent the epigenetic state of neural crest progenitors (e.g. active Sox10) (FIG. 78I). Additionally, two subclusters with distinct spatial distributions were found in the chondrocytes & osteoblasts, and genes related to developing teeth (e.g. Barx1) had higher expression in subcluster 2 (FIG. 84B).

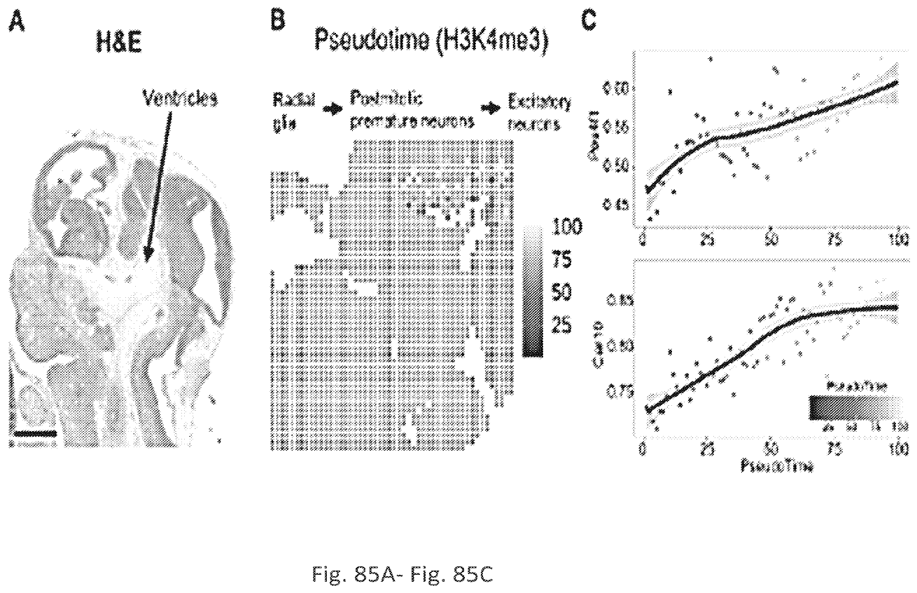

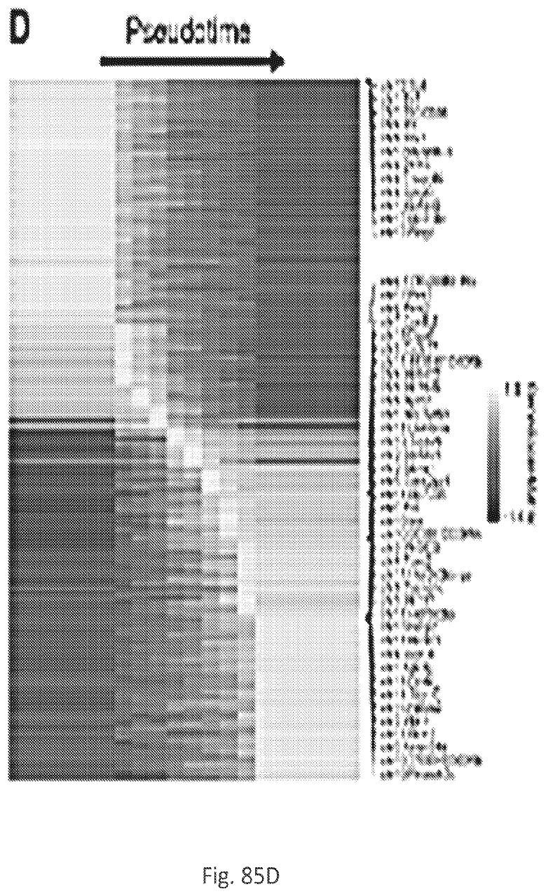

During embryonic development, dynamic changes in chromatin states across time and space help regulate the formation of complex tissue architectures and terminally differentiated cell types (Stickels et al., 2020, Nature Biotechnology). In the embryonic CNS, radial glia function as primary progenitors or neural stem cells, which give rise to various cell types in the CNS (Kriegstein et al., 2009, Annual Review of Neuroscience 32, 149-184). Therefore, it was tested whether the spatial-CUT&Tag data could be exploited to recover the spatially organized developmental trajectory and examine how developmental processes proceed across the tissue space. The course of a developmental process from radial glia to excitatory neurons was studied with postmitotic premature neurons as the immediate state after the radial glial differentiation and ordered these cells in pseudo-time using ArchR. Spatial projection of each pixel's pseudo-time value revealed the spatially organized developmental trajectory in neurons (FIG. 85). Interestingly, it was observed that the chromatin state of cells early in differentiation clustered around the ventricles in the developing brainstem whereas those farther away exhibited a more differentiated phenotype (FIG. 85B). Changes in gene activity were identified based on H3K4me3 across this developmental process, and many genes recovered are important in neuron development, including Pou4f1, which regulates the expression of specific genes involved in differentiation and survival of neurons (Stelzer et al., 2016, Current Protocols in Bioinformatics, 54:1.30.31-31.30.33), and Car 10, which was reported to be involved in neuron differentiation (Luo et al., 2021, BMC Biology 19, 135) (FIGS. 85C and D).

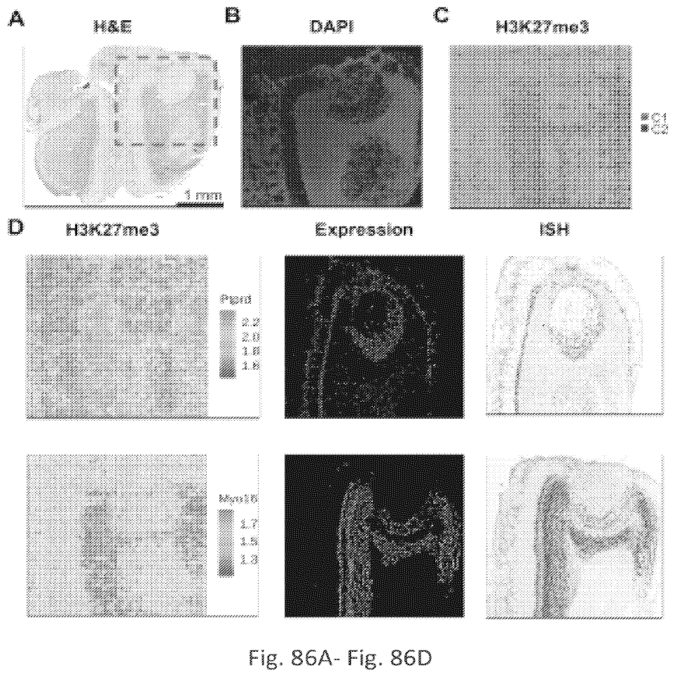

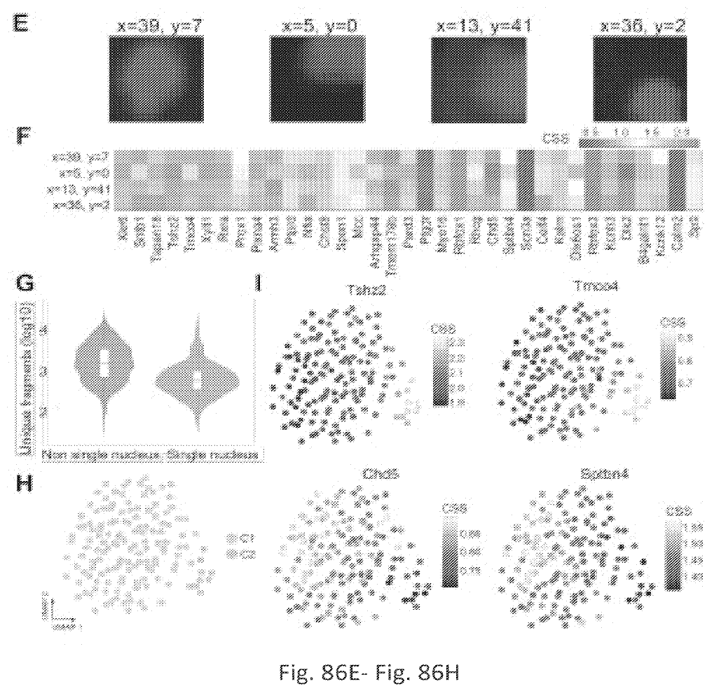

The combination of spatial-CUT&Tag with immunofluorescence staining in the same tissue section (FIG. 86) was demonstrated. A mouse olfactory bulb tissue section was stained with DAPI (4′,6-diamidino-2-phenylindole), a blue nuclear DNA dye (FIGS. 86A and B). Then, spatial-CUT&Tag was performed against H3K27me3 with 20 μm pixel size, which distinguished the major cell types, including glomerular layer (cluster 1) and granular layer (cluster 2) (FIG. 86). Spatial patterns of H3K27me3 modification state revealed by spatial-CUT&Tag were validated by data from in situ hybridization as shown in FIG. 86D. With DAPI staining for nucleus, the pixels of interest were selected such as those containing only one nucleus or those showing specific chromatin modifications. Combining immunofluorescence with spatial-CUT&Tag at the cellular level (20 μm pixel size) on the same tissue slide allowed for extracting single-cell epigenome data in situ without tissue dissociation (FIG. 86E to I).

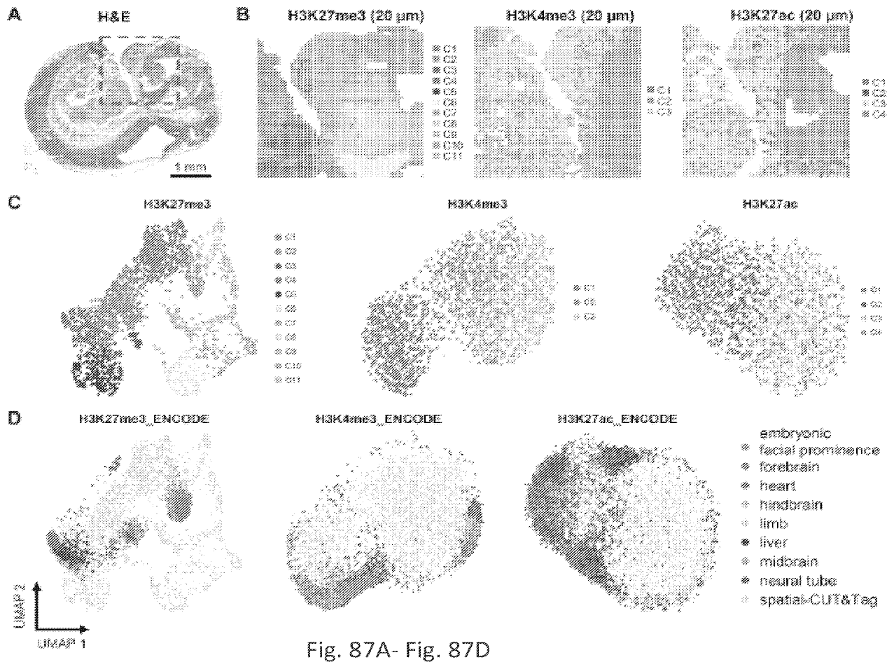

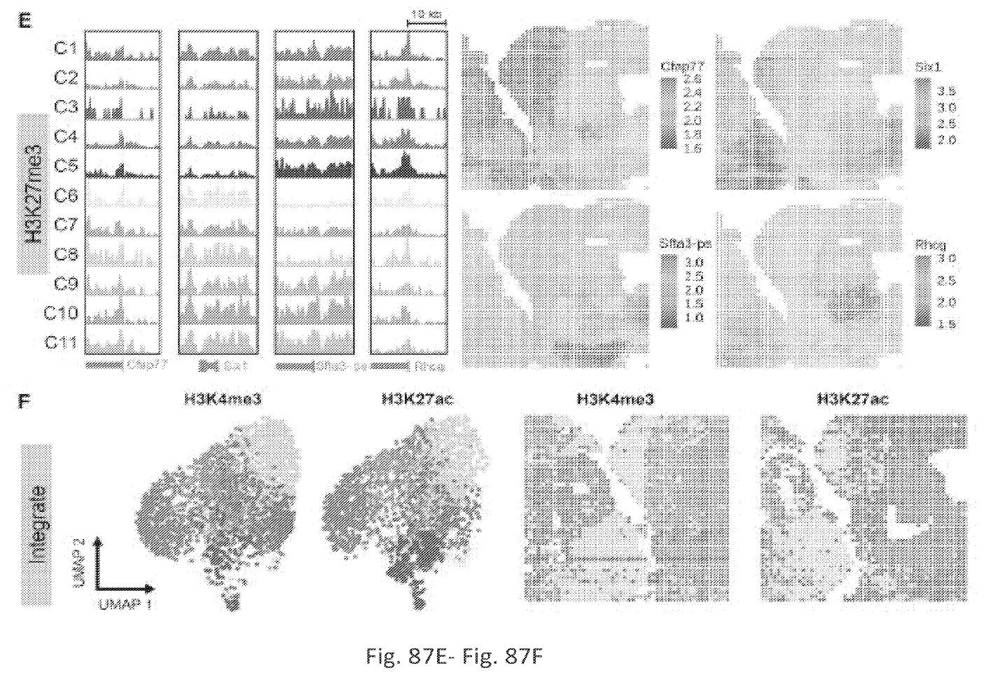

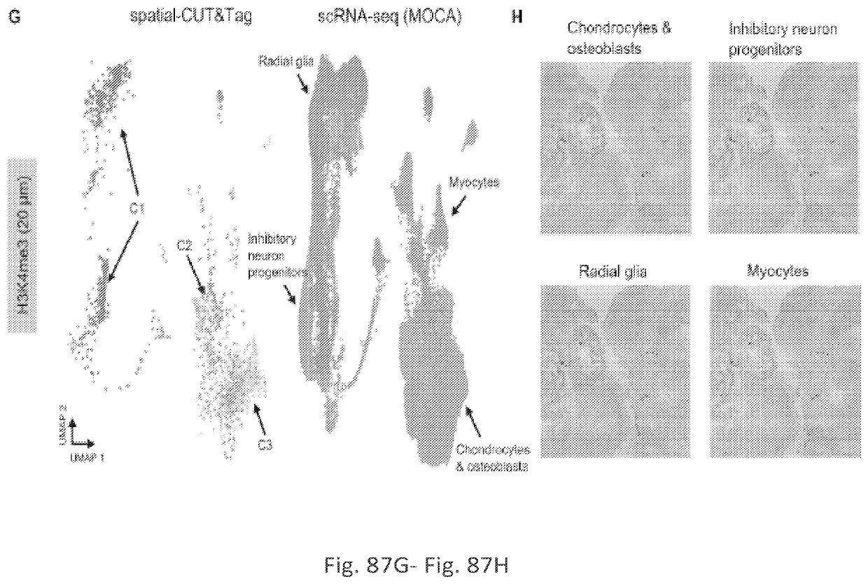

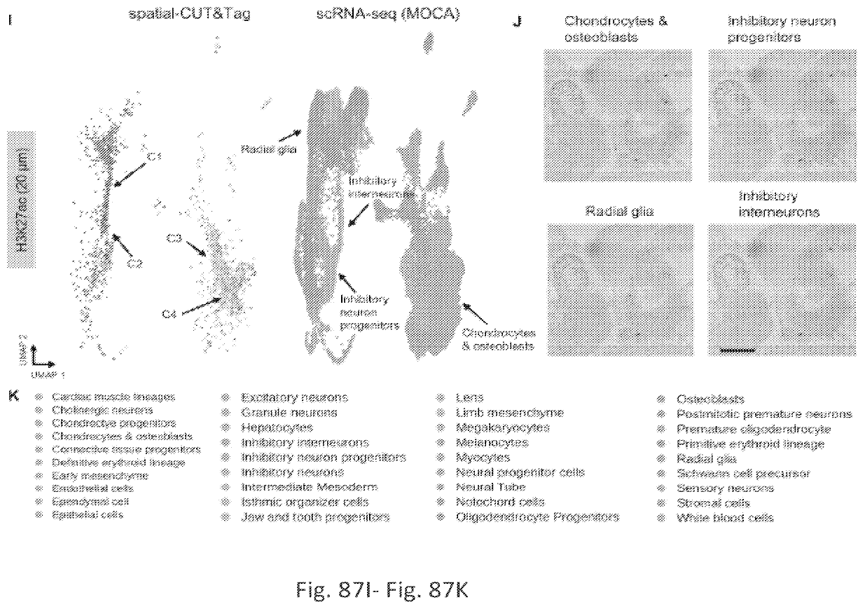

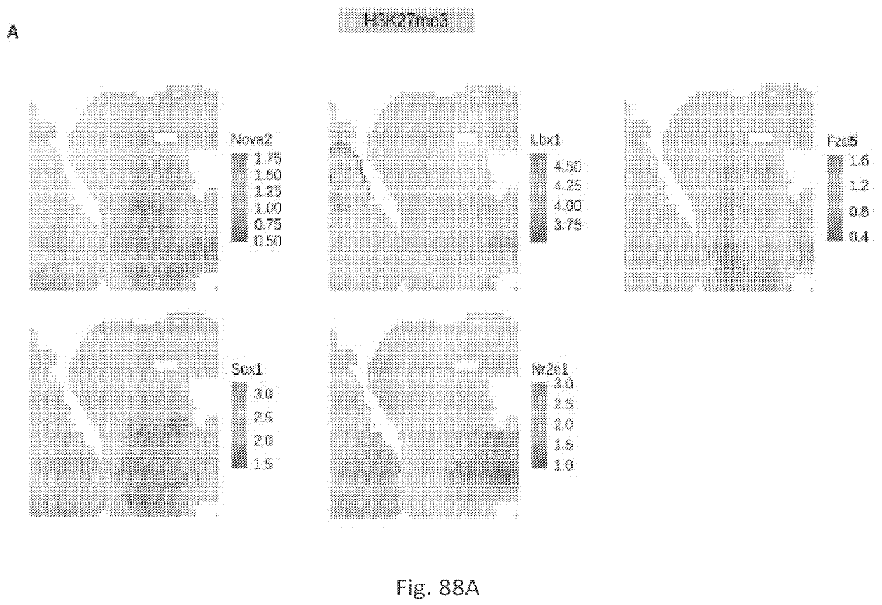

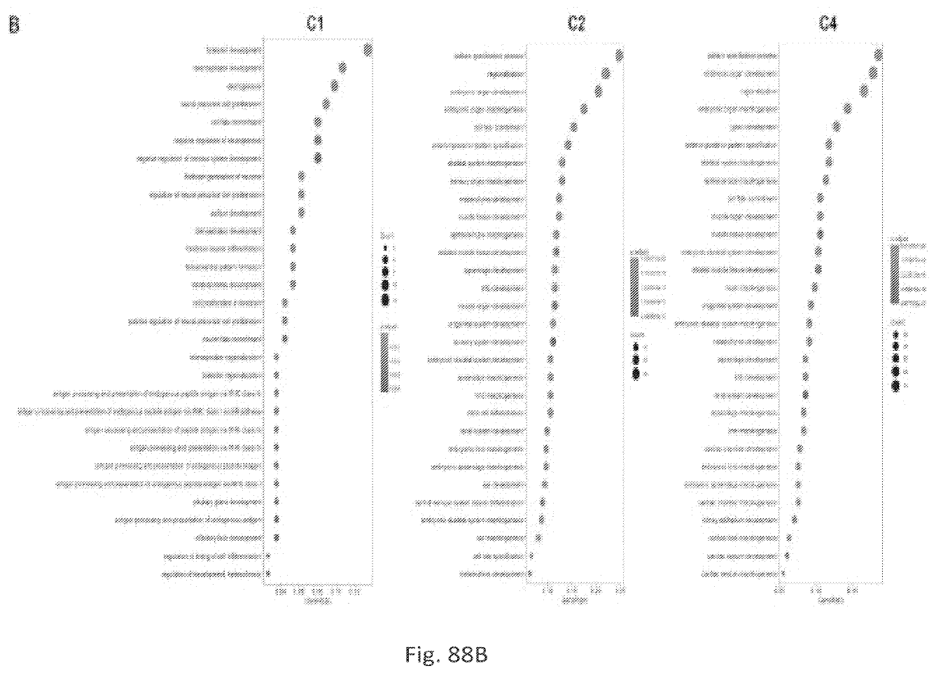

Spatial-CUT&Tag was conducted with 20 μm pixel size to analyze the brain region of an E11 mouse embryo (FIG. 87). Unsupervised clustering showed distinct spatial patterns for different histone modifications and H3K27me3 is associated with the most clusters (FIG. 87A to C). Cluster identification matched ENCODE organ-specific bulk ChIP-seq projection onto the UMAP embedding (FIGS. 87C and D). H3K27me3 modifications were surveyed and distinct modification patterns were observed across clusters (FIG. 87E and FIG. 88A). Cfap77 was repressed extensively except in a portion of the forebrain. Six1, which is involved in limb development, had low CSS in Cluster 5. Although both Sfta3-ps and Rhcg lack H3K27me3 enrichment only in the forebrain, they had distinct spatial patterns. Pathway analysis of marker genes revealed that cluster 1 was mainly involved in forebrain development, cluster 2 corresponded to anterior/posterior pattern specification, and cluster 4 was associated with heart morphogenesis, all in good agreement with anatomical annotations (FIG. 87A and FIG. 88B). Next, the clustering resolution was improved by integrating data across H3K4me3 and H3K27ac histone marks. For this purpose, the canonical correlation analysis (CCA) was used and the data was integrated at gene resolution. The granularity of two-dimensional representation of the data obtained from the integrated analysis was further improved (FIG. 87F). To assign cell types to each cluster, the spatial-CUT&Tag data (H3K4me3 and H3K27ac) was integrated with the scRNA-seq atlas of the mouse embryos (Cao et al., 2019, Nature, 566:496-502) (FIG. 86G to K). For example, chondrocytes & osteoblasts were mainly in the embryonic facial prominence, and radial glia and inhibitory neuron progenitors were observed in the forebrain (FIGS. 87H and J). Although H3K4me3 and H3K27ac had fewer clusters than H3K27me3 at the 20 μm resolution, it was found that the clusters that appeared to be homogenous could be further deconvoluted into sub-populations, indicating that integrative analyses using single-cell or spatial transcriptomics data with well annotated cell types can further refine the definition of cell identity and correlate with spatial distribution of chromatin modification states (Stuart et al., 2019, Cell, 177:1888-1902 e1821).

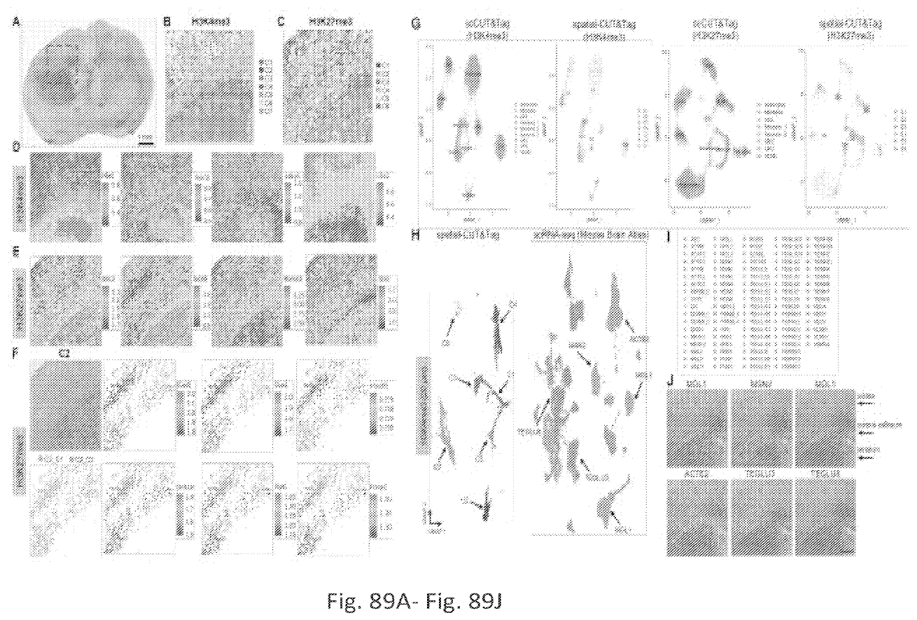

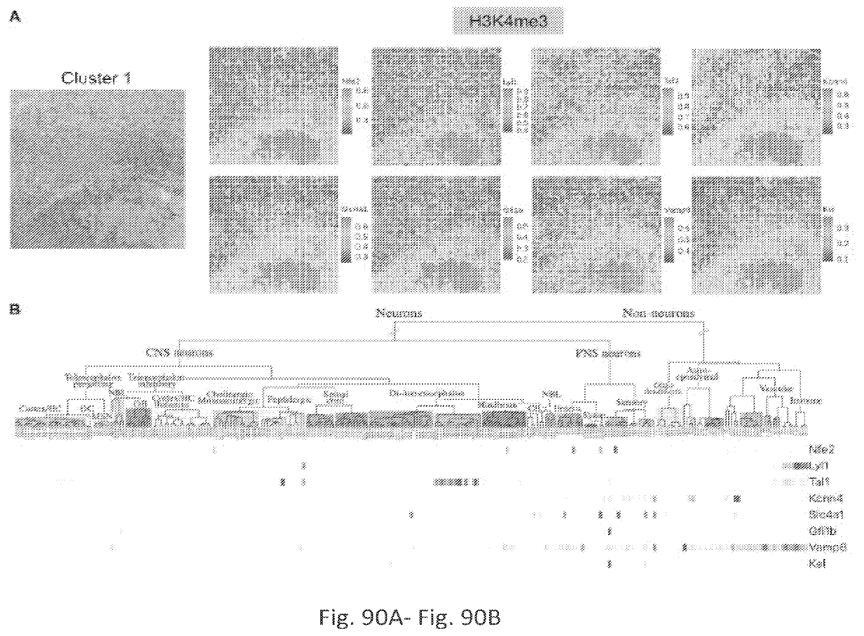

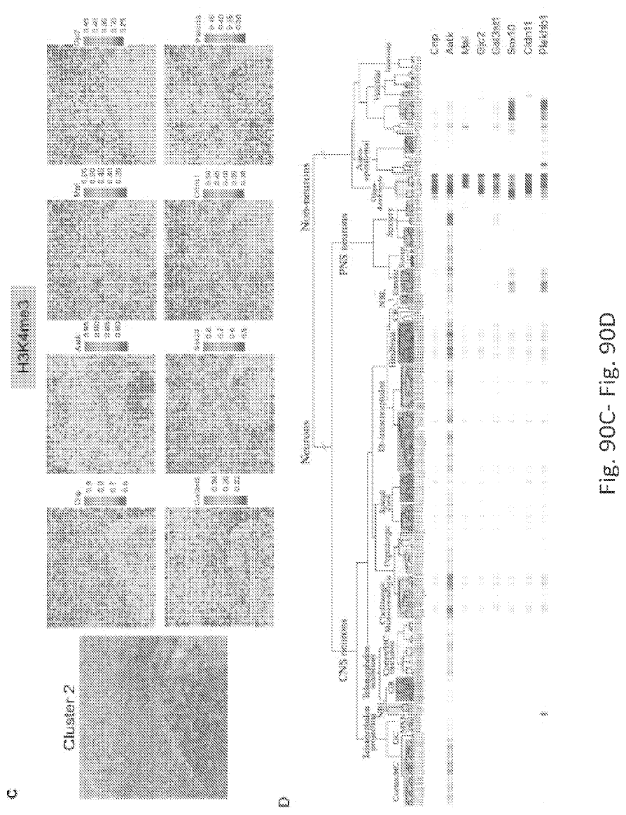

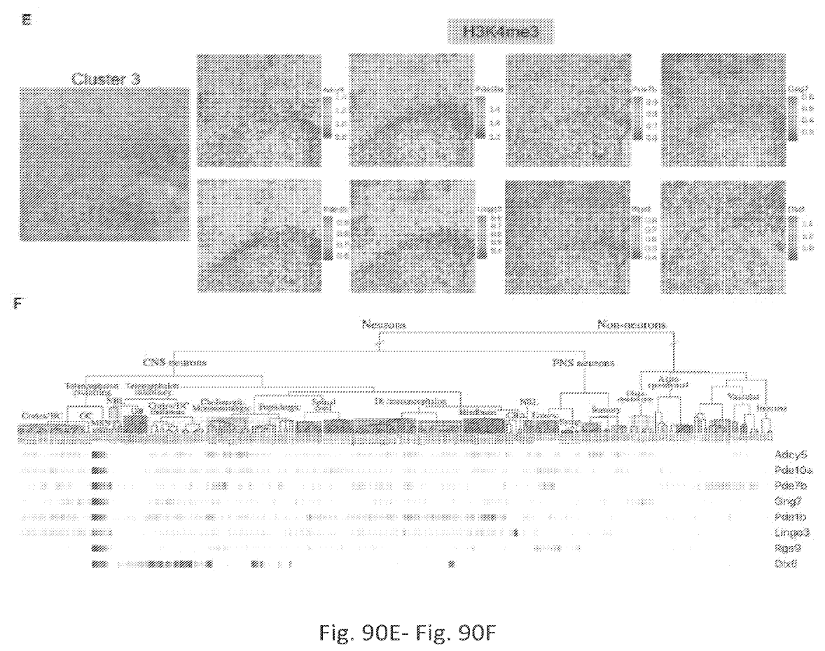

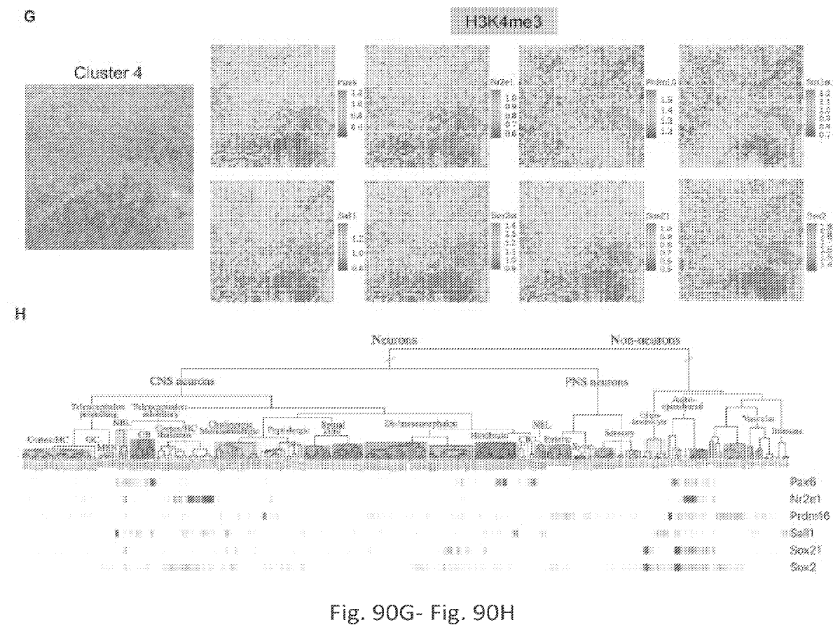

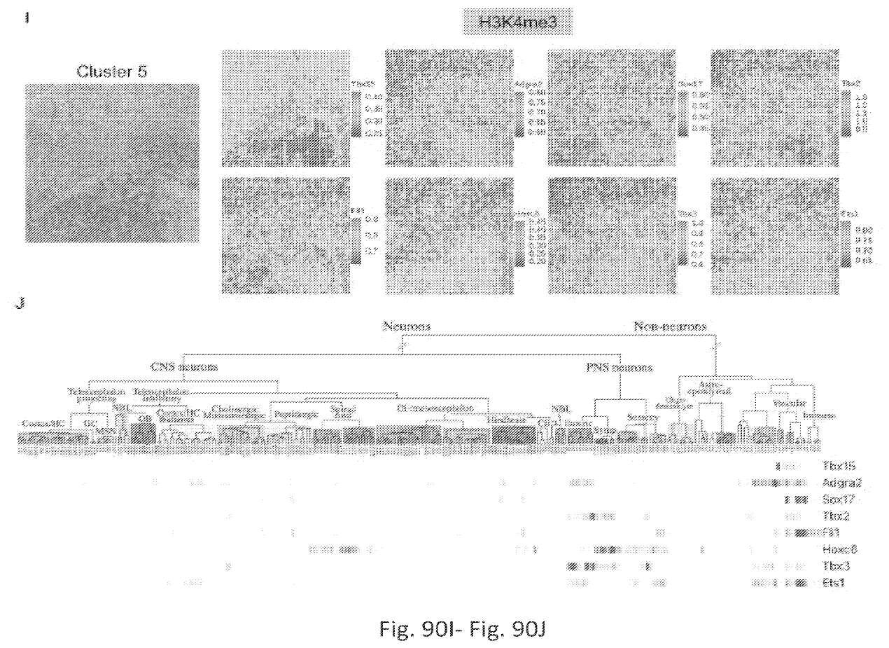

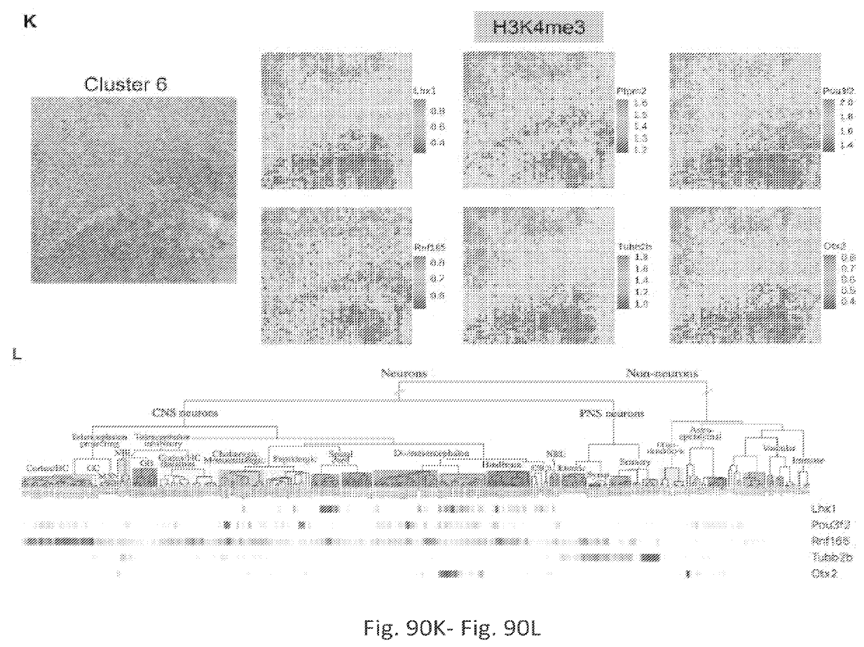

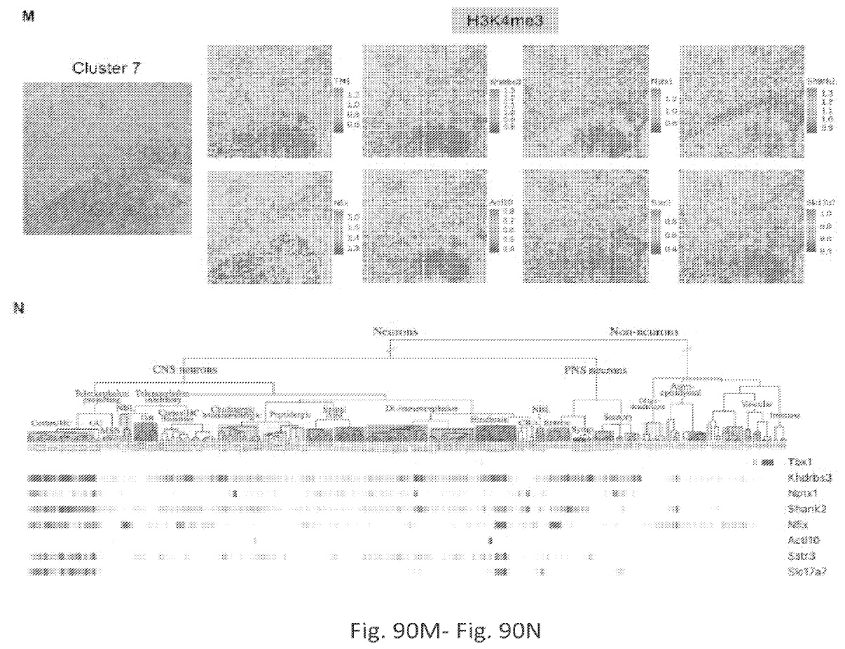

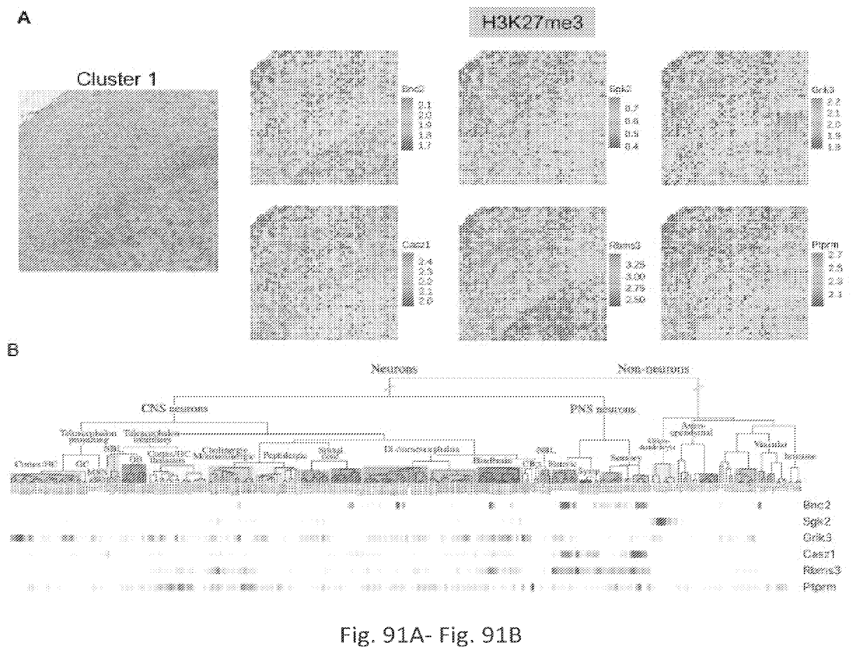

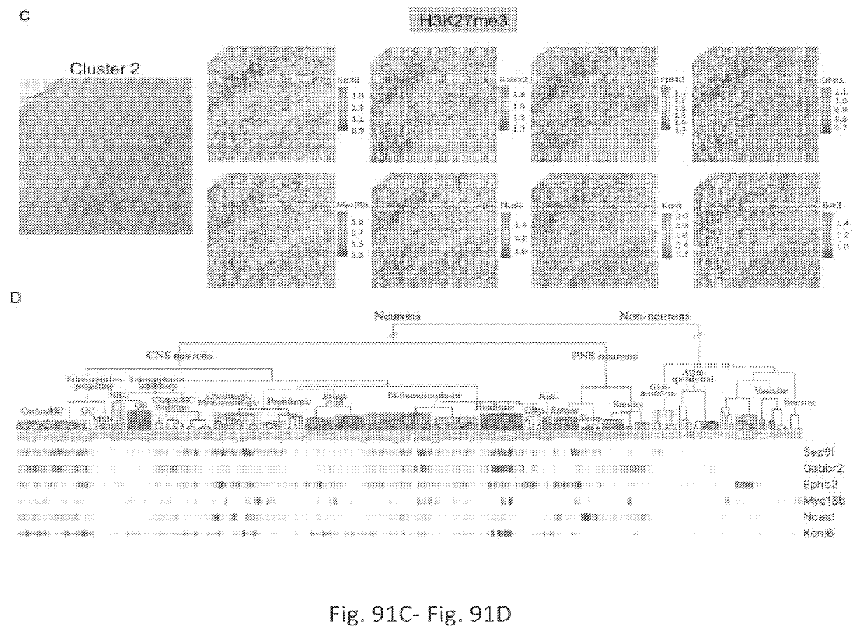

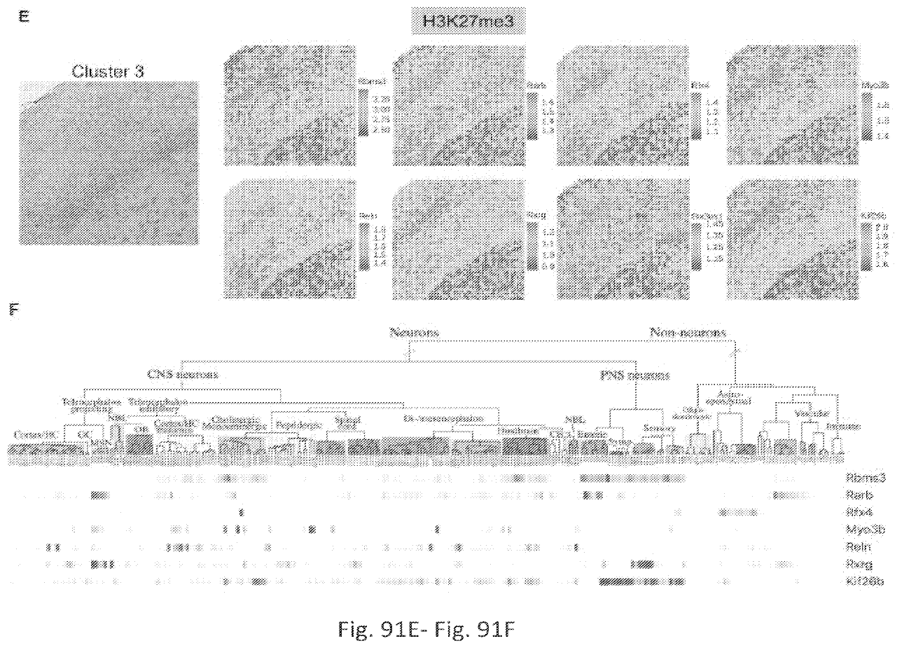

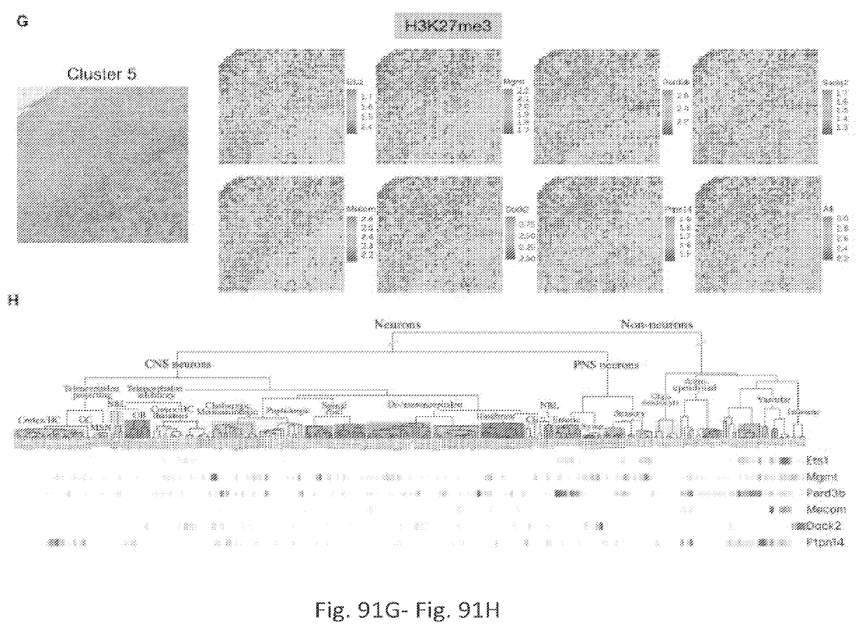

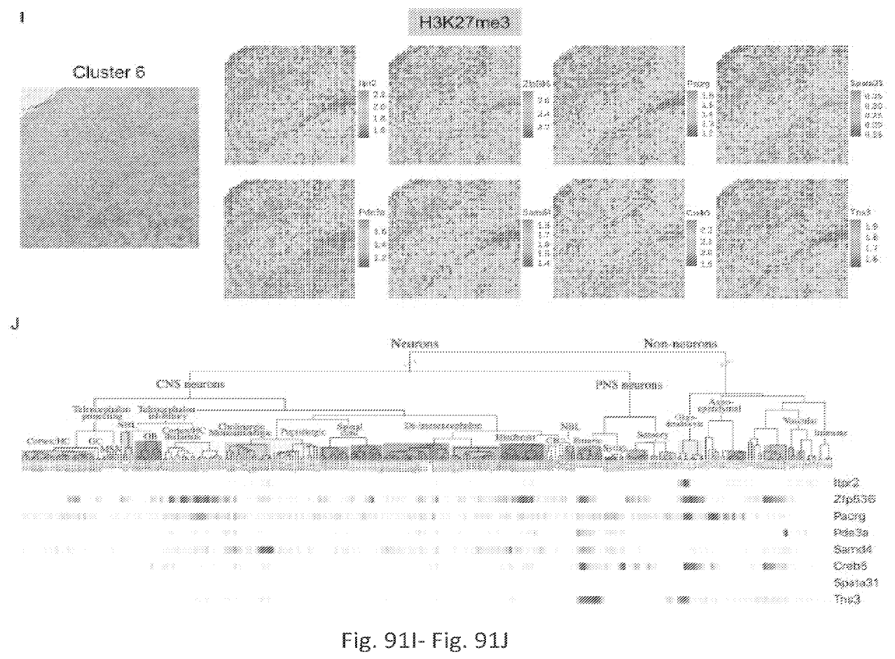

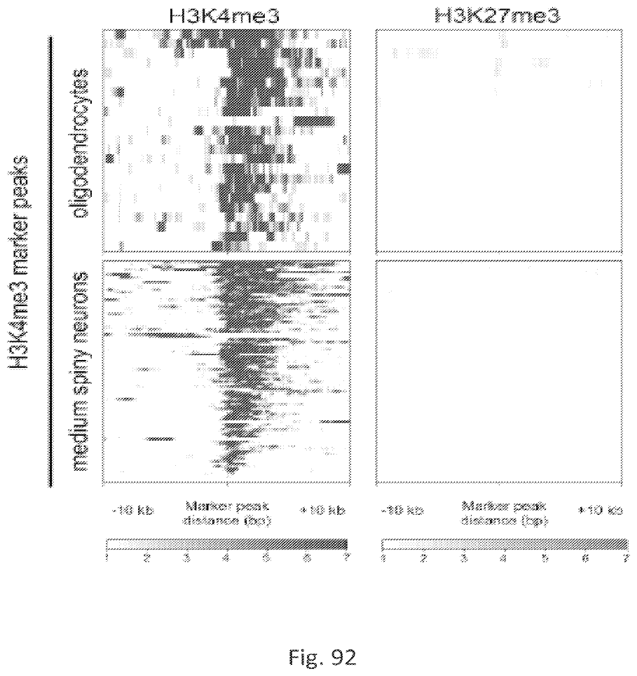

Lastly, to demonstrate the ability for spatially resolved chromatin state profiling in different tissue types, spatial-CUT&Tag was applied with 20 μm pixel size to the P21 mouse brain tissue sections. Unsupervised clustering revealed distinct spatial features (FIG. 89A to C). The spatial patterns of specific marker genes were explored to distinguish cell types and compared to the distribution of gene expression in the mouse CNS single-cell transcriptomic atlas (Zeisel et al., 2018, Cell, 174:999-1014.e1022) (FIGS. 89D and E, FIG. 90 and FIG. 91). For example, Sox10 showed high GAS in cluster 2 of H3K4me3 data, and Itpr 2 had low CSS in cluster 6 of H3K27me3 data, indicating these clusters were enriched with oligodendrocyte lineage cells. Cells of these clusters were particularly enriched in a stripe-like structure that corresponds to the corpus callosum (FIGS. 89D and E). For cluster 3 of H3K4me3 and H3K27me3 data, it was observed that Adcy5 was activated and Rbms3 was repressed, suggesting the epigenetic state of medium spiny neurons was enriched in these clusters. Further subclustering showed that some clusters that appeared to be homogenous could be further deconvoluted into sub-populations with distinct spatial distributions (FIG. 89F). For example, cluster 2 in the H3K27me3 data could be further subset into two clusters. Cux2, a marker of the superficial cortical layers 2 and 3, had lower H3K27me3 signal in subcluster 1. In contrast, Blc11b, a marker of the deeper cortical layers 4-6 presented higher H3K27me3 in subcluster 1. While Polycomb has been previously shown to play a role in the establishment of the cortical layers at embryonic stages (Hirabayashi et al., 2009, Neuron, 63:600-613; Morimoto-Suzki et al., 2014, Development, 141:4343-4353; Oishi et al., 2018, bioRxiv, 431684; Pereira et al., 2010, Proceedings of the National Academy of Sciences 107, 15957), the data suggests that H3K27me3 is also involved in maintaining cortical layer identity at the postnatal stages. To examine the interplay between active and repressive marks and infer the potential H3K4me3/H3K27me3 bivalency, all active promoters specific for individual populations marked by H3K4me3 were identified and plotted the signals of H3K4me3 and H3K27me3 per cell type (FIG. 92). As expected, H3K27me3 signals were depleted when the promoter is enriched in H3K4me3 in the respective population. However, H3K27me3 signals were also observed around few marker genes in oligodendrocytes and medium spiny neurons.

To further identify which cell types might be associated to each cluster, the spatial-CUT&Tag data was integrated with the mouse brain scCUT&Tag dataset that was recently generated (Bartosovic et al., 2021, Nature Biotechnology) and the publicly available mouse brain scRNA-seq dataset (Zeisel et al., 2018, Cell, 174:999-1014.e1022). The integrative data analysis revealed that microglia, mature oligodendrocytes, medium spiny neurons, astrocytes, and excitatory neurons were enriched in cluster 1, 2, 3, 4, and 7 respectively in the H3K4me3 dataset, and furthermore sub-populations of neurons could be identified (FIG. 89G to J, FIG. 90 and FIG. 91). Moreover, the integration of spatial-CUT&Tag with scRNA-seq or scCUT&Tag could allow for predicting which region a specific cell in scRNA-Seq or scCUT&Tag is localized in (FIG. 89G to J). Mature oligodendrocytes (MOL1) were identified to be abundant in the corpus callosum, while medial spinal neurons (MSN2) are present in the striatum, and TEGLU3 excitatory neurons in deeper cortical layer 6, in agreement with previously reported data (Zeisel et al., 2018, Cell, 174:999-1014.e1022), and determined herein by epigenetic modification states. TEGLU8 excitatory neurons have been shown to populate cortical layer 4 (Zeisel et al., 2018, Cell, 174:999-1014.e1022), and indeed it was observed that the corresponding epigenetic state of this neuronal population is distributed in a more superficial cortical layer than TEGLU3 (FIG. 89J). Interestingly, it was found that a sub-population of non-activated microglia (MGL1) mainly populates the striatum, but not the corpus callosum or cortex. In addition, the epigenetic state associated with protoplasmic astrocytes (ACTE2) is mainly localized in the corpus callosum although also observed in the cortex and striatum in lower frequency. Thus, the data from spatial-CUT&Tag could serve as a spatial atlas of epigenetic state with which one can map cell types to from single-cell transcriptomic or epigenomic dataset to spatial distribution.

Both a 50 μm device and a 20 μm device were able to perform spatial epigenome mapping of epigenomic markers. The 50 μm devices can cover larger tissue area. The 20 μm devices provide higher spatial resolution, which is at the near-cellular resolution.

### Example 5: High-Spatial-Resolution Multi-Omics Profiling

FIG. 93 shows the chemistry workflow of high-spatial-resolution multi-omics profiling. A tissue section on a standard aminated glass slide was lightly fixed with formaldehyde. Afterwards, a cocktail of antibody-DNA tags (ADTs) were first added to the tissue surface to capture target membrane proteins. After permeabilization, primary antibody binds to the target histone modifications or chromatin-interacting proteins, ADTs for intracellular proteins and ADTs for metabolites were added, followed by a secondary antibody binding for enhancing tethering of pA-Tn5 transposome. pA-Tn5 transposome and Tn5 proteins linked to methylation sensitive restriction enzyme were then activated by adding Mg++ and incubating at 37° C., and adapters containing ligation linker 1 was inserted at antibody bound sites. Then, RT mix combined with ligation linker 1 was added for mRNA capturing, reverse transcription and template switch. Afterwards, a set of DNA barcode A solutions were introduced to perform in situ ligation reaction for appending a distinct spatial barcode Ai (i=1-50) and ligation linker 2. A second set of barcodes Bj (j=1-50) were then introduced perpendicularly to those in the first flow barcoding, which were ligated at the intersections, resulting in a mosaic of tissue pixels, each containing a distinct combination of barcodes Ai and Bj (i=1-50, j=1-50). After DNA fragments and cDNA were collected by reversing cross-linking, PCR amplification and library construction were performed.

### Example 6: Spatial-ATAC-Seq: Spatially Resolved Chromatin Accessibility Profiling of Tissues at Genome Scale and Cellular Level

Spatial-ATAC-seq was developed for spatially resolved unbiased and genome-wide profiling of chromatin accessibility in intact tissue sections with the pixel size (20 μm) at cellular level. The data quality was excellent with ˜15,000 unique fragments detected per 20 μm pixel and up to ˜100,000 unique fragments per 50 μm pixel. It was applied to mouse embryos (E11 and E13) to delineate the epigenetic landscape of organogenesis, identified all major tissue types with distinct chromatin accessibility state, and revealed the spatiotemporal changes in development. It was also applied to mapping the epigenetic state of different immune cells in human tonsil and revealed the dynamics of B cell activation to GC reaction. The limitations or the areas for further development include the following. First, seamless integration with high-resolution tissue images, i.e., multicolor immunofluorescence image, to identify the cells in each pixel. It was observed that a significant number of pixels (20 μm) contained single nuclei and the extraction of sequencing reads from these pixels can give rise to spatially-defined single-cell ATAC-seq data. Second, integration with other spatial omics measurements such as transcriptome and proteins, to provide a comprehensive picture of cell types and cell states within the spatial context of tissue. Reagents for DBiT-seq (Liu et al., 2020, Cell, 183: 1665-1681 e1618) and spatial-ATAC-seq are combined in the same microfluidic barcoding step to achieve spatial multi-omics profiling, which should work in theory but does require further optimization for tissue fixation and reaction conditions to make these assays compatible. Third, it is yet to be further extended to human disease tissues to realize the full potential of spatial-ATAC-seq in clinical research. Spatial-ATAC-seq adds a new dimension to spatial biology, which may transform multiple biomedical research fields including developmental biology, neuroscience, immunology, oncology, and clinical pathology, thus empowering scientific discovery and translational medicine in human health and disease.

The Materials and Methods are now described

### Fabrication and Assembly of Microfluidic Device

The molds for microfluidic devices were fabricated in the cleanroom with standard photo lithography. The manufacturer's guidelines were followed to spin coat SU-8 negative photoresist (SU-2010, SU-2025, Microchem) on a silicon wafer (C04004, WaferPro). The feature heights of 50-μm-wide and 20-μm-wide microfluidic channel device were about 50 μm and 23 μm, respectively. During UV light exposure, chrome photomasks (Front Range Photomasks) were used. Soft lithography was used for polydimethylsiloxane (PDMS) microfluidic devices fabrication. Base and curing agent were mixed at a 10:1 ratio and added over the SU-8 masters. The PDMS was cured (65° C., 2 hours) after degassing in vacuum (30 minutes). After solidification, PDMS slab was cut out. The outlet and inlet holes were punched for further use.

### Preparation of Tissue Slides

Mouse C57 Embryo Sagittal Frozen Sections (MF-104-11-C57) and Human Tonsil Frozen Sections (HF-707) were purchased from Zyagen (San Diego, CA). Tissues were snapped frozen in OCT (optimal cutting temperature) compounds, sectioned (thickness of 7-10 μm) and put at the center of poly-L-lysine covered glass slides (63478-AS, Electron Microscopy Sciences).

### H&E Staining

The frozen slide was warmed at room temperature for 10 min and fixed with 1 mL 4% formaldehyde (10 min). After being washed once with 1×DPBS, the slide was quickly dipped in water and dried with air. Isopropanol (500 μl) was then added to the slide and incubate for 1 minute before being removed. After completely dry in the air, the tissue section was stained with 1 mL hematoxylin (Sigma) for 7 min and cleaned in DI water. The slide was then incubated in 1 mL bluing reagent (0.3% acid alcohol, Sigma) for 2 min and rinsed in DI water. Finally, the tissue slide was stained with 1 mL eosin (Sigma) for 2 min and cleaned in DI water.

### Preparation of Transposome

Unloaded Tn5 transposase (C01070010) was purchased from Diagenode, and the transposome was assembled following manufacturer's guidelines. The oligos used for transposome assembly were as follows:

Tn5ME-A:
(SEQ ID NO: 115)
5′-/5Phos/CATCGGCGTACGACTAGATGTGTATAAGAGACAG-3′
Tn5ME-B:
(SEQ ID NO: 102)
5′-GTCTCGTGGGCTCGGAGATGTGTATAAGAGACAG-3′
Tn5MErev:
(SEQ ID NO: 101)
5′-/5Phos/CTGTCTCTTATACACATCT-3′

### DNA Oligos, DNA Barcodes Sequences, and Other Key Reagents

DNA oligos used for sequencing library construction and PCR are listed in Table 1, other key reagents are given in Table 2, DNA barcodes sequences are shown in Table 3 (Example 7).

TABLE 1
DNA oligos used for PCR and preparation of sequencing library.

| | SEQ ID | |
|---|---|---|
| Sequence Name | NO: | Sequence |
| Tn5MErev | 101 | /5Phos/CTGTCTCTTATACACATCT |
| Tn5ME-B | 102 | GTCTCGTGGGCTCGGAGATGTGTATAA |
| | | GAGACAG |
| Ligation linker 1 | 103 | AGTCGTACGCCGATGCTGTCTCTTATAC |
| | | ACATCT |
| Ligation linker 2 | 104 | CGAATGCTCTGGCCTCTCAAGCACGTG |
| | | GAT |
| New-P5-SMART PCR | 105 | AATGATACGGCGACCACCGAGATCTAC |
| hybrid oligo | | ACGCCTGTCCGCGGAAGCAGTGGTATC |
| | | AACGCAGAGT*A*C |
| Ad2.1_TAAGGCGA | 106 | CAAGCAGAAGACGGCATACGAGATTCG |
| | | CCTTAGTCTCGTGGGCTCGGAGATGTG |
| Ad2.2_CGTACTAG | 107 | CAAGCAGAAGACGGCATACGAGATCTA |
| | | GTACGGTCTCGTGGGCTCGGAGATGTG |
| Ad2.3_AGGCAGAA | 108 | CAAGCAGAAGACGGCATACGAGATTTC |
| | | TGCCTGTCTCGTGGGCTCGGAGATGTG |
| Ad2.4_TCCTGAGC | 109 | CAAGCAGAAGACGGCATACGAGATGCT |
| | | CAGGAGTCTCGTGGGCTCGGAGATGTG |
| Ad2.5_GGACTCCT | 110 | CAAGCAGAAGACGGCATACGAGATAGG |
| | | AGTCCGTCTCGTGGGCTCGGAGATGTG |
| Ad2.6_TAGGCATG | 111 | CAAGCAGAAGACGGCATACGAGATCAT |
| | | GCCTAGTCTCGTGGGCTCGGAGATGTG |
| Ad2.7_CTCTCTAC | 112 | CAAGCAGAAGACGGCATACGAGATGTA |
| | | GAGAGGTCTCGTGGGCTCGGAGATGTG |
| Ad2.8_CAGAGAGG | 113 | CAAGCAGAAGACGGCATACGAGATCCT |
| | | CTCTGGTCTCGTGGGCTCGGAGATGTG |
| Custom Read 1 Primer | 114 | GCCTGTCCGCGGAAGCAGTGGTATCAA |
| | | CGCAGAGTAC |

TABLE 2
Chemicals and reagents.

| Name | Catalog number | Vender |
|---|---|---|
| Formaldehyde solution | PI28906 | Thermo Fisher Scientific |
| Glycine | 50046 | Sigma-Aldrich |
| Tris-HCl (pH 7.4) | T2663 | Sigma-Aldrich |
| NaCl | AM9760G | Thermo Fisher Scientific |
| MgCl2 | AM9530G | Thermo Fisher Scientific |
| Bovine Serum Albumin | A8806 | Sigma-Aldrich |
| (BSA) | | |
| Tween-20 | 11332465001 | Sigma-Aldrich |
| Digitonin | G9441 | Promega |
| NP-40 | 11332473001 | Sigma-Aldrich |
| EDTA Solution pH 8 | AB00502 | AmericanBio |
| Tn5 Transposase - | C01070010 | Diagenode |
| unloaded | | |
| Tagmentation Buffer (2x) | C01019043 | Diagenode |
| EZ-Tn5 Transposase | TNP92110 | Lucigen |
| Triton X-100 | T8787 | Sigma-Aldrich |
| T4 DNA Ligase | M0202L | New England Biolabs |
| T4 DNA Ligase Reaction | B0202S | New England Biolabs |
| Buffer | | |
| NEBuffer 3.1 | B7203S | New England Biolabs |
| DPBS | 14190144 | Thermo Fisher Scientific |
| Proteinase K | EO0491 | Thermo Fisher Scientific |
| Sodium dodecyl sulfate | 71736 | Sigma-Aldrich |
| DNA Clean & | D4014 | Zymo Research |
| Concentrator-5 | | |
| NEBNext High-Fidelity 2X | M0541L | New England Biolabs |
| PCR Master Mix | | |
| SYBR Green I Nucleic | S7563 | Thermo Fisher Scientific |
| Acid Gel Stain | | |
| Ampure XP beads | A63880 | Beckman Coulter |

### Spatial ATAC-Seq Profiling

The frozen slide was warmed at room temperature for 10 min. The tissue was fixed with formaldehyde (0.2%, 5 min) and quenched with glycine (1.25 M, 5 min) at room temperature. After fixation, the tissue was washed twice with 1 mL 1×DPBS and cleaned in DI water. The tissue section was then permeabilized with 500 μL lysis buffer (10 mM Tris-HCl, pH 7.4; 10 mM NaCl; 3 mM MgCl2; 0.01% Tween-20; 0.01% NP-40; 0.001% iDigitonin; 1% BSA) for 15 min and was washed by 500 μL wash buffer (10 mM Tris-HCl pH 7.4; 10 mM NaCl; 3 mM MgCl2; 1% BSA; 0.1% Tween-20) for 5 min. 100 μL transposition mix (50 μL 2× tagmentation buffer; 33 μL 1×DPBS; 1 μL 10% Tween-20; 1 μL 1% Digitonin; 5 μL transposome; 10 μL Nuclease-free H₂O) was added followed by incubation at 37° C. for 30 min. After removing transposition mix, 500 μL 40 mM EDTA was added for incubation at room temperature for 5 min to stop transposition. Finally, the EDTA was removed, and the tissue section was washed with 500 μL 1× NEBuffer 3.1 for 5 min.

For barcodes A in situ ligation, the 1st PDMS slab was used to cover the region of interest, the brightfield image was taken with 10× objective (Thermo Fisher EVOS fl microscope) for further alignment. The tissue slide and PDMS device were then clamped with an acrylic clamp. First, DNA barcodes A was annealed with ligation linker 1, 10 μL of each DNA Barcode A (100 μM), 10 μL of ligation linker (100 μM) and 20 μL of 2× annealingbuffer (20 mM Tris, pH 7.5-8.0, 100 mM NaCl, 2 mM EDTA) were added together and mixed well. Then, 5 μL ligation reaction solution (50 tubes) was prepared by adding 2 μL of ligation mix (72.4 μL of RNase free water, 27 μL of T4 DNA ligase buffer, 11 μL T4 DNA ligase, 5.4 μL of 5% Triton X-100), 2 μL of 1×NEBuffer 3.1 and 1 μL of each annealed DNA barcode A (A1-A50, 25 μM) and loaded into each of the 50 channels with vacuum. The chip was kept in a wet box for incubation (37° C., 30 min). After flowing through 1×NEBuffer 3.1 for washing (5 min), the clamp and PDMS were removed. The slide was quickly dipped in water and dried with air.

For barcodes B in situ ligation, the 2nd PDMS slab with channels perpendicular to the 1st PDMS was attached to the dried slide carefully. A brightfield image was taken and the acrylic clamp was used to press the PDMS against the tissue. The annealing of DNA barcodes B with ligation linker 2 were the same with DNA barcodes A and ligation linker 1 annealing. The preparation and addition of ligation reaction solution for DNA barcode B (B1-B50, 25 μM) were also the same with DNA barcode A (A1-A50, 25 μM). The chip was kept in a wet box for incubation (37° C., 30 min). After flowing through 1×DPBS for washing (5 min), the clamp and PDMS were removed, the tissue section was dipped in water and dried with air. The final brightfield image of the tissue was taken.

For tissue digestion, the interest region of the tissue was covered with a square PDMS well gasket and 100 μL reverse crosslinking solution (50 mM Tris-HCl, pH 8.0; 1 mM EDTA; 1% SDS; 200 mM NaCl; 0.4 mg/mL proteinase K) was loaded into it. The lysis was conducted in a wet box (58° C., 2 h). The final tissue lysate was collected into a 200 μL PCR tube for incubation with rotation (65° C., overnight).

For library construction, the lysate was first purified with Zymo DNA Clean & Concentrator-5 and eluted to 20 μL of DNA elution buffer, followed by mixing with the PCR solution (2.5 μL 25 μM new P5 PCR primer; 2.5 μL 25 μM Ad2 primer; 25 μL 2× NEBNext Master Mix). Then, PCR was conducted with following the program: 72° C. for 5 min, 98° C. for 30 s, and then cycled 5 times at 98° C. for 10 s, 63° C. for 10 s, and 72° C. for 1 min. To determine additional cycles, 5 μL of the pre-amplified mixture was first mixed with the qPCR solution (0.5 μL 25 μM new P5 PCR primer; 0.5 μL 25 μM Ad2 primer; 0.24 μl 25×SYBR Green; 5 μL 2× NEBNext Master Mix; 3.76 μL nuclease-free H₂O). Then, qPCR reaction was carried out at the following conditions: 98° C. for 30 s, and then 20 cycles at 98° C. for 10 s, 63° C. for 10 s, and 72° C. for 1 min. Finally, the remainder 45 μL of the pre-amplified DNA was amplified by running the required number of additional cycles of PCR (cycles needed to reach ⅓ of saturated signal in qPCR).

To remove PCR primers residues, the final PCR product was purified by 1× Ampure XP beads (45 μL) following the standard protocol and eluted in 20 μL nuclease-free H₂O. Before sequencing, an Agilent Bioanalyzer High Sensitivity Chip was used to quantify the concentration and size distribution of the library. Next Generation Sequencing (NGS) was performed using the Illumina HiSeq 4000 sequencer (pair-end 150 bp mode with custom read 1 primer).

### Data Preprocessing

Two constant linker sequences (linker 1 and linker 2) were used to filter Read 1, and the filtered sequences were transformed to Cell Ranger ATAC format (10× Genomics). The genome sequences were in the new Read 1, barcodes A and barcodes B were included in new Read 2. Resulting fastq files were aligned to the mouse reference (mm10) or human reference (GRCh38), filtered to remove duplicates and counted using Cell Ranger ATAC v1.2. The BED like fragments file were generated for downstream analysis. The fragments file contains fragments information on the genome and tissue location (barcode A×barcode B). A preprocessing pipeline developed using Snakemake workflow management system is shared at github.com/dyxmvp/Spatial_ATAC-seq.

### Data Visualization

Pixels were identified on tissue with manual selection from microscope image using Adobe Illustrator (github.com/rongfan8/DBiT-seq), and a custom python script was used to generate metadata files that were compatible with Seurat workflow for spatial datasets.

The fragment file was read into ArchR as a tile matrix with the genome binning size of 5 kb, and pixels not on tissue were removed based on the metadata file generated from the previous step. Data normalization and dimensionality reduction was conducted using iterative Latent Semantic Indexing (LSI) (iterations=2, resolution=0.2, varFeatures=25000, dimsToUse=1:30, sampleCells=10000, n.start=10), followed by graph clustering and Uniform Manifold Approximation and Projection (UMAP) embeddings (nNeighbors=30, metric=cosine, minDist=0.5) (Granja et al., 2020, bioRxiv, 2020.2004.2028.066498).

Gene Score model in ArchR was employed to gene accessibility score. Gene Score Matrix was generated for downstream analysis. The getMarkerFeatures and getMarkers function in ArchR (testMethod=“wilcoxon”, cutOff=“FDR<=0.05 & Log 2FC>=0.25”) was used to identify the marker regions/genes for each cluster, and gene scores imputation was implemented with addImputeWeights for data visualization. The enrichGO function in the clusterProfiler package was used for GO enrichment analysis (qvalueCutoff=0.05) (Yu et al., 2012, Omics: a journal of integrative biology, 16:284-287). For spatial data visualization, results obtained in ArchR were loaded to Seurat V3.2.3 to map the data back to the tissue section (Stuart et al., 2019, Cell, 177:1888-1902 e1821; Butler et al., 2018, Nat Biotechnol, 36:411-420).

In order to project bulk ATAC-seq data, raw sequence data aligned to mm10 (BAM files) was downloaded from ENCODE. After counting the reads in 5 kb tiled genomes using getCounts function in chrom VAR (Schep et al., 2017, Nature Methods, 14:975-978), the projectBulkATAC function in ArchR was used.

Cell type identification and pseudo-scRNA-seq profiles was added through integration with scRNA-seq reference data (Cao et al., 2019, Nature, 566:496-502). FindTransferAnchors function (Seurat V3.2 package) was used to align pixels from spatial ATAC-seq with cells from scRNA-seq by comparing the spatial ATAC-seq gene score matrix with the scRNA-seq gene expression matrix. GeneIntegrationMatrix function in ArchR was used to add cell identities and pseudo-scRNA-seq profiles.

Pseudobulk group coverages based on cluster identities were generated with addGroupCoverages and used for peak calling with macs2 using addReproduciblePeakSet function in ArchR. To compute per-cell motif activity, chrom VAR (Schep et al., 2017, Nature Methods, 14:975-978) was run with addDeviationsMatrix using the cisbp motif set after a background peak set was generated using addBgdPeaks. Cell type-specific marker peaks were identified with getMarkerFeatures (bias=c(“TSSEnrichment”, “log 10(nFrags)”), testMethod=“wilcoxon”) and getMarkers (cutOff=“FDR<=0.05 & Log 2FC>=0.1”). Pseudotemporal reconstruction was implemented by addTrajectory function in ArchR.

Published data for data quality comparison and integrative data analysis 10× scATAC-seq (Flash frozen): Flash frozen cortex, hippocampus, and ventricular zone from embryonic mouse brain (E18). (Single Cell ATAC Dataset by Cell Ranger ATAC 1.2.0) ENCODE (bulk): Public bulk ATAC-seq datasets were downloaded from ENCODE (E11.5 and E13.5).

The Experimental Results are now described

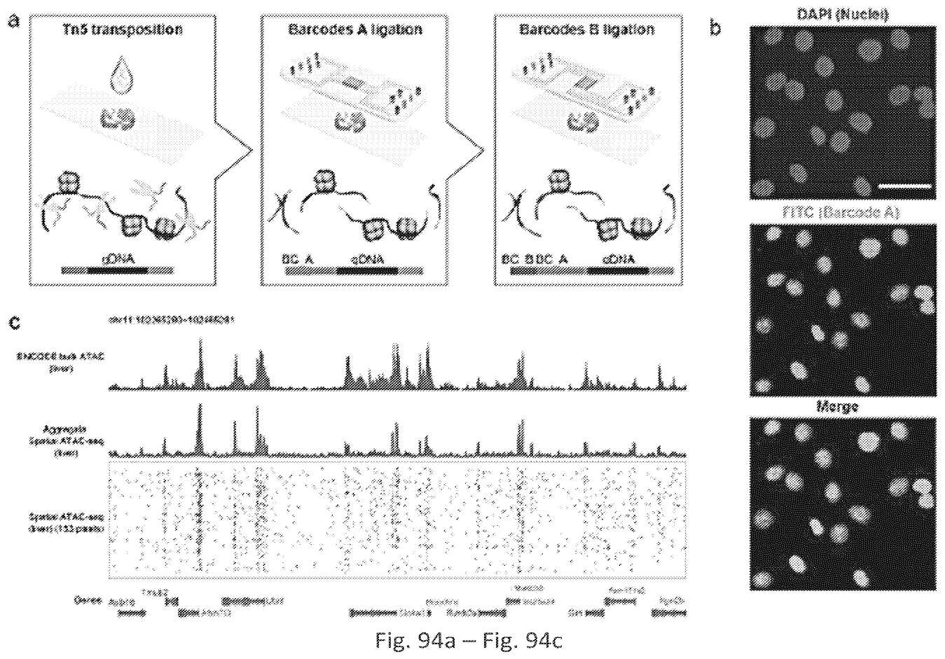

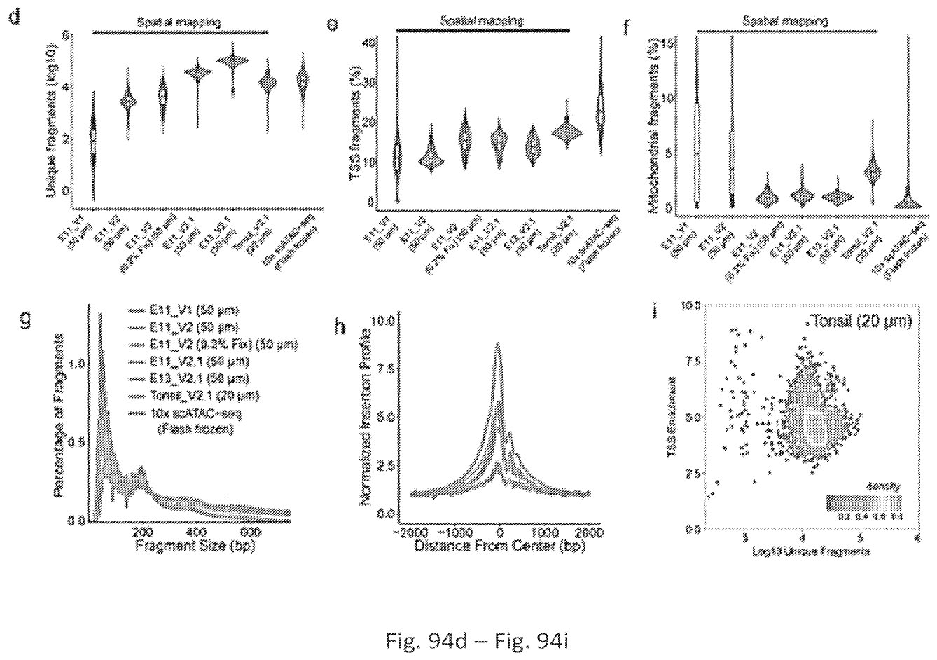

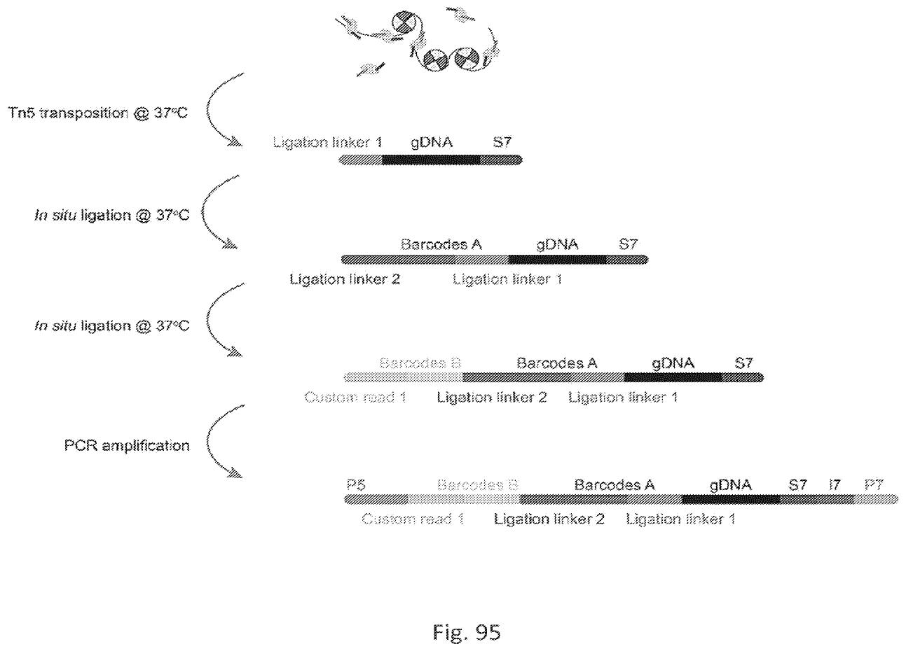

Spatial-ATAC-seq is presented for mapping chromatin accessibility in a tissue section at cellular level via combining the strategy of microfluidic deterministic barcoding in tissue (Liu et al, 2020, Cell, 183(6): 1665-1681) and the chemistry of the assay for transposase-accessible chromatin (Buenrostro et al., 2013, Nat Methods, 10:1213-1218, Corces et al., 2017, Nat Methods, 14:959-962) (FIG. 94a and FIG. 95). The main workflow for spatial ATAC-seq is shown in FIG. 94a. The fresh frozen tissue section on a standard aminated glass slide was fixed with formaldehyde. Tn5 transposition was then performed and the adapters containing a ligation linker were inserted to transposase accessible genomic DNA loci. Afterwards, a set of DNA barcode A solutions were introduced to the tissue surface using an array of microchannels for in situ ligation of distinct spatial barcode Ai (i=1-50) to the adapters. Then, a second set of barcodes Bj (j=1-50) were introduced over the tissue surface in microchannels perpendicularly to those in the first flow barcoding step. They were subsequently ligated at the intersections, resulting in a 2D mosaic of tissue pixels, each of which contains a distinct combination of barcodes Ai and Bj (i=1-50, j=1-50). During each flow or afterward, the tissue slides were imaged under an optical microscope such that spatially barcoded accessible chromatin can be correlated with the tissue morphology. After forming a spatially barcoded tissue mosaic (n=2500), reverse crosslinking was performed to release barcoded DNA fragments, which were amplified by PCR for sequencing library preparation. To evaluate the performance of in situ transposition and ligation, the 4′, 6-diamidino-2-phenylindole (DAPI) stained adherent NIH 3T3 cells were fixed by formaldehyde on a glass slide. The cells were then transposed by Tn5 transposase followed by ligation of a dummy barcode A labeled with fluorescein isothiocyanate (FITC) to evaluate the chemistry with fluorescence microscopy. The resulting images revealed a strong overlap between nucleus (blue) and FITC signal (green), indicating the successful insertion of adaptors into accessible chromatin loci with ligated barcode A in nuclei only (FIG. 94b).

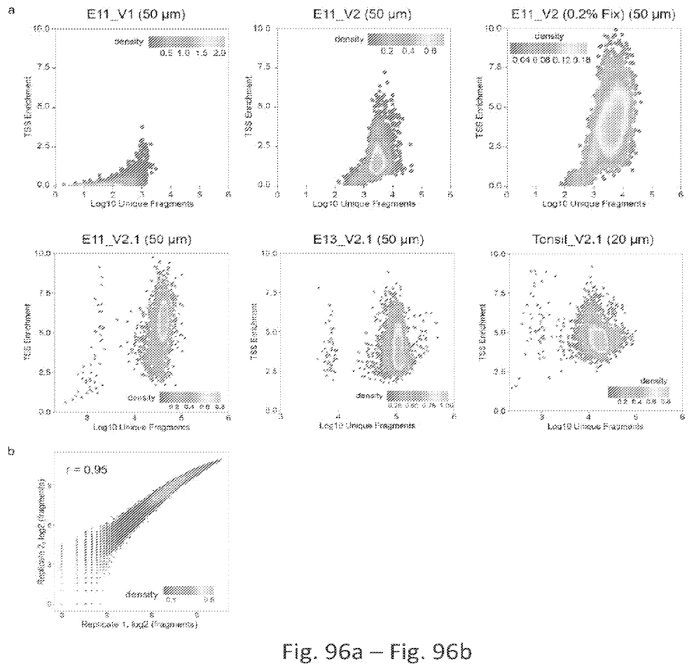

Several versions of chemistry were gone through to develop spatial-ATAC-seq, and to optimize the protocol in order to achieve high yield and high signal-to-noise ratio for the mapping of tissue sections (FIG. 94d-i and FIG. 96a). In chemistry V1, a set of 50 DNA oligomers containing both barcode A and adapter were introduced in microchannels to a tissue section for in situ transposition but the efficiency was low due in part to limited amounts of Tn5-DNA in microchannels. In chemistry V2, bulk transposition was conducted followed by two ligation steps to introduce spatial barcodes A-B. The fixation condition was optimized by reducing formaldehyde concentration from 4% in chemistry V1 to 0.2% in chemistry V2. The sensitivity of different Tn5 transposase enzymes was tested (Diagenode (C01070010) in chemistry V2.1 vs Lucigen (TNP92110) in chemistry V2). The performance measured by the unique fragments detected and the transcription start site (TSS) enrichment score from V1, V2, to V2.1 was summarized in FIG. 96a. The optimized spatial-ATAC-seq protocol V2.1 was applied to mouse embryos (E11 and E13) and human tonsil, and the data quality was assessed by comparison to non-spatial scATAC-seq data from the commercialized platform (10× Genomics). In 50 μm spatial ATAC-seq experiments, a median of 36,303 (E11) and 100,786 (E13) unique fragments were obtained per pixel of which 15% (E11) and 14% (E13) of fragments overlap with TSS regions. In addition, proportion of mitochondrial fragments is low for both E11 and E13 (1%). As for the 20 μm spatial-ATAC-seq experiment with human tonsil, a median of 14,939 unique fragments per pixel was obtained of which 18% of fragments fell within TSS regions. The fraction of read-pairs mapping to mitochondria is 3%. Overall, the data quality of spatial-ATAC-seq from the tissue section is equivalent to non-spatial scATAC-seq (17,321 unique fragments per cell, 23% TSS fragments, and 0.4% mitochondrial reads). Moreover, the insert size distribution of spatial-ATAC-seq fragments was consistent with the capture of nucleosomal and subnucleosomal fragments for all tissue types (FIG. 94g). A correlation analysis was performed between biological replicates of serial tissue sections for spatial-ATAC-seq, which showed high reproducibility with the Pearson correlation coefficient of 0.95 (FIG. 96b). Using spatial-ATAC-seq, DNA accessibility profiles of individual tissue pixels in the fetal liver of an E13 mouse embryo were generated. Aggregate profiles of spatial ATAC-seq data accurately reproduced the bulk measurement of accessibility obtained from the ENCODE reference database (FIG. 94c).

### Spatial Chromatin Accessibility Mapping of E13 Mouse Embryo

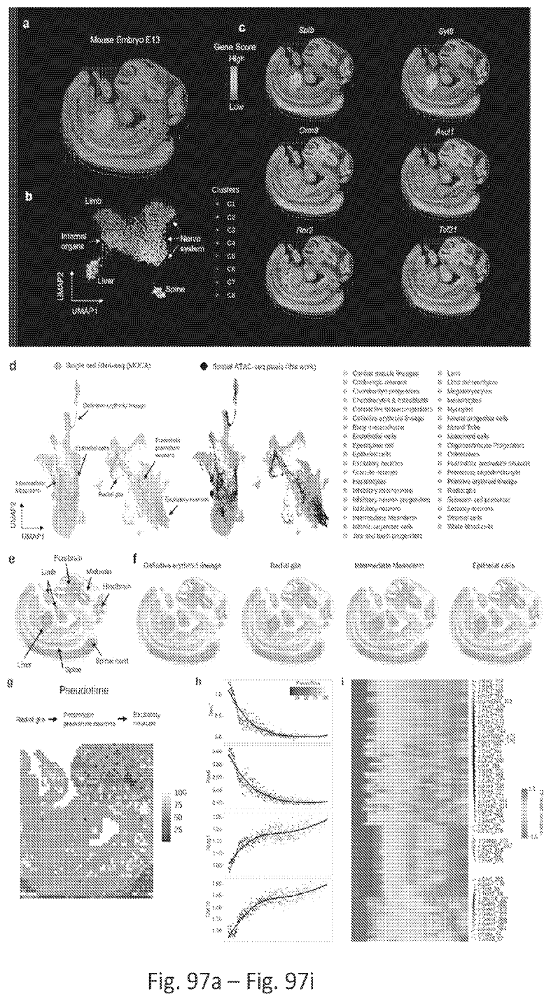

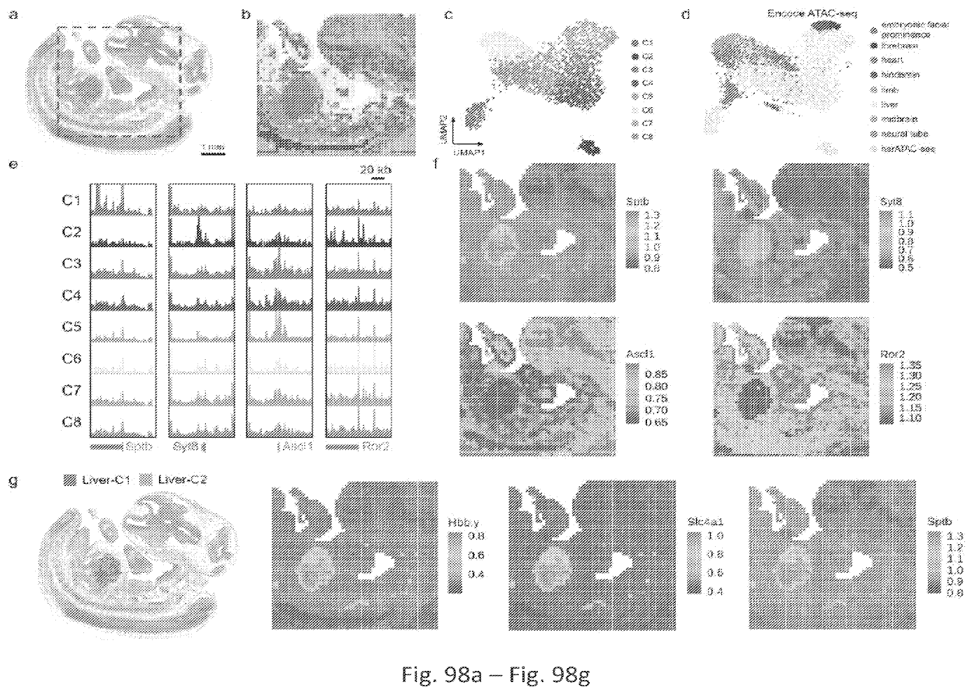

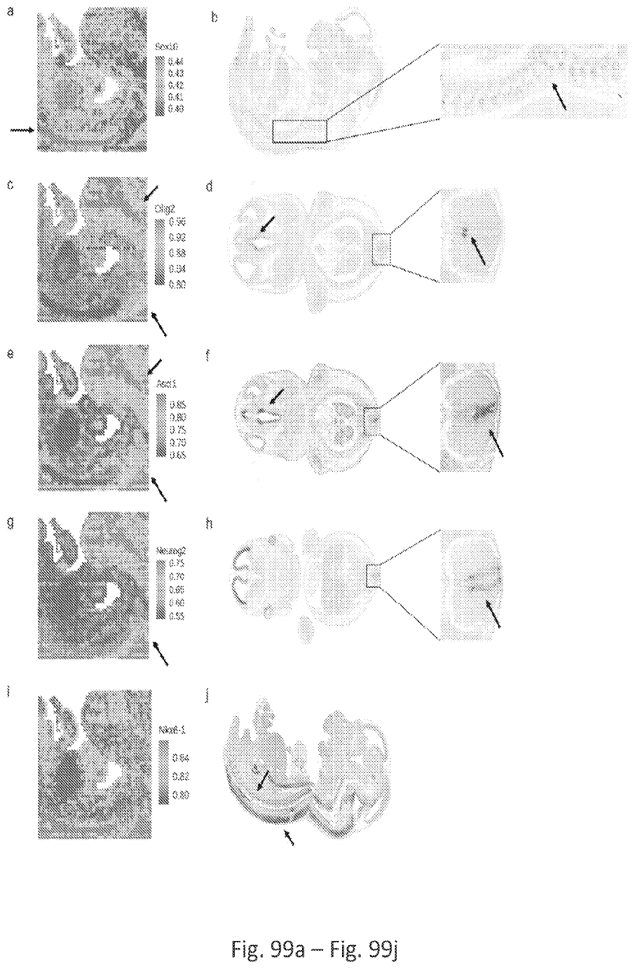

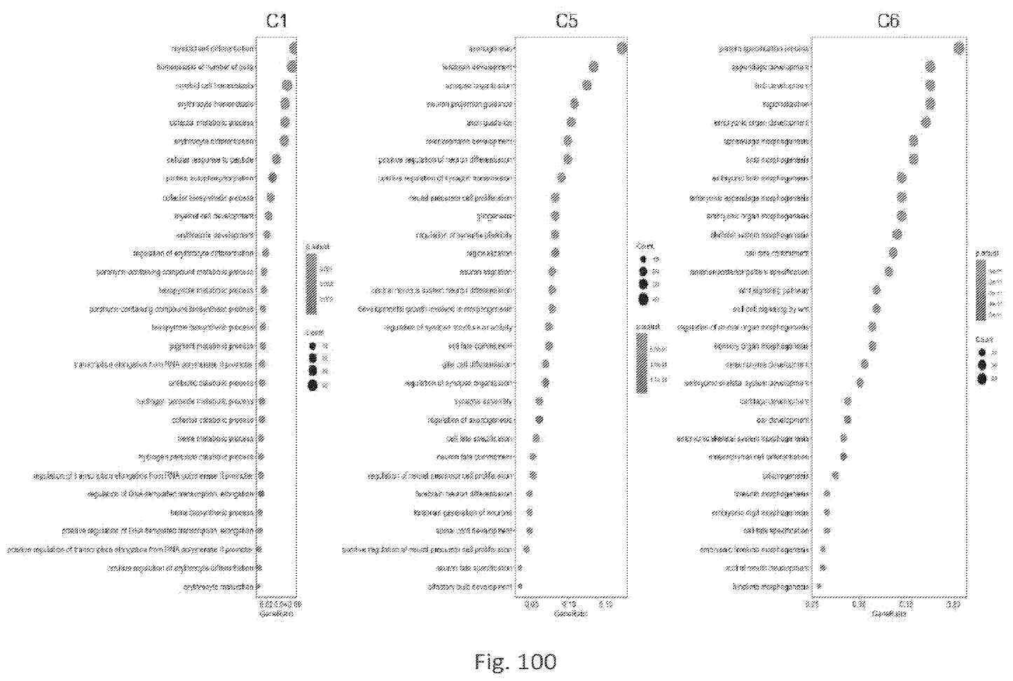

Next it was sought to identify cell types de novo by chromatin accessibility from the E13 mouse embryo. A pixel by tile matrix was generated by aggregating reads in 5 kilobase bins across the mouse genome. Latent semantic indexing (LSI) and uniform manifold approximation and projection (UMAP) were then applied for dimensionality reduction and embedding, followed by Louvain clustering using the ArchR package (Granja et al., 2021, Nature Genetics, 53:403-411). Unsupervised clustering identified 8 main clusters and the spatial map of these clusters revealed distinct patterns that agreed with the tissue histology shown in an adjacent H&E stained tissue section (FIG. 97a to c, FIG. 98). For example, cluster 1 represents the fetal liver in the mouse embryo, and cluster 2 is specific to the spine region, including the dorsal root ganglia (FIG. 99a, b, i, j). Cluster 3 to cluster 5 are associated with the peripheral and central nervous system (PNS and CNS). Cluster 6 includes several cell types present in the developing limbs, and cluster 8 encompasses several developing internal organs. To benchmark spatial-ATAC-seq data, the ENCODE organ-specific ATAC-seq data was projected onto the UMAP embedding using the UMAP transform function (Gorkin et al., 2020, Nature, 583:744-751). In general, the cluster identification matched well with the bulk ATAC-seq projection (FIG. 98b-d) and distinguished all major developing tissues and organs in a E13 mouse embryo. Further, cell type-specific marker genes were examined and the expression of these genes wsas estimated from chromatin accessibility data based on the overall signal at a given locus (Granja et al., 2021, Nature Genetics, 53:403-411) (FIG. 97c, FIG. 98e, f). Sptb, which plays a role in stability of erythrocyte membranes, was activated extensively in the liver. Syt8, which is important in neurotransmission, had a high level of gene activity in the spine. Ascl1 showed strong enrichment in the mouse brain, which is known to be involved in the commitment and differentiation of neuron and oligodendrocyte (FIG. 97c, FIG. 99e, f). Sox 10 marks oligodendrocyte progenitor cells (OPCs). It was expressed at a high level in the dorsal root ganglia (DRGs), which are adjacent to the spinal cord (FIG. 99a, b). Olig2 is a marker of neural progenitors, pre-OPCs and OPCs. Olig2 is expressed in a small domain of the spinal cord, in the ventral domains of the forebrain, and in some posterior regions (brain stem, midbrain and hindbrain), which is consistent with the high gene score in the spatial ATAC-seq data (FIG. 99c, d). However, its expressionin forebrain is confined at the dorsal side at this developmental stage as detected by in situ hybridization (FIG. 99c), but the chromatin accessibility is open in both dorsal and ventral side, suggesting the possibility of epigenetic priming. Ror2 correlates with the early formation of the chondrocytes and cartilage, and it was highly expressed in the limb (Stelzer et al., 2016, Current Protocols in Bioinformatics, 54:1.30.31-31.30.33). Pathway analysis of marker genes revealed that cluster 1 was associated with in erythrocyte differentiation, cluster 5 corresponded to forebrain development, and cluster 6 was involved in limb development, all in agreement with anatomical annotations (FIG. 100). Interestingly, it was found that the clusters that appeared to be homogenous could be further deconvoluted into sub-populations with distinct spatial distributions (FIG. 98g). For example, the fetal liver could be further subset to two clusters, and it was found that some genes related to hematopoiesis (e.g. Hbb-y, Slc-4a1, Sptb) had higher expression in subcluster 1 (FIG. 98g). Moreover, the expression patterns in the spine of the E13 mouse embryo were further investigated and the genes showing epigenetic gradients along the anterior-posterior axis were selected (FIG. 101).

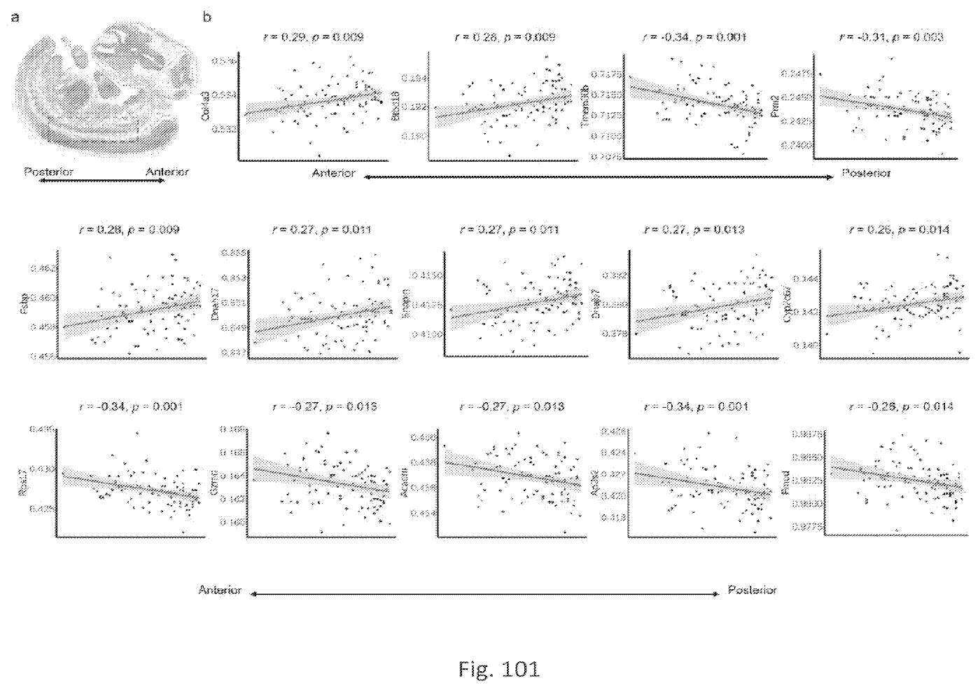

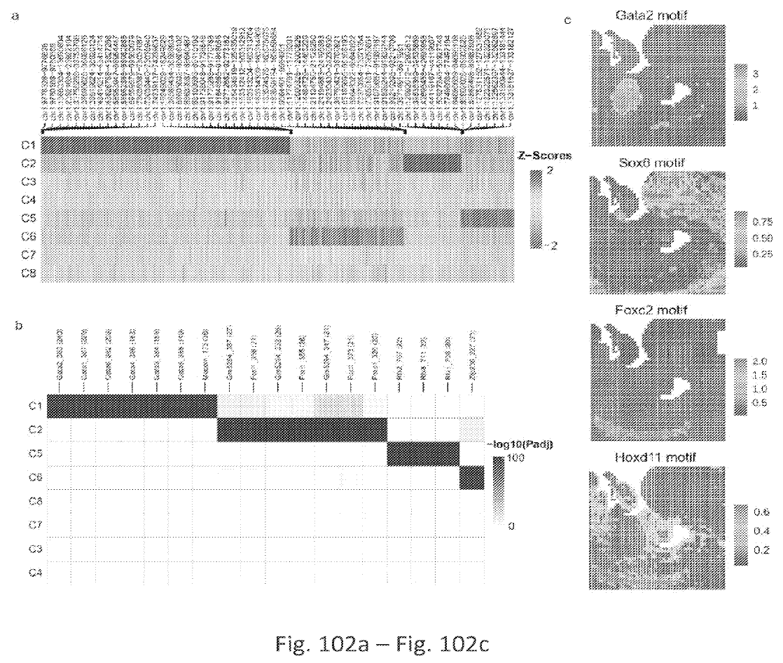

In addition to the inference of cell type-specific marker genes, this approach also enabled the unbiased identification of cell type-specific chromatin regulatory elements (FIG. 102), which provides a resource for defining regulatory elements as cell type-specific reporters. To further utilize the underlying chromatin accessibility data, it was sought to examine cell type-specific transcription factor (TF) regulators within each cluster using deviations of TF motifs. It was found that the most enriched motifs in the peaks that are more accessible in fetal liver correspond to Gata transcription factors, consistent with their well-studied role in erythroid differentiation (FIG. 102b, c). Cluster 5 enriched for Sox6 motif that supports its role for the CNS development. Hoxd11, which marks the posterior patterning and plays a role in limb morphogenesis, was enriched in the limb (FIG. 102c).

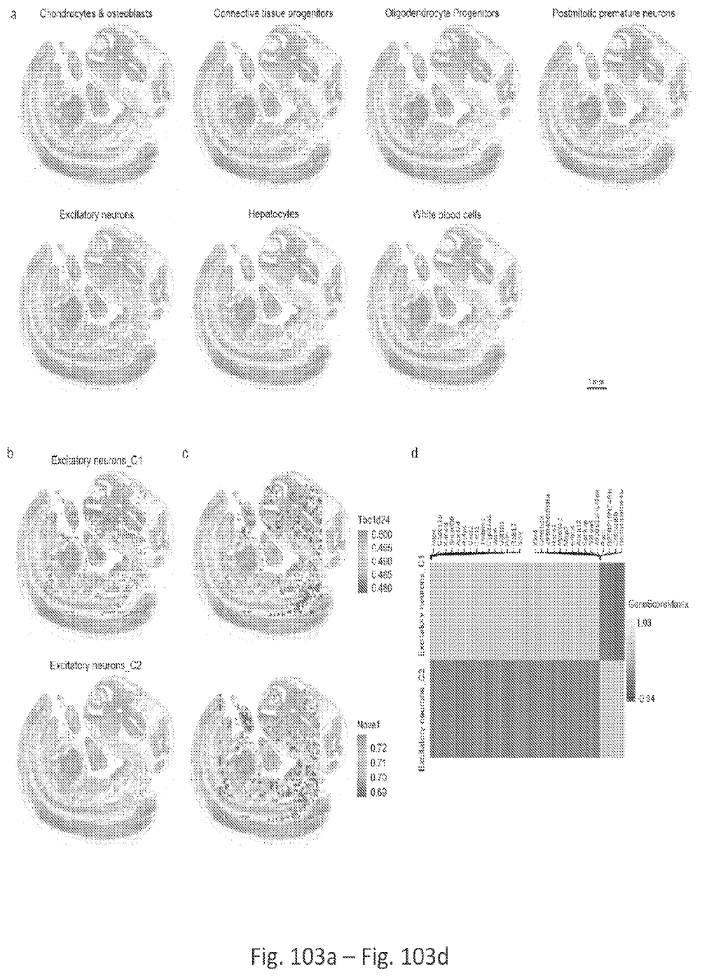

The spatial ATAC-seq data was integrated with the scRNA-seq data to assign cell types to each cluster (Cao et al., 2019, Nature, 566:496-502) (FIG. 97d-f, FIG. 103a). For example, the definitive erythroid cells were exclusively enriched in the liver. Additionally, few hepatocytes and white blood cells were found in this region, which could not be identified in the E11 data, suggesting that these cell types emerged at the later developmental time points. Intermediate mesoderm was identified in the internal organ region, and radial glia was mainly distributed in the CNS. A refined clustering process also enabled identification of sub-populations in excitatory neurons with distinct spatial distributions, marker genes and chromatin regulatory elements (FIG. 103b-d). During embryonic development, dynamic changes in chromatin accessibility across time and space help regulate the formation of complex tissue architectures and terminally differentiated cell types (Stickels et al., 2020, Nature Biotechnology, doi: 10.1038/s41587-020-0739-1). In the embryonic CNS, radial glia function as primary progenitors or neural stem cells (NSCs), which give rise to various types of neurons (Kriegstein et al., 2009, Annual Review of Neuroscience, 32:149-184). Therefore, spatial ATAC-seq data was exploited to recover the spatially organized developmental trajectory and examine how developmental processes proceed across the tissue space. Here, the course of a developmental process was studied from radial glia to excitatory neurons with postmitotic premature neurons as the immediate state after the radial glial differentiation, and ordered these cells in pseudo-time using ArchR. Spatial projection of each pixel's pseudo-time value revealed the spatially organized developmental trajectory in neurons (FIG. 97g). Changes were identified in gene expression and TF deviations across this developmental process, and many genes recovered are important in neuron development, including Sox2, which is required for stem cell maintenance in the central nervous system, and Ning1, which is involved in controlling patterning and neuronal circuit formation (FIG. 97h, i).

Spatial Chromatin Accessibility Mapping of E11 Mouse Embryo and Comparison with E13 to Investigate the Spatiotemporal Relationship

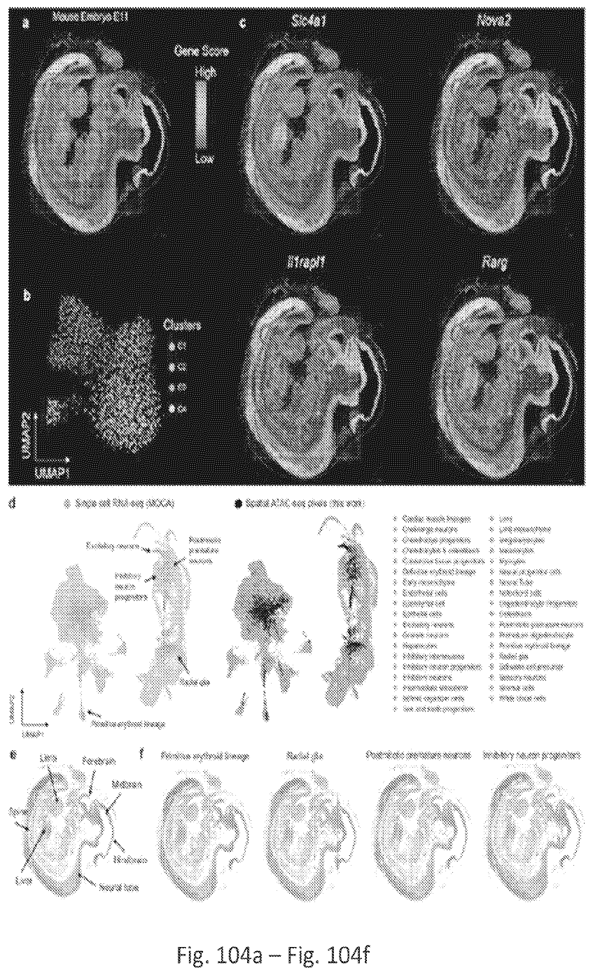

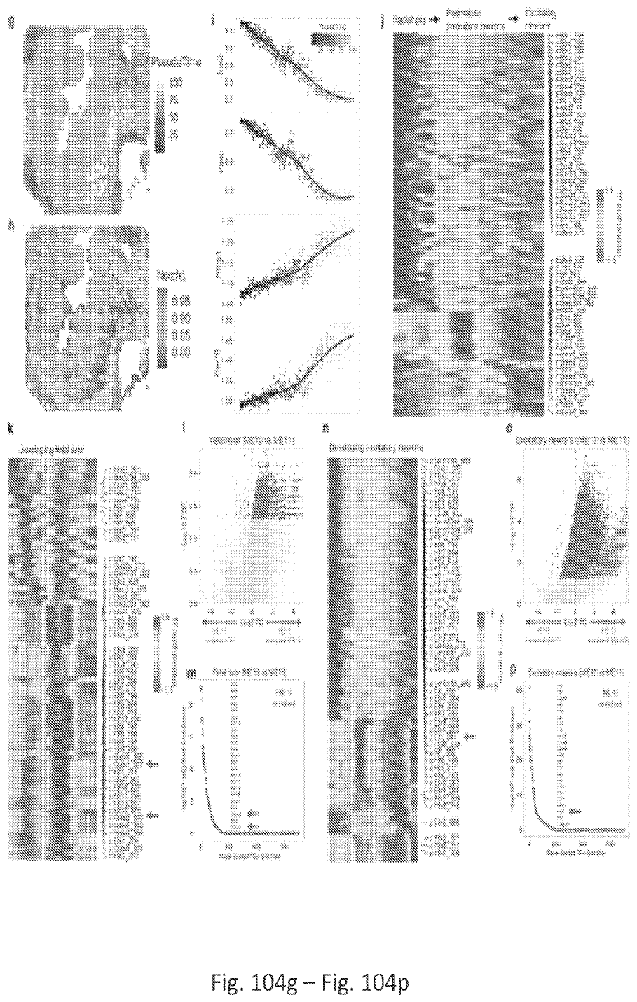

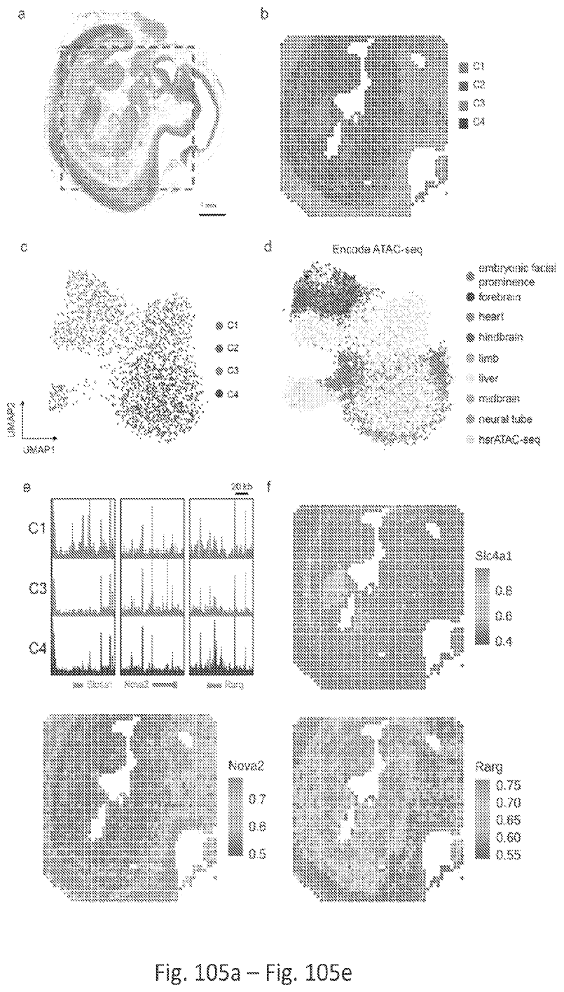

To map chromatin accessibility during mouse fetal development, a mouse embryo was profiled at E11. Unsupervised clustering identified 4 main clusters with distinct spatial patterns, which showed good agreement with the anatomy in an adjacent H&E stained tissue section (FIG. 104a-c, FIG. 105a-c). Cluster 1 is located in the fetal liver and aorta-gonad-mesonephros (AGM), which are related to embryonic hematopoiesis. It should be noted that spatial ATAC-seq can resolve the fine structure in mouse embryo such as AGM, showing its capability to profile chromatin accessibility in a high spatial resolution manner. Cluster 2 and cluster 3 consist of tissues associated with neuronal development such as mouse brain and neural tube. Cluster 4 includes the embryonic facial prominence, internal organs and limb. In addition, cluster identification matched the ENCODE organ-specific bulk ATAC-seq projection onto the UMAP embedding (FIG. 105d).

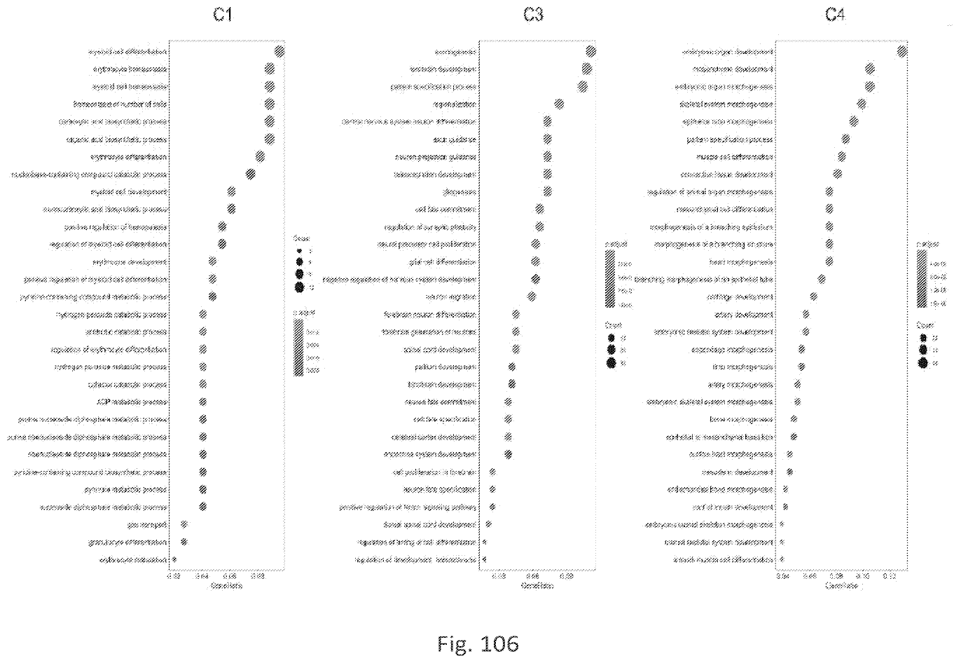

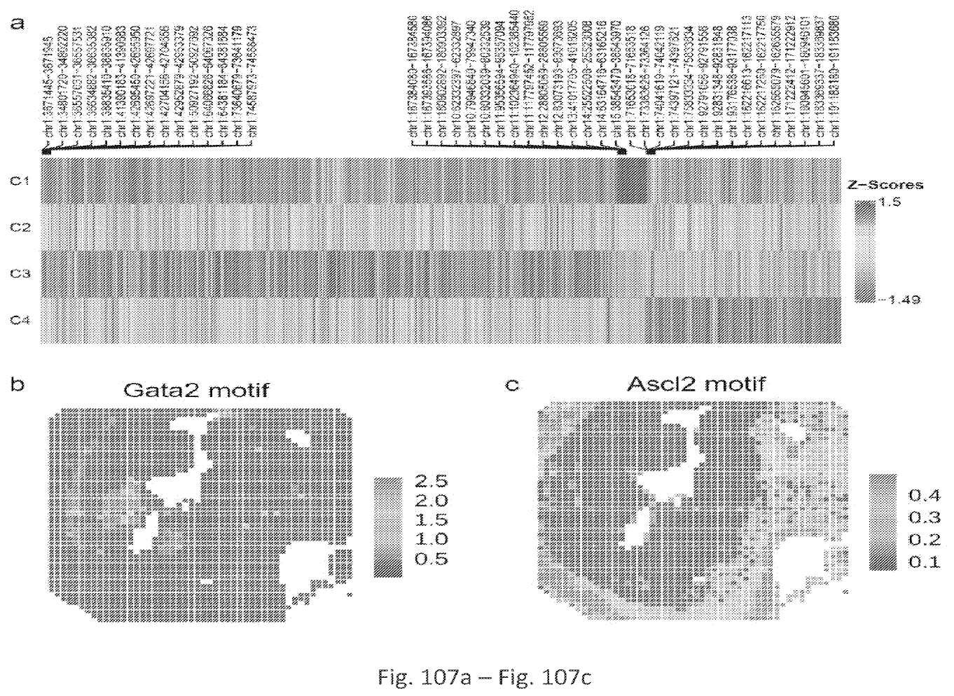

The chromatin accessibility patterns that distinguished each cluster (FIG. 104c, FIG. 105e, f) were surveyed. For example, Slc-4a1, which are required for normal flexibility and stability of the erythrocyte membrane and for normal erythrocyte shape, were highly active in liver and AGM. Nova2, which is involved in RNA splicing or metabolism regulation in a specific subset of developing neurons, was highly enriched in the brain and neural tube. Rarg, which plays an essential role in limb bud development, skeletal growth, and matrix homeostasis, was activated extensively in the embryonic facial prominence and limb (Stelzer et al., 2016, Current Protocols in Bioinformatics, 54:1.30.31-31.30.33). Moreover, Gene Ontology (GO) enrichment analysis were conducted for each cluster, and the GO pathways identified the development processes consistent with the anatomical annotation (FIG. 106). To gain deeper insights into the regulatory factors in each tissue, chromatin regulatory elements were clustered and enrichment for TF binding motifs, and expression patterns were examined of those motifs (FIG. 107). Strong enrichment of the motifs for Gata2 (FIG. 106b) and Ascl2 (FIG. 107c) was observed in the clusters associated with embryonic hematopoiesis and neuronal development, respectively. These master regulators further validated the unique identity of each cluster.

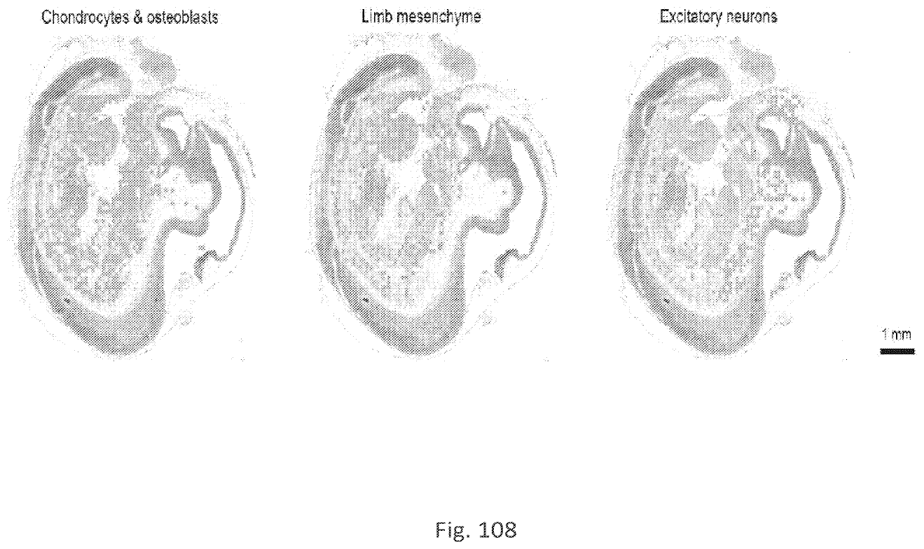

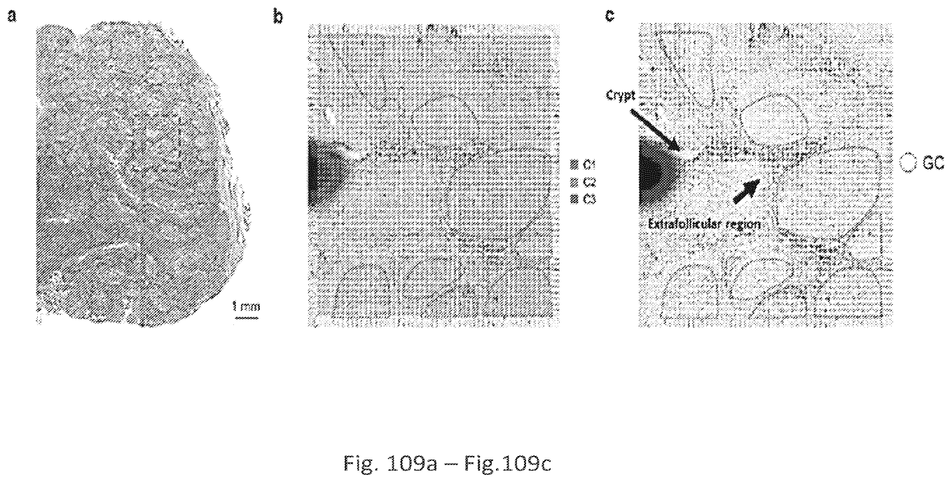

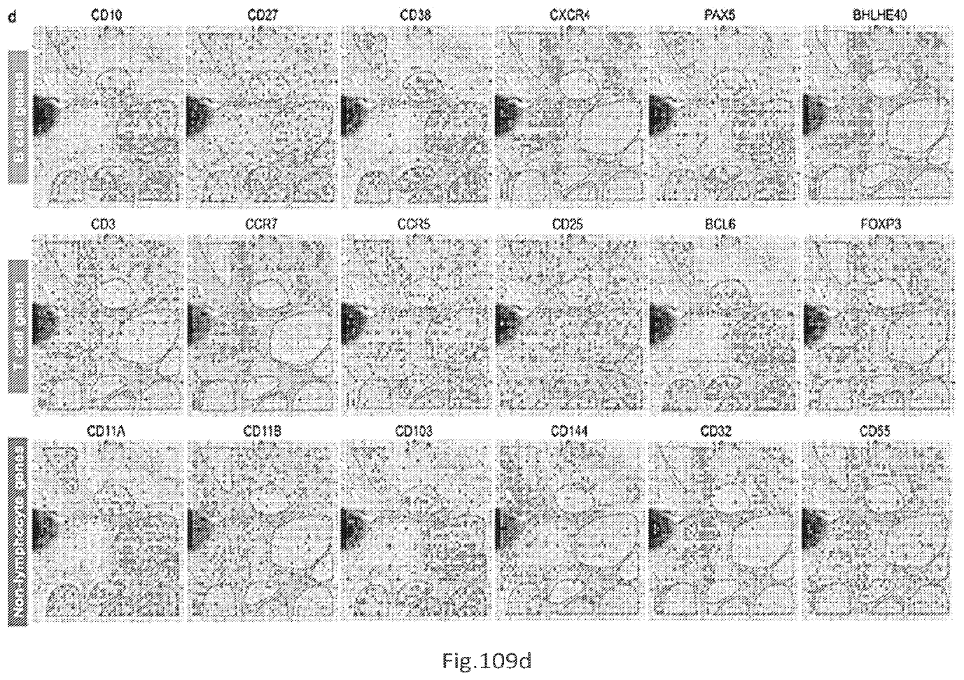

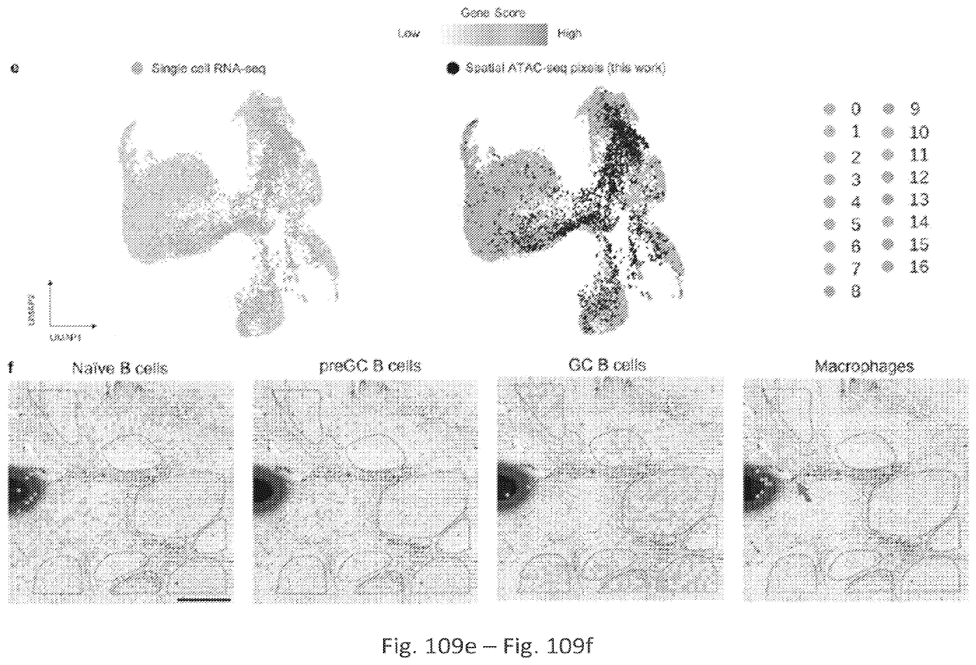

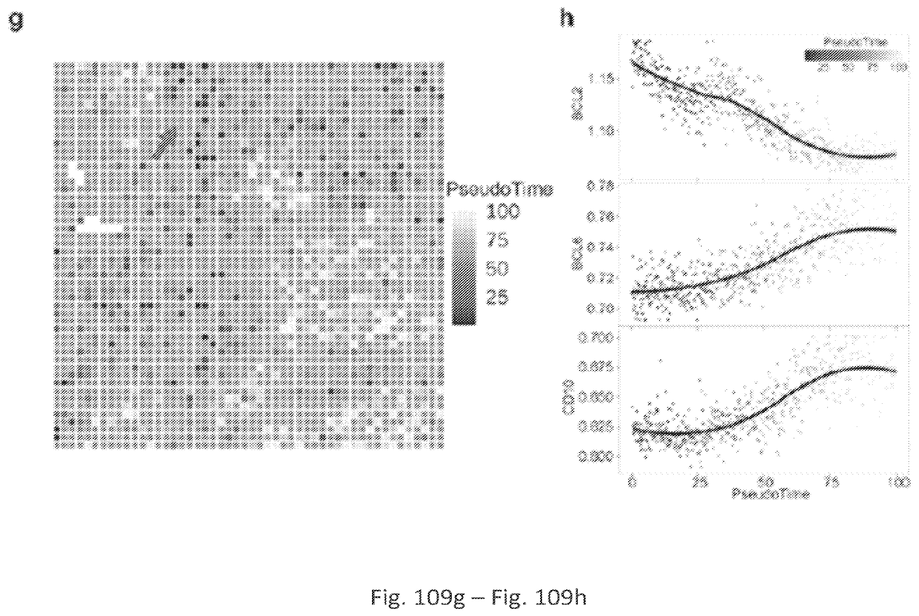

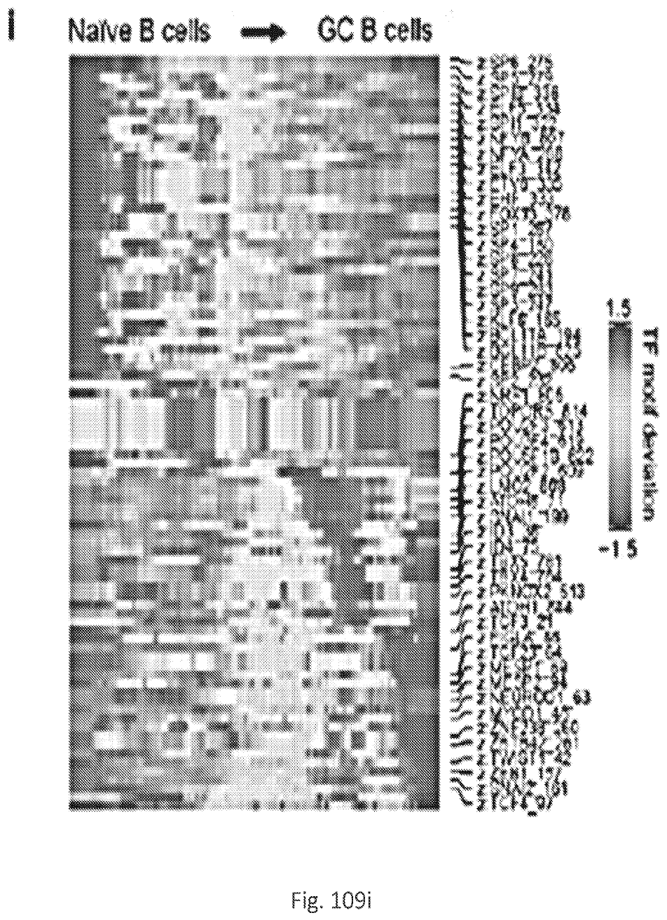

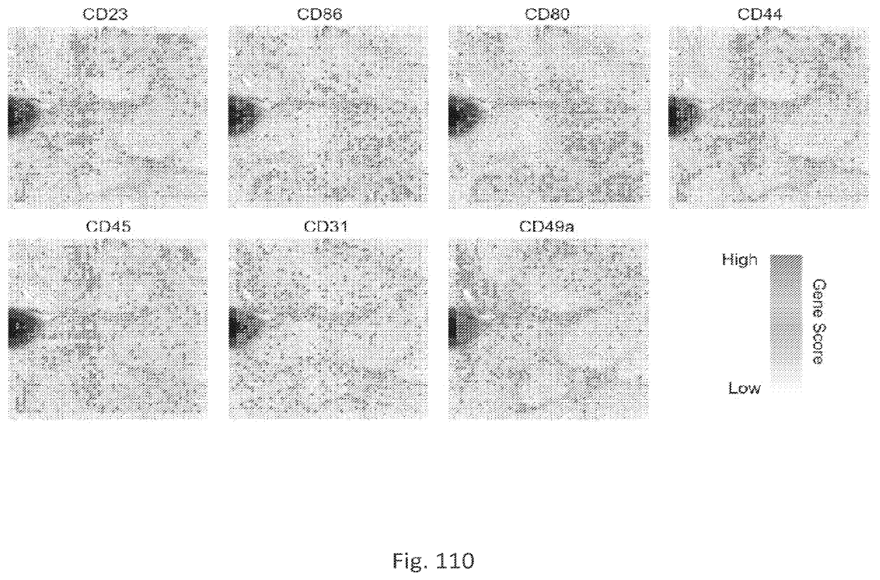

To assign cell types to each cluster, the spatial ATAC-seq data was integrated with the scRNA-seq atlas of the mouse embryos (Cao et al., 2019, Nature, 566:496-502), and several organ-specific cell types were identified (FIG. 104d-f, FIG. 108). The primitive erythroid cells, crucial for early embryonic erythroid development, were strongly enriched in the liver and AGM in agreement with the anatomical annotation. Radial glia, postmitotic premature neurons, and inhibitory neuron progenitors were found in the brain and neural tube. Compared to E13, higher proportion of radial glia were identified in E11 mouse embryo, suggesting their transient nature during CNS development (Li et al., 2008, Glia, 56:646-658). Abundant chondrocytes & osteoblasts were observed in the embryonic facial prominence, and the limb mesenchyme was highly enriched in the limb region. The spatially organized neuronal development trajectory from radial glia to excitatory neurons in E11 mouse embryo (FIG. 104g-j) was reconstructed and the changes in neuron development-related genes and TF deviations were identified across this developmental process, including Notch1 that is highly expressed in the radial glia and regulates neural stem cell number and function during development (Li et al., 2008, Glia, 56:646-658; Patten et al., 2006, The Journal of Neuroscience, 26:3102) (FIG. 104h).

To assess the temporal dynamics of chromatin accessibility more directly during development, dynamic peaks that exhibit a significant change in accessibility from E11 to E13 mouse embryo were identified within fetal liver and excitatory neurons. Significant differences were observed sin the chromatin accessibility of fetal liver and excitatory neurons between different developmental stages (FIG. 104k-p). In particular, chromatin accessibility profiles of fetal liver at E13 were enriched with Gata motif sequences (FIG. 104k, m), the TFs known to be important in the erythroid differentiation (Granja et al., 2021, Nature Genetics, 53:403-411). In addition, Egr1 motif was enriched in the excitatory neurons at E13, which has the functional implication during brain development, particularly for the specification of excitatory neurons (Yin et al., 2020, Computational and Structural Biotechnology Journal, 18:942-952).

### Spatial Chromatin Accessibility Mapping of Human Tonsil and Immune Cell States

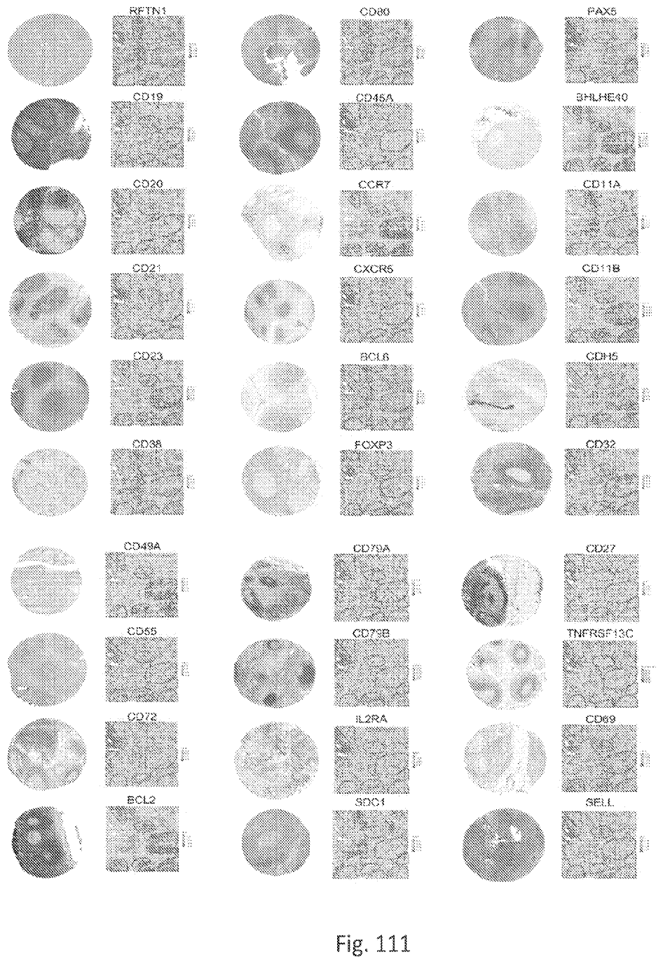

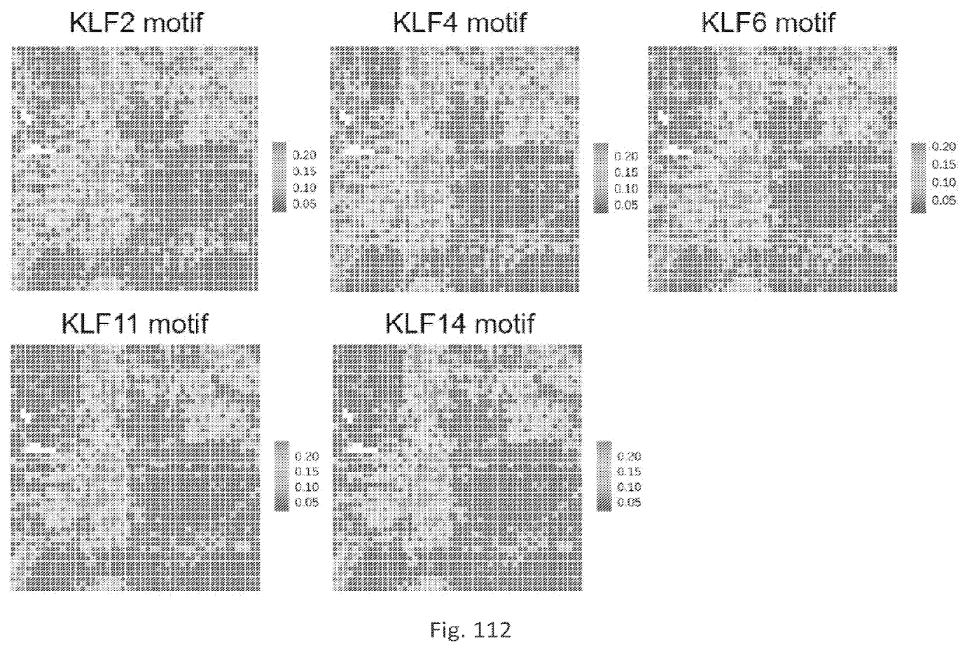

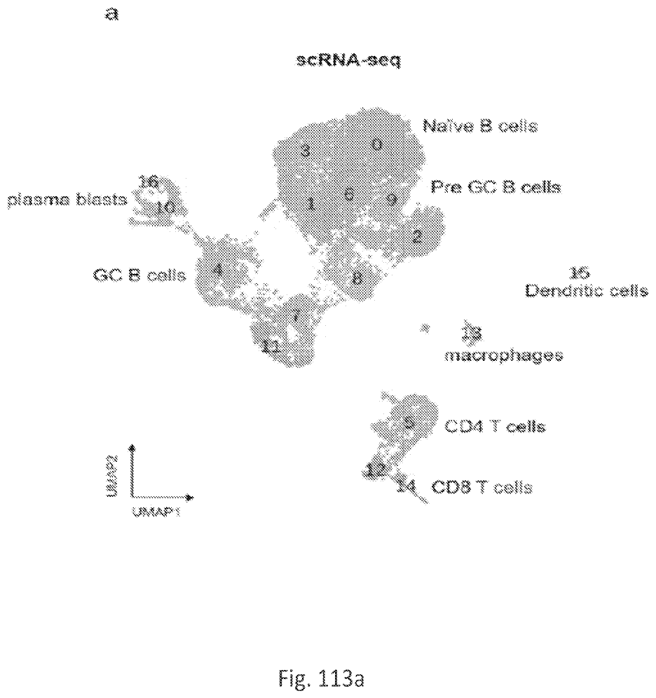

To demonstrate the ability to profile spatial chromatin accessibility in different tissue types and species, spatial-ATAC-seq with 20 μm pixel size was then applied to the human tonsil tissue. Unsupervised clustering revealed distinct spatial features with the germinal centers (GC) identified mainly in cluster 1 (FIG. 109a-c). Next the spatial patterns of specific marker genes were explored to distinguish cell types (FIG. 109d, FIG. 110) and compared to the distribution of protein expression in tonsil (FIG. 111). For B cell-related genes, the accessibility of CD10, a marker for mature GC B cells, was enriched in the GC regions. CD27, a marker for memory B cells, was active in GC and the extrafollicular regions. CD38, which marks activated B cells, was found to be enriched in GC. CXCR4, which is expressed in the centroblasts in the GC dark zone, unexpectedly showed high accessibility only in non-GC cells. This discordance between epigenetic state and protein expression may suggest epigenetic priming of pre-GC B cells prior to entering GC. It could also be due to the presence of CXCR4+ T cells supporting extra-follicular B-cell responses in the setting of inflammation³². PAX5, a transcription factor for follicular and memory B cells, was enriched in GC but also observed in the extrafollicular zones where the memory B cells migrated to. BHLHE40, a poorly understood transcription factor that can bind to the major regulatory regions of the IgH locus, was found to be enriched in the extrafollicular region but completely depleted in GC, suggesting the potential role in the regulation of class switch recombination in the pre-GC state. This supports a model of epigenetic control for class switch recombination that occurs before formation of the GC response. For T cell related genes, CD3 corresponded to T cell zones and also found active in GC. It is known that follicular helper T cells (T_{FH}) trafficking into GC requires downregulation of CCR7 and upregulation of CXCR5. Significantly reduced CCR7 accessibility was observed in GC while strong enrichment outside GC, indicating this T_{FH} function is indeed epigenetically regulated. CXCR5 accessibility was extensively detected in GC but also observed outside GC, indicating a possible early epigenetic priming of T_{FH} cells prior to GC entry for B cell help. The locus accessibility of BCL6, a THE master transcription factor, was strongly enriched in GC as expected. FOXP3, a master transcription factor for follicular regulatory T cells (T_{FR}), is mainly in the extrafollicular zone but at low frequency according to human protein atlas data (FIG. 111). Interestingly, it showed extensive open locus accessibility, suggesting extensive epigenetic priming of pre-GC T cells to potentially develop T_{FR} function as needed to balance GC activity. CD25, a surface marker for regulatory T cells, was active in both GC and the extrafollicular zone. For non-lymphoid cells, CD11B, a macrophage marker, was inactive in GC, on contrast to CD11A, which was more active in GC lymphocytes. CD103 was enriched in GC follicular dendritic cells. CD144, which encodes vascular endothelial cadherin (VE-cadherin), corresponded to endothelial microvasculature near the crypt or between follicles. CD32, a surface receptor involved in phagocytosis and clearing of immune complexes, and CD55, a complement decay-accelerating factor, were both active in the same region such that the cells not supposed to be cleared can be protected against phagocytosis by blocking the formation of the membrane attack complex. Cell type-specific TF regulators were examined within each cluster and the data revealed that KLF family transcription factors were highly enriched in non-germinal center cells, consistent with previous study (King et al., 2021, bioRxiv, 2021.2003.2016.435578) (FIG. 112).

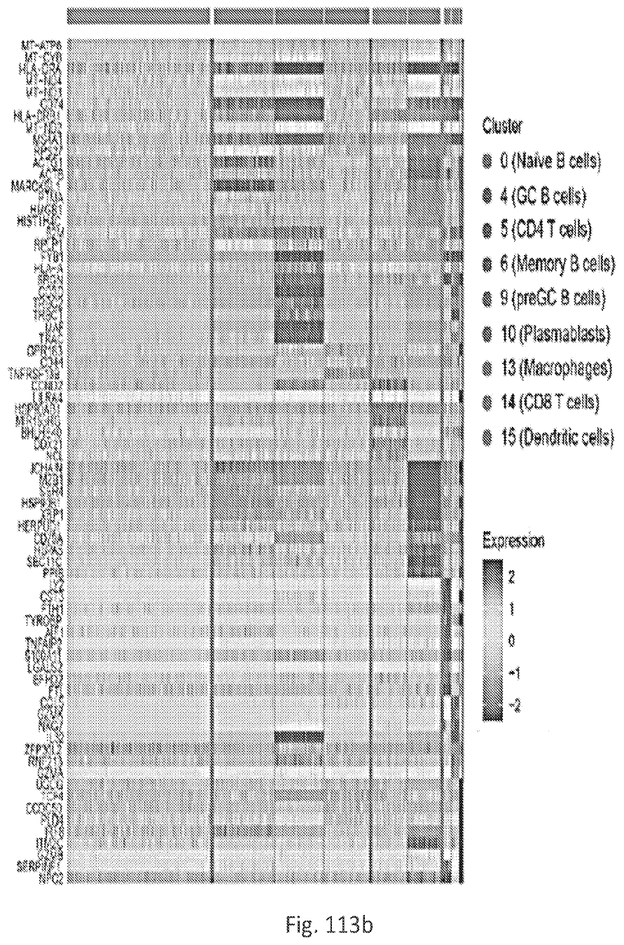

To map cell types onto each cluster, spatial-ATAC-seq data was integrated with the publicly available tonsillar scRNA-seq datasets (King et al., 2021, bioRxiv, 2021.2003.2016.435578). After unsupervised clustering for scRNA-seq data and label transfer to the spatial-ATAC-seq data, it was found that cells from cluster 0 were widely distributed in the non-GC region, while cells from cluster 4 were enriched in GC (FIG. 109e, f, FIG. 113a). A small region with cells enriched from cluster 13 was identified (FIG. 109f, FIG. 113a). To define the cell identities for scRNA-seq clusters, the marker genes for each cluster were examined and it was found that cluster 0 comprised of Naïve B cells, cluster 4 corresponded to GC B cells, and cluster 13 were macrophages (FIG. 113b), in agreement with the tissue histology (FIG. 109f).

Lymphocyte activation, maturation, and differentiation are regulated by the gene networks under the control of transcription factors (King et al., 2021, bioRxiv, 2021.2003.2016.435578). To understand the dynamic regulation process, a pseudotemporal reconstruction of B cell activation to the GC reaction (FIG. 109g-i) was implemented. Meanwhile, the projection of each pixel's pseudo-time value onto spatial coordinates revealed spatially distinct regions in this dynamic process. Interestingly, it was found that the enriched macrophage population was co-localized with inactivated B cell, consistent with the fact that B cells are activated through acquiring antigen from the antigen presenting macrophages before GC entry or formation (Kleshchevnikov et al., 2020, bioRxiv, 2020.2011.2015.378125) (FIG. 109g). In addition, pseudotemporal ordering of B cell activation revealed dynamic expression and chromatin activity before commitment to the GC state (FIG. 109h, i), including an early activity of BCL2 and reduced accessibility within GC B cells as compared to naïve populations, suggesting that this anti-apoptotic molecule may be actively repressed to ensure that GC B cells are eliminated by apoptosis if they are not selected and rescued by survival signals. In contrast, LMO2 exhibited increased accessibility at the target sites within GC B cells, which agreed with the previous finding that LMO2 is specifically upregulated in the GC (Cubedo et al., 2012, Blood, 119:5478-5491).

### Example 7: SEQUENCES

TABLE 3
Barcode Sequences

| SEQ ID | | |
|---|---|---|
| NO: | Sequence Name | Sequence |
| 1 | Tn5ME-A_BC1_1 | /5Phos/AGGCCAGAGCATTCGAACGTGATAGATGTGTATAAGAGACAG |
| 2 | Tn5ME-A_BC1_2 | /5Phos/AGGCCAGAGCATTCGAAACATCGAGATGTGTATAAGAGACAG |
| 3 | Tn5ME-A_BC1_3 | /5Phos/AGGCCAGAGCATTCGATGCCTAAAGATGTGTATAAGAGACAG |
| 4 | Tn5ME-A_BC1_4 | /5Phos/AGGCCAGAGCATTCGAGTGGTCAAGATGTGTATAAGAGACAG |
| 5 | Tn5ME-A_BC1_5 | /5Phos/AGGCCAGAGCATTCGACCACTGTAGATGTGTATAAGAGACAG |
| 6 | Tn5ME-A_BC1_6 | /5Phos/AGGCCAGAGCATTCGACATTGGCAGATGTGTATAAGAGACAG |
| 7 | Tn5ME-A_BC1_7 | /5Phos/AGGCCAGAGCATTCGCAGATCTGAGATGTGTATAAGAGACAG |
| 8 | Tn5ME-A_BC1_8 | /5Phos/AGGCCAGAGCATTCGCATCAAGTAGATGTGTATAAGAGACAG |
| 9 | Tn5ME-A_BC1_9 | /5Phos/AGGCCAGAGCATTCGCGCTGATCAGATGTGTATAAGAGACAG |
| 10 | Tn5ME-A_BC1_10 | /5Phos/AGGCCAGAGCATTCGACAAGCTAAGATGTGTATAAGAGACAG |
| 11 | Tn5ME-A_BC1_11 | /5Phos/AGGCCAGAGCATTCGCTGTAGCCAGATGTGTATAAGAGACAG |
| 12 | Tn5ME-A_BC1_12 | /5Phos/AGGCCAGAGCATTCGAGTACAAGAGATGTGTATAAGAGACAG |
| 13 | Tn5ME-A_BC1_13 | /5Phos/AGGCCAGAGCATTCGAACAACCAAGATGTGTATAAGAGACAG |
| 14 | Tn5ME-A_BC1_14 | /5Phos/AGGCCAGAGCATTCGAACCGAGAAGATGTGTATAAGAGACAG |
| 15 | Tn5ME-A_BC1_15 | /5Phos/AGGCCAGAGCATTCGAACGCTTAAGATGTGTATAAGAGACAG |
| 16 | Tn5ME-A_BC1_16 | /5Phos/AGGCCAGAGCATTCGAAGACGGAAGATGTGTATAAGAGACAG |
| 17 | Tn5ME-A_BC1_17 | /5Phos/AGGCCAGAGCATTCGAAGGTACAAGATGTGTATAAGAGACAG |
| 18 | Tn5ME-A_BC1_18 | /5Phos/AGGCCAGAGCATTCGACACAGAAAGATGTGTATAAGAGACAG |
| 19 | Tn5ME-A_BC1_19 | /5Phos/AGGCCAGAGCATTCGACAGCAGAAGATGTGTATAAGAGACAG |
| 20 | Tn5ME-A_BC1_20 | /5Phos/AGGCCAGAGCATTCGACCTCCAAAGATGTGTATAAGAGACAG |
| 21 | Tn5ME-A_BC1_21 | /5Phos/AGGCCAGAGCATTCGACGCTCGAAGATGTGTATAAGAGACAG |
| 22 | Tn5ME-A_BC1_22 | /5Phos/AGGCCAGAGCATTCGACGTATCAAGATGTGTATAAGAGACAG |
| 23 | Tn5ME-A_BC1_23 | /5Phos/AGGCCAGAGCATTCGACTATGCAAGATGTGTATAAGAGACAG |
| 24 | Tn5ME-A_BC1_24 | /5Phos/AGGCCAGAGCATTCGAGAGTCAAAGATGTGTATAAGAGACAG |
| 25 | Tn5ME-A_BC1_25 | /5Phos/AGGCCAGAGCATTCGAGATCGCAAGATGTGTATAAGAGACAG |
| 26 | Tn5ME-A_BC1_26 | /5Phos/AGGCCAGAGCATTCGAGCAGGAAAGATGTGTATAAGAGACAG |
| 27 | Tn5ME-A_BC1_27 | /5Phos/AGGCCAGAGCATTCGAGTCACTAAGATGTGTATAAGAGACAG |
| 28 | Tn5ME-A_BC1_28 | /5Phos/AGGCCAGAGCATTCGATCCTGTAAGATGTGTATAAGAGACAG |
| 29 | Tn5ME-A_BC1_29 | /5Phos/AGGCCAGAGCATTCGATTGAGGAAGATGTGTATAAGAGACAG |
| 30 | Tn5ME-A_BC1_30 | /5Phos/AGGCCAGAGCATTCGCAACCACAAGATGTGTATAAGAGACAG |
| 31 | Tn5ME-A_BC1_31 | /5Phos/AGGCCAGAGCATTCGGACTAGTAAGATGTGTATAAGAGACAG |
| 32 | Tn5ME-A_BC1_32 | /5Phos/AGGCCAGAGCATTCGCAATGGAAAGATGTGTATAAGAGACAG |
| 33 | Tn5ME-A_BC1_33 | /5Phos/AGGCCAGAGCATTCGCACTTCGAAGATGTGTATAAGAGACAG |
| 34 | Tn5ME-A_BC1_34 | /5Phos/AGGCCAGAGCATTCGCAGCGTTAAGATGTGTATAAGAGACAG |
| 35 | Tn5ME-A_BC1_35 | /5Phos/AGGCCAGAGCATTCGCATACCAAAGATGTGTATAAGAGACAG |
| 36 | Tn5ME-A_BC1_36 | /5Phos/AGGCCAGAGCATTCGCCAGTTCAAGATGTGTATAAGAGACAG |
| 37 | Tn5ME-A_BC1_37 | /5Phos/AGGCCAGAGCATTCGCCGAAGTAAGATGTGTATAAGAGACAG |
| 38 | Tn5ME-A_BC1_38 | /5Phos/AGGCCAGAGCATTCGCCGTGAGAAGATGTGTATAAGAGACAG |
| 39 | Tn5ME-A_BC1_39 | /5Phos/AGGCCAGAGCATTCGCCTCCTGAAGATGTGTATAAGAGACAG |
| 40 | Tn5ME-A_BC1_40 | /5Phos/AGGCCAGAGCATTCGCGAACTTAAGATGTGTATAAGAGACAG |
| 41 | Tn5ME-A_BC1_41 | /5Phos/AGGCCAGAGCATTCGCGACTGGAAGATGTGTATAAGAGACAG |
| 42 | Tn5ME-A_BC1_42 | /5Phos/AGGCCAGAGCATTCGCGCATACAAGATGTGTATAAGAGACAG |
| 43 | Tn5ME-A_BC1_43 | /5Phos/AGGCCAGAGCATTCGCTCAATGAAGATGTGTATAAGAGACAG |
| 44 | Tn5ME-A_BC1_44 | /5Phos/AGGCCAGAGCATTCGCTGAGCCAAGATGTGTATAAGAGACAG |
| 45 | Tn5ME-A_BC1_45 | /5Phos/AGGCCAGAGCATTCGCTGGCATAAGATGTGTATAAGAGACAG |
| 46 | Tn5ME-A_BC1_46 | /5Phos/AGGCCAGAGCATTCGGAATCTGAAGATGTGTATAAGAGACAG |
| 47 | Tn5ME-A_BC1_47 | /5Phos/AGGCCAGAGCATTCGCAAGACTAAGATGTGTATAAGAGACAG |
| 48 | Tn5ME-A_BC1_48 | /5Phos/AGGCCAGAGCATTCGGAGCTGAAAGATGTGTATAAGAGACAG |
| 49 | Tn5ME-A_BC1_49 | /5Phos/AGGCCAGAGCATTCGGATAGACAAGATGTGTATAAGAGACAG |
| 50 | Tn5ME-A_BC1_50 | /5Phos/AGGCCAGAGCATTCGGCCACATAAGATGTGTATAAGAGACAG |
| 51 | BC2_R1_1 | GCCTGTCCGCGGAAGCAGTGGTATCAACGCAGAGTACAACGTGATATCCACGTGCTT |
| | | GAG |
| 52 | BC2_R1_2 | GCCTGTCCGCGGAAGCAGTGGTATCAACGCAGAGTACAAACATCGATCCACGTGCTT |
| | | GAG |
| 53 | BC2_R1_3 | GCCTGTCCGCGGAAGCAGTGGTATCAACGCAGAGTACATGCCTAAATCCACGTGCTTG |
| | | AG |
| 54 | BC2_R1_4 | GCCTGTCCGCGGAAGCAGTGGTATCAACGCAGAGTACAGTGGTCAATCCACGTGCTT |
| | | GAG |
| 55 | BC2_R1_5 | GCCTGTCCGCGGAAGCAGTGGTATCAACGCAGAGTACACCACTGTATCCACGTGCTTG |
| | | AG |
| 56 | BC2_R1_6 | GCCTGTCCGCGGAAGCAGTGGTATCAACGCAGAGTACACATTGGCATCCACGTGCTT |
| | | GAG |
| 57 | BC2_R1_7 | GCCTGTCCGCGGAAGCAGTGGTATCAACGCAGAGTACCAGATCTGATCCACGTGCTT |
| | | GAG |
| 58 | BC2_R1_8 | GCCTGTCCGCGGAAGCAGTGGTATCAACGCAGAGTACCATCAAGTATCCACGTGCTTG |
| | | AG |
| 59 | BC2_R1_9 | GCCTGTCCGCGGAAGCAGTGGTATCAACGCAGAGTACCGCTGATCATCCACGTGCTTGI |
| | | AG |
| 60 | BC2_R1_10 | GCCTGTCCGCGGAAGCAGTGGTATCAACGCAGAGTACACAAGCTAATCCACGTGCTT |
| | | GAG |
| 61 | BC2_R1_11 | GCCTGTCCGCGGAAGCAGTGGTATCAACGCAGAGTACCTGTAGCCATCCACGTGCTTG |
| | | AG |
| 62 | BC2_R1_12 | GCCTGTCCGCGGAAGCAGTGGTATCAACGCAGAGTACAGTACAAGATCCACGTGCTT |
| | | GAG |
| 63 | BC2_R1_13 | GCCTGTCCGCGGAAGCAGTGGTATCAACGCAGAGTACAACAACCAATCCACGTGCTT |
| | | GAG |
| 64 | BC2_R1_14 | GCCTGTCCGCGGAAGCAGTGGTATCAACGCAGAGTACAACCGAGAATCCACGTGCTT |
| | | GAG |
| 65 | BC2_R1_15 | GCCTGTCCGCGGAAGCAGTGGTATCAACGCAGAGTACAACGCTTAATCCACGTGCTTG |
| | | AG |
| 66 | BC2_R1_16 | GCCTGTCCGCGGAAGCAGTGGTATCAACGCAGAGTACAAGACGGAATCCACGTGCTT |
| | | GAG |
| 67 | BC2_R1_17 | GCCTGTCCGCGGAAGCAGTGGTATCAACGCAGAGTACAAGGTACAATCCACGTGCTT |
| | | GAG |
| 68 | BC2_R1_18 | GCCTGTCCGCGGAAGCAGTGGTATCAACGCAGAGTACACACAGAAATCCACGTGCTT |
| | | GAG |
| 69 | BC2_R1_19 | GCCTGTCCGCGGAAGCAGTGGTATCAACGCAGAGTACACAGCAGAATCCACGTGCTT |
| | | GAG |
| 70 | BC2_R1_20 | GCCTGTCCGCGGAAGCAGTGGTATCAACGCAGAGTACACCTCCAAATCCACGTGCTTG |
| | | AG |
| 71 | BC2_R1_21 | GCCTGTCCGCGGAAGCAGTGGTATCAACGCAGAGTACACGCTCGAATCCACGTGCTT |
| | | GAG |
| 72 | BC2_R1_22 | GCCTGTCCGCGGAAGCAGTGGTATCAACGCAGAGTACACGTATCAATCCACGTGCTTG |
| | | AG |
| 73 | BC2_R1_23 | GCCTGTCCGCGGAAGCAGTGGTATCAACGCAGAGTACACTATGCAATCCACGTGCTTG! |
| | | AG |
| 74 | BC2_R1_24 | GCCTGTCCGCGGAAGCAGTGGTATCAACGCAGAGTACAGAGTCAAATCCACGTGCTT |
| | | GAG |
| 75 | BC2_R1_25 | GCCTGTCCGCGGAAGCAGTGGTATCAACGCAGAGTACAGATCGCAATCCACGTGCTT |
| | | GAG |
| 76 | BC2_R1_26 | GCCTGTCCGCGGAAGCAGTGGTATCAACGCAGAGTACAGCAGGAAATCCACGTGCTT |
| | | GAG |
| 77 | BC2_R1_27 | GCCTGTCCGCGGAAGCAGTGGTATCAACGCAGAGTACAGTCACTAATCCACGTGCTTG |
| | | AG |
| 78 | BC2_R1_28 | GCCTGTCCGCGGAAGCAGTGGTATCAACGCAGAGTACATCCTGTAATCCACGTGCTTG |
| | | AG |
| 79 | BC2_R1_29 | GCCTGTCCGCGGAAGCAGTGGTATCAACGCAGAGTACATTGAGGAATCCACGTGCTT |
| | | GAG |
| 80 | BC2_R1_30 | GCCTGTCCGCGGAAGCAGTGGTATCAACGCAGAGTACCAACCACAATCCACGTGCTT |
| | | GAG |
| 81 | BC2_R1_31 | GCCTGTCCGCGGAAGCAGTGGTATCAACGCAGAGTACGACTAGTAATCCACGTGCTT |
| | | GAG |
| 82 | BC2_R1_32 | GCCTGTCCGCGGAAGCAGTGGTATCAACGCAGAGTACCAATGGAAATCCACGTGCTT |
| | | GAG |
| 83 | BC2_R1_33 | GCCTGTCCGCGGAAGCAGTGGTATCAACGCAGAGTACCACTTCGAATCCACGTGCTTG |
| | | AG |
| 84 | BC2_R1_34 | GCCTGTCCGCGGAAGCAGTGGTATCAACGCAGAGTACCAGCGTTAATCCACGTGCTT |
| | | GAG |
| 85 | BC2_R1_35 | GCCTGTCCGCGGAAGCAGTGGTATCAACGCAGAGTACCATACCAAATCCACGTGCTTG |
| | | AG |
| 86 | BC2_R1_36 | GCCTGTCCGCGGAAGCAGTGGTATCAACGCAGAGTACCCAGTTCAATCCACGTGCTTG |
| | | AG |
| 87 | BC2_R1_37 | GCCTGTCCGCGGAAGCAGTGGTATCAACGCAGAGTACCCGAAGTAATCCACGTGCTT |
| | | GAG |
| 88 | BC2_R1_38 | GCCTGTCCGCGGAAGCAGTGGTATCAACGCAGAGTACCCGTGAGAATCCACGTGCTT |
| | | GAG |
| 89 | BC2_R1_39 | GCCTGTCCGCGGAAGCAGTGGTATCAACGCAGAGTACCCTCCTGAATCCACGTGCTTG |
| | | AG |
| 90 | BC2_R1_40 | GCCTGTCCGCGGAAGCAGTGGTATCAACGCAGAGTACCGAACTTAATCCACGTGCTTG |
| | | AG |
| 91 | BC2_R1_41 | GCCTGTCCGCGGAAGCAGTGGTATCAACGCAGAGTACCGACTGGAATCCACGTGCTT |
| | | GAG |
| 92 | BC2_R1_42 | GCCTGTCCGCGGAAGCAGTGGTATCAACGCAGAGTACCGCATACAATCCACGTGCTT |
| | | GAG |
| 93 | BC2_R1_43 | GCCTGTCCGCGGAAGCAGTGGTATCAACGCAGAGTACCTCAATGAATCCACGTGCTTG |
| | | AG |
| 94 | BC2_R1_44 | GCCTGTCCGCGGAAGCAGTGGTATCAACGCAGAGTACCTGAGCCAATCCACGTGCTT |
| | | GAG |
| 95 | BC2_R1_45 | GCCTGTCCGCGGAAGCAGTGGTATCAACGCAGAGTACCTGGCATAATCCACGTGCTT |
| | | GAG |
| 96 | BC2_R1_46 | GCCTGTCCGCGGAAGCAGTGGTATCAACGCAGAGTACGAATCTGAATCCACGTGCTT |
| | | GAG |
| 97 | BC2_R1_47 | GCCTGTCCGCGGAAGCAGTGGTATCAACGCAGAGTACCAAGACTAATCCACGTGCTT |
| | | GAG |
| 98 | BC2_R1_48 | GCCTGTCCGCGGAAGCAGTGGTATCAACGCAGAGTACGAGCTGAAATCCACGTGCTT |
| | | GAG |
| 99 | BC2_R1_49 | GCCTGTCCGCGGAAGCAGTGGTATCAACGCAGAGTACGATAGACAATCCACGTGCTT |
| | | GAG |
| 100 | BC2_R1_50 | GCCTGTCCGCGGAAGCAGTGGTATCAACGCAGAGTACGCCACATAATCCACGTGCTT |
| | | GAG |
| 101 | Tn5MErev | /5Phos/CTGTCTCTTATACACATCT |
| 102 | Tn5ME-B | GTCTCGTGGGCTCGGAGATGTGTATAAGAGACAG |
| 103 | Ligation linker 1 | AGTCGTACGCCGATGCTGTCTCTTATACACATCT |
| 104 | Ligation linker 2 | CGAATGCTCTGGCCTCTCAAGCACGTGGAT |
| 105 | New-P5-SMART PCR | AATGATACGGCGACCACCGAGATCTACACGCCTGTCCGCGGAAGCAGTGGTATCAAC |
| | hybrid oligo | GCAGAGT*A*C |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method, comprising:
(a) delivering to a region of interest in a tissue sample mounted on a substrate a transposase and a linker adaptor sequence;
(b) delivering to the region of interest a first set of barcoded polynucleotides, wherein the barcoded polynucleotides comprise a first region for ligation to the linker adaptor sequence, a second unique region for spatial barcoding and a third linker region for ligation to a region of the second barcode or a universal ligation linker, wherein the first set of barcoded polynucleotides is delivered through a first microfluidic device clamped to the region of interest;
(c) delivering to the region of interest ligation reagents to join the ligation adaptor to the barcoded polynucleotides of the first set;
(d) delivering to the region of interest a second set of barcoded polynucleotides, wherein the barcoded polynucleotides comprise a first region for ligation to the linker region of the first barcode or a universal ligation linker, a second unique region for spatial barcoding and a third ligation region comprising a sequence for recognition by a primer for DNA amplification, wherein the second set of barcoded polynucleotides is delivered through a second microfluidic device clamped to the region of interest, wherein the second microfluidic device is oriented on the region of interest perpendicular to the direction of the microchannels of the first microfluidic device;
(e) delivering to the region of interest ligation reagents to join barcoded polynucleotides of the first set to barcoded polynucleotides of the second set;
(f) imaging the region of interest to produce a sample image;
(g) delivering to the region of interest lysis buffer or denaturation reagents to produce a lysed or denatured tissue sample; and
(h) extracting the DNA from the lysed or denatured tissue sample.

2. The method of claim 1, further comprising a step of permeabilizing the tissue sample prior to delivering the transposase and linker adaptor sequence.

3. The method of claim 1, wherein step (a) comprises delivering to the region of interest in a tissue sample mounted on a substrate (i) a primary antibody specific for binding to an epigenomic marker of interest (ii) a secondary antibody and (iii) a transposase and a linker adaptor sequence.

4. The method of claim 3, wherein the primary antibody is selected from whole antibodies, Fab antibody fragments, F(ab′)₂ antibody fragments, monospecific Fab₂ fragments, bispecific Fab₂ fragments, trispecific Fab₃ fragments, single chain variable fragments (scFvs), bispecific diabodies, trispecific diabodies, scFv-Fc molecules, nanobodies, and minibodies.

5. The method of claim 3, wherein the epigenomic marker is selected from the group consisting of H2AK5ac, H2AK9ac, H2BK120ac, H2BK12ac, H2BK15ac, H2BK20ac, H2BK5ac, H2Bub, H3, H3ac, H3K14ac, H3K18ac, H3K23ac, H3K23me2, H3K27me1, H3K27me2, H3K36ac, H3K36me1, H3K36me2, H3K4ac, H3K56ac, H3K79me1, H3K79me3, H3K9acS10ph, H3K9me2, H3S10ph, H3T11ph, H4, H4ac, H4K12ac, H4K16ac, H4K5ac, H4K8ac, H4K91ac, H3F3A, H3K27me3, H3K36me3, H3K4me1, H3K79me2, H3K9me1, H3K9me2, H3K9me3, H4K20me1, H2AFZ, H3K27ac, H3K4me2, H3K4me3, and H3K9ac.

6. The method of any one of the preceding claims, wherein the method further comprises delivering to the biological sample a ligation linker sequence, wherein the ligation linker is selected from the group consisting of:
a) a nucleic acid molecule comprising a sequence complementary to the ligation linker sequence of the ligation adaptor associated with the transposon and a sequence complementary to the ligation linker sequence of the barcoded polynucleotides of the first set; and
b) a nucleic acid molecule comprising a sequence complementary to the ligation linker sequence of the barcoded polynucleotides of the first set and a sequence complementary to the ligation linker sequence of the barcoded polynucleotides of the second set.

7. The method of any one of the preceding claims further comprising step (i) sequencing the DNA to produce DNA reads.

8. The method of claim 7 further comprising constructing a spatial map of the tissue section by matching the spatially addressable barcoded conjugates to corresponding sequencing reads.

9. The method of claim 8 further comprising identifying the anatomical location of the nucleic acids by correlating the spatial map to the sample image.

10. The method of any one of the preceding claims, wherein the tissue section mounted on a slide is produced by:
sectioning a formalin fixed paraffin embedded (FFPE) tissue, optionally into a 5-10 μm section and mounting the tissue section onto a substrate, optionally a poly-L-lysine-coated slide;
applying to the tissue section a wash solution, optionally a xylene solution, to deparaffinize the tissue section;
applying to the tissue section a rehydration solution to rehydrate the tissue section;
applying to the tissue section an enzymatic solution to permeabilize the tissue section; and
applying formalin to the tissue section to post-fix the tissue section.

11. The method of any one of the preceding claims, wherein the first and/or second microfluidic device is fabricated from polydimethylsiloxane (PDMS).

12. The method of any one of the preceding claims, wherein the first and/or second microfluidic device comprises 10 to 1000 microchannels.

13. The method of any one of the preceding claims, wherein the first and/or second microfluidic device comprises serpentine microchannels.

14. The method of claim 13, further comprising delivering to the region of interest a third set of barcoded polynucleotides, wherein the third set of barcoded polynucleotides is delivered to specific zones, such that each zone distinguishes a specific region of overlap of the first and second barcode sequences; wherein the third set of barcoded polynucleotides are delivered directly to the tissue section, optionally through a set of holes in a device clamped to the substrate, wherein each hole is positioned directly above a zone of overlap of the first and second barcode sequences.

15. The method of any one of the preceding claims, wherein delivery of the first set of barcoded polynucleotides is delivered through the first microfluidic device using a negative pressure system and/or delivery of the second set of barcoded polynucleotides is delivered through the second microfluidic device using a negative pressure system.

16. The method of any one of the preceding claims, wherein the lysis buffer or denaturation reagents are delivered directly to the tissue section, optionally through a hole in a device clamped to the substrate, wherein the hole is positioned directly above the region of interest.

17. The method of any one of the preceding claims, wherein the first and/or second set of barcoded polynucleotides comprises at least 10 barcoded polynucleotides.

18. The method of any one of the preceding claims, wherein the imaging is with an optical or fluorescence microscope.

19. The method of any one of the preceding claims, wherein the substrate is selected from the group consisting of a glass slide and a plastic slide.
